(19) United States
(12) Patent Application Publication
(10) Pub. No.: US 2019/0048312 A1
(43) Pub. Date: Feb. 14, 2019

(54) Dry Powder Cell Culture Products and Methods of Production Thereof

(71) Applicants: Christine M. Biddle, Northbrook, IL (US); Jeffrey W. Biddle, Buffalo, NY (US); Laurel Biddle, Buffalo, NY (US); LIFE TECHNOLOGIES CORPORATION, Carlsbad, CA (US)

(72) Inventors: Richard Fike, Clarence, NY (US); William Whitford, Logan, UT (US); Richard Hassett, Tonawanda, NY (US); Robert Radominski, Tonawanda, NY (US); Barbara Dadey, East Aurora, NY (US)

(21) Appl. No.: 16/035,344

(22) Filed: Jul. 13, 2018

Related U.S. Application Data
(63) Continuation of application No. 11/669,827, filed on Jan. 31, 2007, now abandoned.
Application No. 11/669,827 is a continuation-in-part of application No. 09/023,790, filed on Feb. 13, 1998, now Pat. No. 6,383,810.
Application No. 11/669,827 is a continuation-in-part of application No. 11/434,513, filed on May 16, 2006, now abandoned.
Application No. 11/434,513 is a continuation of application No. 10/307,451, filed on Dec. 2, 2002, now abandoned.
Application No. 11/669,827 is a continuation-in-part of application No. 11/502,546, filed on Aug. 11, 2006, now abandoned.
Application No. 11/502,546 is a division of application No. 09/705,940, filed on Nov. 6, 2000, now abandoned.
Application No. 11/669,827 is a continuation-in-part of application No. 10/617,377, filed on Jul. 11, 2003, now abandoned.
Application No. 10/617,377 is a continuation of application No. 09/576,900, filed on May 23, 2000, now Pat. No. 6,627,426.
Application No. 09/576,900 is a continuation of application No. 09/343,686, filed on Jun. 30, 1999, now abandoned.
(60) Provisional application No. 60/040,314, filed on Feb. 14, 1997.
(60) Provisional application No. 60/058,716, filed on Sep. 12, 1997.
(60) Provisional application No. 60/062,192, filed on Oct. 16, 1997.
(60) Provisional application No. 60/337,117, filed on Dec. 7, 2001.
(60) Provisional application No. 60/334,115, filed on Nov. 30, 2001.
(60) Provisional application No. 60/091,275, filed on Jun. 30, 1998.
(60) Provisional application No. 60/863,917, filed on Nov. 1, 2006.

(51) Int. Cl. C12N 5/00; C12N 1/00

(52) U.S. Cl. CPC C12N 5/0018; C12N 2500/60; C12N 2500/36; C12N 1/00

(57) ABSTRACT

The present invention relates to nutritive medium, medium supplement, media subgroup and buffer formulations. The present invention provides powder nutritive medium, medium supplement and medium subgroup formulations, e.g., cell culture medium supplements (including powdered sera such as powdered fetal bovine serum (FBS)), medium subgroup formulations and cell culture media comprising all of the necessary nutritive factors that facilitate the in vitro cultivation of cells. The invention further provides powder buffer formulations that produce particular ionic and pH conditions upon reconstitution with a solvent. The invention provides methods for production of media, media supplement, media subgroup and buffer formulations, and also provides kits and methods for cultivation of prokaryotic and eukaryotic cells, particularly bacterial cells, yeast cells, plant cells and animal cells (including human cells) using these dry powder nutritive media, media supplement, media subgroup and buffer formulations.

### CROSS REFERENCE TO RELATED APPLICATIONS

This application is a continuation of U.S. patent application Ser. No. 11/669,827, filed Jan. 31, 2007, which is a continuation-in-part of U.S. patent application Ser. No. 09/023,790, filed Feb. 13, 1998, now U.S. Pat. No. 6,383,810, which claims the benefit of U.S. Provisional Application No. 60/040,314, filed Feb. 14, 1997, U.S. Provisional Application No. 60/058,716, filed Sep. 12, 1997, and U.S. Provisional Application No. 60/062,192, filed Oct. 16, 1997, the disclosures of which are incorporated herein by reference in their entireties.

This application is also a continuation of U.S. patent application Ser. No. 11/669,827, filed Jan. 31, 2007, which is a continuation-in-part of U.S. patent application Ser. No. 11/434,513, filed on May 16, 2006, now abandoned, which is a continuation of application Ser. No. 10/307,451, filed on Dec. 2, 2002, now abandoned, which claims the benefit of U.S. Provisional application No. 60/337,117, filed on Dec. 7, 2001, and U.S. Provisional application No. 60/334,115, filed on Nov. 30, 2001, the disclosures of which are incorporated herein by reference in their entireties.

This application is also a continuation of U.S. patent application Ser. No. 11/669,827, filed Jan. 31, 2007, which is a continuation-in-part of U.S. application Ser. No. 11/502,546, filed on Aug. 11, 2006 now abandoned, which is a division of application Ser. No. 09/705,940, filed on Nov. 6, 2000, now abandoned, the disclosures of which are incorporated herein by reference in their entireties.

This application is also a continuation of U.S. patent application Ser. No. 11/669,827, filed Jan. 31, 2007, which is a continuation-in-part of U.S. application No. of Ser. No. 10/617,377, filed Jul. 11, 2003, now abandoned, which is a continuation of Ser. No. 09/576,900, filed May 23, 2000, now U.S. Pat. No. 6,627,426, which is a continuation of U.S. patent application Ser. No. 09/343,686, filed Jun. 30, 1999, now abandoned, which claims the benefit of U.S. Provisional Application No. 60/091,275, filed Jun. 30, 1998, the disclosures of which are all incorporated herein by reference in their entireties.

This application is a continuation of U.S. patent application Ser. No. 11/669,827, filed Jan. 31, 2007, which is a continuation-in-part of, and claims the benefit of U.S. Provisional Application No. 60/863,917, filed Nov. 1, 2006, the disclosure of which is incorporated herein by reference in its entirety.

### BACKGROUND OF THE INVENTION

### Field of the Invention

The present invention relates generally to cells, nutritive media, media supplements, media subgroups and buffer formulations. One aspect of the present invention provides dry powder nutritive medium formulations. Another aspect of the invention provides cell culture medium formulations comprising all of the necessary nutritive factors that facilitate the in vitro cultivation of cells. Some embodiments of the invention provide methods and means of producing these media formulations. Some embodiments of the invention provide methods and means of supplementing these or other media formulations. The invention also relates to methods of producing dry powder media supplements, such as dry powder sera (e.g., fetal bovine serum), dry powder nutrient supplements, concentrated supplements and methods of making and using same. The present invention also relates to methods of incorporating lipids and/or other components poorly soluble in inorganic or polar solvents such as water. The invention also relates to methods of producing dry powder media supplements, such as dry powder sera (e.g., fetal bovine serum) and optionally with supplemental ingredients such as lipids or other ingredients useful for supporting cell culture. The invention also relates to dry powder media, dry powder media supplement, dry powder media subgroup and dry powder buffer formulations that produce particular ionic and pH conditions upon rehydration, e.g., without the need for adjustment of such conditions prior to use. The invention also relates to methods of producing dry powder cells, such as prokaryotic (e.g., bacterial) and eukaryotic (e.g., fungal (especially yeast), animal (especially mammalian) and plant cells).

### Related Art

### Cell Culture Media

Cell culture media provide the nutrients for maintaining and/or growing cells in a controlled, artificial and in vitro environment. Characteristics and compositions of the cell culture media vary depending on the particular cellular requirements and any functions for which the cells are cultured. Important parameters include osmolality, pH, and nutrient formulations. The normal environment of a cell in culture is an aqueous medium in which nutrients and other culture components are dissolved or suspended. Especially advantageous is incorporation of useable quantities of lipid or other components that are only sparsely soluble in water.

Media formulations have been used to cultivate a number of cell types including animal, plant, yeast and prokaryotic cells including bacterial cells. Some cells are capable of growing on a solid or semi-solid medium, but cells derived from other than the simplest life forms generally are cultured in liquid phase. Cells cultivated in culture media catabolize available nutrients and can thereby produce useful biological substances such as monoclonal antibodies, hormones, growth factors, viruses, antigenic factors, enzymes, cytokines and the like. Such products have industrial and/or therapeutic applications and, with the advent of recombinant DNA technology, cells can be engineered to produce large quantities of these products. Thus, the ability to cultivate cells in vitro is not only important for the study of cell physiology, but is also necessary for the production of useful substances which may not otherwise be obtained by cost-effective means.

As the cells catabolize nutrients the environment in which the cells grow is constantly being altered. Catabolic products may remain in culture or may require the cultured cells to catabolize these also to maintain cell health. The medium is thus constantly changing. The requirements of the cultured cells may be changing also. Especially for media optimized for a particular cell type or especially a particular production task as the cells grow (and produce) the medium becomes less conducive to the desired result. Supplementation of medium has been effectively used to prolong culture or to maintain or improve production. Several supplementation programs have been used. For example, a single bolus or multiple boli have been added to culture to replenish or sometimes modify medium constituents. Continuous feed programs have also been tried. Supplementation of the growing culture can maintain growth and productivity of the cultured cells over extended time periods.

Simple supplementation might entail adding original medium to provide the same nutrients, but at a different final concentration (as diluted by the partially spent medium). However, since not all media components, e.g., sodium and chloride, are altered, preferably supplements will comprise a set of ingredients less than that of the original medium. Although some ingredients are preferably omitted, a supplement might contain ingredients not present in the original medium being supplemented.

Cell culture media formulations have been well documented in the literature and a number of media are commercially available. In early cell culture work, media formulations were based upon the chemical composition and physicochemical properties (e.g., osmolality, pH, etc.) of blood and were referred to as “physiological solutions” (Ringer, S., J. Physiol. 3:380-393 (1880); Waymouth, C., In: Cells and Tissues in Culture, Vol. 1, Academic Press, London, pp. 99-142 (1965); Waymouth, C., In vitro 6:109-127 (1970)). However, cells in different tissues of muticellular organisms, e.g., plants, invertebrates including insects, vertebrates including fish and mammals are exposed to different microenvironments with respect to oxygen/carbon dioxide partial pressure and concentrations of nutrients, vitamins, and trace elements; accordingly, successful in vitro culture of different cell types will often require the use of different media formulations. Typical components of cell culture media include amino acids, organic and inorganic salts, vitamins, trace metals, sugars, lipids and nucleic acids, the types and amounts of which may vary depending upon the particular requirements of a given cell or tissue type and the purpose to which the cell is applied. Often, particularly in complex media compositions, stability problems result in toxic products and/or lower effective concentrations of required nutrients, thereby limiting the functional life-span of the culture media. For instance, glutamine is a constituent of almost all media that are used in culturing of mammalian cells in vitro. Glutamine decomposes spontaneously into pyrolidone carboxylic acid and ammonia. The rate of degradation can be influenced by pH and ionic conditions but in cell culture media, formation of these breakdown products often cannot be avoided (Tritsch et al., Exp. Cell Res. 28:360-364(1962)).

Wang et al. (In vitro 14(8):715-722 (1978)) have shown that photoproducts such as hydrogen peroxide, which are lethal to cells, are produced in Dulbecco's Modified Eagle's Medium (DMEM). Riboflavin and tryptophan or tyrosine are components necessary for formation of hydrogen peroxide during light exposure. Since most mammalian culture media contain riboflavin, tyrosine and tryptophan, toxic photoproducts are likely produced in most cell culture media.

To avoid these problems, researchers make media on an “as needed” basis, and avoid long term storage of the culture media. Commercially available media, typically in dry power form, serves as a convenient alternative to making the media from scratch, i.e., adding each nutrient individually, and also avoids some of the stability problems associated with liquid media. However, only a limited number of commercial culture media are available, except for those custom formulations supplied by the manufacturer.

Liquid (aqueous media) are often supplemented with lipid concentrate, e.g., Lipid Concentrate (100×), lipid, available from GIBCO of Invitrogen Corporation, Carlsbad, Calif. Conventionally powdered media could not efficiently contain components not readily soluble in water, the most common solvent used for reconstitution. Thus, after a powder is reconstituted to form a medium, additional components are frequently added with a small quantity of organic solvent such as alcohols (e.g., methanol, ethanol, glycols, etc.), ethers (e.g., MEK), ketones (e.g., acetone), DMSO, etc. These solvents must be used sparingly as they generally elicit undesired or toxic effects in the cells being cultured. Toxicity and solubility interact to limit the amount of desired component that can be added to the culture.

Although dry powder media formulations may increase shelf-life of some media, there are a number of problems associated with dry powdered media, especially in large scale application. Production of large media volumes requires storage facilities for the dry powder media, not to mention the specialized media kitchens necessary to mix and weigh the nutrient components. Due to the corrosive nature of dry powder media, mixing tanks must be periodically replaced.

Typically, cell culture media formulations are supplemented with a range of additives, including undefined components such as fetal bovine serum (FBS) (e.g., 10-20%, 5-10%, 1-5%, 0.1-1% v/v) or extracts or hydrolysates from plants, animal embryos, organs or glands (e.g., 0.5-10%, 0.1-1% v/v). While FBS is the most commonly applied supplement in animal cell culture media, other serum sources are also routinely used, including newborn calf, horse and human. Organs or glands that have been used to prepare extracts for the supplementation of culture media include submaxillary gland (Cohen, S., J. Biol. Chem. 237:1555-1565 (1961)), pituitary (Peehl, D. M., and Ham, R. G., In vitro 16:516-525 (1980); U.S. Pat. No. 4,673,649), hypothalamus (Maciag, T., et al., Proc. Natl. Acad. Sci. USA 76:5674-5678 (1979); Gilchrest, B. A., et al., J. Cell. Physiol. 120:377-383 (1984)), ocular retina (Barretault, D., et al., Differentiation 18:29-42 (1981)) and brain (Maciag, T., et al., Science 211:1452-1454 (1981)). Cell culture media may also contain other animal-derived products, including but not limited to blood-derived products (e.g., serum, albumin, antibodies, fibrinogen, factor VIII, etc.), tissue or organ extracts and/or hydrolysates (e.g., bovine pituitary extract (BPE), bovine brain extract, chick embryo extract and bovine embryo extract), and animal-derived lipids, fatty acids, proteins, amino acids, peptones, Excyte™, sterols (e.g., cholesterol) and lipoproteins (e.g., high-density and low-density lipoproteins (HDLs and LDLs, respectively)). Cell culture media may also contain specific purified or recombinant growth factors for example: insulin, fibroblast growth factor (FGF), epidermal growth factors (EGF), transferrin, hematopoietic growth factors like erythropoietin, IL, 1, 2, 3, 4, 5, 6, 7, 8, 9, 10, 11, etc., colony stimulating factors like G-CSF, GM-CSF, histotypic specific growth factors like neural growth factors, specific regulators of cAMP or other signal transductive pathways etc. These types of supplements (e.g., chemically undefined) serve several useful functions in cell culture media (Lambert, K. J. et al., In: Animal Cell Biotechnology, Vol. 1, Spier, R. E. et al., Eds., Academic Press New York, pp. 85-122 (1985)). For example, these supplements provide carriers or chelators for labile or water-insoluble nutrients; bind and neutralize toxic moieties; provide hormones and growth factors, protease inhibitors and essential, often unidentified or undefined low molecular weight nutrients; and protect cells from physical stress and damage. Thus, serum or organ/gland extracts or animal derived products are commonly used as relatively low-cost supplements to provide an improved or optimal culture medium for the cultivation of animal cells.

For food or therapeutic uses, there is a movement to reduce and even eliminate undefined components, particularly of animal origin, because of cost and safety concerns. Improved culture media can also be produced using small amounts of components having low solubility in water.

Unfortunately, the use of such animal derived components or nutrients in tissue or cell culture applications has several drawbacks (Lambert, K. J., et al., In: Animal Cell Biotechnology, Vol. 1, Spier, R. E., et al., Eds., Academic Press New York, pp. 85-122 (1985)). Foremost is the potential to contaminate tissue or cell cultures with adventitious agents or toxins. Indeed, supplementation of media with animal or human derived components may introduce infectious agents (e.g., mycoplasma and/or viruses) or toxins which can seriously undermine the health of the cultured cells when these contaminated supplements are used in cell culture media formulations, and may result in the production of biological substances (e.g. antibodies, hormones, growth factors etc.) which are contaminated with infectious agents or toxins. Thus, contamination of cell or tissue cultures with adventitious agents or toxins may pose a health risk in cell therapy and in other clinical applications. A major fear is the presence of non-cellular soluble or insoluble proteins or other classes of bioactive components that may have disease pathogenesis, and in particular the presence of prions causing spongiform encephalopathy in humans or animals.

Thus, there exists a current need to reduce or eliminate adventitious agents (e.g. infectious agents) and toxins from cell culture reagents (e.g. nutritive media, media supplements, media subgroups, buffers and any nutritive components or solutions which may be found in cell culture media including proteins, carbohydrates, lipids, amino acids, vitamins, nucleic acids, DNA, RNA, trace metals and buffers either alone or in combination). Such cell culture reagents having reduced or eliminated adventitious agents or toxins will be particularly important to the pharmaceutical and medical industry.

### Methods of Production of Culture Media

Culture media are typically produced in liquid form or in powdered form (See for example GIBCO BRL Products 2000-2001 catalogue). Each of these forms has particular advantages and disadvantages.

For example, liquid culture medium has the advantage that it is provided ready-to-use (unless supplementation with nutrients or other components is necessary or desired), and that the formulations have been optimized for particular cell types. Liquid media have the disadvantages, however, that they often do require the addition of supplements (e.g., L-glutamine, serum, extracts, cytokines, lipids, vitamins, nutrients (including amino acids, nuclosides and/or nucleotides, carbon sources, one or more sugar, alcohol or other carbon containing compounds), etc.) for optimal performance in cell cultivation. Furthermore, liquid medium is often difficult to sterilize economically, since many of the components are heat labile (thus obviating the use of autoclaving, for example) and bulk liquids are not particularly amenable to penetrating sterilization methods such as gamma or ultraviolet irradiation; thus, liquid culture media are most often sterilized by filtration, which can become a time-consuming and expensive process. Furthermore, production and storage of large batch sizes (e.g., 1000 liters or more) of liquid culture media are impractical, and the components of liquid culture media often have relatively short shelf lives.

To overcome some of these disadvantages, liquid culture medium can be formulated in concentrated form; these media components may then be diluted to working concentrations prior to use. This approach provides the capability of making larger and variable batch sizes than with standard culture media, and the concentrated media formulations or components thereof often have longer shelf-life (see U.S. Pat. No. 5,474,931, which is directed to culture media concentrate technology). Despite these advantages, however, concentrated liquid media still have the disadvantages of their need for the addition of supplements (e.g., FBS, L-glutamine or organ/gland extracts), and may be difficult to sterilize economically.

Additional supplements, such as nutrient feeds or supplements to replace exhausted or diminished media components may also be desired. Supplements can be liquid supplements, such as liquid concentrate format, but preferably are in a dry format powder (e.g., agglomerated).

As an alternative to liquid media and/or supplements, powdered culture media are often used. The powders are reconstituted by dilution in solvent to produce a reconstituted liquid, e.g., a liquid medium or medium supplement.

Powdered media are typically produced by admixing dried components of the culture medium via a mixing process, e.g., ball-milling, or by lyophilizing pre-made liquid culture medium. This approach has the advantages that even larger batch sizes may be produced, the powdered media typically have longer shelf lives than liquid media, and the media can be sterilized by irradiation (e.g., gamma or ultraviolet irradiation) or ethylene oxide permeation after formulation. However, powdered media (e.g., conventional powdered media) have several distinct disadvantages. For example, some of the components of powdered media become insoluble or aggregate upon lyophilization such that resolubilization is difficult or impossible. Furthermore, powdered media typically comprise fine dust particles which can be hazardous to personnel and equipment and make the media particularly difficult to reconstitute without some loss of material, and which may further make the media impractical for use in many biotechnology production facilities operating under, e.g., GMP/GLP, USP or ISO 9000 settings. Additionally, many of the conventional supplements used in culture media, e.g., L-glutamine and FBS, cannot be added to culture medium prior to lyophilization or ball-milling due to their instability or propensity to aggregate upon concentration or due to their sensitivity to shearing by processes such as ball-milling. Furthermore, many of these supplements, particularly serum supplements such as FBS, show a substantial loss of activity or are rendered completely inactive if attempts are made to produce powdered supplements by processes such as lyophilization. Finally, powdered media and supplements often do not contain bicarbonate buffering systems and require post-reconstitution adjustment of pH, while components required in μg/ml amounts, or less, are typically added post-reconstitution because of homogeneity concerns.

Supplements, since they are used in conjunction with and often share multiple ingredients with media, also share the same concerns of the various formats. Liquid components generally are a combination of acidic solutions (to keep amino acids in solution), neutral solutions for acid-sensitive chemicals and basic solutions (which the customer must use to adjust the pH back to neutral prior to admitting to the bioreactor). Multi-component dry forms require the customer to add acid to dissolve the amino acids and base to re-adjust to neutral pH prior to adding to the bioreactor. Similarly to the liquid supplementation, a neutral component may be required.

Thus, there exists a current need for rapidly dissolving nutritionally complex stable dry powder nutritive media, media supplements, media subgroups and buffers, which can be prepared in variable bulk quantities and which are amenable to sterilization particularly by ionizing or ultraviolet irradiation. With the present single component dry form nutrient supplement, no acid or base solutions need to be used since no pH adjustment is needed. Water is added and mixed and the single component supplement is ready for perfusion into the bioreactor.

Nutrient supplementation generally involves multiple liquids which must be shipped at greater expense and under hazardous protocol or multiple dry components which require acid and base for preparation prior to addition to the bioreactor. With single component dry form supplementation, the customer does not have to make any adjustments to the supplement, reducing concern over use of hazardous components. In addition, shipping weight and storage is much less problematic. Using the inventive format, once water is added, dissolution occurs quickly and the resultant liquid single component can be filtered and added directly into the bioreactor without any pH adjustment.

In particular there is a need to provide dry powder nutritive media manufactured such that no additional manipulations are complete, i.e., do not require or substantially reduce the need for supplementation e.g., with a lipid supplement, after reconstitution.

By use of the present invention, cell culture media and media supplements can be manufactured such that no additional manipulations are needed other than adding solvent and solubilizing the media components.

### BRIEF SUMMARY OF THE INVENTION

The present invention provides methods for the production of nutritive media, media supplement, media subgroup and buffer powders comprising agglomerating a dry powder nutritive media, media supplement, media subgroup or buffer with a solvent or solvents. The invention also relates to methods for the production of powdered nutritive media, media supplements, media subgroups, and buffers, comprising spray-drying a liquid nutritive medium, medium supplement, medium subgroup or buffer under conditions sufficient to produce their dry powder counterparts. Such conditions may, for example, comprise controlling heat, humidity and/or partial pressure(s) of the solvent(s) until the powdered media, media supplement, media subgroup or buffer is formed. The powder may be formed in one step or in multiple steps. When more than one solvent is used the solvents may be introduced through the same port or nozzle or may be introduced though separate nozzles. Compatible solvents, e.g., those soluble in each other or sufficiently miscible may share a port or nozzle while a separate nozzle may be used for one or more solvents incompatible with the first solvent.

According to the invention, the method may further comprise sterilizing the nutritive media, media supplement, media subgroup or buffer powder, which may be accomplished prior to or after packaging the powder. In particularly preferred methods, the sterilization is accomplished after packaging of the powder by irradiation of the packaged powder with gamma rays.

Particularly preferred nutritive medium powders that may be produced according to the invention include culture medium powders selected from the group consisting of a bacterial culture medium powder, a yeast culture medium powder, a plant culture medium powder and an animal culture medium powder. In one aspect, such culture media are produced in dry powdered form, although they may be produced in liquid form (e.g., by admixing with one or more solvents).

Particularly preferred media supplements that may be produced by the methods of the invention include: blood derived products, powdered animal sera, such as bovine sera (e.g., fetal bovine, newborn calf or normal calf sera), human sera, equine sera, porcine sera, monkey sera, ape sera, rat sera, murine sera, rabbit sera, ovine sera and the like; cytokines (including growth factors (such as EGF, aFGF, bFGF, KGF, HGF, IGF-1, IGF-2, NGF and the like), interleukins, colony-stimulating factors and interferons); attachment factors or extracellular matrix components (such as collagens, laminins, proteoglycans, glycosaminoglycans, fibronectin, vitronectin and the like); lipids (such as phospholipids, cholesterol, bovine cholesterol concentrate, fatty acids, Excyte™, sphingolipids and the like); glycans and extracts of animal tissues, extracts or hydrolysates of tissues, organs or glands (e.g., from animals, plants, insects, fish, yeast, bacteria or any other prokaryotic or eukaryotic source such as bovine pituitary extract, bovine brain extract, chick embryo extract, bovine embryo extract, yeast extract, chicken meat extract, achilles tendon and extracts thereof) and the like). Other media supplements that may be produced by the present methods include a variety of proteins (such as serum albumins, particularly bovine or human serum albumins; immunoglobulins and fragments or complexes thereof; aprotinin; hemoglobin; haemin or haematin; enzymes (such as trypsin, collagenases, pancreatinin or dispase); lipoproteins; ferritin; etc.) which may be natural or recombinant; vitamins (including but not limited to vitamins A, B₁, B₂, B₃, B₆, B₁₂, C, D, E, K and H (biotin)); amino acids and variants thereof (including, but not limited to, L-glutamine and cystine), enzyme co-factors, trace elements (such as calcium, copper, iron, magnesium, manganese, nickel, potassium, tin, zinc, selenium, vanadium and the like), sugars, polysaccharides and other components useful in cultivating cells in vitro that will be familiar to one of ordinary skill. In some embodiments, such supplements are produced in dry powdered form but may be produced in liquid form by, for example, mixing one or more solvents with the dry powdered supplement of interest.

The invention also provides a dry format supplement powder which requires only addition of a solvent such as water. Preferably no pH adjusting is necessary. Preferably the dry format powder is prepared by at least one method selected from the group consisting of milling, impacting, extruding and cutting or breaking, wet granulation, high shear granulation, pan granulation and fluidized bed agglomeration.

The invention also provides complete dry powder culture media formulations that support the cultivation of cells in vitro upon reconstitution of the medium with a solvent, without the need for the addition of any supplemental nutrient components to the medium prior to use. In accordance with the invention, such complete media may be automatically pH-adjusting media, and may comprise one or more components such as serum (preferably those described herein), one or more culture medium supplements, L-glutamine, insulin, transferrin, one or more hormones, one or more lipids (preferably one or more phospholipids, sphingolipids, fatty acids or cholesterol), one or more growth factors, one or more cytokines (preferably those described herein), one or more neurotransmitters, one or more extracts of animal tissues, one or more extracts or hydrolysates of tissues, organs or glands (preferably those described herein), organs or glands, one or more enzymes, one or more proteins (preferably those described herein), one or more trace elements, one or more extracellular matrix components, one or more antibiotics, one or more viral inhibitors, and or one or more buffers (preferably sodium bicarbonate or phosphate) or any combination thereof.

Buffer powders particularly suitable for preparation according to the methods of the invention include buffered saline powders, most particularly phosphate-buffered saline powders or Tris-buffered saline powders and buffers used in clinical or electrolyte solutions (i.e. Ringer's, Ringer's lactate, parenteral nutrition solutions or powders). Some embodiments of the invention provide methods of preparing “auto-pH” buffer powders which automatically are at a desired pH upon rehydration/reconstitution with a solvent. In accordance with the invention, such buffers may be in powdered or liquid form.

The invention also provides nutritive medium powders, medium supplement powders (including powders of the herein-described supplements) and buffer powders, particularly auto-pH medium, medium supplement and buffer powders, prepared according to these methods.

The invention also relates to methods of preparing dried cells, including prokaryotic (e.g., bacterial) and eukaryotic (e.g., fungal (especially yeast, including filamentous yeast), animal (especially mammalian, including human) and plant) cells, comprising obtaining a cell to be dried, contacting the cell with one or more stabilizers (e.g., a polysaccharide such as trehalose), forming an aqueous suspension comprising the cell, and spray-drying the cell suspension under conditions favoring the production of a dried powder. Also, see U.S. patent application Ser. No. 10/832,461. Optionally, lipid components may be added to stabilize the dry cell composition. The invention also relates to dried cell powders produced by these methods.

The invention also relates to methods of preparing cells, cell cultures, or cell preparations in which the level of toxins, adventitious agents or other detrimental components are reduced or eliminated. Such cells include prokaryotic (e.g., bacterial) and eukaryotic (e.g., fungal (especially yeast), animal (especially mammalian, including human) and plant cells. This method of the invention thus may comprise obtaining one or more cells and subjecting said cells to the methods of the invention under conditions sufficient to reduce, substantially reduce, inactivate or eliminate one or more toxins and/or one or more adventitious agents. In this aspect of the invention, the conditions (e.g. temperature, humidity, atmospheric pressure, type of gases, gas flow and gas flow pattern (e.g., volatile or turbulent stream) etc.) used may be optimized or adjusted to avoid or substantially avoid adversely affecting the cells of interest. Preferably, conditions are used such that the viability of such cells are not reduced or substantially reduced. Thus, the invention relates to exposing a sample comprising cells with air or gas (or combination of gases) to reduce, eliminate or inactivate toxins and/or adventitious agents in said sample. The invention also relates to cells produced by these methods, which may be in dry (preferably powdered) or liquid form.

The invention further relates to methods of preparing sterile or substantially sterile samples or powders (preferably cell culture reagents and particularly culture media, media supplements, media subgroups and buffers). One such method comprises exposing the sample (e.g. powdered culture media, media supplements, media subgroups and buffers) to irradiation (e.g., preferably gamma irradiation) such that unwanted bacteria, fungi, spores and, viruses etc. that may be resident in the sample are rendered incapable or substantially incapable of replication or growth. In a preferred such method, the powder or sample (e.g. cell culture reagent including media, media supplements, media subgroups and buffers) are gamma-irradiated at a total dosage of about 10-100 kilograys (kGy), preferably a total dosage of about 15-75 kGy, 15-50 kGy, 15-40 kGy or 20-40 kGy, more preferably a total dosage of about 20-30 kGy, and most preferably a total dosage of about 25 kGy, for about 1 hour to about 7 days, preferably for about 1 hour to about 5 days, more preferably for about 1 hour to about 3 days, about 1 hour to about 24 hours or about 1-5 hours, and most preferably about 1-3 hours. With proper shielding for more powerful sources higher exposures may be delivered in shortened times. The invention also relates to sterile powdered samples such as culture media, media supplements, media subgroups and buffers produced by these methods, Preferably, powdered samples such as culture media, media supplements, media subgroups and buffers are subjected to such irradiation before or after packaging. Other sterilization processes may also be used alone or in combination with the invention, for example, filtration, ethylene oxide sterilization, autoclaving, and chemical or physical processes such as heat, pH treatment, chemical treatment, treatment with iodine, or photoactive compounds like porphyrin, psoralens, etc.

The invention further provides methods of culturing a cell comprising reconstituting the nutritive media, media supplement, media subgroup or buffer of the invention with a solvent, which preferably comprises serum or water, and contacting the cell with the reconstituted nutritive media, media supplement, media subgroup or buffer under conditions favoring the cultivation of the cell. Any cell may be cultured according to the present methods, particularly bacterial cells, yeast cells, plant cells or animal cells. Preferable animal cells for culturing by the present methods include insect cells (most preferably Drosophila cells, Spodoptera cells and Trichoplusa cells), nematode cells (most preferably C. elegans cells) and mammalian cells (most preferably CHO cells, COS cells, VERO cells, BHK cells, AE-1 cells, SP2/0 cells, L5.1 cells, PerC6, hybridoma cells or other human cells). Cells cultured according to this aspect of the invention may be normal cells, diseased cells, transformed cells, mutant cells, somatic cells, germ cells, stem cells, precursor cells or embryonic cells, any of which may be established or transformed cell lines or obtained from natural sources. Cells may be used for experimental purposes or for production of useful components.

The invention also provides compositions comprising one or more of the culture media, media supplement, media subgroup or buffer powders of the invention and at least one cell. Such compositions may comprise, for example, an automatically pH-adjusting culture medium powder of the invention or a complete dry powder medium of the invention and one or more cells, such as one or more bacterial cells, one or more plant cells, one or more yeast cells, and one or more animal cells (including but not limited to one or more mammalian cells such as one or more human cells). Compositions according to this aspect of the invention may be in powder form which, upon reconstitution with a solvent, produce an active culture of the one or more cells contained in the composition.

The invention is further directed to kits for use in the cultivation of a cell. Kits according to the invention may comprise one or more containers containing one or more of the nutritive media powders, media supplement powders, media subgroup powders or buffer powders of the invention, solvent(s) or any combination thereof. The kits may also comprise one or more cells or cell types, including the dried cell powders of the invention.

The invention additionally provides the following aspects:

Aspect 1. A method for producing an automatically pH-adjusting dry powdered culture medium, comprising: (a) determining the ratio of pH-opposing forms of buffer salts required to be added to said powder to automatically provide a desired final pH upon reconstitution of said powder with a solvent; and (b) adding amounts of pH-opposing forms of buffer salts to said powder in the ratio determined in step (a).

Aspect 2. The method of aspect 1, further comprising packaging said dry powdered medium.

Aspect 3. The method of aspect 1, further comprising sterilizing said dry powdered medium.

Aspect 4. The method of aspect 3, wherein said sterilization accomplished by irradiating said dry powdered medium with gamma rays until said medium is sterile.

Aspect 5. The method of any one of aspects 1-3, wherein said medium comprises at least one monobasic and/or dibasic buffering salt.

Aspect 6. The method of aspect 5, wherein said monobasic and/or dibasic buffering salt is a monobasic and/or dibasic phosphate salt.

Aspect 7. The method of aspect 6, wherein at least one of said monobasic and/or dibasic phosphate salts is a sodium phosphate salt.

Aspect 8. The method of aspect 6, wherein at least one of said monobasic or dibasic phosphate salts is a potassium phosphate salt.

Aspect 9. The method of aspect 1, wherein said dry powder medium contains sodium bicarbonate but does not liberate CO₂ upon storage.

Aspect 10. An automatically pH-adjusting dry powdered culture medium produced by the method of any one of aspects 1-3 and 9.

Aspect 11. A complete dry powder culture medium that supports the cultivation of a cell in vitro upon reconstitution of the medium with a solvent without the addition of any supplemental nutrient components to said medium.

Aspect 12. The medium of aspect 11, wherein said medium is an automatically pH-adjusting medium.

Aspect 13. The medium of aspect 11, wherein said medium comprises one or more components selected from the group of components consisting of serum, one or more culture medium supplements, L-glutamine, insulin, transferrin, one or more hormones, one or more lipids, one or more growth factors, one or more cytokines, one or more neurotransmitters, one or more extracts of animal tissues, organs or glands, one or more enzymes, one or more proteins, one or more trace elements, one or more extracellular matrix components, one or more antibiotics, one or more viral inhibitors, and or one or more buffers.

Aspect 14. A method of cultivating a cell, comprising reconstituting an automatically pH-adjusting dry powdered medium with a solvent to form a culture medium solution, and contacting the cell with said liquid solution under conditions favoring the cultivation of the cell.

Aspect 15. A method of cultivating a cell comprising preparing an automatically pH-adjusting dry powdered culture medium prepared according to the method any one of aspects 1-3 and 9, reconstituting the medium with at least one solvent to form a culture medium solution, and contacting a cell with said solution under conditions favoring cultivation of the cell.

Aspect 16. A method of cultivating a cell, comprising reconstituting the culture medium of aspect 10 with a solvent to form a culture medium solution, and contacting the cell with said solution under conditions favoring the cultivation of the cell.

Aspect 17. A method of cultivating a cell, comprising reconstituting the culture medium of aspect 11 with a solvent to form a culture medium solution, and contacting the cell with said solution under conditions favoring the cultivation of the cell.

Aspect 18. The method of any one of aspects 14, 16 and 17, wherein said cell is a bacterial cell.

Aspect 19. The method of aspect 15, wherein said cell is a bacterial cell.

Aspect 20. The method of any one of aspects 14, 16 and 17, wherein said cell is a eukaryotic cell.

Aspect 21. The method of aspect 15, wherein said cell is a eukaryotic cell.

Aspect 22. The method of aspect 20, wherein said eukaryotic cell is a yeast cell, a plant cell, or a cell line derived therefrom.

Aspect 23. The method of aspect 21, wherein said eukaryotic cell is a yeast cell, a plant cell, or a cell line derived therefrom.

Aspect 24. The method of aspect 20, wherein said eukaryotic cell is an animal cell or a cell line derived therefrom.

Aspect 25. The method of aspect 21, wherein said eukaryotic cell is an animal cell or a cell line derived therefrom.

Aspect 26. The method of aspect 24 or aspect 25, wherein said animal cell is a mammalian cell or a cell line derived therefrom.

Aspect 27. The method of aspect 26, wherein said mammalian cell is a human cell or a cell line derived therefrom.

Aspect 28. A kit for culturing a cell, comprising one or more containers containing an automatically pH-adjusting dry powdered culture medium prepared according to the method of any one of aspects 1-3 and 9.

Aspect 29. A kit for culturing a cell, comprising one or more containers containing the automatically pH-adjusting dry powdered culture medium of aspect 10.

Aspect 30. A kit for culturing a cell, comprising one or more containers containing the complete dry powdered culture medium of aspect 11.

Aspect 31. The kit of aspect 28, wherein said kit further comprises one or more additional containers containing at least one additional component selected from the group consisting of at least one growth factor, at least one culture medium supplement, at least one animal tissue extract, at least one animal organ extract, at least one animal gland extract, at least one enzyme, at least one protein, at least one vitamin, at least one cytokine, at least one lipid, at least one trace element, at least one extracellular matrix component, at least one buffer, at least one antibiotic, and at least one viral inhibitor.

Aspect 32. The kit of aspect 29 or aspect 30, wherein said kit further comprises one or more additional containers containing at least one additional component selected from the group consisting of at least one growth factor, at least one culture medium supplement, at least one animal tissue extract, at least one animal organ extract, at least one animal gland extract, at least one enzyme, at least one protein, at least one vitamin, at least one cytokine, at least one lipid, at least one trace element, at least one extracellular matrix component, at least one buffer, at least one antibiotic, and at least one viral inhibitor.

Aspect 33. A composition comprising the automatically pH-adjusting culture medium of any aspect above and at least one cell.

Aspect 34. The composition of aspect 33, wherein said composition is a powder.

Aspect 35. A composition comprising the complete culture medium of any aspect above and at least one cell.

Aspect 36. The composition of aspect 33 or aspect 35, wherein said cell is selected from the group consisting of a bacterial cell, a yeast cell, a plant cell and an animal cell.

Aspect 37. The composition of aspect 36, wherein said animal cell is a mammalian cell.

Aspect 38. The composition of aspect 37, wherein said mammalian cell is a human cell.

Aspect 39. The composition of aspect 36, wherein said cell is an established or transformed cell line.

Some embodiments of the invention, relate to treating any sample to reduce, substantially reduce, inactivate, or eliminate adventitious agents or toxins present in the sample of interest. In some embodiments, the invention relates to cell culture reagents such as nutritive media, media supplements, media subgroups and buffers (or any ingredient used to make them).

In accordance with the invention, such reduction, inactivation, or elimination of contaminating adventitious agents or toxins is accomplished by drying or substantially drying the sample of interest. Preferably, the sample of interest is exposed to air or other gas (or combination of gases) under conditions sufficient to reduce, substantially reduce, inactivate or eliminate toxins and/or adventitious agents present in the sample. The sample exposed to the air or gas can be in dry (e.g. powdered) or liquid form. Preferably, such conditions involve increasing the surface area of the sample exposed to the air or gas or combination of gases. Increasing the surface area of the sample exposed to air or other gas (or combination of gases) may involve any method in which the particle size of the sample (e.g. in liquid or dry form) in the air or gas is decreased and/or the volume of the sample exposed to the air or gas is increased. Increasing surface area exposure of the sample may be accomplished by atomizing, pulverizing, grinding, dispensing, spraying, misting, dripping, pouring, spreading etc. the dry or liquid sample in and/or through the air or gases. Alternatively, the air or gas may be injected, bubbled, sprayed, etc. through the dry or liquid sample. Preferably, the air/gas is introduced as a volatile, turbulent stream which promotes uniform or homogeneous dispersion and/or agglomeration.

In accordance with the invention, other environmental conditions such as temperature (e.g. heating or cooling or freezing), humidity, atmospheric pressure, gas or air content, time of exposure etc. may be adjusted or optimized during exposure of the sample to the air or gases to facilitate reduction or removal of adventitious agents and toxins. Preferably, heat is applied during exposure of the sample to air or gas (or combination of gases) to facilitate reduction or removal of adventitious agents or toxins from the sample and/or to facilitate drying of the sample, although cooling or freezing temperatures may be applied during exposure. In another aspect, the type of gas or combination of gases as well as the amount (e.g. percentage) of each gas present can be changed or optimized to further assist in reduction or elimination of adventitious agents or toxins. Such gas or gases include but are not limited to ozone, nitrogen, helium, air, carbon dioxide, argon, oxygen, hydrogen etc. In another aspect, chemical or biological compounds or conditions which are toxic or inhibitory to adventitious agents or toxins may be added during or after the process to neutralize or inactivate such agents or toxins. Such compounds or conditions which may be added or varied include but are not limited to antibiotics, hydrochloric acid, sodium hydroxide, antibodies (monoclonal or polyclonal antibodies or fragments thereof), iodine, pH treatment, ozone, α-gamma rays, psoralen or like reagents, porphyrins or derivatives of chlorins or other photoactive reagents or compounds.

Preferably, the sample of interest (which is preferably any cell culture reagent, particularly a media, media supplement, media subgroup or buffer) is dispersed or sprayed into a chamber or other container containing air or gas (or a combination of gases) and most preferably the sample (e.g. dry or liquid form) is subjected to spray drying or agglomeration by procedures well known in the art. Such procedures may involve, for example, the use of a spray drying apparatus and/or a fluid bed apparatus or combinations thereof or similar technology available in the art. In a preferred aspect, a liquid sample is sprayed in the presence of heat under conditions sufficient to dry or substantially dry the sample while a dry or substantially dry sample (preferably in powdered or granular form) is dispersed (e.g. in a chamber) with blowing or pressurized air or gas in the presence of heat. Preferably, such dispersing or spraying is performed under conditions sufficient to reduce, substantially reduce, inactivate or eliminate adventitious agents or toxins in the sample. Such conditions may include, for example, controlling humidity, atmospheric pressure, the content and/or type of gas used, time of exposure, and addition of compounds, to facilitate reduction, inactivation or elimination of toxins or adventitious agents.

Thus, the present invention comprises exposing a sample to air or gas (or combination of gases) under conditions sufficient to reduce, substantially reduce, inactivate or eliminate adventitious agents and/or toxins in said sample. More specifically, the invention comprises:
exposing a sample (preferably a medium, a medium subgroup, a medium supplement or a buffer) to air or gas (or combination of gases) which may contain one or more cellular or non-cellular adventitious agents and/or one or more toxins, preferably by spraying or dispersing said sample in or through said air or gas (or combination of gases), and preferably in the presence of heat; and
obtaining a sample having reduced, substantially reduced, inactivated or eliminated adventitious agents and/or toxins compared to the untreated sample. Such sample produced is preferably in dry form (e.g. powdered).

To further facilitate reduction, substantial reduction, inactivation or elimination of adventitious agents or toxins in the sample of interest, the invention may further comprise sterilizing the sample produced by the methods of the invention. Such sterilization may be accomplished by irradiation or other sterilization methods well known to those of ordinary skill in the art. Preferably, the sample produced by the invention (for example by spray drying or agglomeration) may be sterilized prior to or after packaging. In particularly preferred embodiments, sterilization is accomplished after packaging by irradiation of the packaged material with gamma rays.

Some embodiments of the invention relate, in part, to a nutritive medium powder comprising with one or more properties selected from the group consisting of an angle of repose between from about 10 to about 40 degrees; a bulk density between from about 0.001 g/cm³ to about 1 g/cm³; wherein 51% to 99% of particles are within a range of 30 to 100 mesh; wherein less than 10% of particles pass through a 200 mesh; and wherein the powder displays a flow measurement of about 3 to 5 kg.

In some embodiments, a dry powder animal cell culture medium of the invention has a bulk density between from about 0.5376 g/ml to about 0.6461 g/ml. In some embodiments, a dry powder animal cell culture medium of the invention has a bulk density selected from the group consisting of a bulk density between from about 0.5449 g/ml to about 0.6461 g/ml, about 0.5669 g/ml to about 0.6048 g/ml, about 0.5449 g/ml to about 0.6148 g/ml, about 0.5784 g/ml to about 0.6461 g/ml, about 0.5928 g/ml to about 0.5726 g/ml, about 0.5475 g/ml to about 0.5953 g/ml and about 0.5856 g/ml to about 0.6341 g/ml, about 0.5676 g/ml to about 0.6088 g/ml, about 0.5450 g/ml to about 0.6142 g/ml, about 0.5790 g/ml to about 0.6454 g/ml, about 0.5685 g/ml to about 0.5969 g/ml, about 0.5549 g/ml to about 0.6461 g/ml, about 0.5376 g/ml to about 0.6052 g/ml and about 0.5756 g/ml to about 0.6442 g/ml.

Some embodiments of the invention relate, in part, to concentrated feed supplement media, methods of producing concentrated feed supplement media and method utilizing concentrated feed supplement media of the invention. Some embodiments of the invention provide a concentrated feed supplement medium comprising at least one component, wherein the concentration of the at least one component is at a concentration at least 3 times higher than the at least one component's desired concentration in a cell culture medium to be supplemented. In some embodiments, a concentration of the at least one component is selected from the group consisting of between from about 3.0 to about 3.5×, about 3.5 to about 4.5×, about 4.5 to about 5.5×, about 5.5 to about 6.5×, about 6.5 to about 7.5×, about 7.5 to about 8.5×, about 8.5 to about 9.5×, and about 9.5 to about 10.5×. In some embodiments, the at least one component is an amino acid. In some embodiments, the at least one component is selected from the group consisting of L-cystine, L-asparagine and L-tyrosine. In some embodiments, a concentrated feed supplement medium does not comprise at least one salt selected from the group consisting of sodium chloride, potassium chloride and sodium bicarbonate.

In some embodiments, a concentrated feed supplement medium comprises at least two components selected from the group consisting of L-cystine, L-asparagine and L-tyrosine, wherein the concentration of the at least two components are at a concentration at least 3 times higher than the at least two component's desired concentration in a cell culture medium to be supplemented. In some embodiments, a concentration of the at least two components is selected from the group consisting of between from about 3.0 to about 3.5×, about 3.5 to about 4.5×, about 4.5 to about 5.5×, about 5.5 to about 6.5×, about 6.5 to about 7.5×, about 7.5 to about 8.5×, about 8.5 to about 9.5×, and about 9.5 to about 10.5×.

In some embodiments, a concentrated feed supplement medium comprises at least three components wherein the three components are L-cystine, L-asparagine and L-tyrosine, and wherein the concentration of the at least three components are at a concentration at least 3 times higher than the at least three component's desired concentration in a cell culture medium to be supplemented. In some embodiments, a concentration of the three components is selected from the group consisting of between from about 3.0 to about 3.5×, about 3.5 to about 4.5×, about 4.5 to about 5.5×, about 5.5 to about 6.5×, about 6.5 to about 7.5×, about 7.5 to about 8.5×, about 8.5 to about 9.5×, and about 9.5 to about 10.5×.

Some embodiments of the invention provide a concentrated feed supplement medium, wherein a concentration of at least one component is higher than the solubility limit of the component(s). In some embodiments, at least two components are higher than the solubility limit of each of the at least two components. In some embodiments, at least three components are higher than the solubility limit of each of the three components.

The invention is further directed to kits for use in the cultivation or manipulation of one or more cells or tissues. Kits according to the invention may comprise one or more containers comprising one or more samples of the invention, preferably one or more cell culture reagents including nutritive media, media supplements, media subgroups or buffers, or any combination thereof. The kits may also comprise one or more cells or cell types or tissues, including the dried cells of the invention.

Another aspect of the invention relates to compositions comprising cell culture reagents, nutritive media, media supplement, media subgroup, or buffers of the invention and one or more cells or tissues. Such composition may be in powdered or liquid form.

Other preferred embodiments of the present invention will be apparent to one of ordinary skill in light of the following drawings and description of the invention, and of the claims.

### BRIEF DESCRIPTION OF THE DRAWINGS

FIG. 1A, FIG. 1B are histograms of densitometric scans of SDS-PAGE of samples of fetal bovine serum (FBS) prepared in powdered form (FIG. 1A) by the methods of the invention and conventional liquid FBS (FIG. 1B).

FIG. 2A, FIG. 2B are composites of line graphs of growth (FIG. 2A) and passage success (FIG. 2B) of SP2/0 cells in Dulbecco's Modified Eagle's Medium (DMEM) supplemented with 2% (w/v) FBS prepared in powdered form by the agglomeration methods of the invention.

FIG. 3A, FIG. 3B are composites of histograms of spectrophotometric scans (λ=200-350 nm) of powdered fetal bovine serum (FBS) prepared by spray-drying according to the methods of the invention (FIG. 3A) or of standard liquid FBS (FIG. 3B).

FIG. 4A, FIG. 4B are composites of line graphs showing the pH titration (buffer capacity), on two different dates (FIGS. 4A and 4B), of various dry powdered media (DPM) prepared by the methods of the invention or by ball-milling, with or without the addition of sodium bicarbonate.

FIG. 5A, FIG. 5B are composites of bar graphs showing the effect of agglomeration on dissolution rates (in water) of Opti-MEM I™ (FIG. 5A) or DMEM (FIG. 5B). Media were agglomerated with water or FBS as indicated.

FIG. 6A, FIG. 6B are composites of line graphs showing growth over seven days of SP2/0 cells in agglomerated Opti-MEM I™ (FIG. 6A) or DMEM (FIG. 6B), both containing 2% FBS.

FIG. 7A, FIG. 7B, FIG. 7C are composites of line graphs showing growth over seven days of SP2/0 cells (FIG. 7A), AE-1 cells (FIG. 7B) and L5.1 cells (FIG. 7C) in agglomerated DMEM containing 10% FBS.

FIG. 8A, FIG. 8B are composites of line graphs showing passage success of SP2/0 cells in Opti-MEM I™ (FIG. 8A) or DMEM (FIG. 8B), agglomerated with either water or FBS, supplemented with 2% FBS.

FIG. 9A, FIG. 9B, FIG. 9C are composites of line graphs showing passage success of SP2/0 cells (FIG. 9A), AE-1 cells (FIG. 9B) and L5.1 cells (FIG. 9C) in DMEM agglomerated with FBS and sodium bicarbonate and supplemented with 10% FBS.

FIG. 10 is a line graph showing the growth of SP2/0 cells over four passages in standard water-reconstituted powdered culture media (control media), or in agglomerated powdered culture media prepared in large-scale amounts according to the methods of the invention. Results are shown for control media (□), water-agglomerated powdered culture media of the invention (♦) and water-agglomerated auto-pH powdered culture media (containing sodium bicarbonate) of the invention (▪).

FIG. 11A, FIG. 11B are line graphs of AE-1 cells cultured over six or seven days in medium containing 2% (▴) or 10% (♦) liquid fetal bovine serum (FBS), or 2% (×) or 10% (▪) powdered FBS prepared by the spray-drying methods of the invention. Duplicate experiments are shown in FIGS. 11A and 11B.

FIG. 12A, FIG. 12B are line graphs of SP2/0 cells cultured over seven days in medium containing 2% (▴) or 10% (♦) liquid FBS, or 2% (×) or 10% (▪) powdered FBS prepared by the spray-drying methods of the invention. Duplicate experiments are shown in FIGS. 12A and 12B.

FIG. 13 is a line graph of AE-1 cell growth over four passages in media containing 5% liquid FBS (♦) or 5% powdered FBS prepared by the spray-drying methods of the invention (▪).

FIG. 14 is a line graph indicating the effect of γ irradiation and agglomeration on the growth of SP2/0 cells over five days.

FIG. 15 is a bar graph indicating the effect of γ irradiation on the growth of VERO cells in agglomerated culture media.

FIG. 16A, FIG. 16B, FIG. 16C, FIG. 16D are a series of line graphs indicating the effect of γ irradiation on the ability of transferrin to support the growth of 293 cells over four passages. In each graph, cells were cultured in standard serum-free 293 medium (♦), in medium without transferrin (▪), in medium containing powdered transferrin that had been γ irradiated at −70° C. (▴) or room temperature ( [symbol] ), or in medium containing powdered transferrin that had not been γ irradiated but that had been stored at −70° C. (×) or at room temperature (●). Results for each data point are the averages of duplicate flasks.

FIG. 16A: passage 1 cells;

FIG. 16B: passage 2 cells;

FIG. 16C: passage 3 cells;

FIG. 16D: passage 4 cells.

FIG. 17A, FIG. 17B, FIG. 17C, FIG. 17D are a series of bar graphs indicating the effect of γ irradiation, under different irradiation conditions, on the ability of FBS to support growth of anchorage-independent cells (FIGS. 17A and 17B) and anchorage-dependent cells (FIGS. 17C and 17D) at first (Px1), second (Px2) and third (Px3) passages.

FIG. 17A: SP2/0 cells;

FIG. 17B: AE-1 cells;

FIG. 17C: VERO cells;

FIG. 17D: BHK cells.

FIG. 18 is a line graph depicting the buffering kinetics of solutions of 5.1 mM sodium phosphate in the dibasic (× - - - ×) or monobasic (∘ - - - ∘) forms upon challenge with various volumes of 5N HCl.

FIG. 19A, FIG. 19B are a series of line graphs depicting the buffering kinetics for RPMI-1640 culture media in various forms, with or without the addition of NaHCO₃.

FIG. 19A: liquid vs. powder media.

♦ - - - ♦: liquid RPMI-1640 containing NaHCO₃ (note that this line is superimposed with that for powder RPMI-1640 containing NaHCO₃);

▪ - - - ▪: liquid RPMI-1640 with no NaHCO₃;

[symbol] : powder RPMI-1640 containing NaHCO₃ (note that this line is superimposed with that for liquid RPMI-1640 containing NaHCO₃);

[symbol] : powder RPMI-1640 containing NaHCO₃, agglomerated but without auto-pH;

[symbol] : powder RPMI-1640 containing NaHCO₃, agglomerated and with auto-pH.

FIG. 19B: powder media, milled or non-milled.

♦ - - - ♦: milled RPMI-1640 containing non-milled NaHCO₃ (note that this line is superimposed with that for non-milled RPMI-1640 containing milled NaHCO₃);

▪ - - - ▪: milled RPMI-1640 with no NaHCO₃;

[symbol] : non-milled RPMI-1640 containing milled NaHCO₃ (note that this line is superimposed with that for milled RPMI-1640 containing non-milled NaHCO₃);

[symbol] : non-milled RPMI-1640 containing milled NaHCO₃, agglomerated but without auto-pH;

[symbol] : non-milled RPMI-1640 containing milled NaHCO₃, agglomerated and with auto-pH.

FIG. 20 shows rEPO production from CHO DG44 and cell densities when grown in CD OptiCHO with a concentrated fed batch supplement or batch control.

FIG. 21 shows IgG production from PER.C6 cells and cell densities when grown in CD OptiCHO with a concentrated fed batch supplement or batch control.

FIG. 22 shows IgG production from PER.C6® EpCAM cells and cell densities when grown in Protein Expression Medium with a concentrated fed batch supplement or batch control.

FIG. 23 shows rEPO production from CHO DG44 and cell densities when grown in CD OptiCHO in a bioreactor with a concentrated fed batch supplement or batch control.

### DETAILED DESCRIPTION OF THE INVENTION

### Definitions

In the description that follows, a number of terms conventionally used in the field of cell culture media are utilized extensively. In order to provide a clear and consistent understanding of the specification and claims, and the scope to be given such terms, the following definitions are provided.

The term “powder” as used herein refers to a composition that is present in granular form, which may or may not be complexed or agglomerated with a solvent such as water or serum. The term “dry powder” may be used interchangeably with the term “powder;” however, “dry powder” as used herein simply refers to the gross appearance of the granulated material and is not intended to mean that the material is completely free of complexed or agglomerated solvent unless otherwise indicated.

The term “ingredient” refers to any compound, whether of chemical or biological origin, that can be used in cell culture media to maintain or promote the growth of proliferation of cells. The terms “component,” “nutrient” and ingredient” can be used interchangeably and are all meant to refer to such compounds. Typical ingredients that are used in cell culture media include amino acids, salts, metals, sugars, carbohydrates, lipids, nucleic acids, hormones, vitamins, fatty acids, proteins and the like. Other ingredients that promote or maintain cultivation of cells ex vivo can be selected by those of skill in the art, in accordance with the particular need.

Mean diameter. Particle size quantified by laser light scattering or by mechanical segregation on relative mesh size calibrated screens.

Homogenous Mixture. As used herein homogenous mixture is a mixture whose composition when samples from various location varies less than 5.0% the relative standard deviation (RDS) of the actual concentration from the theoretical concentration. Each sample may be as large as desired, for example, up to 1% 10% or 20% of the total, but can be quite small, for example an 20 gm sample of 140 kg lot (0.015%) or 20 gm sample of 250 kg lot (0.008%) or as small a samples as 20 gm sample of 500 kg lot (0.0004%). Analytical limits and particle size will put practical limits the minimum desired sample size.

Physiologic pH. As used herein physiologic pH is greater than about 4 and less than about 9. Other or particular pH values or ranges, e.g., minimum or maximum pHs of greater than 4.2, 4.5, 4.8, 5.0, 5.2, 5.5, 5.7, 5.8, 6.0, 6.2, 6.5, 6.7, 6.8, 7.0, 7.2, 7.4, 7.5, 7.8, 8.0, 8.2, 8.4, 8.5, 8.7, 8.8, etc or from about 4.0 to about 9.0, from about 4.0 to about 5.0, from about 5.0 to about 6.0, from about 6.0 to about 7.0, from about 8.0 to about 9.0, from about 4.0 to about 6.0, from about 5.0 to about 7.0, from about 6.0 to about 8.0, from about 7.0 to about 9.0, from about 6.0 to about 9.0, or from about 4.0 to about 7.0 may also be used for dissolving supplements. Some supplements, though not preferred, may only be entirely soluble outside these ranges.

Polar solvent. As used herein polar solvent may include water, saline, water with soluble acid or base ions, with a pH range of 1.0-10.0, stabilizers, surfactants, preservatives, and alcohols and other non polar organic solvents.

The term “cytokine” refers to a compound that induces a physiological response in a cell, such as growth, differentiation, senescence, apoptosis, cytotoxicity, synthesis or transport, immune response or antibody secretion. Included in this definition of “cytokine” are growth factors, interleukins, colony-stimulating factors, interferons, thromboxanes, prostaglandins, hormones and lymphokines.

By “cell culture” or “culture” is meant the maintenance of cells in an artificial, e.g., an in vitro environment. It is to be understood, however, that the term “cell culture” is a generic term and may be used to encompass the cultivation not only of individual prokaryotic (e.g., bacterial) or eukaryotic (e.g., animal, plant and fungal) cells, but also of tissues, organs, organ systems or whole organisms, for which the terms “tissue culture,” “organ culture,” “organ system culture” or “organotypic culture” may occasionally be used interchangeably with the term “cell culture.”

By “cultivation” is meant the maintenance of cells in an artificial environment under conditions favoring growth, differentiation, or continued viability, in an active or quiescent state, of the cells. Thus, “cultivation” may be used interchangeably with “cell culture” or any of its synonyms described above.

By “culture vessel” is meant a glass, plastic, or metal container that can provide an aseptic environment for culturing cells.

The phrases “cell culture medium,” “culture medium,” and “medium formulation” (plural “media” in each case) refer to a nutritive solution that supports the cultivation and/or growth of cells; these phrases may be used interchangeably.

By “extract” is meant a composition comprising a or concentrated preparation of the subgroups of a substance, typically formed by treatment of the substance either mechanically (e.g., by pressure treatment) or chemically (e.g., by distillation, precipitation, enzymatic action or high salt treatment).

By “enzymatic digest” is meant a composition comprising a specialized type of extract, namely one prepared by treating the substance to be extracted (e.g., plant components or yeast cells) with at least one enzyme capable of breaking down the components of the substance into simpler forms (e.g., into a preparation comprising mono- or disaccharides and/or mono-, di- or tripeptides). In this context, and for the purposes of the present invention, the term “hydrolysate” may be used interchangeably with the term “enzymatic digest.”

“Lipid” will have its meaning as generally understood in biochemistry. “Lipid” also means a portion of the cell or an ingredient of a medium that is soluble in non-polar or non-aqueous solvent. The lipid may be sparsely soluble or insoluble in water in the presence or absence of other medium ingredients. Lipid may be soluble in a solvent mixture that includes water and one or more organic solvents. Lipids may comprise fatty acids, hormones, metabolites, cytokines, vitamins, indicators, stimulators or inhibitors. “Lipid” in some contexts may refer to ingredients that are normally insoluble or sparsely soluble in water, but that have been converted, e.g., by saponification hydroxylation, etc., to form a compound or ion that is water soluble. Thus, for example, a fatty acid is a lipid, but also a salt of a fatty acid is to be included in the definition. Additionally, “lipid” is used generically to mean generally any component that is advantageously introduced using organic or non-polar solvents or that is not normally soluble in water or aqueous media. Lipids may be present as dissolved molecules, or in other forms such as micelles or other loose associations of molecules. A lipid may be used as a free molecule or may be bound to one or more other molecules. For example, proteins or peptides may be associated with one or more other lipids for stability and/or to aid in delivery to the agglomerated powder. Lipid may also refer to an ingredient that might act as a drug to inhibit or activate one or more functions of a cell or cell component.

By “adventitious agents” is meant any agent such as one or more bacteria, one or more pathogenic microorganisms, one or more microbial pathogens, one or more viruses, one or more mycoplasma, one or more yeast cells, one or more fungi, one or more non cellular compounds that result in acute or chronic toxicity or disease, and the like which may contaminate a sample of interest. Adventitious agents may be present in any number of animal derived products or components used in cell culture reagents. Preferred adventitious agents reduced, eliminated, inactivated or killed by the invention are viruses which may be animal, human, plant, fish, insect, mammalian, DNA, RNA, envelope and non-envelope viruses, regardless of size. Such viruses include Adenoviruses, Herpesviruses, Poxviruses, Papovaviruses, Retroviruses, Orthomyxoviruses (influenza viruses), Paramyxoviruses (parainfluenza, mumps, measles, and respiratory syncytial virus), Picornaviruses (Enteroviruses, Cardioviruses, Rhinoviruses, and Aphthoviruses), Togaviruses, Arenaviruses, Reoviruses, Rotaviruses, Orbiviruses, Rhabdoviruses, Coronaviruses, Marburg Viruses, Ebola Viruses, and Hepatitis Viruses (see “Comparative Diagnosis of Viral Diseases”, (E. Kurstak and C. Kurstak, eds.), Vol. I-IV, Academic Press, New York, and “Medical Microbiology and Infectious Diseases”, (A. Samiy, L. Smith, Jr., J. Wyngaarden, eds.), Vol II, W.B. Saunders Co., Philadelphia, Pa.). Examples of such viruses included but are not limited to those shown in the following tables:

TABLE 1
Some Animal Viruses

| | | | Approximate | |
|---|---|---|---|---|
| Virus | Genome | Envelope | Size (mM) | Comment |
| BVDV | ss-RNA | + | 40-60 | Bovine virus diarrhea |
| IBR | ds-DNA | + | 120-200 | Infect. Bovine |
| | | | | Rhinotrachetis |
| PI-3 | ss-RNA | + | 80-160 | Parainfluenza |
| BPV | ss-DNA | − | 25 | Bovine Parvovirus |
| BAV | ds-DNA | − | 70-80 | Bovine Adenoviruses |
| BpoV | ds-DNA | − | 25-35 | Bovine Polyomavirus |
| BMV | ds-DNA | + | 80 | Bovine Mammilitis |
| | | | | virus |
| Vaccinia | ds-DNA | + | 120 | |
| virus | | | | |
| FMD virus | ss-RNA | − | 25 | Foot & Mouth Disease |
| | | | | Virus |
| VSV | ss-RNA | + | 40 × 120 | Vesicular Stomatitis |
| | | | | Virus |
| Orf Virus | ds-DNA | + | 70-90 | |
| BEV | ss-RNA | − | 25 | Bovine Enterovirus |
| PEV | ss-RNA | − | 25 | Porcine Enterovirus |
| PPV | ss-DNA | − | 20 | Porcine Parvovirus |
| Rabies | ss-RNA | + | 40 × 120 | |
| Virus | | | | |
| REO-3 | ds-RNA | − | 60 | |
| BRSV | ss-RNA | + | 80-120 | Bovine Respiratory |
| | | | | Syncytial Virus |
| PHV-1 | ds-DNA | + | 120-200 | Porcine Herpes |
| | | | | virus-1 |
| Rhinovirus | ss-RNA | − | 25 | |
| Calicivirus | ss-RNA | − | 25 | |
| Rotavirus | ds-RNA | − | 60 | |
| Hog Cholera | ss-RNA | + | 40-60 | |
| Border Dis. | ss-RNA | + | 40-60 | |
| EEE | ss-RNA | + | 60-80 | Eastern Equine |
| | | | | Encephalitis Virus |
| WEE | ss-RNA | + | 60-80 | Western Equine |
| | | | | Encephalitis Virus |
| VEE | ss-RNA | + | 60-80 | Venezuelan Equine |
| | | | | Encephalitis Virus |
| JEE | ss-RNA | + | 60-80 | Japanese Equine |
| | | | | Encephalitis Virus |
| Akabane | ss-RNA | − | 60 | |
| BTV | ds-RNA | − | 60 | Blue tongue virus |

TABLE 2
Some Human Viruses

| Virus | Genome | Envelope |
|---|---|---|
| HSV-1,2 | ds-DNA | + |
| HAV (Hepatitis A) | ss-RNA | − |
| HBV (Hepatitis B) | ds-DNA | + |
| HCV (Hepatitis C) | ss-RNA | + |
| HEV (Hepatitis E) | ds-DNA | − |
| HIV-1,2 (AIDS) | ss-RNA | + |
| B-19 | ss-DNA | − |
| Adeno viruses | ds-DNA | − |
| Poxviruses (Smallpox, vaccinia) | ds-DNA | + |
| RSV (Respiratory Syntitial) | ss-RNA | + |
| Measles | ss-RNA | + |
| Rubella | ss-RNA | + |
| Influenza A, B | ss-RNA | + |
| Parainfluenza | ss-RNA | + |
| Mumps | ss-RNA | + |
| Rabies | ss-DNA | + |
| HTLV (T-Leuk.) | ss-RNA | + |
| CMV (cytomegalovirus) | ds-DNA | + |
| Poliomielitos | ss-RNA | − |
| Arboviruses | ss-RNA | + |
| Hantaan virus | ss-RNA | + |
| MFV (Marburg fever) | ss-RNA | − |
| Ebola | ss-RNA | + |
| Lassa | ss-RNA | + |
| Calicivirus | ss-RNA | − |
| Coxsackie virus | ss-RNA | − |
| ROTA | ds-RNA | − |
| REO-3 | ds-RNA | − |
| SV-40 | ds-DNA | − |
| Polyomaviruses | ds-DNA | − |
| Papillomavirus | ds-DNA | − |
| Rhinovirus | ss-RNA | − |
| Yellow Fever | ss-RNA | + |
| Dengue | ss-RNA | + |
| Encephalitis viruses | ss-RNA | + |
| Corona virus | ss-RNA | + |
| Varicella-Zoster | ss-DNA | + |
| Epstein-Barr virus | ds-DNA | + |

Examples of bacteria include but are not limited to gram negative and gram positive bacteria, preferably of the genus Staphylococcus, Streptococcus, Corynebacterium, Bacillus, Neisseria, Shigella, Escherichia, Salmonella, Klebsiella, Proteus, Erwinia, Vibrio, Pseudomonas, Brucella, Bordetella, Haemophilus, Yersinia, and particuarly Corynebacterium diphtheriae, Eschericia coli, Streptococcus pyogenes, Staphylococcus aureus, and Mycobacteria tuberculosis. Examples of mycoplasma include but are not limited to M. bovimastitidis, M. canis, M. hominis, M. hyorhinis, M. urealyticum, M. orale, M. salivarium, M. laidlawi, and M. pneumoniae. Examples of yeast cells include but are not limited to Saccharomyces cerevisiae, Cryptococcus neoformans, Blastomyces dermatitidis, Histoplasma capsulatum, Paracoccidioides brasiliensis, and Candida albicaus. Examples of fungi include but are not limited to Coccidioides immitis, Aspergillus fumigatis, Microsporum audouini, Trichophyton mentagrophytes, and Epidermophyton floccosum. See “Medical Microbiology and Infectious Diseases”, (A. Samiy, L. Smith, Jr., J. Wyngaarden, eds.), Vol II, W.B. Saunders Co., Philadelphia, Pa.

By “toxins” is meant any biological or chemical compound (including proteins) or combinations thereof that inhibit cell function or cell growth. Thus, the presence of one or more toxins in cell culture results in inhibition of cell growth or function or may kill all or a number of cells in such culture. Examples of toxins include but are not limited to endotoxin, exotoxins, snake venom, cholera toxin, Staphylococcal enterotoxin, leukocidin, Ricin A, poisions derived from animals, neurotoxin, and erythrogenic toxin. See “Medical Microbiology and Infectious Diseases”, (A. Samiy, L. Smith, Jr., J. Wyngaarden, eds.), Vol II, W.B. Saunders Co., Philadelphia, Pa.

The term “substantially reduced” refers to a reduction in the amount of adventitious agents and/or toxins in a sample (particularly cell culture reagents, nutrient media, media supplements, media subgroups and buffers). Such reduction is preferably a reduction of greater than 50%, more preferably greater than 60%, still more preferably greater than 70%, still more preferably greater than 80%, still more preferably greater than 90% and most preferably greater than 95% compared to the level of adventitious agents and/or toxins in the sample prior to treatment in accordance with the invention. The invention provides at least a one log, preferably at least a two log, more preferably at least a three log, still more preferably at least a four log, still more preferably at least a five log and most preferably at least a six log reduction in the level of toxin and/or adventitious agents in a sample of interest.

The term “contacting” refers to the placing of cells to be cultivated into a culture vessel with the medium in which the cells are to be cultivated. The term “contacting” encompasses inter alia mixing cells with medium, perfusing cells with medium, pipetting medium onto cells in a culture vessel, and submerging cells in culture medium.

The term “combining” refers to the mixing or admixing of ingredients in a cell culture medium formulation. Combining can occur in liquid or powder form or with one or more powders and one or more liquids.

The term “pillowing” refers to the event which occurs when any moisture, including atmospheric water, infiltrates a container and moistens the powder contained therein. Such moistening may result in acidic conditions within the container that will cause the liberation of CO₂ gas from the powder (“off-gassing”). When dry powder “pillows” in a sealed container, the off-gassing may cause the container to swell to the point of bursting.

The term “small-quantity” refers to components present in the medium in μg/ml, g/L, or lower amounts.

A cell culture medium is composed of a number of ingredients and these ingredients vary from one culture medium to another. A “1× formulation” is meant to refer to any aqueous solution that contains some or all ingredients found in a cell culture medium at working concentrations. The “1× formulation” can refer to, for example, the cell culture medium or to any subgroup of ingredients for that medium. The concentration of an ingredient in a 1× solution is about the same as the concentration of that ingredient found in a cell culture formulation used for maintaining or cultivating cells in vitro. A cell culture medium used for the in vitro cultivation of cells is a 1× formulation by definition. When a number of ingredients are present, each ingredient in a 1× formulation has a concentration about equal to the concentration of those ingredients in a cell culture medium. For example, RPMI-1640 culture medium contains, among other ingredients, 0.2 g/L L-arginine, 0.05 g/L L-asparagine, and 0.02 g/L L-aspartic acid. A “1× formulation” of these amino acids contains about the same concentrations of these ingredients in solution. Thus, when referring to a “1× formulation,” it is intended that each ingredient in solution has the same or about the same concentration as that found in the cell culture medium being described. The concentrations of ingredients in a 1× formulation of cell culture medium are well known to those of ordinary skill in the art. See Methods For Preparation of Media, Supplements and Substrate For Serum-Free Animal Cell Culture Allen R. Liss, N.Y. (1984), which is incorporated by reference herein in its entirety. The osmolality and/or pH, however, may differ in a 1× formulation compared to the culture medium, particularly when fewer ingredients are contained in the 1× formulation. The 1× concentration of any component is not necessarily constant across various media formulations. 1× might therefore indicate different concentrations of a single component when referring to different media. However, when used generally, 1× will indicate a concentration commonly found in the types of media being referenced. A 1× amount is the amount of an ingredient that will result in a 1× concentration for the relevant volume of medium.

A “10× formulation” is meant to refer to a solution wherein each ingredient in that solution is about 10 times more concentrated than the same ingredient in the cell culture medium. For example, a 10× formulation of RPMI-1640 culture medium may contain, among other ingredients, 2.0 g/L L-arginine, 0.5 g/L L-asparagine, and 0.2 g/L L-aspartic acid (compare 1× formulation, above). A “10× formulation” may contain a number of additional ingredients at a concentration about 10 times that found in the 1× culture medium. As will be readily apparent, “20× formulation,” “25× formulation,” “50× formulation” and “100× formulation” designate solutions that contain ingredients at about 20-, 25-, 50- or 100-fold concentrations, respectively, as compared to a 1× cell culture medium. Again, the osmolality and pH of the media formulation and concentrated solution may vary. See U.S. Pat. No. 5,474,931, which is directed to culture media concentrate technology.

An “auto-pH” powder of the invention (e.g., auto-pH medium, medium supplement or buffer powder) is a powder which has been formulated such that, upon rehydration with a solvent, the resulting medium, medium supplement or buffer solution is at a desired pH and does not require adjustment of the pH with acid or base prior to use. For example, an auto-pH culture medium that is formulated to be used at pH 7.4 will, upon rehydration with a solvent, be at pH 7.4 and therefore will be ready for immediate use without adjustment of pH. Such auto-pH powders of the invention may also be referred to herein interchangeably as “automatically pH-adjusting” powders.

By “without significant loss of biological and biochemical activity” is meant a decrease of less than about 30%, preferably less than about 25%, more preferably less than about 20%, still more preferably less than about 15%, and most preferably less than about 10%, of the biological or biochemical activity of the nutritive media, media supplement, media subgroup, buffer or sample of interest when compared to a freshly made nutritive media, media supplement, media subgroup, buffer or sample of the same formulation.

A “solvent” is a liquid that dissolves or has dissolved another ingredient of the medium. Solvents may be used in preparing media, in preparing media powders, in preparing subgroups or supplements or other formulations, especially powders of the present invention and in reconstituting a powder or diluting a concentrate in preparation for culturing cells. Solvents may be polar, e.g., an aqueous solvent, or non-polar, e.g., an organic solvent. Solvents may be complex, i.e., requiring more than one ingredient to solubilize an ingredient. Complex solvents may be simple mixtures of two liquids such as alcohol and water or may be mixtures of salts or other solids in a liquid. Two, three, four, five or six or more components may be necessary in some cases to form a soluble mixture. Simple solvents such as mixtures of ethanol or methanol and water are preferred because of their ease of preparation and handling. Because of environmental, toxicity and/or fire concerns, it is preferred to use aqueous mixtures wherein the quantity of organic solvent is the minimum quantity in the mixture to sufficiently dissolve the relevant ingredient or ingredients.

By an “extended period of time” is meant a period of time longer than that for which the sample (e.g. pharmaceutical composition, nutritive medium, medium supplement, medium subgroup or buffer) is stored when prepared by traditional methods such as ball-milling. As used herein, an “extended period of time” therefore means about 1-36 months, about 2-30 months, about 3-24 months, about 6-24 months, about 9-18 months, or about 4-12 months, under a given storage condition, which may include storage at temperatures of about −70° C. to about 25° C., about −20° C. to about 25° C., about 0° C. to about 25° C., about 4° C. to about 25° C., about 10° C. to about 25° C., or about 20° C. to about 25° C. Assays for determining the biological or biochemical activity of pharmaceutical or clinical compositions, cell culture reagents, nutrients, nutritive media, media supplement, media subgroup or buffers are well-known in the art and are familiar to one of ordinary skill.

### Overview

The present invention is directed to methods of producing nutritive media, media supplements, media subgroups or buffers and the media produced thereby. Nutritive media, media supplements and media subgroups produced by the present methods are any media, media supplement or media subgroup (serum-free or serum-containing) which may be used to support the growth of a cell, which may be a bacterial cell, a fungal cell (particularly a yeast cell), a plant cell or an animal cell (particularly an insect cell, a nematode cell or a mammalian cell, most preferably a human cell), any of which may be a somatic cell, a germ cell, a normal cell, a diseased cell, a transformed cell, a mutant cell, a stem cell, a precursor cell or an embryonic cell. Preferred such nutritive media include, but are not limited to, cell culture media, most preferably a bacterial cell culture medium, plant cell culture medium or animal cell culture medium. Preferred media supplements include, but are not limited to, undefined supplements such as extracts of bacterial, animal or plant cells, glands, tissues or organs (particularly bovine pituitary extract, bovine brain extract and chick embryo extract); and biological fluids (particularly animal sera, and most preferably bovine serum (particularly fetal bovine, newborn calf or normal calf serum), horse serum, porcine serum, rat serum, murine serum, rabbit serum, monkey serum, ape serum or human serum, any of which may be fetal serum) and extracts thereof (more preferably serum albumin and most preferably bovine serum albumin or human serum albumin). Medium supplements may also include defined replacements such as LipoMAX®, OptiMAb®, Knock-Out™ SR (each available from Invitrogen Corporation, Carlsbad, Calif.), and the like, which can be used as substitutes for the undefined media supplements described above. Such supplements may also comprise defined components, including but not limited to, hormones, cytokines, neurotransmitters, lipids, attachment factors, proteins and the like.

Nutritive media can also be divided into various subgroups (see U.S. Pat. No. 5,474,931) which can be prepared by, and used in accordance with, the methods of the invention. Such subgroups can be combined to produce the nutritive media of the present invention.

By the methods of the present invention, any nutritive media, media supplement, media subgroup or buffer may be produced and stored for an extended period of time without significant loss of biological and biochemical activity. By some methods of the present invention significant improvement in the incorporation of lipids and/or ingredients poorly soluble in water is achieved. A lipid component can be incorporated in a subgroup, supplement, etc., but a lipid component as well as all other ingredients to be reconstituted is contained in a single mixture/composition. When plural compositions are used for reconstituting a medium preferably a small number of different powders are needed, for example, 2, 3, 4 or 5.

### Formulation of Media, Media Supplements, Media Subgroups, Buffers, Pharmaceutical Compositions and Solutions

Any nutritive medium, medium supplement, medium subgroup or buffer may be prepared by the methods of the present invention. Particularly preferred nutritive media, media supplements and media subgroups that may be prepared according to the invention include cell culture media, media supplements and media subgroups that support the growth of animal cells, plant cells, bacterial cells or yeast cells. Particularly preferred buffers that may be prepared according to the invention include balanced salt solutions which are isotonic for animal cells, plant cells, bacterial cells or yeast cells.

Examples of animal cell culture media that may be prepared according to the present invention include, but are not limited to, DMEM, RPMI-1640, MCDB 131, MCDB 153, MDEM, IMDM, MEM, M199, McCoy's 5A, Williams' Media E, Leibovitz's L-15 Medium, Grace's Insect Medium, IPL-41 Insect Medium, TC-100 Insect Medium, Schneider's Drosophila Medium, Wolf & Quimby's Amphibian Culture Medium, F10 Nutrient Mixture, F12 Nutrient Mixture, those culture media described in U.S. patent application Ser. No. 11/151,647 (e.g., as in Tables 1 and 2), Ser. Nos. 10/105,937 and 09/390,634, and cell-specific serum-free media (SFM) such as those designed to support the culture of keratinocytes, endothelial cells, hepatocytes, melanocytes, CHO cells, 293 cells, PerC6, hybridomas, hematopoetic cells, embryonic cells, neural cells etc. Other media, media supplements and media subgroups suitable for preparation by the invention are available commercially (e.g., from Invitrogen Corporation, Carlsbad Calif., and Sigma; St. Louis, Mo.). Formulations for these media, media supplements and media subgroups, as well as many other commonly used animal cell culture media, media supplements and media subgroups are well-known in the art and may be found, for example, in the GIBCO/BRL Catalogue and Reference Guide (Invitrogen Corporation Carlsbad Calif.) and in the Sigma Animal Cell Catalogue (Sigma; St. Louis, Mo.).

Examples of plant cell culture media that may be prepared according to the present invention include, but are not limited to, Anderson's Plant Culture Media, CLC Basal Media, Gamborg's Media, Guillard's Marine Plant Culture Media, Provasoli's Marine Media, Kao and Michayluk's Media, Murashige and Skoog Media, McCown's Woody Plant Media, Knudson Orchid Media, Lindemann Orchid Media, and Vacin and Went Media. Formulations for these media, which are commercially available, as well as for many other commonly used plant cell culture media, are well-known in the art and may be found for example in the Sigma Plant Cell Culture Catalogue (Sigma; St. Louis, Mo.).

Examples of bacterial cell culture media that may be prepared according to the present invention include, but are not limited to, Trypticase Soy Media, Brain Heart Infusion Media, Yeast Extract Media, Peptone-Yeast Extract Media, Beef Infusion Media, Thioglycollate Media, Indole-Nitrate Media, MR-VP Media, Simmons' Citrate Media, CTA Media, Bile Esculin Media, Bordet-Gengou Media, Charcoal Yeast Extract (CYE) Media, Mannitol-salt Media, MacConkey's Media, Eosin-methylene blue (EMB) media, Thayer-Martin Media, Salmonella-Shigella Media, and Urease Media. Formulations for these media, which are commercially available, as well as for many other commonly used bacterial cell culture media, are well-known in the art and may be found for example in the DIFCO Manual (DIFCO; Norwood, Mass.) and in the Manual of Clinical Microbiology (American Society for Microbiology, Washington, D.C.).

Examples of fungal cell culture media, particularly yeast cell culture media, that may be prepared according to the present invention include, but are not limited to, Sabouraud Media and Yeast Morphology Media (YMA). Formulations for these media, which are commercially available, as well as for many other commonly used yeast cell culture media, are well-known in the art and may be found for example in the DIFCO Manual (DIFCO; Norwood, Mass.) and in the Manual of Clinical Microbiology (American Society for Microbiology, Washington, D.C.).

As the skilled artisan will appreciate, any of the above media or other media that can be prepared according to the present invention may also include one or more additional components, such as indicating or selection agents (e.g., dyes, antibiotics, amino acids, enzymes, substrates and the like), filters (e.g., charcoal), salts, polysaccharides, ions, detergents, stabilizers, and the like. The invention is not limited in its application to presently formulated media, but is broadly applicable to any formulation for culturing cells.

In a particularly preferred embodiment of the invention, the herein-described culture media may comprise one or more buffer salts, preferably sodium bicarbonate, at concentrations sufficient to provide optimal buffering capacity for the culture medium. According to one aspect of the invention, a buffer salt, such as sodium bicarbonate, may be added in powdered form to the powdered medium prior to, during or following agglomeration of the medium. In one example of this aspect of the invention, the sodium bicarbonate may be added to the culture medium prior to, during or following agglomeration with an appropriate solvent (such as water, serum or a pH-adjusting agent such as an acid (e.g., HCl at a concentration of IM to 5M, 0.1M to 5M, or preferably at IM) or a base (e.g., NaOH at a concentration of 1 M to 5M, 0.1M to 5M, or preferably at IM)) such that, upon reconstitution of the agglomerated medium the culture medium is at the optimal or substantially optimal pH for cultivation of a variety of cell types. For example, bacterial cell culture media prepared by the present methods will, upon reconstitution, preferably have a pH of about 4-10, more preferably about 5-9 or about 6-8.5. Fungal (e.g., yeast) cell culture media prepared by the present methods will, upon reconstitution, preferably have a pH of about 3-8, more preferably about 4-8 or about 4-7.5; animal cell culture media prepared by the present methods will, upon reconstitution, preferably have a pH of about 6-8 or about 7-8, more preferably about 7-7.5 or about 7.2-7.4; and plant cell culture media prepared by the present methods will, upon reconstitution, preferably have a pH of about 4-8, preferably about 4.5-7, 5-6 or 5.5-6. Of course, optimal pH for a given culture medium to be used on a particular cell type may also be determined empirically by one of ordinary skill using art-known methods. For example gastric cells may be cultured at pHs well below those of other cells, for example, pH 1-3. One of ordinary skill appreciates that other cells adapted to harsh environments may have special tolerances or needs that might be outside the normal ranges that satisfy culture conditions for commonly cultured cells.

In another example, one or more buffer salts, e.g., sodium bicarbonate, may be added directly to a powdered nutritive medium by agglomerating the buffer(s) into the medium using a fluid bed apparatus, or by spray-drying the buffer(s) onto a dry or agglomerated powdered medium (using a spray-drying apparatus as described herein). In a related aspect, a pH-adjusting agent such as an acid (e.g., HCl) or a base (e.g., NaOH) may be added to a powdered nutritive medium, which may contain one or more buffer salts (such as sodium bicarbonate), by agglomeration of the pH-adjusting agent into the powdered nutritive medium in a fluid bed apparatus, by spray-drying the pH-adjusting agent onto the powdered or agglomerated nutritive medium, or by a combination thereof; this approach obviates the subsequent addition of a pH-adjusting agent after reconstitution of the powdered medium. Thus, the invention provides a powdered nutritive culture medium useful in cultivation or growth of cells in vitro that, upon reconstitution with a solvent (e.g., water or serum), has a pH that is optimal for the support of cell cultivation or growth without a need for adjustment of the pH of the liquid medium. This type of medium, defined herein as “automatically pH-adjusting medium,” therefore obviates the time-consuming and error-prone steps of adding buffer(s) to the medium after reconstitution and adjusting the pH of the medium after dissolution of the buffer(s). For example, a mammalian cell culture medium prepared according to these methods may, upon reconstitution, have a pH of between about 7.1 to about 7.5, more preferably between about 7.1 to about 7.4, and most preferably about 7.2 to about 7.4 or about 7.2 to about 7.3. The preparation of one example of such an automatically pH-adjusting culture medium is shown in more detail below in Examples 3 and 6.

In accordance with certain methods of the present invention, automatically pH adjusting media can be produced by preparing reconstituted media without the addition of any buffering systems or pH-adjusting agents (an “auto-pH medium” of the invention). In a preferred such aspect, an auto-pH medium may be provided by adjusting the buffering systems present in the medium. For example, as one of ordinary skill is aware, culture media typically contain buffers or buffering systems. By adjusting the pH-opposing forms of such buffers in the medium, the invention provides for production of an auto-pH medium, avoiding the requirement to add additional buffers or pH-adjusting agents to achieve a proper pH level prior to or upon reconstitution of the medium and prior to use. In one such aspect of the invention, pH-opposing forms of certain media components (particularly phosphate or other buffer salts) are then used in the culture medium to provide a desired pH upon reconstitution of the powdered media. (pH-opposing forms of components are conjugate acid-base pairs in which the members of the pair can either raise the pH or lower it to achieve the desired pH of the solution. Sodium HEPES (pH raising) and HEPES-HCl (pH lowering) are examples of pH opposing components.) For example, if a reconstituted media having a pH of between 4.5 and 7.2 is to be prepared, the first step is to determine the correct balance of monobasic (to lower the pH) to dibasic (to raise the pH) phosphate in order to yield the desired pH. Typically, mono- and dibasic phosphate salts are used at concentrations of about 0.1 mM to about 10 mM, about 0.2 mM to about 9 mM, about 0.3 mM to about 8.5 mM, about 0.4 mM to about 8 mM, about 0.5 mM to about 7.5 mM, about 0.6 mM to about 7 mM, or preferably about 0.7 mM to about 7 mM. If other buffer systems are used in the formulations, the proper ratio or balance of the basic (typically sodium or monobasic) buffer salt and the corresponding acidic (or pH-opposing; typically HCl or dibasic) buffer salt is similarly determined to ensure that the formulation will be at the desired final pH upon reconstitution with a solvent. Because the actual phosphate molecular species that is present in a solution is the same at a given pH whether the basic (e.g., sodium or monobasic) or acidic (e.g., HCl or dibasic) form is added, this adjustment would not be expected to impact buffering capacity. Once an appropriate ratio of pH-opposing forms of an appropriate buffer is determined, these components may be added to the medium (for example, a dry powder medium) to provide a culture medium that is of the appropriate pH level upon reconstitution and prior to use (i.e., an auto-pH medium of the invention). The preparation of one example of such an automatically pH-adjusting culture medium is described in more detail below in Examples 3, 6 and 17.

In a related aspect, the invention provides for methods of preparing culture media in such a way as to prevent the interaction of media components that adversely affect the stability, solubility, structure and/or performance of the medium. In one such preferred aspect, the methods of the invention prevent the adverse interaction between buffering components that are present in the culture medium. For example, such methods of the invention may be used to prevent off-gassing in the culture medium, which is the release of gas from one or more medium components upon storage of the medium in dry form prior to use. In particular, these methods of the invention may be used to prevent off-gassing of carbon dioxide from the medium, typically resulting from liberation of carbon dioxide from a bicarbonate (particularly sodium bicarbonate) buffer used in the medium. Sodium bicarbonate is generally not included in powdered media because, depending on the type of phosphate buffer used in the media, significant amounts of carbon dioxide gas may be generated by off-gassing of the sodium bicarbonate, which may swell a sealed container of the medium to the bursting point, thus reducing the storage stability of the finished product. To minimize this undesirable condition, dibasic sodium phosphate (Na₂HPO₄) instead of monobasic sodium phosphate (NaH₂PO₄) may be used in the formulations comprising sodium bicarbonate. However, if monobasic sodium phosphate is used in the formulation of the medium, monobasic potassium phosphate (KH₂PO₄) can be used instead, which does not result in gas formation, and thus does not cause pillowing, while having the identical buffering capabilities as monobasic sodium phosphate. According to this aspect of the invention, the ratio of monobasic to dibasic phosphate (or other buffer) salts to be used (or present) in the culture medium is determined, and then the monobasic sodium phosphate (or other monobasic buffer salt) is replaced with equal molar amount of monobasic potassium phosphate, to prevent off-gassing of carbon dioxide from the sodium bicarbonate in the medium. Since the buffering capacity of monobasic sodium phosphate is identical to that of monobasic potassium phosphate, this replacement would not be expected to affect the buffering system present in the medium. Thus, the invention provides culture media that prevent the adverse interaction between components of the medium, particularly preventing off-gassing, while still providing for auto-pH forms of the culture media. The preparation of one example of such an automatically pH-adjusting culture medium where the components of the medium have been adjusted to minimize or prevent off-gassing is described in more detail in Example 17.

Hence, as one of ordinary skill will recognize from the description provided herein, the present invention also provides complete dry powder culture media formulations that support the cultivation of cells in vitro upon reconstitution of the medium with a solvent, without the need for the addition of any supplemental nutrient components to the medium prior to use. Media according to this aspect of the invention thus will preferably comprise the nutritional components necessary for cultivation of a cell in vitro, such that no additional nutritional components need be included in the solvent or added to the medium upon reconstitution and prior to use. Accordingly, such complete media of the invention will be suitable for use in cultivating cells in vitro upon reconstitution with water or with an alternative non-nutrient-containing solvent such as a buffered saline solution. In accordance with the invention, such complete media may be automatically pH-adjusting media, and may comprise one or more components such as one or more culture medium supplements (including but not limited to serum), one or more amino acids (including but not limited to L-glutamine), insulin, transferrin, one or more hormones, one or more lipids, one or more growth factors, one or more cytokines, one or more neurotransmitters, one or more extracts of animal tissues, organs or glands, one or more enzymes, one or more proteins, one or more trace elements, one or more extracellular matrix components, one or more antibiotics, one or more viral inhibitors, and or one or more buffers.

Examples of media supplements that may be prepared as powders by the present methods, or that may be included in the culture media of the invention, include, without limitation, animal sera (such as bovine sera (e.g., fetal bovine, newborn calf and calf sera), human sera, equine sera, porcine sera, monkey sera, ape sera, rat sera, murine sera, rabbit sera, ovine sera and the like), defined replacements such as LipoMAX®, OptiMAb®, Knock-Out™ SR (each available from Invitrogen Corporation, Carlsbad-California), hormones (including steroid hormones such as corticosteroids, estrogens, androgens (e.g., testosterone) and peptide hormones such as insulin, cytokines (including growth factors (e.g., EGF, aFGF, bFGF, HGF, IGF-1, IGF-2, NGF and the like), interleukins, colony-stimulating factors, interferons and the like), neurotransmitters, lipids (including phospholipids, sphingolipids, fatty acids, Excyte™, cholesterol and the like), attachment factors (including extracellular matrix components such as fibronectin, vitronectin, laminins, collagens, proteoglycans, glycosaminoglycans and the like), and extracts or hydrolysates of animal, tissues (e.g., plant or bacteria tissues), cells, organs or glands (such as bovine pituitary extract, bovine brain extract, chick embryo extract, bovine embryo extract, chicken meat extract, chicken tissue extract, achilles tendon and extracts thereof) and the like). Other media supplements that may be produced by the present methods or that may be included in the culture media of the invention include a variety of proteins (such as serum albumins, particularly bovine or human serum albumins; immunoglobulins and fragments or complexes thereof; aprotinin; hemoglobin; haemin or haematin; enzymes (such as trypsin, collagenases, pancreatinin or dispase); lipoproteins; fetuin; ferritin; etc.), which may be natural or recombinant; vitamins; amino acids and variants thereof (including, but not limited to, L-glutamine and cystine), enzyme co-factors; polysaccharides; salts or ions (including trace elements such as salts or ions of molybdenum, vanadium, cobalt, manganese, selenium, and the like); and other supplements and compositions that are useful in cultivating cells in vitro that will be familiar to one of ordinary skill. Media supplements produced by the methods of the invention include animal or mammalian (e.g. human, fish, bovine, porcine, equine, monkey, ape, rat, murine, rabbit, ovine, insect, etc.) derived supplements, ingredients or products. These sera and other media supplements are available commercially (for example, from Invitrogen Corporation, Carlsbad, Calif. and Sigma Cell Culture, St. Louis, Mo.); alternatively, sera and other media supplements described herein may be isolated from their natural sources or produced recombinantly by art-known methods that will be routine to one of ordinary skill (see Freshney, R. I., Culture of Animal Cells, New York: Alan R. Liss, Inc., pp. 74-78 (1983), and references cited therein; see also Harlow, E., and Lane, D., Antibodies: A Laboratory Manual, Cold Spring Harbor, N.Y.: Cold Spring Harbor Laboratory, pp. 116-120 (1988)). Components that are often present in the final formulation in μg/ml or even μg/L amounts have typically been left out of standard powdered media due to homogeneity and/or stability concerns, and instead are typically added to the reconstituted 1× media as a concentrate, thereby increasing storage costs and causing production of a finished culture medium to become more costly and less efficient. Thus, in one preferred aspect of the present invention, such low-level components may be added to standard powdered media by first making a concentrate of the components and then spraying them into a portion of the powdered media that would be granulated with the concentrate (See U.S. application Ser. No. 09/023,790, filed Feb. 13, 1998, which is incorporated herein by reference in its entirety). This would then be milled (e.g., via Fitzmilling) to a particle size in the same general size range as that of the bulk for blending. The ability to spray-in components in small amounts may be especially helpful in developing media that include trace elements, vitamins, viral inhibitors, growth factors, cytokines and the like. Specifically, among others, the components to be added to a powdered medium include but are not limited to calcium, choline chloride, folic acid, inositol, lipoic acid, riboflavin, thiamine hydrochloride, sodium selenite and vitamins A, B₁, B₂, B₃, B₆, B₁₂, C, D, E, K and H (biotin). Additional components to be added in low amounts to the culture media of the invention may include, for example, growth factors (e.g., EGF, aFGF, bFGF, KGF, HGF, IGF-1, IGF-2, NGF, insulin, and the like), interleukins, colony-stimulating factors, interferons, attachment factors, extracellular matrix components (e.g., collagens, laminins, proteoglycans, glysoaminoglycans, fibronectin, vitronectin, and the like), lipids (such as phospholipids, cholesterol, bovine cholesterol concentrate, fatty acids, sphingolipids and the like); extracts of animal tissues, glands or organs; antibiotics such as Geneticin™ carbenicillin, cefotaxime, anti-PPLO, Fungizone™, hygromycin, kanamycin, neomycin, nystatin, penicillin, or streptomycin, etc.; and viral inhibitors (e.g., protease inhibitors, nucleoside analogues, and the like, which are well-known in the art).

Examples of buffers that may be prepared according to the present invention and/or that may be included in the culture media of the present invention-include, but are not limited to, buffered saline solutions, phosphate-buffered saline (PBS) formulations, Tris-buffered saline (TBS) formulations, HEPES-buffered saline (HBS) formulations, Hanks' Balanced Salt Solutions (HBSS), Dulbecco's PBS (DPBS), Earle's Balanced Salt Solutions, Puck's Saline Solutions, Murashige and Skoog Plant Basal Salt Solutions, Keller's Marine Plant Basal Salt Solutions, Provasoli's Marine Plant Basal Salt Solutions, and Kao and Michayluk's Basal Salt Solutions, and the like. Formulations for these buffers, which are commercially available, as well as for many other commonly used buffers, are well-known in the art and may be found for example in the GIBCO/BRL Catalogue and Reference Guide (Life CorporationTechnologies, Rockville, CaliforniaMaryland), in the DIFCO Manual (DIFCO; Norwood, Mass.), and in the Sigma Cell Culture Catalogues for animal and plant cell culture (Sigma; St. Louis, Mo.).

Examples of pharmaceutical compositions or solutions which may be prepared in accordance with the invention include any composition with pharmaceutical properties such as the ability to treat, alleviate or reduce pain, infection, fever, nervous disorders, circulatory disorders, respiratory disorders, nutritional disorders, metabolical disorders and the like. Such pharmaceutical compositions may comprise one or more drugs, chemicals, proteins, antibodies or fragments thereof, antibiotics, etc., or combinations thereof. Such pharmaceutical compositions may further comprise one or more pharmaceutical carriers including lipids, adjuvants, stabilizers and the like. The invention also relates to clinical solutions, particularly those used for parenteral nutrition, electrolyte balance or intravenous (IV) solutions. Such clinical solutions may be found for example in the Baxter catalog (Deerfield, Ill.) and include but are not limited to Ringer's, Ringer's lactate, 5% Dextrose in water, normal saline (0.9% NaCl), hypotonic saline (0.45% NaCl), 5% Dextrose in saline, and the like. Clinical solutions may further comprise one or more pharmaceutical compositions or components thereof described herein.

### Preparation of Powdered Media, Media Supplements, Media Subgroups and Buffers

In one aspect of the invention, the powdered nutritive media, media supplements, media subgroups and buffers are prepared using fluid bed technology to agglomerate the solutions of media, media supplements, media subgroups or buffers, thereby producing their dry powdered forms. Fluid bed technology is a process of producing agglomerated powders having altered characteristics (particularly, for example, solubility) from the starting materials. In general applications of the technology, powders are suspended in an upwardly moving column of air while at the same time a controlled and defined amount of liquid is injected into the powder stream to produce a moistened state of the powder; mild heat is then used to dry the material, producing an agglomerated powder.

Apparatuses for producing and/or processing particulate materials by fluid bed technology are available commercially (e.g., from Niro, Inc./Aeromatic-Fielder; Columbia, Md.), and are described, for example, in U.S. Pat. Nos. 3,771,237; 4,885,848; 5,133,137; 5,357,688; and 5,392,531; and in WO 95/13867; the disclosures of all of the foregoing patents and applications are incorporated by reference herein in their entireties. A number of instruments are commercially available for processing dry powder. Examples of such instruments include Processall Mixmill mixers, Extrud-O-Mix Mixer/Extruder, Turbulizer Mixer/Coater, and Bextruder Extruder/Granulator. See, e.g., products of Hosokawa Bepex Corporation, 333 NE Taft St., Minneapolis, Minn. 55413-2810 and their competitors.

Such apparatuses have been used to prepare agglomerated powders of various materials, including milk whey (U.S. Pat. No. 5,006,204), acidulated meat emulsions (U.S. Pat. No. 4,511,592), proteases (U.S. Pat. No. 4,689,297) and other proteins (DK 167090 B1), and sodium bicarbonate (U.S. Pat. No. 5,325,606).

According to this aspect of the invention, fluid bed technology may be used to prepare bulk agglomerated nutritive media, media supplements, media subgroups and buffers. In the practice of this aspect of the invention, a dry powdered sample (e.g. nutritive medium, medium supplement, media subgroup, or buffer or mixtures or combinations thereof) is placed into a fluid bed apparatus and is subjected to agglomeration therein. Powdered nutritive media (particularly powdered cell culture media), powdered media supplements (particularly powdered animal sera) and powdered buffers (particularly powdered buffered salines), may be obtained pre-made from commercial sources (e.g., Invitrogen Corporation, Carlsbad, Calif.). Alternatively, powdered samples including nutritive media, media supplements, media subgroups or buffers may be made by admixing individual components or sets of components according to the formulations described herein. Such formulations may include components which typically are not present in powdered nutritive media, media supplement, media subgroup and buffer formulations due to their instability, such as serum, L-glutamine, cystine, insulin, transferrin, lipids (particularly phospholipids, sphingolipids, Excyte™, fatty acids and cholesterol) certain carbohydrates, cytokines (particularly growth factors, interleukins, colony-stimulating factors and interferons), neurotransmitters and buffers (particularly sodium bicarbonate). If L-glutamine is added to the formulation, it may be in the form of a complex with divalent cations such as calcium or magnesium (see U.S. Pat. No. 5,474,931). In another example, two or more powdered components may be admixed and then agglomerated to produce a complex mixture such as media, media supplements, media subgroups or buffers. For example, a powdered nutritive medium may be mixed with a powdered serum (produced, for example, by spray-drying as described herein) such as FBS at a serum concentration of about 0.1%, 0.2%, 0.5%, 1%, 2%, 2.5%, 5%, 7.5%, 10%, 15%, 20%, 25%, 50% or higher (w/w as a percentage of the powdered medium); the resulting powdered medium-serum mixture may then be agglomerated to produce an agglomerated medium-serum complex that will readily dissolve in a reconstituting solvent and thus be ready for use without further supplementation.

Once the powdered sample such as nutritive media, media supplement, media subgroup or buffer (or mixture or combinations thereof) is placed into the fluid bed apparatus, it is subjected to suspension in an upwardly moving column of a gas, preferably atmospheric air or an inert gas such as nitrogen, and is passed through one or more particle filters. Alternatively, the gas or combination of gases used may be toxic or inhibitory to adventitious agents or toxins present in the sample. Since most dry powder, non-agglomerated nutritive media, media supplements, media subgroups and buffers are of a relatively small particle size, filters to be used in the invention should be mesh screens that allow air to flow through but that retain the powders, for example filters of about 1-100 mesh, preferably about 2-50 mesh, more preferably about 2.5-35 mesh, still more preferably about 3-20 mesh or about 3.5-15 mesh, and most preferably about 4-6 mesh. Other filters may be used depending on the need and sample used, and can be determined by one skilled in the art.

After placement within the fluid bed chamber, the dry powder sample including nutritive media, media supplement, media subgroup or buffer (or mixtures or combinations thereof) is then optionally treated by injecting, preferably using a spray nozzle on the fluid bed apparatus, a defined and controlled amount of solvent into the powder, to produce a moistened powder. Preferred solvents for use in the present invention are any solvent that is compatible with the formulation of the nutritive media, media supplement, media subgroup, buffer or other sample of interest. In another aspect, the solvent used may be a solvent toxic or inhibitory to adventitious agents or toxins to assist in reducing the content of such agents or toxins in the sample. By “compatible” is meant that the solvent does not induce irreversible deleterious changes in the physical or performance characteristics of the nutritive media, media supplement, media subgroup, buffer or sample, such as breakdown or aggregation of the nutrient components of the nutritive medium or changes in the ionic characteristics of the buffer. Particularly preferred solvents for use in the invention are water (most particularly distilled and/or deionized water), serum (particularly bovine or human serum and most particularly fetal bovine serum or calf serum), organic solvents (particularly dimethylsulfoxide, alcohols (e.g., methanol, ethanol, glycols, etc.), ethers (e.g., MEK), ketones (e.g., acetone), and the like), blood derived products, extracts or hydrolysates of tissues, organs, glands, or cells, animal derived products or any other media supplement or ingredients, buffers, acids or bases (pH adjusting agents), any of which may contain one or more additional components (e.g., salts, polysaccharides, ions, detergents, stabilizers, etc.).

In some aspects of the invention, it may be desirable or advantageous to include in the solvent one or more ingredients that, due to the concentrations of the components desired or required in the final product, cannot be optimally incorporated into the product by other methods such as ball-milling. In one such aspect, the component(s) may be dissolved, suspended, colloided or otherwise introduced into the solvent at the desired concentration, prior to use of the solvent in agglomeration of the powdered sample (e.g. a media, media supplement, media subgroup or buffer of the invention). Components that may be advantageously incorporated into the solvent in accordance with this aspect of the invention include, but are not limited to, one or more of the herein-described sera, hormones, cytokines, neurotransmitters, lipids, carbohydrates, attachment factors, proteins, amino acids, vitamins, enzyme cofactors, animal derived products, blood derived products, extracts or hydrolysates of tissues, organs, glands or cells, polysaccharides, salts, ions, buffers and the like.

The solvent(s) should be introduced into the dry powder in a volume that is dependent upon the mass of powdered media, media supplement, media subgroup, buffer or sample to be agglomerated. Preferred volumes of solvent per 500 grams of sample (e.g. a nutritive media, media supplement, media subgroup or buffer) are about 5-100 ml, more preferably about 10-50 ml, still more preferably about 25-50 ml, and most preferably about 35 ml. Preferred solvent introduction rates per 500 grams of sample (e.g. a nutritive media, media supplement, media subgroup or buffer) are a rate of about 1-10 ml/min, preferably about 2-8 ml/min, more preferably about 4-8 ml/min and most preferably about 6 ml/min. In some situations, it may be desirable to cycle between adding solvent for about one minute and then not adding solvent for about one minute (allowing drying of the powder within the apparatus chamber), so as to prevent clumping of the powder during agglomeration. In some situations it may be desirable to cycle between adding a first solvent and a second or third solvent, with or without a period where no solvent is added. In some situations it may be desirable to add plural solvents coincidently from separate ports within the apparatus.

Once agglomeration of the powder is complete, as evidenced by a larger particle size than that of the original, unagglomerated powder and by the absence of fine dust particles in the agglomerated powder, the powder is substantially dried and preferably thoroughly dried in the apparatus. In some situations it may be desirable to partially or thoroughly dry a powder before adding additional ingredients with a second or third solvent. In some situations it may be desirable to use a previous solvent, e.g., a first solvent as a later solvent, e.g., a third solvent. In some situations it may be desirable to use a simple solvent as, e.g., a first solvent and a complex solvent, e.g., as a second solvent. One of ordinary skill will appreciate that many orders and sequences are possible and optimal conditions can be determined by simple procedures known in the art. Preferred apparatus temperatures for drying of the agglomerated powder are about 50-80° C., more preferably about 55-75° C., and most preferably about 60-65° C.; powder is preferably dried in the apparatus for about 3-10 minutes and most preferably for about 5-7 minutes, per 500 grams of powder. Temperature is chosen so as to avoid deleterious effects such as irreversible denaturation or ingredients. Higher temperatures, e.g., 80-150° C., or higher or lower temperatures, e.g., 20-40° C. may be especially advantageous when less volatile or more volatile solvents respectively are used.

Starting formulations for making some of the nutritive media, media supplements, media subgroups, buffers, samples or pharmaceutical or clinical compositions of the invention, may contain concentrations of ingredients/components that are different than final effective concentrations. In some embodiments of the invention, the amounts of certain ingredients inputted into a process of the invention (e.g., agglomeration) may differ from the amounts in the final products. In some embodiments, ingredients may be “lost” prior to, during and/or after the process (e.g., agglomeration), but before final use of the product. This “loss” of an ingredient(s) may occur by any means.

In some instances a loss of an ingredient(s) may be the result, for example, of an ingredient or portion thereof being “volatilized off” during the process, e.g., during a drying step as part of a fluid bed agglomeration procedure.

In some embodiments, a percentage of the ingredient lost will be determined and an appropriate amount will be added into the process to result in the desired amount at the end of the process. For example, if a 20% loss is noted at the end of the process, then 125% of the desired final amount is added during the process. In some cases, the amount added will be experimentally optimized, e.g., various starting amounts will be tested to determine the required starting amount of an ingredient(s) to achieve the desired final amount/concentration of the ingredient(s). In some embodiments, an amount of an ingredient greater than the amount present at the end of the process (e.g., an agglomeration process) will be added to/into the process. In some embodiments, an ingredient may be added later or after the process (e.g., an agglomeration process), e.g., to prevent or reduce the loss of the ingredient during the process. In some embodiments, an ingredient is added at or toward the end of the process, e.g., to minimize volatilization of the ingredient. In some embodiments, an ingredient is added after the process (e.g., an agglomeration process), for example, by spraying and/or adsorbing the ingredient onto, e.g., an agglomerated product. In some embodiments, an ingredient is added as a supplement (e.g., liquid or powder) to the processed (e.g., agglomerated) powder. In some embodiments, the product is transferred to another entity (e.g., a customer), who adds in at least one ingredient, e.g., in powder and/or liquid form.

In some aspects of the invention, powdered nutritive media, media supplements, media subgroups and buffers of the invention may be prepared by tumble granulation, which produces an agglomerated product analogous to that described above, referred to herein as “tumble granulation agglomerated product.” In such a process, dry powder media, media supplements, media subgroups and/or buffers, or combinations thereof, are introduced into tumble granulator or a tumble blender such as those that are commercially available from Gemco (Middlesex, N.J.) and Patterson Kelley (East Stroudsburg, Pa.). A solvent (e.g., water, buffered saline, or other desirable solvent that is described herein or that will be familiar to one of ordinary skill) is then introduced into the powder under controlled conditions according to manufacturer's specifications in the tumble granulator and the batch is then dried according to manufacturer's specifications to form granulated powder (i.e. granules of powder containing solvent), which may then be used as described herein for agglomerated powders.

In some aspects of the invention, powdered nutritive media, media supplements, media subgroups and buffers of the invention may be prepared by fluid bed agglomeration. In some aspects of the invention, air flow is chosen to maintain fluid conditions in the bed. Temperature may be set to retain liquid introduced into the apparatus for a period of time to allow sufficient agglomeration. Agglomeration is generally sufficient when particles are larger in size than the powders to be agglomerated and when ingredients introduced with solvent are assimilated into the larger size particles. For example, when using more volatile solvents, a lower temperature, e.g., −10° C., 0° C., 5° C., 10° C., 20° C., 25° C., 35° C., or 40° C. may be used. One of ordinary skill will appreciate that as the solvent(s) are volatilized, energy is required which will tend to cool the agglomerating mixture. Temperature can thus be controlled by controlling the type and rate of solvent delivery and the rate of heating the mixture. Agglomeration of dissolved ingredients is preferably accomplished when liquid can act as an agent to bind, e.g., by surface forces, smaller powders, dissolved ingredients or suspended or colloided ingredients to the agglomeration mix in the bed. Thus the agglomeration temperature will vary with the solvent in use, with the rate of flow maintaining the fluidized bed, the rate of delivery of solvents(s), the rate of volatilization of solvent(s) and the rate of heating. Temperature may range, e.g., from a lower bound, e.g., −20° C., −10° C., 0° C., 5° C., 10° C., 20° C., 25° C., 35° C., 40° C. or 50° C. when using volatile solvents or for longer residence time of liquid to effect agglomeration, to a higher bound, e.g., 40° C., 50° C., 60° C., 65° C., 75° C., 85° C., 90° C., 95° C., 100° C., 110° C., 120° C., 125° C., 140° C., 150° C., 175° C., 200° C., 220° C., 240° C., 250° C., 275° C., 300° C. or more for less volatile solvents, for more rapid volatilization and when less agglomeration time is necessary. For example, when multiple solvents are being used either coincidentally or sequentially, the less volatile solvent may be sufficient for agglomeration allowing for more rapid volatilization of a more volatile solvent.

A mixture of solvents may be used to control volatilization time so that liquid is resident in the apparatus for sufficient time to effect agglomeration. For example, a mixture of a more volatile solvent, e.g., an organic solvent such as alcohol, especially ethanol, and a less volatile solvent, e.g., a polar solvent such as water may be used. For example, an ingredient insoluble or poorly soluble in polar solvent may be soluble in an organic solvent. The ingredient may be soluble in a mixture of polar and organic solvent. Thus one aspect of the invention uses a mixture of organic and polar solvent to deliver one or more ingredients. The mixture of solvents, i.e., the ratio of polar to organic solvent will vary with the ingredient(s) to be assimilated into the bed. Parameters to be used in choosing the mixture will include solubility, e.g., the ratio might be set to contain the minimum organic solvent that will deliver the desired quantity of ingredient(s) for agglomeration; volatility, e.g., the ratio may be set to contain a less volatile solvent to result in sufficient agglomeration; safety or regulatory concerns, e.g., the ratio might be set to contain a minimum organic solvent that is sufficient for solvation and agglomeration in the bed but that does not present undue hazards to the workplace or the environment or specific solvents may be chosen or avoided to comply with regulations; conditions of the bed, e.g., the mixture may be chosen so that a desired temperature and/or flow sufficient agglomeration is accomplished; specific uses of the media powder, e.g., for some uses manufacturing protocols will preferably include one or more solvents, while preferably excluding or prohibiting other solvents; and compatibility with the apparatus, e.g., solvents or solvent mixtures to permit facile introduction through a port or nozzle and that do not unacceptably damage the components of the apparatus. The mixture can be introduced in a number of ways. For example, a mixture of solvents may be prepared, optionally with one or more soluble, colloided or suspended ingredients, and delivered as a mixture through a port or nozzle. Another way a mixture may be accomplished is to introduce separate solvents or solvent mixtures through separate routes. For example, the separation may be spatial, plural ports or nozzles might be used; the separation might be temporal, the solvents or mixtures might be introduced sequentially through a single or through separate ports or nozzles; the separation may involve different phases, a solvent may be introduced as a vapor before, during and/or after introduction of a solvent on a liquid phase, or a solvent may be delivered a solid component to the bed and volatilized during bed operation; etc. Any means for introduction will apply equally to delivering solvents or mixtures of solvents.

In some embodiments, a nutritive medium, media supplement, media subgroup, buffer, sample or pharmaceutical or clinical composition is produced by producing at least two separate agglomerated products that may be later combined prior to the final use. In some embodiments, the composition of at least one of the at least two separate agglomerated products contains at least one ingredient not present in at least one other separate agglomerated product. In some embodiments, at least two of the separate agglomerated products contain at least one ingredient each, which is exclusive to that separate agglomerated product, e.g., the at least one ingredient in each of the at least two separate agglomerated product is not found in the other separated agglomerated product.

In some embodiments, the multiple agglomerated products can be combined in dry (e.g., powder) form, combined in reconstituted form or a combination of both. In some embodiments, at least one of the multiple agglomerated products may be reconstituted and this reconstituted product may be utilized to reconstitute at least one of the other agglomerated products. In some embodiments, multiple agglomerated products may be produced and transferred to another entity (e.g., a customer), who then combines the multiple agglomerated products, e.g., as described herein. In some embodiments, the end user will weigh out the multiple agglomerated products and add the solvent at the time of reconstitution.

In some embodiments of the invention, the multiple agglomerated products contain 2, 3, 4, 5, 6, 7, 8, 9, 10, 11, 12, 13, 14, 15, 20 or more agglomerated products. In some embodiments, the multiple agglomerated products may be combined in the agglomerated powder form or may be combined after reconstitution in a liquid state or a combination thereof.

The present invention also provides a method for preparing nutritive media, media supplements, media subgroups, buffers or samples in a liquid or dry powder form which contains a desired or effective amount or concentration of an ingredient(s), wherein at least one ingredient (e.g., a sugar (e.g., glucose, trehalose) vitamins, an amino acid, a salt, a trace element, a growth factor and/or an amine (e.g., ethanolamine, spermine, putrescene and/or paraaminobenzoic acid) is inputted into the agglomeration process at a higher amount as compared to the final product. The present invention also provides a method of compensating for a loss or decrease in effective concentration of at least one ingredient during an agglomeration process comprising calculating or determining the amount of the ingredient to be added to the process (e.g., as described herein) to result in the final desired or effective amount.

By “effective amount” or “effective concentration” is meant an amount of an ingredient which is available for use. One example is the amount of a vitamin in a culture medium which is available to cells for use in biological processes normally associated with that vitamin. Thus, an effective amount includes the amount of a cell culture ingredient (e.g., a vitamin or sugar) available for a cell to metabolize. An effective amount of an ingredient can be determined, for example, from the knowledge available to one skilled in the art and/or by experimental determination.

Effective amounts or effective concentrations can be determined using bioassays known to those skilled in the art, e.g., assays similar to assays for GM-CSF activity or Colony Stimulating Factor as described in U.S. Pat. No. 5,532,341.

One method for determining the effective concentration of a compound (e.g., a vitamin) in a test culture medium is as follows. Using a vitamin for the purposes of illustration, a known concentration of the vitamin is serially diluted into a culture medium lacking the vitamin. A second set of serial dilutions are set-up where the test culture medium is serially diluted into a culture medium also lacking the vitamin. Cells that require the vitamin for growth are then added to both sets of serially diluted samples and cultured under appropriate conditions. After a period of time, cell replication is measured (e.g., by cell counting or by measuring optical density). The measurements of the known concentrations are graphed to form a standard curve, to which the measurements from the test culture medium dilutions are compared to determine the effective concentration of the vitamin in the test culture medium. Any number of similar assays may be used to determine the amount of a metabolite(s) in a sample which are available for cellular metabolism.

### Characteristics of Agglomerated Products of the Invention

Various methods are available and known to those skilled in the art for characterizing particles, as well as other materials, such as solid materials (e.g., those of the dry powder nutritive media, media supplements, media subgroups, buffers or samples of the invention). For examples and as a general reference see, Jillavenkatesa et al. “Particle Size Characterization”, NIST Recommended Practice Guide, Special Publication 960-1 (2001); Bernhardt, “Particle Size Analysis: Classification and Sedimentation Methods”, 1st Eng. Lang. ed., Chapman and Hall, London (1994); Allen, “Particle Size Measurement”, 4th ed., Chapman and Hall, London (1990); ASTM E1617-97 “Standard Practice for Reporting Particle Size Characterization Data”, American Society for Testing and Materials, West Conshohocken, Pa. (1997); ISO 9276-1, “Representation of Results of Particle Size Analysis-Part 1: Graphical representation, International Organization for Standardization”, Geneva (1998); Svarovsky et al., “Characterization of Powders, in Principles of Powder Technology”, M. J. Rhodes, ed., John Wiley & Sons, Chichester (1990), e.g., p. 35; and/or Heywood, “Pharmaceutical Aspects of Fine Particles and Their Evaluation. II Evaluation of Powders”, Pharm. J., 191 (5211):291 (1963). Examples of agglomerated material, which may have one or more characteristics described in this section, include material generated using methods set forth in Examples 1 and 29.

“Bulk density” is a property of particulate materials and is the mass of particles divided by the volume they occupy. This volume includes the space between particles as well as the space occupied by the particles. There are many techniques known in the art for measuring bulk density. These include indirect or direct measures. Direct measures, e.g., comprise determining the bulk density by weighing a volume of a sample. Typically, the weight is divided by the volume to arrive at the bulk density.

The invention further provides dry powder nutritive media, media supplements, media subgroups, buffers and samples (e.g., material generated using methods set forth in Example 1) with particular bulk densities or ranges of bulk densities. For example, some dry powder nutritive media, media supplements, media subgroups, buffers or samples of the invention will have a bulk density between from about 0.01 g/cm³ to about 0.8 g/cm³, from about 0.4 g/cm³ to about 0.9 g/cm³, from about 0.05 g/cm³ to about 0.8 g/cm³, from about 0.07 g/cm³ to about 0.8 g/cm³, from about 0.1 g/cm³ to about 0.8 g/cm³, from about 0.2 g/cm³ to about 0.8 g/cm³, from about 0.3 g/cm³ to about 0.8 g/cm³, from about 0.4 g/cm³ to about 0.8 g/cm³, from about 0.5 g/cm³ to about 0.8 g/cm³, from about 0.6 g/cm³ to about 0.8 g/cm³, from about 0.7 g/cm³ to about 0.8 g/cm³, from about 0.1 g/cm³ to about 0.7 g/cm³, from about 0.1 g/cm³ to about 0.6 g/cm³, from about 0.1 g/cm³ to about 0.5 g/cm³, from about 0.1 g/cm³ to about 0.4 g/cm³, from about 0.1 g/cm³ to about 0.3 g/cm³, from about 0.1 g/cm³ to about 0.2 g/cm³, from about 0.4 g/cm³ to about 0.8 g/cm³, from about 0.4 g/cm³ to about 0.6 g/cm³, from about 0.5 g/cm³ to about 0.8 g/cm³, from about 0.5 g/cm³ to about 0.7 g/cm³, from about 0.45 g/cm³ to about 0.75 g/cm³, from about 0.55 g/cm³ to about 0.65 g/cm³, from about 0.55 g/cm³ to about 0.75 g/cm³, from about 0.65 g/cm³ to about 0.75 g/cm³, from about 0.05 g/cm³ to about 0.1 g/cm³, from about 0.1 g/cm³ to about 0.15 g/cm³, from about 0.15 g/cm³ to about 0.2 g/cm³, from about 0.2 g/cm³ to about 0.25 g/cm³, from about 0.25 g/cm³ to about 0.3 g/cm³, from about 0.35 g/cm³ to about 0.4 g/cm³, from about 0.4 g/cm³ to about 0.45 g/cm³, from about 0.45 g/cm³ to about 0.5 g/cm³, from about 0.5 g/cm³ to about 0.55 g/cm³, from about 0.55 g/cm³ to about 0.6 g/cm³, from about 0.6 g/cm³ to about 0.65 g/cm³, from about 0.65 g/cm³ to about 0.7 g/cm³, from about 0.7 g/cm³ to about 0.75 g/cm³, from about 0.75 g/cm³ to about 0.8 g/cm³, from about 0.8 g/cm³ to about 0.85 g/cm³, from about 0.85 g/cm³ to about 0.9 g/cm³, from about 0.9 g/cm³ to about 0.95 g/cm³, from about 0.95 g/cm³ to about 1.0 g/cm³, from about 0.1 g/cm³ to about 0.2 g/cm³, from about 0.2 g/cm³ to about 0.3 g/cm³, from about 0.3 g/cm³ to about 0.4 g/cm³, from about 0.4 g/cm³ to about 0.5 g/cm³, from about 0.5 g/cm³ to about 0.6 g/cm³, from about 0.6 g/cm³ to about 0.7 g/cm³, from about 0.8 g/cm³ to about 0.9 g/cm³, from about 0.9 g/cm³ to about 1.0 g/cm³, from about 0.50 g/cm³ to about 0.52 g/cm³, from about 0.51 g/cm³ to about 0.53 g/cm³, from about 0.52 g/cm³ to about 0.54 g/cm³, from about 0.53 g/cm³ to about 0.55 g/cm³, from about 0.54 g/cm³ to about 0.56 g/cm³, from about 0.55 g/cm³ to about 0.57 g/cm³, from about 0.56 g/cm³ to about 0.58 g/cm³, from about 0.57 g/cm³ to about 0.59 g/cm³, from about 0.58 g/cm³ to about 0.60 g/cm³, from about 0.59 g/cm³ to about 0.61 g/cm³, from about 0.60 g/cm³ to about 0.62 g/cm³, from about 0.61 g/cm³ to about 0.63 g/cm³, from about 0.62 g/cm³ to about 0.64 g/cm³, from about 0.63 g/cm³ to about 0.65 g/cm³, from about 0.64 g/cm³ to about 0.66 g/cm³, from about 0.57 g/cm³ to about 0.58 g/cm³, from about 0.58 g/cm³ to about 0.61 g/cm³, from about 0.57 g/cm³ to about 0.60 g/cm³, from about 0.58 g/cm³ to about 0.65 g/cm³, from about 0.54 g/cm³ to about 0.61 g/cm³, from about 0.54 g/cm³ to about 0.64 g/cm³, from about 0.55 g/cm³ to about 0.63 g/cm³, from about 0.54 g/cm³ to about 0.65 g/cm³, from about 0.54 g/cm³ to about 0.61 g/cm³, from about 0.5449 g/cm³ to about 0.6461 g/cm³, from about 0.5475 g/cm³ to about 0.6341 g/cm³, or from about 0.5376 g/cm³ to about 0.6052 g/cm³. In some embodiments, some dry powder nutritive media, media supplements, media subgroups, buffers or samples of the invention will have a bulk density within the standard deviations set out in Example 31 below. In some embodiments, the invention provides an OptiMEM dry powder nutritive medium with a bulk density between from about 0.57 g/cm³ to about 0.60 g/cm³, about 0.5669 g/cm³ to about 0.6048 g/cm³, or about 0.5684 g/cm³ to about 0.5970 g/cm³. In some embodiments, the invention provides a DMEM dry powder nutritive medium with a bulk density between from about 0.58 g/cm³ to about 0.65 g/cm³, about 0.5784 g/cm³ to about 0.6461 g/cm³, or about 0.5756 g/cm³ to about 0.6441 g/cm³. In some embodiments, the invention provides a IMDM dry powder nutritive medium with a bulk density between from about 0.54 g/cm³ to about 0.61 g/cm³, about 0.5449 g/cm³ to about 0.6148 g/cm³, or about 0.5376 g/cm³ to about 0.6052 g/cm³.

In one embodiment, the bulk density of a dry powder nutritive medium, media supplement, media subgroup, buffer or sample is measured by measuring out a certain volume of the dry powder, weighing the measured out dry powder and calculating the bulk density, e.g., as grams/cm³, e.g., as described in Example 30. Volume can be measured, for example, utilizing a graduated cylinder or beaker, wherein each ml represents 1 cm³.

Methods for measuring “Wet-ability” are described in Example 32. The invention provides dry powder nutritive media, media supplements, media subgroups, buffers and samples with particular or ranges of wet-ability characteristics. For example, some dry powder nutritive media, media supplements, media subgroups, buffers or samples of the invention will have a wet-ability characteristic of between from about 0.5 seconds to about 1000 seconds, about 0.5 seconds to about 500 seconds, about 0.5 seconds to about 400 seconds, about 0.5 seconds to about 300 seconds, about 0.5 seconds to about 200 seconds, about 0.5 seconds to about 100 seconds, about 0.5 seconds to about 75 seconds, about 0.5 seconds to about 50 seconds, about 0.5 seconds to about 25 seconds, about 0.5 seconds to about 20 seconds, about 0.5 seconds to about 15 seconds, about 0.5 seconds to about 10 seconds, about 0.5 seconds to about 5 seconds, about 10 seconds to about 500 seconds, about 25 seconds to about 500 seconds, about 50 seconds to about 500 seconds, about 75 seconds to about 500 seconds, about 100 seconds to about 500 seconds, about 150 seconds to about 500 seconds, about 200 seconds to about 500 seconds, about 250 seconds to about 500 seconds, about 300 seconds to about 500 seconds, about 350 seconds to about 500 seconds, about 400 seconds to about 500 seconds, about 450 seconds to about 500 seconds, about 1 second to about 10 seconds, about 1 second to about 15 seconds, about 5 seconds to about 15 seconds, about 5 seconds to about 10 seconds, about 10 seconds to about 15 seconds, about 15 seconds to about 20 seconds, about 10 seconds to about 20 seconds, about 15 seconds to about 25 seconds, about 20 seconds to about 30 seconds, about 25 seconds to about 35 seconds, about 30 seconds to about 40 seconds, about 35 seconds to about 45 seconds, about 40 seconds to about 50 seconds, about 45 seconds to about 55 seconds, about 50 seconds to about 60 seconds, about 50 seconds to about 75 seconds, about 75 seconds to about 100 seconds, about 100 seconds to about 150 seconds, about 150 seconds to about 200 seconds, about 200 seconds to about 250 seconds, about 250 seconds to about 300 seconds, about 350 seconds to about 400 seconds, about 450 seconds to about 500 seconds, about 1 second to about 12 seconds, about 1 second to about 2 seconds, about 2 seconds to about 3 seconds, about 3 seconds to about 4 seconds, about 4 seconds to about 5 seconds, about 5 seconds to about 6 seconds, about 6 seconds to about 7 seconds, about 7 seconds to about 8 seconds, about 8 seconds to about 9 seconds, about 9 seconds to about 10 seconds, about 10 seconds to about 11 seconds, about 11 seconds to about 12 seconds, about 12 seconds to about 13 seconds, about 13 seconds to about 14 seconds, about 14 seconds to about 15 seconds, about 15 seconds to about 16 seconds, about 0.5 second to about 1.5 seconds, about 1.5 second to about 2.5 seconds, about 2.5 seconds to about 3.5 seconds, about 3.5 seconds to about 4.5 seconds, about 4.5 seconds to about 5.5 seconds, about 5.5 seconds to about 6.5 seconds, about 6.5 seconds to about 7.5 seconds, about 7.5 seconds to about 8.5 seconds, about 8.5 seconds to about 9.5 seconds, about 9.5 seconds to about 10.5 seconds, about 10.5 seconds to about 11.5 seconds, about 11.5 seconds to about 12.5 seconds, about 12.5 seconds to about 13.5 seconds, about 13.5 seconds to about 14.5 seconds, about 14.5 seconds to about 15.5 seconds, about 15.5 seconds to about 16.5 seconds, about 1.2 second to about 1.7 seconds, about 1.7 second to about 2.2 seconds, about 1.2 second to about 2.2 seconds, about 1.0 second to about 1.4 seconds, about 1.0 second to about 1.2 seconds, about 1.2 second to about 1.4 seconds, about 8 seconds to about 12 seconds, about 12 seconds to about 16 seconds, about 8 seconds to about 16 seconds, or about 9 seconds to about 18 seconds.

Methods for “sieve analysis”, also known as screen analysis, are a determination of the proportions of particles in a sample which are within certain size ranges. Typically, particles subjected to sieve analysis are those of a granular material. Further, size ranges of these particles may be determined by separating the particles using sieves with different sized openings. Sieve analysis can be used to determine the relative proportions of different grain sizes as they are distributed among certain size ranges.

The following are U.S. standard sieve sizes and their corresponding open dimension.

TABLE 11

| U.S. Standard Sieve No. | Sieve Opening (mm) |
|---|---|
| 4 | 4.75 |
| 5 | 4.00 |
| 6 | 3.35 |
| 7 | 2.80 |
| 8 | 2.36 |
| 10 | 2.00 |
| 12 | 1.7 |
| 14 | 1.4 |
| 16 | 1.118 |
| 18 | 1.00 |
| 20 | 0.85 |
| 25 | 0.710 |
| 30 | 0.60 |
| 35 | 0.500 |
| 40 | 0.425 |
| 45 | 0.355 |
| 50 | 0.300 |
| 60 | 0.250 |
| 70 | 0.212 |
| 80 | 0.180 |
| 100 | 0.15 |
| 120 | 0.125 |
| 140 | 0.106 |
| 170 | 0.090 |
| 200 | 0.075 |
| 230 | 0.063 |
| 270 | 0.053 |
| 325 | 0.045 |
| 400 | 0.038 |
| 450 | 0.032 |
| 500 | 0.025 |
| 635 | 0.020 |

Other U.S. Standard Sieve Nos. that can be utilized are known to those skilled in the art. As an example, particles larger than 0.85 mm, but smaller than 2.0 mm will collect somewhere between sieve 10 and 20.

Sieve analysis is typically conducted by placing a set of sieves in a sieve shaker, e.g., as described below and setting the shaker to shake the sieves for a period of time. Sieve shakers are readily available in the art, e.g., a Retsch Sieve Shaker AS 200; a Ro-Tap Test Sieve Shaker, Tyler (e.g., model RX-29, RX-29-10, or RX-30); or a Sonic Sifter Separator, ATM, all available from VWR LABshop, Batavia, Ill.

In one embodiment, the sieve analysis of a dry powder nutritive medium, media supplement, media subgroup, buffer or sample may be measured by the following procedure.

1) Take a representative sample, e.g., that weighs about 100 g. Determine the mass of the sample accurately.

2) Weigh all empty sieves and the empty pan separately.

3) Prepare a stack of sieves, e.g., eight sizes mentioned above such as 30, 35, 45, 60, 80, 100, 120, 140 and 200. Sieves having larger opening sizes (i.e., lower numbers) are placed above the ones having smaller opening sizes (i.e., higher numbers). A pan is placed under the very last sieve (e.g., #200) to collect the portion of particles passing through the last sieve.

4) Pour the sample from step 1 into the stack of sieves from the top, place the cover, place the stack in the sieve shaker and fix the clamps, adjust the time for the shaker (e.g., between 5 minutes to 15 minutes) and then start the shaker.

5) Stop the sieve shaker and measure the mass of each sieve with any retained sample. Subtract the original weight of the sieve from the empty weight of the sieves from step 2 to calculate the mass of the retained sample in each sieve.

6) Calculate the % of particles at each mesh size by dividing the mass of the particles at the particular mesh screen by the total mass of the starting sample.

Interpretation and reporting of the results may include a graph of log sieve size versus % fines. The graph is known as a grading curve. Interpretation and reporting may include a bar graph with a bar for each sieve and pan depicting the percentage of particles collected in that sieve.

The invention provides dry powder nutritive media, media supplements, media subgroups, buffers and samples with particular sieve analysis characteristics or ranges of characteristics. For example, in some embodiments a dry powder nutritive media, media supplements, media subgroups, buffers or samples of the invention will have sieve analysis characteristics wherein between from about 20% to about 80%, from about 40% to about 80%, from about 60% to about 80%, from about 20% to about 40%, from about 20% to about 60%, from about 40% to about 60%, from about 45% to about 55%, from about 47% to about 53%, from about 49% to about 51%, from about 50% to about 51%, or from 51% to 99% of the particles by mass are within the 30 to 200 mesh range, 40 to 200 mesh range, the 60 to 200 mesh range, the 100 to 200 mesh range, the 140 to 200 mesh range, 40 to 60 mesh range, 30 to 60 mesh range, 30 to 100 mesh range, 40 to 100 mesh range, 40 to 140 mesh range, 60 to 140 mesh range, 60 to 100 mesh range, 60 to 70 mesh range, 70 to 80 mesh range, 80 to 100 mesh range, 60 to 80 mesh range, 70 to 100 mesh range, 80 to 120 mesh range, 100 to 120 mesh range, 60 to 120 mesh range, 50 to 60 mesh range, 40 to 50 mesh range, 50 to 70 mesh range, 50 to 80 mesh range, 50 to 100 mesh range, 50 to 120 mesh range, 100 to 140 mesh range or 100 mesh. In some embodiments, a dry powder nutritive media, media supplements, media subgroups, buffers or samples of the invention will have sieve analysis characteristics wherein between from about 95% to about 99%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100% of the particles are greater than or retained at the 200 mesh size (e.g., cumulative % retained). In some embodiments, a dry powder nutritive media, media supplements, media subgroups, buffers or samples of the invention will have sieve analysis characteristics wherein between from about 70% to about 100%, about 70% to about 97%, about 72% to about 97%, about 70% to about 94%, about 72% to about 94%, about 94% to about 97%, about 70% to about 80%, about 75% to about 85%, about 80% to about 90%, or about 85% to about 95%, or about 90% to about 100% of the particles are greater than or retained at the 100 mesh size.

In some embodiments, a dry powder nutritive media, media supplements, media subgroups, buffers or samples of the invention will have sieve analysis characteristics wherein between from about 60% to about 100%, about 60% to about 97%, about 62% to about 96%, about 62% to about 89%, about 89% to about 96%, about 60% to about 70%, about 65% to about 75%, about 70% to about 80%, about 75% to about 85%, about 80% to about 90%, or about 85% to about 95%, or about 90% to about 100% of the particles are greater than or retained at the 80 mesh size.

In some embodiments, a dry powder nutritive media, media supplements, media subgroups, buffers or samples of the invention will have sieve analysis characteristics wherein between from about 40% to about 95%, about 40% to about 90%, about 44% to about 90%, about 40% to about 89%, about 44% to about 89%, about 70% to about 95%, about 70% to about 90%, about 72% to about 90%, about 72% to about 89%, about 40% to about 75%, about 40% to about 72%, about 44% to about 72%, about 44% to about 75%, about 40% to about 45%, about 45% to about 50%, about 50% to about 55%, about 55% to about 60%, about 60% to about 65%, about 65% to about 70%, about 70% to about 75%, or about 75% to about 80%, about 80% to about 85%, about 85% to about 90% or about 90% to about 95% of the particles are greater than or retained at the 60 mesh size.

In some embodiments, a dry powder nutritive media, media supplements, media subgroups, buffers or samples of the invention will have sieve analysis characteristics wherein between from about 10% to about 38%, about 12% to about 38%, about 10% to about 35%, about 12% to about 35%, about 10% to about 15%, about 15% to about 20%, about 20% to about 25%, about 25% to about 30%, about 30% to about 35%, or about 35% to about 40% of the particles are greater than or retained at the 45 mesh size.

In some embodiments, a dry powder nutritive media, media supplements, media subgroups, buffers or samples of the invention will have sieve analysis characteristics wherein between from about 7% to about 31% retained at the 30 mesh size and above; about 18% to about 73% retained at the 45 mesh size and above; about 33% to about 92% retained at the 60 mesh size and above; about 56% to about 97% retained at the 80 mesh size and above; about 68% to about 98% retained at the 100 mesh size and above; about 96% to about 100% retained at the 200 mesh size and above; about 0.15% to about 3.7% retained below the 200 mesh size.

In some embodiments, between from about 40% to about 60% of the particles by mass will be between the 60-100 mesh range. In some embodiments, between from about 40% to about 60% of the particles by mass will be between the 40-100 mesh range. In some embodiments, between from about 40% to about 60% of the particles by mass will be between the 60-140 mesh range. In some embodiments, between from about 40% to about 60% of the particles by mass will be between the 50-120 mesh range. In some embodiments, between from about 40% to about 60% of the particles by mass will be between the 50-100 mesh range. In some embodiments, between from about 40% to about 60% of the particles by mass will be between the 60-120 mesh range.

In some embodiments, the dry powder nutritive media, media supplements, media subgroups, buffers or samples of the invention will have sieve analysis characteristics wherein equal to or less than 0.001%, 0.01%, 0.1%, 0.2%, 0.3%, 0.4%, 0.5%, 0.6%, 0.7%, 0.8%, 0.9%, 1%, 2%, 3%, 4%, 4.1%, 5%, 6%, 7%, 8%, 9%, or 10%, or between from about 0.001% to about 0.005%, from about 0.001% to about 0.0025%, from about 0.0025% to about 0.005%, from about 0.005% to about 0.01%, from about 0.005% to about 0.0075%, from about 0.0075% to about 0.01%, from about 0.01% to about 0.05%, from about 0.01% to about 0.025%, from about 0.025% to about 0.05%, from about 0.05% to about 0.1%, from about 0.05% to about 0.075%, from about 0.075% to about 0.1%, from about 0.1% to about 0.5%, from about 0.1% to about 0.25%, from about 0.25% to about 0.5%, from about 0.5% to about 1%, from about 0.5% to about 0.75%, from about 0.75% to about 1%, from about 1% to about 10%, from about 2% to about 10%, from about 3% to about 10%, from about 4% to about 10%, from about 5% to about 10%, from about 6% to about 10%, from about 7% to about 10%, from about 8% to about 10%, from about 9% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 2% to about 8%, from about 3% to about 7%, from about 4% to about 6%, from about 5% to about 6%, from about 4% to about 5%, from about 3% to about 4%, from about 2% to about 3%, from about 6% to about 7%, from about 7% to about 8%, from about 8% to about 9%, from about 3% to about 5%, from about 5% to about 7%, from about 6% to about 8%, or from about 7% to about 9% of the particles, by mass, pass through the 140, 170, 200, 230, 270, 325, 400, 450, 500 or 635 mesh.

Mass flow rate is the movement of mass per time. Its unit is mass over time, e.g., kilogram per second. The formula m=pVA can be used wherein: m=mass flow rate; p=density; V=velocity; A=area flow. The mass flow rate can also be calculated by multiplying the volume flow rate by the density, e.g., m=pQ wherein: p=density and Q=volume flow-rate. Mass flow rate can be determined by various methods known in the art utilizing equipment readily available in the art, e.g., as described in U.S. Pat. Nos. 6,176,647 and 4,109,524. In some embodiments, the flow rate may be determined in a defined system.

Some embodiments of the invention provide dry powder nutritive media, media supplements, media subgroups, buffers and samples with particular flow rates or ranges of flow rates. For example, some dry powder nutritive media, media supplements, media subgroups, buffers or samples of the invention will have a flow rate between from about 0.1 kg/sec to about 20 kg/sec; from about 1 kg/sec to about 20 kg/sec; from about 10 kg/sec to about 20 kg/sec; from about 0.1 kg/sec to about 1 kg/sec; from about 0.1 kg/sec to about 5 kg/sec; from about 0.1 kg/sec to about 10 kg/sec; from about 0.5 kg/sec to about 5 kg/sec; from about 1 kg/sec to about 5 kg/sec; from about 2 kg/sec to about 5 kg/sec; from about 3 kg/sec to about 5 kg/sec; from about 4 kg/sec to about 5 kg/sec; from about 1 kg/sec to about 4 kg/sec; from about 1 kg/sec to about 3 kg/sec; from about 1 kg/sec to about 2 kg/sec; from about 2 kg/sec to about 3 kg/sec; from about 2 kg/sec to about 4 kg/sec; from about 3 kg/sec to about 4 kg/sec; from about 1.5 kg/sec to about 2.5 kg/sec; from about 1.5 kg/sec to about 2 kg/sec; from about 2 kg/sec to about 2.5 kg/sec; from about 2 kg/sec to about 2.25 kg/sec; from about 1.75 kg/sec to about 2 kg/sec; from about 1.75 kg/sec to about 2.25 kg/sec; about 1.0 kg/sec; about 1.1 kg/sec; about 1.2 kg/sec; about 1.3 kg/sec; about 1.4 kg/sec; about 1.5 kg/sec; about 1.6 kg/sec; about 1.7 kg/sec; about 1.8 kg/sec; about 1.9 kg/sec; about 2.0 kg/sec; about 2.1 kg/sec; about 2.2 kg/sec; about 2.3 kg/sec; about 2.4 kg/sec; about 2.5 kg/sec; about 2.6 kg/sec; about 2.7 kg/sec; about 2.8 kg/sec; about 2.9 kg/sec; or about 3.0 kg/sec.

The “angle of repose” is an engineering property of particulate solids and is sometimes used as a synonym for the tipping point. One example of the angle of repose is when bulk particles are poured onto a horizontal surface and forms a conical pile. The angle between the edge of the pile and the horizontal surface is known as the angle of repose. This angle can be measured, e.g., with a protractor. The angle of repose can be related to the density, surface area, and coefficient of friction of the material. Material with a low angle of repose typically forms flatter piles than material with a high angle of repose.

There are numerous methods for measuring angle of repose. An alternative measurement, useful for many of the same purposes, is testing with a shear cell. Additionally an angle of repose can be measured using a Johanson Indicizer (Johanson Innovations, San Luis Obispo, Calif.) by following the manufacture's instructions, e.g., The Indicizer Application Guide, ver 1.20. The angle of repose segregation occurs whenever solids slide across each other as they are introduced into a container. Material producing a steeper angle of repose holds back and allows a material (with a less steep angle of repose) to slide freely to the bottom of the slope or pile. The angle of repose segregation can be measured using a Johanson Indicizer (Johanson Innovations, San Luis Obispo, Calif.) by following the manufacture's instructions.

The invention further provides dry powder nutritive media, media supplements, media subgroups, buffers and samples with particular angles of repose or ranges of angles of repose. For example, some dry powder nutritive media, media supplements, media subgroups, buffers or samples of the invention will have an angle of repose between from about 10 to about 45 degrees; about 20 to about 45 degrees; from about 25 to about 30 degrees; from about 25 to about 40 degrees; from about 25 to about 45 degrees; from about 30 to about 45 degrees; from about 35 to about 45 degrees; from about 40 to about 45 degrees; from about 30 to about 40 degrees; from about 30 to about 35 degrees; from about 35 to about 40 degrees; from about 25 to about 35 degrees; from about 25 to about 27 degrees; from about 26 to about 27 degrees; from about 27 to about 28 degrees; from about 28 to about 29 degrees; from about 29 to about 30 degrees; from about 30 to about 31 degrees; from about 31 to about 32 degrees; from about 32 to about 33 degrees; from about 33 to about 34 degrees; from about 34 to about 35 degrees; about 20 degrees; about 21 degrees; about 22 degrees; about 23 degrees; about 24 degrees; about 25 degrees; about 26 degrees; about 27 degrees; about 28 degrees; about 29 degrees; about 30 degrees; about 31 degrees; about 32 degrees; about 33 degrees; about 34 degrees; about 35 degrees; about 36 degrees; about 37 degrees; about 38 degrees; about 39 degrees; or about 40 degrees.

### Embodiments Related to Spray-Drying

In another aspect of the invention, powdered samples including nutritive media, media supplements, media subgroups, buffers and samples of interest may be prepared by spray-drying. In this aspect of the invention, the nutritive medium, media supplement, media subgroups, buffer or sample of interest in its liquid form is placed into a spray-drying apparatus; this liquid is then converted into the corresponding powder by spraying the solution into a chamber in the apparatus under appropriate conditions to produce the powders, such as under controlled temperature and humidity, until a powder is formed. In some situations, it may be desirable or advantageous to spray-dry complex mixtures of two or more of the media, media supplements, media subgroups, buffers, samples or components or combinations thereof, e.g., as described herein. For example, liquid nutritive media containing animal sera at a desired concentration, or liquid animal sera containing nutritive media components at desired concentrations, may be mixed and then prepared as spray-dried powders according to the methods of the invention. Spray drying or other methods for obtaining powders may provide powder ingredients for agglomeration.

In a typical spray-drying approach, the liquid sample including nutritive media, media supplements, media subgroups and buffers are aspirated into the apparatus and are atomized into a spray with a rotary- or nozzle-type atomizer. The resulting atomized liquid spray is then mixed with a gas (e.g., nitrogen or more preferably air) and sprayed into a drying chamber under conditions sufficient to promote evaporation and production of a powdered product. In a preferred aspect of the invention, these conditions may comprise electronic control of the temperature and humidity within the chamber such that final drying of the product is promoted. Under these conditions, the solvent in the liquid evaporates in a controlled manner, thereby forming free-flowing particles (i.e., powder) of the sample of interest (e.g. nutritive media, media supplements, media subgroups or buffers). The powder is then discharged from the drying chamber, passed through a cyclone separation system or one or more filters (such as the mesh screens described herein for fluid bed preparation) and collected for further processing (e.g., packaging, sterilization, etc.). In some applications, particularly when producing powders from heat-sensitive formulations of nutritive media, media supplements, media subgroups, buffers or samples, the spray-drying apparatus may be combined with a fluid bed apparatus integrated within the drying chamber, which allows the introduction of agglomerating solvents such as those described herein into the spray-dried powder to produce agglomerated spray-dried powdered nutritive media, media supplements, media subgroups, buffers or samples. Such combination of processes may facilitate removal or inactivation of toxins or adventitious agents in the sample.

Apparatuses for producing particulate materials from liquid materials by spray-drying (with or without integrated fluid bed technology) are available commercially (e.g., from Niro, Inc./Aeromatic-Fielder; Columbia, Md.), and are described, for example, in the “Spray Drying,” “Powdered Pharmaceuticals by Spray Drying” and “Fresh Options in Drying” technical brochures of Niro, Inc./Aeromatic-Fielder, the disclosures of which are incorporated by reference herein in their entireties. According to this manufacturer, such apparatuses have been used to prepare powders of various materials, including dairy products, analgesics, antibiotics, vaccines, vitamins, yeasts, vegetable protein, eggs, chemicals, food flavorings and the like. In the present invention, spray-drying has been found to be particularly useful for the preparation of powdered media supplements, such as sera and in particular those sera described herein, most particularly human and bovine sera (such as fetal bovine serum and calf serum). It is also particularly suited to prepare powdered pharmaceutical or clinical compositions or solutions.

In the practice of this aspect of the invention, the liquid sample (e.g. nutritive media, media supplements, media subgroups, buffers or pH-adjusting agents) should be sprayed into the chamber through the atomizer at a spray rate of about 25-100 g/min, preferably at a spray rate of about 30-90 g/min, 35-85 g/min, 40-80 g/min, 45-75 g/min, 50-75 g/min, 55-70 g/min, or 60-65 g/min, and more preferably at about 65 g/min. The inlet air temperature in the atomizer is preferably set at about 100-300° C., more preferably at about 150-250° C., and most preferably at about 200° C., with an outlet temperature of about 50-100° C., more preferably about 60-80° C., and most preferably about 70° C. Air flow in the atomizer is preferably set at about 50-100 kg/hr, more preferably about 75-90 kg/hr, and most preferably about 80.0 kg/hr, at a nozzle pressure of about 1-5 bar, more preferably about 2-3 bar, and most preferably about 2.0 bar. These conditions and settings have been found in the present invention to be preferable for production of a variety of nutritive media, media supplements, media subgroups and buffer powders by spray-drying, particularly for the production of the herein-described powdered sera. Following drying, the spray-dried powdered sample (e.g. nutritive media, media supplements, media subgroups or buffers) may be collected in the drying chamber through a cyclone system or one or more filters, preferably such as those described herein for fluid bed technology. In some instances, a fluid bed apparatus may be used wherein the airflow may be 60-120 CMH, e.g., for bench-, process- and production-scale apparatuses. For example, this setting of 60-120 CMH can be used with the methods described in Example 1 below.

Following this preparation, the powders of the invention prepared by the herein-described fluid bed and/or spray-drying methods (or combinations thereof) have altered physical characteristics from the starting powders or from powdered media, supplements, subgroups and buffers prepared by lyophilizing the corresponding liquids. For example, non-processed or lyophilized powders often produce significant dust when used, and dissolve poorly or slowly in various solvents, while agglomerated or some spray-dried powders are substantially dust-free and/or dissolve rapidly. Typically, the powdered media, media supplements, media subgroups, buffers, and pharmaceutical or clinical compositions of solutions of the invention will exhibit both reduced dusting and more rapid dissolution than their powdered counterparts prepared by standard techniques such as ball-milling. In some powders which are substantially dust-free but which may not demonstrate enhanced dissolution, the powders may be rapidly dissolved by rapid mechanical solvation of the powder, such as using a mechanical impeller, or by first providing a solvent mist over the powder such as by spray solvation. Moreover, in accordance with the invention, the powdered samples produced have reduced, substantially reduced, or inactivated or eliminated adventitious agents and/or toxins. Such reagents advantageously provide components for manipulating or growing cells which may be used in industrial or biomedical processes and provide pharmaceutical or clinical compositions or solutions important to the medical field.

In one aspect of the invention, the spray-drying and agglomeration approaches described herein may be combined to produce agglomerated spray-dried samples (e.g. nutritive media, media supplement, media subgroup and buffer powders). In this aspect, a powdered medium, supplement, subgroup, buffer or sample that has been prepared by spray-drying may, after having been spray-dried, then be agglomerated with a solvent (such as those described herein) to further improve the performance and physical characteristics of the resultant product (e.g. a medium, supplement, subgroup or buffer). For example, an animal serum powder may be prepared by spray-drying liquid animal serum as described herein, and this spray-dried serum powder may then be mixed into dry powder nutritive media (prepared by spray-drying or by standard techniques such as ball-milling); this mixed powder may then be agglomerated as described herein. Alternatively, a spray-dried nutritive medium, medium supplement, medium subgroup or buffer powder may be agglomerated as described herein, to improve the dissolution properties of the powder. This approach may be particularly advantageous when spray-drying liquids with low (about 1-10%) solids content, such as liquid animal sera. As one of ordinary skill will appreciate, these approaches will facilitate preparation of a large batch of one or more components (e.g., sera or other media supplements) to be used as a stock for addition to a powdered medium, supplement, subgroup or buffer at a desired concentration, while also obtaining the herein-described benefits of agglomeration. In addition, this approach may reduce inter-lot variability which may be a problem with certain media supplements (particularly animal sera) and will facilitate reduction of toxins and/or adventitious agents in accordance with the invention.

The agglomerated and/or spray-dried powdered samples, particularly nutritive media, media supplements, media subgroups, buffers, or pharmaceutical or clinical compositions or solutions prepared as described herein, may then be packaged, for example into containers such as vials, tubes, bottles, bags, pouches, boxes, cartons, drums and the like, prior to or following optimal sterilization as described herein. In one such aspect of the invention, the powdered sample including media, media supplements, media subgroups or buffers may be packaged into a compact, vacuum-packed form, such as that known in the art as a “brick-pack” wherein the powder is packaged into a flexible container (such as a bag or a pouch) that is sealed while being evacuated. Other such packages may advantageously comprise one or more access ports (such as valves, luer-lock ports, etc.) allowing the introduction of a solvent (e.g., water, sera, media or other aqueous or organic solvents or solutions) directly into the package to facilitate rapid dissolution of the powder. In a related aspect, the package may comprise two or more adjacent compartments, one or more of which may contain one or more of the dry powder samples (e.g. media, media supplements, media subgroups or buffers) of the invention and one or more other of which may contain one or more aqueous or organic solvents which may be sterile. In this aspect, the dry powder may then be dissolved by simply removing or breaking the barrier between the compartments, ideally without loss of sterility, to allow admixture of the powder and the solvent such that the powder dissolves and produces a sterile sample such as nutritive medium, medium supplement, medium subgroup or buffer at a desired concentration.

### Agglomeration of Lipid or Non-Aqueous Solutes

A particular advantage of some embodiments of the present invention is methods that accomplish agglomeration of lipids and ingredients not sufficiently soluble in common aqueous solvent preparations into dry powdered media. Conventionally, such ingredients have been added in less than optimal procedures, for example, as concentrates dissolved in organic solvent. By the methods of the present invention, dry powder media that contain desired non-aqueous solutes are achievable.

Examples of such non-aqueous solutes are: fatty acids, neutral fats waxes, steroids and steroidal compounds, phosphatides, glycolipids (e.g., sphingosines, cerebrosides, ceramides, gangliosides), lipoproteins, phospholipids, phosphoglycerides (e.g., ethanolamines such as phosphatidyl ethanolamine or ethanolamine phosphoglyceride, cholines such as phosphatidyl choline or choline phosphoglyceride), lipoamino acids, cardiolipin and related compounds, plasmalogens, sterols (e.g., cholesterol, lanosterol) terpenes, fat soluble vitamins (e.g., vitamin A and its vitamers, vitamin E and its vitamers, vitamin K and its vitomers, Vitamin D and its vitamers. Fat soluble proteins are also examples of lipids as used in media in aspects of the present invention.

One aspect of the present invention comprises methods for incorporating one or more lipids into a dry powder. Lipids may be introduced by delivering a solvent containing the lipid(s) to an agglomeration bed. For example, an organic solvent containing the lipid(s) may be introduced into the agglomeration apparatus. Preferably, a solvent of low toxicity is used. Depending on the cell type for which the medium is being prepared, solvents such as alcohols, e.g., methanol or ethanol may be preferred. The solvents may neatly dissolve the lipid component(s) or may dissolve the component(s) in the presence of other solvent(s) or solute(s). After dissolution, another component, e.g., another lipid or solvent may be added.

The solvent mixture to be introduced into the apparatus may be introduced before after and/or during delivery of another solvent or mixture. The another solvent or mixture may contain some of the same solvent(s) or ingredient(s) as the solvent mixture. Thus a solvent mixture may contain any ratio of solvents. For example, preferred mixtures of solvents to be used in the solvent mixture may contain water and alcohol, more preferably, e.g., for most mammalian cells, water and ethanol. The ratio will be selected according to the parameters described herein and for example may be as little as about e.g., 1, 5, 7, 10, 15, 20, 25, 30, 33, 40, 50, 60, 67, 70, 75 or as much as 80, 85, 90, 95 98 or 99% ethanol (v/v) the remainder being predominantly water. Occasionally lipids may themselves act partially as solvents. Other organic solvents such as those exemplified herein may be used in similar ratios. One of ordinary skill will appreciate that different lipids may require different solvents, solvent mixtures and ratios of solvent mixtures for the agglomeration process. When plural organic solvents are used they may be used sequentially or may be mixed together in liquid form. The concentration of each may be similar to the percentages exemplified above.

Unexpectedly, the inventors have found that a mixture of water and ethanol works better than either alone for delivering lipids to the dry powder agglomeration. It is believed that parameters discussed herein, e.g., relating to solubility temperature and drying time are behind this unexpected finding. Following the example of ethanol and water, the inventors believe that one of ordinary skill will appreciate the benefits and compromises imposed by other mixtures of solvents and solutes.

The invention also includes aspects wherein lipids are agglomerated into the dry powder after modification to enhance solubility in water. For example the lipid may be rendered ionic by conversion to a salt, e.g., a fatty acid may be saponified. One of ordinary skill will appreciate other means such as hydroxylation or esterification that will improve solubility in water. The lipid whose solubility has been improved may be added in aqueous solvent of may be added in a mixture of solvents. For example, improving solubility may allow a lesser amount of organic solvent to be used.

Another aspect of the present invention involves use of chemicals that can associate or complex with lipid structures to result in lipid solubility in aqueous environments. Such interactions may be due to micelle formation where the hydrophobic part of the molecule causing formation of the micelle will contain the lipid moiety and the hydrophilic part of the molecule causing formation of the micelle will dissolve in an aqueous environment resulting in lipid solubilization in an aqueous environment. (Example: Pluronic F-68 or other surface-active agents). Other similar interactions may result from compounds such as cyclodextrins that can solubilize (partition, physical complexation) lipid within the cyclodextrin structure and maintain that physical complexation upon addition to aqueous environments thus effecting solubility of said lipid in said aqueous environment. (Example: B-methyl cyclodextrin).

### Sterilization and Packaging

The invention also provides methods for sterilizing the nutritive media, media supplements, media subgroups and buffers of the invention, as well as for sterilizing powdered nutritive media, media supplements, media subgroups and buffers prepared by standard methods such as ball-milling or lyophilization. The invention also provides additional methods for sterilizing or substantially sterilizing the samples including nutritive media, media supplements, media subgroups and buffers of the invention. Such additional methods may include filtration, heat sterilization, irradiation or other chemical or physical methods. Preferably, nutritive media, media supplements, media subgroups or buffers (preferably powders prepared as described herein by spray-drying and/or by agglomeration) may be irradiated under conditions favoring sterilization. Since nutritive media, media supplements, media subgroups and buffers are usually prepared in large volume solutions and frequently contain heat labile components, they are not amenable to sterilization by irradiation or by heating. Thus, nutritive media, media supplements, media subgroups and buffers are commonly sterilized by contaminant-removal methods such as filtration, which significantly increases the expense and time required to manufacture such media, media supplements, media subgroups and buffers.

Powdered nutritive media, media supplements, media subgroups and buffers prepared according to the methods of the invention (e.g., by spray-drying of liquid media, media supplements, media subgroups or buffers, or by agglomeration of powdered media, media supplements, media subgroups or buffers), or by standard methods such as ball-milling (of powdered components) or lyophilization (of liquid forms of the media, supplements, subgroups or buffers), however, can be sterilized by less expensive and more efficient methods. For example, powdered nutritive media, media supplements, media subgroups or buffers (prepared as described herein by spray-drying or lyophilization of a liquid form, or by agglomeration of a powdered form, of the media, supplements, subgroups or buffers) may be irradiated under conditions favoring sterilization of these powders. Preferably, this irradiation is accomplished in bulk (i.e., following packaging of the sample, nutritive media, media supplement, media subgroup or buffer), and most preferably this irradiation is accomplished by exposure of the bulk packaged sample, media, media supplement, media subgroup or buffer of the invention to a source of gamma rays under conditions such that bacteria, fungi, spores or viruses that may be resident in the powdered sample media, media supplements, media subgroups or buffers are inactivated (i.e., prevented from replicating). Alternatively, irradiation may be accomplished by exposure of the sample, powdered media, media supplement, media subgroup or buffer, prior to packaging, to a source of gamma rays or a source of ultraviolet light. The sample, media, media supplements, media subgroups and buffers of the invention may alternatively be sterilized by heat treatment (if the subgroups or components of the sample, nutritive media, media supplement, media subgroup or buffer are heat stable), for example by flash pasteurization or autoclaving. As will be understood by one of ordinary skill in the art, the dose of irradiation or heat, and the time of exposure, required for sterilization will depend upon the bulk of the materials to be sterilized, and can easily be determined by the ordinarily skilled artisan without undue experimentation using art-known techniques, such as those described herein.

In a particularly preferred aspect of the invention, the bulk sample (e.g. nutritive media, media supplements, media subgroups or buffers) (which are preferably in powdered form) are exposed to a source of irradiation (e.g., γ) at a total dosage of about 10-100 kilograys (kGy), preferably a total dosage of about 15-75 kGy, 15-50 kGy, 15-40 kGy, 20-40 kGy or 25-45 kGy, more preferably a total dosage of about 20-30 kGy, and most preferably a total dosage of about 25-35 kGy, for about 1 hour to about 7 days, more preferably about 1 hour to about 5 days, 1 hour to about 3 days, about 1-24 hours or about 1-5 hours, and most preferably about 1-3 hours (“normal dose rate”). Alternatively, the bulk powders of the invention or sample may be sterilized at a “slow dose rate” of a total cumulative dosage of about 25-100 kGy over a period of about 1-5 days. During irradiation, the sample including nutritive media, media supplements, media subgroups or buffers (which are preferably in powdered form) are preferably stored at a temperature of about −70° C. to about room temperature (about 20-25° C.), most preferably at about −70° C. One of ordinary skill will appreciate, of course, that radiation dose and exposure times may be adjusted depending upon the bulk and/or mass of material to be irradiated; typical optimal irradiation dosages, exposure times and storage temperatures required for sterilization of bulk powdered materials by irradiation or heat treatment are well-known in the art.

Following sterilization, unpackaged samples including nutritive media, media supplements, media subgroups and buffers may be packaged under aseptic conditions, for example by packaging into containers such as sterile tubes, vials, bottles, bags, pouches, boxes, cartons, drums and the like, or in the vacuum packaging or integrated powder/solvent packaging described herein. Sterile packaged samples such as media, media supplements, media subgroups and buffers may then be stored for extended periods of time as described herein.

### Use of the Nutritive Media, Media Supplements, Media Subgroups and Buffers

The present invention thus provides powdered nutritive media, media supplements, media subgroups and buffers that are readily soluble in a rehydrating solvent and that are substantially dust free. For use, the agglomerated or spray-dried media, media supplement, media subgroup or buffer may be hydrated (or “reconstituted”) in a volume of a solvent sufficient to produce the desired nutrient, electrolyte, ionic and pH conditions required for the particular use of the solvated media, media supplement, media subgroup or buffer. This reconstitution is particularly facilitated in the present invention, since the present media, media supplements, media subgroups and buffers will rapidly go into solution and will produce little if any dust or insoluble material, unlike lyophilized or ball-milled nutritive media, media supplements, media subgroups or buffers.

Preferred solvents for use in reconstituting the powdered nutritive media, media supplements, media subgroups, buffers or samples of the invention include, but are not limited to, the solvents described herein such as water (most particularly distilled and/or deionized water), serum (particularly bovine or human serum and most particularly fetal bovine serum or calf serum), organic solvents (particularly dimethylsulfoxide, acetone, ethanol and the like), or any combination thereof, any of which may contain one or more additional components (e.g., salts, polysaccharides, ions, detergents, stabilizers, etc.). For example, powdered media supplements (such as animal sera) and buffers are preferably reconstituted in water to a 1× final concentration, or optionally to a higher concentration (e.g., 2×, 2.5×, 5×, 10×, 20×, 25×, 50×, 100×, 500×, 1000×, etc.) for the preparation of stock solutions or for storage. Alternatively, powdered culture media may be reconstituted in a solution of media supplements (e.g., sera such as FBS) in water, such as those solutions wherein the media supplement is present at a concentration, for example, of 0.5%, 1%, 2%, 2.5%, 5%, 7.5%, 10%, 15%, 20%, 25%, 50%, or higher, vol/vol in the water.

Reconstitution of the powdered sample (e.g. nutritive media, media supplements, media subgroups or buffers) is preferably accomplished under aseptic conditions to maintain the sterility of the reconstituted sample, although the reconstituted sample may be sterilized, preferably by filtration or other sterilization methods that are well-known in the art, following rehydration. Following their reconstitution, media, media supplements, media subgroups and buffers or other samples should be stored at temperatures below about 10° C., preferably at temperatures of about 0-4° C., until use.

The reconstituted nutritive media, media supplements, media subgroups and buffers may be used to culture or manipulate cells according to standard cell culture techniques which are well-known to one of ordinary skill in the art. In such techniques, the cells to be cultured are contacted with the reconstituted media, media supplement, media subgroup or buffer of the invention under conditions favoring the cultivation or manipulation of the cells (such as controlled temperature, humidity, lighting and atmospheric conditions). Cells which are particularly amenable to cultivation by such methods include, but are not limited to, bacterial cells, fish cells, yeast cells, plant cells and animal cells. Such bacterial cells, yeast cells, plant cells and animal cells are available commercially from known culture depositories, e.g., American Type Culture Collection (Manassas, Va.), Invitrogen (Carlsbad, Calif.) and others that will be familiar to one of ordinary skill in the art. Preferred animal cells for cultivation by these methods include, but are not limited to, insect cells (most preferably Drosophila cells, Spodoptera cells and Trichoplusa cells), nematode cells (most preferably C. elegans cells) and mammalian cells (most preferably CHO cells, COS cells, VERO cells, BHK cells, AE-1 cells, SP2/0 cells, L5.1 cells, hybridoma cells and human cells, such as 293 cells, PER-C6 cells and HeLa cells), any of which may be a somatic cell, a germ cell, a normal cell, a diseased cell, a transformed cell, a mutant cell, a stem cell, a precursor cell or an embryonic cell, embryonic stem cells (ES cells), cells used for virus or vector production (i.e. 293, PerC 6), cells derived from primary human sites used for cell or gene therapy, i.e., lymphocytes, hematopoietic cells, other white blood cells (WBC), macrophage, neutriophils, dendritic cells, and any of which may be an anchorage-dependent or anchorage-independent (i.e., “suspension”) cell. The invention also pertains to manipulation or cultivation of cells and/or tissues for tissue or organ transplantation or engineering, i.e. hepatocyte, pancreatic islets, osteoblasts, osteoclasts/chondrocytes, dermal or muscle or other connective tissue, epithelial cells, tissues like keratinocytes, cells of neural origin, cornea, skin, organs, and cells used as vaccines, i.e. blood cells, hematopoietic cells other stem cells or progenitor cells, and inactivated or modified tumor cells of various histotypes.

The invention further provides methods of manipulating or culturing one or more cells comprising contacting said cells with the cell culture reagents of the invention, particularly nutritive media, media supplement, media subgroup or buffer and incubating said cell or cells under conditions favoring the cultivation or manipulation of the cell or cells. Any cell may be cultured or manipulated according to the present methods, particularly bacterial cells, yeast cells, plant cells, animal cells and other cells or cell lines described herein. Cells cultured or manipulated according to this aspect of the invention may be normal cells, diseased cells, transformed cells, mutant cells, somatic cells, germ cells, stem cells, precursor cells or embryonic cells, any of which may be established cell lines or obtained from natural sources.

Nutritive media, media supplements and media subgroups produced by the present methods are any media, media supplement or media subgroup (serum-free or serum-containing) which may be used to manipulate or support the growth of a cell, which may be a bacterial cell, a fungal cell (particularly a yeast cell), a plant cell or an animal cell (particularly an insect cell, a nematode cell or a mammalian cell, most preferably a human cell), any of which may be a somatic cell, a germ cell, a normal cell, a diseased cell, a transformed cell, a mutant cell, a stem cell, a precursor cell or an embryonic cell. Preferred such nutritive media include, but are not limited to, cell culture media, most preferably a bacterial cell culture medium, plant cell culture medium or animal cell culture medium. Preferred media supplements include, but are not limited to, undefined supplements such as extracts or hydrolysates of bacterial, animal or plant cells, glands, tissues or organs (particularly bovine pituitary extract, bovine brain extract and chick embryo extract); and biological fluids or blood derived products (particularly animal sera, and most preferably bovine serum (particularly fetal bovine, newborn calf or normal calf serum), horse serum, porcine serum, rat serum, murine serum, rabbit serum, monkey serum, ape serum or human serum, any of which may be fetal serum) and extracts thereof (more preferably serum albumin and most preferably bovine serum albumin or human serum albumin). Medium supplements may also include defined replacements such as LipoMAX®, OptiMAb®, Knock-Out™ SR (each available from Life Technologies, Inc., Rockville, Md.), and the like, which can be used as substitutes for the undefined media supplements described above. Such supplements may also comprise defined components, including but not limited to, hormones, cytokines, neurotransmitters, lipids, attachment factors, proteins, amino acids and the like.

Nutritive media can also be divided into various subgroups (see for example U.S. Pat. No. 5,474,931) which can be prepared by, and used in accordance with, the methods of the invention. Such subgroups can be combined to produce the nutritive media of the present invention. In another aspect of the invention, individual ingredients (or combinations of ingredients) particularly ingredients of animal origin may be used in the invention. Such ingredients or samples may then be used in the preparation of any nutritive media, media supplements, media subgroups or buffers.

The dry powdered media of the present invention, upon being reconstituted with a solvent, can be used for the growth and/or cultivation of organisms such as, e.g., filamentous fungi, transgenic plants (e.g., tobacco, rice and Lemna), lichens and algae, and cells derived from any of the aforementioned organisms. In addition, the aforementioned organisms and cells may be grown and/or cultivated in media produced by any of the methods set forth in U.S. Patent Application Publication Nos. 2001-10049141, 2002-0015999, 2003-0153079, and 2004-0022666, the contents of which are hereby incorporated by reference in their entireties.

### Supplements and Supplement Feeds

This section provides various embodiments of the invention related to supplements in addition to the embodiments described elsewhere herein. In some aspects of the invention a supplement feed formulation is chosen. The skilled artisan may use knowledge available in the art to choose which ingredients are desired. Preferably, analytical methods such as those used to analyze spent media are employed to arrive at the supplementation formulation.

Preferably the formulation includes one or more amino acids. Preferably a salt of an amino acid is used for the dry format formulation. Preferably the salt is a sodium salt.

Preferably monobasic and dibasic phosphate salts are used. A preferred cation is sodium. Preferably the monobasic and dibasic salts are provided such that a resultant pH, for example, about 8 pH is obtained. Depending on the formulation, while the ratio of monobasic to dibasic salts may be dictated by desired pH, different total salt concentrations should be tried to optimize solubility, especially when concentrated or highly concentrated supplements are to be used. pH can also be confirmed when assessing the salt concentration. When an amino acid is not provided as a salt, preferably the pH effect of the acid is countered by a tribasic phosphate, preferably a sodium tribasic phosphate. While sodium is preferred as a cation other metals, such as potassium, calcium, magnesium may be used. If a specific counterion is desired, it may be available as a phosphate salt. Preferably the supplement powder dissolves rapidly. Preferably the supplement can be prepared and used as a highly concentrated mixture, for example, with one or more components at a concentration about 2 or more, preferably 3, 5, 8, 10, 12, 15, 20, 25, 50, 75, 85, 95, or even about 100 or more times the concentration of that component in the medium being supplemented. The concentration of each desired ingredient of the supplement can be independently selected. Preferably the supplement is prepared simply by reconstituting with water under sterile conditions for addition to a bioreactor. Preferably sterilization is provided by filtration.

A supplement may have no ingredients in common with the medium being supplemented or may have one or more ingredients in common. The supplement may differ from the medium being supplemented in at least one manner, such as a different concentration of one or more ingredients, for example a different ratio of two ingredients, a different ingredient mix, additional ingredients or omitted ingredients in the supplement. For example a supplement may omit salts to the extent feasible and may contain, for example, significantly enhanced concentrations of growth factors or amino acids. A preferred supplement formulation contains at least 2, more preferably 3, but perhaps at least 4, 5, 6, 7, 8, 9, 10, 11, 12, 13, 14, 15, or more amino acids including salts or dimers thereof.

In some embodiments, feed supplements of the invention are utilized to supplement a medium that has or is being used to culture cells, e.g., as the cells are cultured, some ingredients are removed from the medium by the cells. In some embodiments of the invention, the feed supplement is used, inter alia, to replace some or all of these ingredients. In some embodiments, the supplement contains the majority of the ingredients that were in the original medium to be supplemented, but the feed medium is lacking at least one ingredient. In some embodiments, the feed supplement is lacking 1, 2, 3, 4, 5, 6, 7, 8, 9, 10, 11, 12, 13, 14, 15, or more ingredients as compared to the concentration in the original culture medium being supplemented. In some embodiments, the feed supplement is added in a concentrated form, e.g., at 2×, 3×, 4×, 5×, 6×, 7×, 8×, 9×, 10×, 15×, 20×, 30×, 40×, 50×, 100×, 200×, 300×, 400×, 500× or 1000×. By concentrated form is meant that at least one of the ingredients in the feed supplement is at a concentration higher than what is the desired concentration in the culture medium. In some embodiments, ingredients for a feed supplement may be divided into multiple feed supplement media, e.g., based upon compatible subgroups. For examples of compatible subgroups and related considerations, see U.S. Pat. No. 5,681,748.

The term “concentrate feed supplement medium” or “concentrated feed supplement medium” are used interchangeably and refer to a medium that comprises at least one component that is at a concentration higher than that desired in the cell culture medium to be supplemented.

In some embodiments of the invention, a feed supplement is in a dry powder form, an agglomerated dry powder form, or a liquid form. If in a dry form, the feed supplement is typically reconstituted prior to the feeding. However, the invention does contemplate the addition of the dry supplement directly to the liquid medium. Typically in this case, the feed is set-up so that the dry powder undergoes sufficient dissolution and/or mixing before contacting the cells.

In some embodiments, a portion of the ingredients of a feed supplement is reconstituted from a DPM, e.g., an agglomerated DPM. In some embodiments, the remainder of the ingredients is added to the reconstituted or a liquid form of the supplement feed. In some embodiments, the remainder of ingredients comprises amino acids.

In some instances, certain ingredients (e.g., amino acids) of a medium or supplement in a concentrated culture medium or concentrated supplement feed can precipitate, e.g., at or above certain concentrations. The inventors have surprisingly discovered that liquid concentrated feed supplements can be produced wherein at least some of the ingredients (e.g., amino acids (e.g., L-Tyrosine, L-Cystine and L-Asparagine)) are soluble and stay in solution at concentrations above their solubility limit, see Table 31 for examples.

TABLE 31

| Amino Acid | Merck Solubility |
|---|---|
| Asparagine | 0.29 g/L (in 1N HCl or 1N NaOH) |
| Cystine | 0.112 g/L (in WFI @ 25° C.) |
| Tyrosine | 0.45 g/L (in WFI @ 25° |

Additionally, the inventors have surprising found a method for producing a concentrated feed supplement medium. In some embodiments, a concentrated feed supplement medium of the invention comprises at least one component at a concentration above its solubility limit. In some embodiments, a concentrated feed supplement medium of the invention comprises at least one amino acid at a concentration above its solubility limit. In some embodiments, the at least one amino acid is selected from the group consisting of L-cystine, L-asparagine and L-tyrosine. In some embodiments, the at least one amino acid is selected from the group consisting of L-Isoleucine, L-Leucine, L-Lysine HCl, L-Proline, L-Serine, L-Arginine F.B., L-Aspartic Acid, L-Glutamic Acid, L-Histidine F.B., L-Methionine, L-Phenylalanine, L-Hydroxyproline, L-Threonine, L-Tryptophan and L-Valine.

Amino acids solubility in the presence of salts, such as NaCl and KCl, can differ significantly depending on the constitution of the hydrocarbon backbone. Generally, amino acids in the zwitter ionic state may form ion-pair complexes with electrolytes, such as NaCl and KCl. The solubility of such complexes in water again may depend upon the hydrocarbon backbone and the size/nature of the ions (ions of electrolyte). There may be a salt-in or salt-out effect of the amino acids due to the nature/size of the ions and hydrocarbon backbone.

Not wishing to be bound by theory, the inventors believe that they were able to achieve higher levels of solubility for medium components such as amino acids possibly because in the absence of certain salts in the medium, the solvation effect of water on the amino acids may keep them dissolved resulting in a clear solution. Ionic interactions between the polar amino acid functional groups and polar water may predominate, keeping the concentrated amino acids (e.g., at 5×) dissolved in the solution for longer periods of time, e.g., at 2° C. to 8° C. In contrast, with salt containing medium, the salvation power of polar water around amino acids is minimized, as a result they may tend to precipitate over a period of time stored @ 2° C. to 8° C. When dissolved in water, individual molecules of any amino acid can be solvated through hydrogen bonds. In a solid form, amino acids have strong ionic interactions between their molecules, but when dissolved in polar water, they lose the solid state interactions and get solvated around the polar function groups. Polar molecules (e.g., amino acids containing polar functional+/− charged groups) are attracted to polar water molecules and are hydrophilic.

Again not wishing to be bound by theory, the inventors present another related mechanism. The zwitter ionic attractions present between the molecules of any given amino acid (e.g., L-Cystine, L-Asparagine, and L-Tyrosine) in the solid state may be replaced by strong attractions between polar water molecules upon dissolution. The solvating power of water and polar functional groups of amino acids may determine the dissolution or solubility. Solvation of amino acids with polar water molecules may involve hydrogen bonds. In the absence of dissolved salts or at low concentrations of dissolved salts (e.g., NaCl, KCl and NaHCO₃) in the medium, the solvating power of water towards the amino acids predominates. As a result, these amino acids can remain solubilized in solution for periods of time, e.g., stored at 2-8° C.

There are other electrolytes' ions (e.g., Mg, Ca, and Zn), which can form ion-pair complexes as well, which may enhance the solubility of our amino acids, such as tyrosine, asparagine and cystine in the absence of NaCl and KCl. In some instances, ion-pair complexes of NaCl, KCl, and/or sodium bicarbonate with amino acids, such as tyrosine, asparagine and cystine may encourage or enhance the inter-molecular hydrophobic interactions thereby causing or enhancing the precipitation of an amino acid or other component out of a liquid, e.g., upon storing at 2-8 degrees centigrade.

In one embodiment of the invention, the medium (e.g., as described in U.S. patent application Ser. No. 11/151,647, Tables 1 or 2) to be fed comprises sodium bicarbonate, potassium chloride, sodium chloride and Pluronic F-68®, whereas the feed supplement comprises all of the ingredients (e.g., at 5×) of the medium except sodium bicarbonate, potassium chloride, sodium chloride and Pluronic F-68® are not present in the feed supplement.

Osmolality (a measure of osmotic pressure) of cell culture medium is important as it helps regulate the flow of substances in and out of the cell. It is typically controlled by the addition or subtraction of salt in a culture medium. Rapid increases in osmolality (e.g., addition of concentrated feed supplement with elevated osmolality relative to the base growth medium) may result in stressed, damaged and/or dead cells. Maintaining an optimal osmolality range during cell culture/growth is desirable for cell function and/or bioproduction success.

Base growth medium osmolality generally range from 250 mOsmo/kg to 350 mOsmo/kg. In some embodiments, addition of a concentrated feed supplement of the invention increases osmolality by about 25 mOsmo/kg or by between from about 0 to about 100, about 0.01 to about 100, about 0.1 to about 100, about 1 to about 100, about 10 to about 100, about 50 to about 100, about 75 to about 100, about 1 to about 10, about 1 to about 50, about 1 to about 75, about 10 to about 50, about 15 to about 35, about 25 to about 50, or about 20 to about 30 mOsmo/kg. In some embodiments, the osmolality of a concentrated feed supplement medium of the invention (e.g., a 5× concentrated feed supplement medium) has an osmolality between from about 0 to about 1500; 1 to about 1000; 1 to about 750; 1 to about 500; 1 to about 400; 1 to about 300; 1 to about 200; 1 to about 100; 1 to about 50; 50 to about 1000; 100 to about 1000; 300 to about 1000; 500 to about 1000; 750 to about 1000; 100 to about 200; 200 to about 300; 300 to about 400; 400 to about 500; 450 to about 500; 500 to about 600; 550 to about 650; 600 to about 700; 750 to about 850; 700 to about 800; 800 to about 900; 900 to about 1000; 1000 to about 1250; or about 1250 to about 1500 mOsmo/kg. In some embodiments, the osmolality of a concentrated feed supplement medium of the invention is between from about 3.0 to about 3.5×, about 3.5 to about 4.5×, about 4.5 to about 5.5×, about 5.5 to about 6.5×, about 6.5 to about 7.5×, about 7.5 to about 8.5×, about 8.5 to about 9.5×, about 9.5 to about 10.5×, about 10.5 to about 11.5×, about 11.5 to about 12.5×, about 12.5 to about 13.5×, about 13.5 to about 14.5×, about 14.5 to 18.5 to about 19.5×, about 19.5 to about 20.5×, about 3 to about 10×, about 5 to about 10×, about 10 to about 15×, about 15 to about 20×, about 20 to about 25×, or about 25 to about 100× as compared to the osmolality of the medium being supplemented or fed.

The present invention also provides methods for producing a concentrated feed supplement medium comprising a) acidification of water for dissolving at least one amino acid; b) adding an amount of at least one amino acid to solution (a) to achieve a desired concentration and optionally adding other components of a feed supplement; c) adding a second at least one amino acid to an appropriate volume of a dilute NaOH solution to achieve a desired concentration and optionally adding other components of a feed supplement; e) mixing the solutions of (b) and (c) together to form a concentrated feed supplement. In some embodiments, the solution of (d) is adjusted to a desired pH, e.g., a neutral pH such as between about 6.9 to about 7.4. In some embodiments, the pH of solutions (b) and (c) are predetermined to give a desired pH upon mixing together. In some embodiments, the solution of (d) is already at a targeted volume upon mixing of solutions (b) and (c) together. In some embodiments, the solution of (d) is brought to a targeted volume (e.g., to 5× feed supplement), e.g., with water after mixing of (b) and (c) together. In some embodiments, the solution of (d) is sterilized, e.g., by filtration.

In some embodiments, other components of a feed supplement are added to solution (a) as described above. In some embodiments, these other components comprises at least one component selected from the group consisting of L-Arginine, L-Aspartic Acid, L-Glutamic Acid, L-Histidine, Hydroxy-L-Proline, L-Isoleucine, L-Leucine, L-Lysine, L-Methionine, L-Phenylalanine, L-Proline, L-Serine, L-Threonine, L-Valine, B-12, Folic Acid, Niacinamide, Riboflavin, Thiamine and L-Tryptophan. In some embodiments, the other components added to solution (a), as described above, comprise L-Arginine, L-Aspartic Acid, L-Glutamic Acid, L-Histidine, Hydroxy-L-Proline, L-Isoleucine, L-Leucine, L-Lysine, L-Methionine, L-Phenylalanine, L-Proline, L-Serine, L-Threonine, L-Valine, B-12, Folic Acid, Niacinamide, Riboflavin, Thiamine and L-Tryptophan. In some embodiments, the other components added to solution (a) are in a liquid solution. In some embodiments, the other components added to solution (a) are in a liquid solution that was reconstituted from a dry powder form. In some embodiments, this dry powder form is an agglomerated dry powder form. In some embodiments, the other components added to solution (a) are in a dry powder form, e.g., an agglomerated dry powder form. In some embodiments, some of the other components added to solution (a) are in a liquid solution and some are in a dry powder form.

In some embodiments, acidification of water comprises adding an appropriate volume of an acid(s) (e.g., HCl) to achieve a desired pH for dissolving the at least one amino acid. In some embodiments, a desired pH for dissolving the at least one amino acid is between from about 0.25 to about 6.0, about 0.25 to about 1, about 0.5 to about 1, about 0.5 to about 1.5, about 1.0 to about 1.5, about 1.5 to about 2, about 1.5 to about 2.5, about 2.0 to about 2.5, about 2.5 to about 3, about 2.5 to about 3.5, about 3.0 to about 3.5, about 3.5 to about 4, about 3.5 to about 4.5, about 4.0 to about 4.5, about 4.5 to about 5, about 4.5 to about 5.5, about 5.0 to about 5.5, about 5.0 to about 6.0, about 5.5 to about 6.0, about 1.0 to about 2.0, about 2.0 to about 3.0, about 3.0 to about 4.0, about 4.0 to about 5.0 or about 5.0 to about 6.0. In some embodiments, the at least one amino acid is an acid soluble amino acid. In some embodiments, the at least one amino acid is selected from the group consisting of L-arginine, L-asparagine, L-aspartic acid, L-cystine.2HCl, L-glutamic acid, glycine, L-histidine, L-hydroxyproline, L-isoleucine, L-leucine, L-lysine.HCl, L-methionine, L-phenylalanine, L-proline, L-serine, L-threonine, L-tryptophan, L-tyrosine, and L-valine. In some embodiments, the at least one amino acid is selected from the group consisting of L-Cystine and L-Asparagine. In some embodiments, (b) comprises adding L-Cystine and L-Asparagine, each to achieve a desired concentration. In some embodiments, the desired concentration of one or more amino acids in (b), (c) and/or (d), as described above, is at a concentration above the solubility limit of the one or mores amino acids at a pH selected from the group consisting of about 6.9, about 7.0, about 7.1, about 7.2, about 7.3, about 7.4, about 7.5, between from about 6.9 to about 7.5, about 7.0 to about 7.5, about 7.1 to about 7.5, about 7.2 to about 7.5, about 7.3 to about 7.5, about 7.4 to about 7.5, about 6.9 to about 7.4, about 6.9 to about 7.3, about 6.9 to about 7.2, about 6.9 to about 7.1, about 6.9 to about 7.0, about 7.0 to about 7.4, about 7.0 to about 7.2, or about 7.2 to about 7.4. In some embodiments, (b) and/or (c), as described above, comprises mixing until the amino acids and/or other components are dissolved (e.g., for 15 minutes). In some embodiments, (b) and (c) above does not include the addition of one or more components selected from the group consisting of sodium bicarbonate, potassium chloride, sodium chloride and Pluronic F-68®. In some embodiments, the second at least one amino acid is a base soluble amino acid. In some embodiments, a base soluble amino acid is selected from the group consisting of L-Tyrosine, Isoleucine, L-Leucine, L-Lysine HCl, L-Proline, L-Serine, L-Arginine F.B., L-Aspartic Acid, L-Glutamic Acid, L-Histidine F.B., L-Methionine, L-Phenylalanine, L-Hydroxyproline, L-Threonine, L-Tryptophan and L-Valine. In some embodiments, the second at least one amino acid is L-Tyrosine. In some embodiments, a desired pH for dissolving the second at least one amino acid is between from about 8.25 to about 13.0, about 8.25 to about 9, about 8.5 to about 9, about 8.5 to about 9.5, about 9.0 to about 10.5, about 9.5 to about 10, about 9.5 to about 10.5, about 10.0 to about 10.5, about 10.5 to about 11, about 10.5 to about 11.5, about 11.0 to about 11.5, about 11.5 to about 12, about 12.5 to about 13.5, about 13.0 to about 13.5, about 13.5 to about 14, about 8.0 to about 9.0, about 9.0 to about 10.0, about 10.0 to about 11.0, about 11.0 to about 12.0 or about 12.0 to about 13.0.

In some embodiments, a concentrated feed supplement of the invention does not contain at least one or more of the following: sodium bicarbonate, potassium chloride, sodium chloride or Pluronic F-68®. In some embodiments, a concentrated feed supplement is produced wherein the final concentrated feed supplement comprises one or more amino acids at a concentration exceeding their usual solubility at the pH of the final concentrated feed supplement. In some embodiments, the one or more amino acids exceeding their normal solubility at the pH of the concentrated feed supplement will remain in solution for a period of time more than 1 week when stored at a temperature less than 37° C., e.g., between from about 0.5° C. to about 36.5° C., from about 2° C. to about 36.5° C., from about 0.5° C. to about 30° C., from about 0.5° C. to about 25° C., from about 0.5° C. to about 20° C., from about 0.5° C. to about 10° C., from about 0.5° C. to about 8° C., from about 0.5° C. to about 6° C., from about 0.5° C. to about 4° C., from about 0.5° C. to about 2° C., from about 2° C. to about 8° C. from about 2° C. to about 4° C., from about 2° C. to about 30° C., from about 2° C. to about 25° C., from about 2° C. to about 20° C., from about 4° C. to about 8° C., or from about 4° C. to about 10° C. In some embodiments, this period of time is selected from the group consisting of between from about 1 week to about 3 years, about 1 week to about 2.5 years, about 1 week to about 2 years, about 1 week to about 1.5 years, about 1 week to about 1 year, about 1 week to about 9 months, about 1 week to about 6 months, about 1 week to about 3 months, about 1 week to about 2 months, about 1 week to about 1 month, about 6 months to about 3 years, about 9 months to about 3 years, about 1 year to about 3 years, about 2 years to about 3 years, about 1 month to about 24 months, about 6 month to about 24 months, about 12 month to about 24 months, about 18 month to about 24 months, about 1 month to about 18 months, about 1 month to about 12 months, about 1 month to about 6 months, about 1 month to about 3 months, about 6 months to about 18 months and about 9 months to about 15 months.

In one embodiment, the liquid feed supplement is a 5× feed supplement lacking sodium bicarbonate, potassium chloride, sodium chloride, Pluronic F-68® e.g., as compared to the formulation in U.S. patent application Ser. No. 11/151,647, Table 2.

One method of the invention for producing a feed supplement medium of the invention comprises:

1) Acidification of the water used for formulation with an appropriate volume of HCL (1N) (e.g., 40 mL/liter equivalent) for dissolving L-Cystine and L-Asparagine.

2) Adding amounts of the amino acids L-Cystine and L-Asparagine (e.g., that are above their solubility limit at the 5× concentration at a neutral pH (7.0)) to the acidified water and mixed until dissolved (e.g., >15 minutes).

3) Adding the remainder of component complement of the medium less the sodium bicarbonate, potassium chloride, sodium chloride, and Pluronic F-68® to the acidified water containing L-Cystine and L-Asparagine. This solution is allowed to mix, e.g., for >15 minutes. In most instances, the solution may be cloudy but will typically clear with the subsequent additions and pH adjustment to neutral (e.g., about 7.0).

4) Adding the amino acid L-Tyrosine (e.g., that is above its solubility limit at the 5× concentration at neutral pH (7.0)) to an appropriate volume (e.g., 30 mL/liter equivalent) of a dilute NaOH solution (1N).

5) The base solubilized amino acid solution (e.g., 30 mL/liter equivalent) is added to the solution from (3) above and mixed, e.g., for >10 minutes. The solution can either be pH adjusted, e.g., to neutral such as 7.0±0.2 or the pH of the previous acidic and basic solutions can be predetermined, so that upon addition of the base solubilized amino acid solution, the desired pH is achieved. In most instances, the solution will clear and/or pH will be neutral.

6) If not already, the 5× feed supplement is brought to the final targeted production volume with water and optionally, sterile filtered for use. This 5× feed supplement now contains the full complement of components at 5× without sodium bicarbonate, potassium chloride, sodium chloride, Pluronic F-68®, e.g., as compared to Table 2 of U.S. patent application Ser. No. 11/151,647. In this feed supplement, several of the amino acids are in a neutral solution (pH of 7.0) at a concentration exceeding their normal solubility limit and will remain in solution for a period of time, (e.g., for up to 18 months or longer) without precipitation, e.g., when stored at 4° C.

In some embodiments, the present invention provides methods comprising reconstituting ingredients for a feed supplement (or a portion thereof) with a first solution comprising at least one of the ingredients of the feed supplement. In some embodiments, a second solution is added to the reconstituted solution. In some embodiments, the first and/or second solution comprises amino acids. In some embodiments, the final feed supplement product does not comprise any one, two, three or four of the ingredients selected from the group consisting of sodium bicarbonate, potassium chloride, sodium chloride, and Pluronic F-68®. In some embodiments, the only feed supplement ingredient (other than water) in the first and/or second solution is an amino acid(s). In some embodiments, the first solution and/or second solution is at an acidic pH, e.g., between about 0.5-6.5, about 0.5-1.0, about 0.5-2.0, about 0.5-3.0, about 0.5-4.0, about 0.75-5.0, about 0.75-6.0, about 1.0-3.0, about 0.1-1.0, about 1.0-2.0, about 2.0-3.0, about 3.0-4.0 or about 4.0-6.0. In some embodiments, the first solution and/or second solution is at a basic pH, e.g., between about 7.5-13.5, about 8.5-12.0, about 9.5-11.0, about 8.0-10.0, about 9.0-12.0, about 10-12, about 9.0-10.0, about 10-11, about 11-12, about 8.0-12.0, or about 8.0-9.0. In some embodiments, the first and/or second solution is at a relatively neutral pH, e.g., about 6.0-8.0, about 6.0-7.0, about 7.0-8.0, about 6.5-7.5, about 7.1, about 7.2, about 7.3, about 7.4, about 7.5, or about 7.6. In some embodiments, the acidic solution comprises L-Cystine and/or L-Asparagine. In some embodiments, the basic solution comprises L-Tyrosine.

These procedures described herein can be performed to produce feed supplement for other similar medium and medium as described herein.

Concentrations of components of a supplement are preferably adjusted to take into account the different rates of catabolism of different components, to bring about, e.g., induce, a desired change or maintain a level in cell metabolism; to ameliorate the buildup of undesired, for example, toxic, products; and/or for manufacturing or stability concerns. Preferred supplements may contain some ingredients in concentrated form; that is at a concentration greater than found in the original medium being supplemented. For example, one or more ingredients of the supplement may be present at a concentration about or exceeding 1.5, 2, 3, 4, 5, 7, 8, 10, 12, 15, 20, 25, 30, 40, 50, 60, 70, 75, 100, 125, 150, 200 times or even greater that of the medium being supplemented; while other ingredients may be present, e.g., at a concentration the same as or about (±10%) the same as the concentration in the medium being supplemented. Preferably one or several ingredients are omitted from the supplement. In some embodiments multiple supplements are used, e.g., for separate storage conditions, to allow for the culturist's special choices, for induction, for different stages of culture, to allow more precise control of individual ingredients or a set of ingredients, and/or to prevent the concentration of one or more ingredients from exceeding that desired.

In some embodiments, a concentrated feed supplement medium of the invention comprises at least one component, wherein the concentration of the component is at least 4×, at least 5×, at least 6×, at least 7×, at least 8×, at least 9×, at least 10×, at least 11×, at least 12×, at least 13×, at least 14×, at least 15×, at least 16×, at least 17×, at least 18×, at least 19×, at least 20×, at least 21×, at least 22×, at least 23×, at least 24×, at least 25× or more. In some embodiments, a concentrated feed supplement medium of the invention comprises at least one component, wherein the concentration of the component is between from about 3.0 to about 3.5×, about 3.5 to about 4.5×, about 4.5 to about 5.5×, about 5.5 to about 6.5×, about 6.5 to about 7.5×, about 7.5 to about 8.5×, about 8.5 to about 9.5×, about 9.5 to about 10.5×, about 10.5 to about 11.5×, about 11.5 to about 12.5×, about 12.5 to about 13.5×, about 13.5 to about 14.5×, about 14.5 to 18.5 to about 19.5×, about 19.5 to about 20.5×, about 3 to about 10×, about 5 to about 10×, about 10 to about 15×, about 15 to about 20×, about 20 to about 25×, or about 25 to about 100×.

Preferably a method such as fluidized bed granulation is used to provide a dry format powder that demonstrates reduced dusting and/or more rapid dissolution than provided when powders are prepared by milling without a production method to decrease dusting and improve dissolution.

Packaged media, media supplements, media subgroups and buffers of the invention are preferably stored for the extended times, and at the temperatures, noted herein, typically for about 1-24 months at temperatures of less than about 30° C., more preferably at temperatures of less than about 20-25° C., until use. Unlike traditional powdered media, media supplements, media subgroups or buffers, storage at reduced temperatures (e.g., 0-4° C.) is not necessary for the maintenance of performance characteristics of the media, media supplements, media subgroups and buffers prepared by the present methods. Of course, other storage temperatures may be required for those aspects of the invention where the packages also comprise separate compartments containing one or more solvents; in these cases, the optimal storage conditions will be dictated by the storage requirements of the solvent(s) which will be known to the skilled artisan.

Another aspect of the present invention features a dry format media supplement comprising at least two powder components. The preferred supplement is reconstitutable for addition to a cell culture medium. The preferred medium is capable of supporting cell growth and/or expansion and/or production biomolecules.

A preferred dry media supplement comprises a mixture of at least one first component selected from the group consisting of an amino acid or salt thereof, a polysaccharide, a solubilizing agent, a vitamin, a lipid, a fatty acid, a hormone, a growth factor, a differentiation factor, an active polypeptide, an iron chelator, a divalent metal salt, a carbon source, a monovalent metal salt, a pH buffer, a polyanion, a polycation, a surfactant, an antioxidant, a trace element salt, a nucleotide, a heterocyclic base and a nucleoside; and at least one second component having a different chemical constitution than said first component, said second component selected from the group consisting of an amino acid or salt thereof, a polysaccharide, a solubilizing agent, a vitamin, a lipid, a fatty acid, a hormone, a growth factor, a differentiation factor, an active polypeptide, an iron chelator, a divalent metal salt, a carbon source, a monovalent metal salt, a pH buffer, a polyanion, a polycation, a surfactant, an antioxidant, a trace element salt, a nucleotide, a heterocyclic base and a nucleoside.

Another preferred supplement of the invention features a supplement comprising at least one amino acid or salt thereof selected from the group consisting of alanine, cysteine, cystine, aspartic acid, glutaminc acid, phenylalanine, glycine, histidine, isoleucine, lysine, leucine, methionine, asparagine, proline, hydroxyproline, glutamine, arginine, serine, threonine, valine, tryptophan and tyrosine.

Another preferred aspect of the invention features a supplement of the present invention reconstituted in a polar solvent, preferably reconstituted in essentially physiologic pH water.

One or more amino acids, preferably 2, 3, 4, 5 or more amino acids comprise a preferred aspect of supplements of the invention. Salts of amino acids may be substituted for amino acids in some aspects of the present invention. Supplements containing at least one amino acid and at least one vitamin are yet another aspect of the invention.

Preferred powder preparation methods include one or more of milling, impacting, extruding and cutting or breaking, wet granulation, high shear granulation, pan granulation and fluidized bed agglomeration.

Preferred milling apparatus include ball mill, roll mill, fitz mill, comill, jet mill and hammer mill, and any other mechanical particle size attrition device.

A preferred dry supplement formulation comprises an amino acid or salt thereof, a vitamin, and a carbon source. Yet another preferred formulation comprises at least one, possibly at least two, preferably three, four five or more ingredients. Exemplary ingredients may be selected from the group consisting of a metal chelator, lipids, fatty acids, a divalent metal salt, a pH buffer, and a carbon source. Other examples may be selected from the group consisting of a polyanion, a polycation, a surfactant, an antioxidant, a carbon source, a trace metal element salt, a nucleotide, a heterocyclic base and a nucleoside.

Yet another aspect of the invention features a method of reconstituting a dry medium supplement powder comprising obtaining a powder according to the present invention and dissolving the powder in essentially neutral pH water and or polar solvent.

An improvement in the art provided by the present invention can be described as follows: a dry powder medium supplement for supplementing cells growing in culture, the improvement being that the supplement powder reconstitutes in essentially neutral pH water and or polar solvent. Preferably, the reconstitution is rapid, preferably less than five minutes per liter, more preferably less than 4, 3, 2, or one minute per liter. Especially for large batch sizes rapid easy reconstitution is preferred, for example, less that 45, 30, 15 or even 10 seconds per liter.

A second improvement for some aspects is that pH adjustment is not required.

A method of culturing cells comprising: growing cells in culture for at least 2 days to form a mature culture; reconstituting a supplement powder using essentially neutral pH water and or polar solvent to form a reconstituted supplement; and adding said reconstituted supplement to said mature culture. A mature culture can be a culture at a desired stage of growth, for example, a culture adjudged to be ready for commencing bioproduction. A preferred aspect provides that growth/expression/product ion of said cells in culture is increased from that of the mature culture.

Another aspect of the invention is in kit form comprising at least two containers of different composition of components wherein at least one container contains a mixture of at least two components. Preferably reconstitution of the components of at least one container is in a polar solvent and/or the reconstitution of the components of at least a second container is in essentially physiologic pH water.

Preferred mixtures of the present invention are homogeneous mixtures.

Other embodiments of the invention provide method wherein the pressure feed is provided by a pump selected from the group consisting radial flow, rotary, axial flow, regenerative, turbine, plunger, diaphragm, cam and piston, peristaltic, gear, lobe, piston, screw, syringe, metering, sliding-vane, hydraulic, jet, volumetric displacement, and other reciprocating or positive displacement pumps.

A preferred adding rate may range from about 1%/day to about 500%/day. Supplementation can be selected for ease of the additions and results obtained. A tradeoff may be found in many aspects. For example a preferred supplementation schedule may be once a day, twice a day 3, 4, 5, 6, 7, or eight times per day, once every several days, for example every two, two and a half, three, 5 or even seven days. Supplementation may also be effected in accordance with a culture monitoring system, wherein threshold values or one or more algorithms are used to determine the feeding schedule. Preferred percentages of supplementation include 1% per addition, 2, 3, 5, 7, 10, 20, 50%, continuous (perfusion) preferred rates include 1 volume per day ranging from 25% to 5 volumes per day. Preferably the adding is metered according to a feed back circuit based on at least one measured concentration or size from a component in the medium. Preferred monitoring includes cell volume, number, size, concentration of glucose, any amino acid, a metabolic product or metabolite, active oxygen, active oxygen products, lactate, pH, Na, K, Ca, Se, viscosity, light absorbance, color change, protein.

A preferred culture is one wherein the active polypeptide is selected from the group consisting of an enzyme, a transport protein, a membrane stabilizer, a neurotransmitter, a differentiating agent and a binding or sequestering agent.

Another preferred medium supplement powder is one wherein the vitamin is selected from the group consisting of ascorbate or ascorbic acid or salt thereof, biotin, pantothenate or pantothenic acid, bitartrate, choline chloride, cyanocobalamin, D- or DL-alpha tocopherol or tocopherol acetate, folic acid, folinic acid or a salt thereof, i-inositol, carnitine, lipoic acid, linoleic acid, menadione or salt thereof, niacinamide, nicotinic acid, para-aminobenzoic acid, pyridoxal 5-phosphate, pyidoxal HCl, pyridoxamine mono or di hydrochloride, retinoic acid, riboflavin, riboflavin-5-phosphate sodium dihydrate, thiamine HCl, Thiamine monophosphate, vitamin A or salt thereof, Vitamin D2 and vitamin D3.

A preferred supplement powder is one wherein the at least two powder components are milled to produce a mean diameter ratio of a largest component to a smallest component of 1:1 to 10:1.

Yet another preferred aspect features a powder supplement wherein the at least two powder components are blended using tumble blender including but not limited to drum blender, V-blender, ribbon blender, cone blender, slant-cone blender and double-cone blender sufficient mixing time and speed to attain a homogenous mixture of components.

The present invention also features methods of producing a medium supplement, said method comprising obtaining a dry format media supplement said supplement comprising at least two powder components and said supplement reconstitutable for addition to a cell culture medium, said medium capable of supporting cell growth, biomolecule production and/or expansion; and dissolving said supplement in a solvent to produce said medium supplement; and optionally adding said medium supplement to said medium.

A preferred method is one wherein at least one of said at least two components is present at a concentration in excess of a 1× concentration with respect to the medium being supplemented. A more preferred method is one wherein at least one of said at least two components is present at a concentration of from greater than a 1× to about a 5×, 10×, 20×, 50×, 75×, 100×, 120×, 150×, 200×, 250×, 500×, 750× or up to a 1000× concentration with respect to the medium being supplemented.

An especially preferred method is one wherein at least one of said at least two components is present at a concentration in excess of about 4× concentration with respect to the medium being supplemented.

Another especially preferred method is one wherein at least one of said at least two components is present at a concentration in excess of about a 9× concentration with respect to the medium being supplemented. Yet another preferred method is one wherein at least one of said at least two components is present at a concentration of about 5× with respect to the medium being supplemented. Yet still another preferred method is one wherein at least one of said at least two components is present at a concentration of about 10× with respect to the medium being supplemented. Perhaps two or more, for example, 3, 4, 5, 6, 7, 8, 9, or more components including essentially all components of the supplement powder may reach any of these concentrations before addition.

Another aspect of the invention is a powder wherein a first component of the medium being supplemented is omitted or is present at a concentration with respect to said medium being supplemented in comparison to a second component with respect to said medium being supplemented at a ratio greater than 0, range 10-6 to 0.9; Exemplary supplements are those wherein said first component is selected from the group consisting of alanine, cysteine, cystine, aspartic acid, glutaminc acid, phenylalanine, glycine, histidine, isoleucine, lysine, leucine, methionine, asparagine, proline, hydroxyproline, glutamine, arginine, serine, threonine, valine, tryptophan and tyrosine, an alkali metal salt, a pH buffer and a surfactant.

Especially preferred powders include those wherein all components are soluble in essentially physiologic pH water at a concentration of from about 2×, about 4×, about 5×, about 10×, about 15×, about 20×, about 25×, about 30×, about 40×, about 45×, to about 48× or about 50× or from about 50× to about 100×, about 120×, about 150×, about 180×, about 190× or about 200× of the medium being supplemented.

Preferably all supplement components are of non-animal origin. More preferably the supplement is chemically defined.

Another aspect is one wherein no component is serum or a product of serum.

A preferred powder format is one that is prepared by fluidized bed agglomeration.

Preferably supplement powders comprise at least one sugar. Another preferred dry format powder supplement comprises a polymeric binder such as methyl cellulose and derivative of starch, ethyl cellulose and polyvinylpyrrolidone (PVP).

Another dry format media supplement is one wherein at least a first powder component is designated as 1× per unit weight based on the medium to be supplemented and a second powder component is greater than 1.4× or less than 0.8× per said unit weight.

Another preferred supplement may be one wherein at least said second powder component is not present in said medium to be supplemented.

Preferably upon reconstitution with a solvent a result is achieved wherein in at least one component concentration greater than about 2× that of said medium component concentration.

A preferred supplement is one wherein at least one component is soluble at at least 2× in physiologic pH water.

Preferred supplements may comprise bicarbonate. Preferably the bicarbonate is a salt of a monovalent metal.

A preferred supplement comprises at least one ingredient selected from the group consisting of trace element salt. Preferrably the one or more trace element salts are homogenously distributed throughout the dry format media supplement.

A preferred result of the products and methods of the present invention is one wherein said culturing results in creation of or an increased production of a desired cell component, product or function.

### Cells

In another aspect, the invention relates to methods for producing dry cell powder compositions comprising one or more cells, and to dry cell powders produced by these methods. In one embodiment, the invention relates to reducing adventitious agents or toxins from a sample containing one or more cells by the methods of the invention. These methods thus produce cell-containing compositions wherein the cells are preserved and may be stored for extended periods of time until use. In some embodiments, these methods produce cell-containing compositions wherein the cells are preserved and may be stored for extended periods of time until use and such cell compositions have reduced or eliminated adventitious agents or toxins. In this way, the methods of the invention overcome some of the drawbacks of traditional methods of cell preservation (e.g., freezing) such as the need for cyropreservation equipment and the use of certain cryopreservatives that may be toxic to the cells.

Methods according to this aspect of the invention may comprise one or more steps. For example, one such method may comprise obtaining one or more cells to be dried, forming an aqueous cell suspension by suspending the one or more cells in an aqueous solution, and spray-drying the cell suspension under conditions favoring the production of a dried powder. In some embodiments of the invention, a method may comprise obtaining one or more cells of interest, forming an aqueous cell suspension by suspending the one or more cells in an aqueous solution, and treating the cells in accordance with the invention under sufficient conditions to reduce or substantially reduce adventitious agents or toxins (without substantially affecting the viability of such cells), preferably by substantially drying the cell suspension under conditions favoring the production of a dried powder (preferably by spray-drying). Another embodiment of the invention includes obtaining one or more cells to be dried, contacting the one or more cells with one or more stabilizers (e.g., a polysaccharide such as trehalose), forming an aqueous suspension comprising the one or more cells, and spray-drying the cell suspension under conditions favoring the production of a dried powder. These methods may further comprise contacting the one or more cells with one or more stabilizing or preserving compounds (e.g., a polysaccharide, including but not limited to trehalose). In one embodiment, the aqueous solution used to form the cell suspension comprises one or more components, such as one or more of the herein-described nutritive media, media supplements, media subgroups, salts or buffers, or one or more of the automatically pH-adjusting culture media, media subgroups, media supplements or buffers of the present invention. In one embodiment, an aqueous suspension comprising the one or more cells preferably comprises an aqueous solution, such as one or more of the herein-described nutritive media, media supplements, media subgroups or buffer solutions, adjusted to optimal or substantially optimal tonicity and osmolarity for the cell type being dried. Preferably, the aqueous solution used to form the cell suspension is adjusted to optimal or substantially optimal tonicity and osmolality for the cell type being dried. The aqueous solution may optionally comprise one or more additional components, such as one or more salts, polysaccharides, ions, detergents, stabilizing or preserving compounds (including trehalose), and the like. In aspects of the invention wherein the one or more cells are contacted with one or more stabilizing or preserving compounds, the stabilizing or preserving compounds may be incorporated into the aqueous solution used to form the aqueous cell suspension. Alternatively, the stabilizing or preserving compounds may be sprayed or agglomerated onto the dry cell powder after formation of the powder. In one embodiment, the stabilizing compounds with, which the one or more cells are to be contacted, may be incorporated into the aqueous solution used to form the aqueous cell suspension.

Once the dry cell powder has been formed by the herein-described methods, the powder may optionally be agglomerated with a solvent according to methods described herein for agglomeration of dry powders. Any solvent that is compatible with the cell type being dried may be used to agglomerate the dry cell powder, including but not limited to water, a nutritive medium solution, a nutritive medium supplement solution (including sera, particularly bovine sera (most particularly fetal bovine and calf sera) and human sera), a buffer solution, a salt solution, and combinations thereof. In another aspect, the cell powder of the invention may be mixed with one or more powdered media, media supplements, media subgroups or buffers (which are produced by the methods of the invention or by standard techniques) and such mixtures may optimally be agglomerated with a solvent by the methods of the invention.

A variety of cells may be dried according to the methods of the invention, including prokaryotic (e.g., bacterial) and eukaryotic (e.g., fungal (especially yeast), animal (especially mammalian, including human) and plant) cells, particularly those cells, tissues, organs, organ systems, and organisms described herein. Once the dried cells have been produced, they may be packaged aseptically and stored for extended periods of time (e.g., several months to several years), preferably at temperatures of about 0-30° C., 4-25° C., 10-25° C., or 20-25° C. (i.e., “room temperature”) until use. For use, the dried cells are preferably aseptically reconstituted with an aqueous solvent (e.g., sterile water, buffer solutions or culture media) and cultured according to standard art-known protocols. For use in preparing cultures of viable cells, the dry cell powder may be aseptically reconstituted, into a cell suspension comprising one or more viable cells, with an aqueous solvent (e.g., sterile water, buffer solutions, media supplements, culture media, or combinations thereof) and cultured according to standard art-known protocols.

Alternatively, the dry cell powder may be reconstituted into a cell suspension where cell viability is not essential, for example for preparation of an immunogen to be used for immunization of an animal. In such cases, the dry cell powder may be reconstituted with any solvent that is compatible with standard immunization protocols, such as aqueous or organic solvents that may comprise one or more detergents, adjuvants, etc.

The invention also provides compositions prepared by such methods. Such compositions may comprise, for example, an automatically pH-adjusting culture medium powder of the invention and one or more cells, such as one or more bacterial cells, one or more plant cells, one or more yeast cells, and one or more animal cells (including but not limited to one or more mammalian cells such as one or more human cells). Compositions according to this aspect of the invention may be in powder form, which upon reconstitution with a solvent, produce an active culture of the one or more cells contained in the composition.

### Kits

The dry powder media, media supplements, media subgroups, buffers, cells and cell-containing compositions provided by the invention are ideally suited for preparation of kits. The pharmaceutical or clinical compositions, cell culture reagents, media, media supplements, media subgroups, buffers and cells provided by the invention are ideally suited for preparation of kits. Such a kit may comprise one or more containers such as vials, test tubes, bottles, packages, pouches, drums, and the like. Each of the containers may contain one or more of the herein-described pharmaceutical or clinical compositions, cell culture reagents, nutritive media, media supplements, media subgroups, cells or buffers of the invention, or combinations thereof. Such pharmaceutical or clinical compositions, cell culture reagents, nutritive media, media supplements, media subgroups, buffers or cells may be hydrated or dehydrated but are typically dehydrated preparations produced by the methods of the invention. Such preparations may, according to the invention, be sterile or substantially sterile.

A first container may contain, for example, a nutritive media, media supplement, media subgroup or a buffer of the invention, or any component or subgroup thereof, such as any of those nutritive media, media supplements, media subgroups or buffers of the invention that are described herein. Additional nutritive media, buffers, extracts, supplements, components or subgroups may be contained in additional containers in the present kits. The kits may also contain, in one or more additional containers, one or more cells such as the herein-described bacterial cells, yeast cells, plant cells or animal cells. Such cells may be lyophilized, dried, frozen or otherwise preserved, or may be spray-dried according to the methods of the invention or treated by the method of the invention. In addition, the kits of the invention may further comprise one or more additional containers, containing, for example, L-glutamine, optionally complexed with one or more divalent cations (see U.S. Pat. No. 5,474,931). The kits may further comprise one or more additional containers containing a solvent to be used in reconstituting the dry powder pharmaceutical or clinical compositions, cell culture reagents, nutritive media, media supplements, media subgroups and/or buffers; such solvents may be aqueous (including buffer solutions, saline solutions, nutritive medium solutions, nutritive medium supplement solutions (including sera such as bovine sera (particularly fetal bovine sera or calf sera) or human sera)), or combinations thereof) or organic. Other ingredients that are not compatible for admixture with the nutritive media, buffers, pharmaceutical compositions, extracts, supplements, components or subgroups of the invention may be contained in one or more additional containers to avoid mixing of incompatible components. An exemplary kit may comprise a container containing dry powder for reconstitution optionally of a volume sufficient to contain the reconstituting solvent, instructions for reconstitution and means for accessing the dry powder such as a tear strip or a port for introducing the reconstituting solvent. Such kits may also comprise transfection reagents (such as lipids or cationic lipids).

The number and types of containers contained in a given kit (e.g., for making a nutritive medium, medium supplement, medium subgroup or buffer) may vary depending on the desired product or the type of pharmaceutical or clinical compositions, media, media supplement, media subgroup or buffer to be prepared. Typically, the kit will contain the respective containers containing the components or supplements necessary to make a particular pharmaceutical or clinical composition, media, media supplement, media subgroup or buffer. However, additional containers may be included in the kit of the invention so that different pharmaceutical or clinical compositions, media, media supplements, media subgroups or buffers can be prepared by mixing different amounts of various components, supplements, subgroups, buffers, solvents, etc., to make different pharmaceutical or clinical compositions, media, media supplement, media subgroup or buffer formulations.

### Advantages for Some Embodiments of the Invention

Unexpectedly, the present invention provides for the preparation of nutritive media, media supplements, media subgroups, buffers and cells at reduced cost. Unexpectedly, the present invention provides for the preparation of lipid containing nutritive media, media supplements, media subgroups, buffers and cells at reduced cost and reduced inconvenience. The cost reductions are due to the several factors. For example, the media, media supplement, media subgroup and buffer formulations of the present invention may be produced with much smaller production facilities since the large stir tanks required for 1× formulations are not required. In addition, the media, media supplement, media subgroup and buffer formulations of the present invention may be prepared on an as needed basis using “just in time” production techniques which reduce inventory, storage and labor costs. The time required for the preparation and shipping of the media, media supplement, media subgroup and buffer formulations may be reduced from 6-8 weeks to as little as one day. The automatically pH-adjusting media of the invention also provide significant cost and time savings, and reduce the tendency for introduction of contamination into reconstituted media that may occur during the pH adjustment process according to standard methods using traditional dry powder or bulk liquid media. The present invention also allows for the preparation of components of nutritive media, media supplements, media subgroups or buffers which may be used to prepare very large quantities of 1× media, media supplements, media subgroups or buffers (e.g., 100,000 liters or more) which would require only one quality control test compared to multiple quality control tests for multiple batches produced according to other commonly used techniques. Importantly, the media, media supplement, media subgroup and buffer formulations of the present invention are more consistent between batches since the individual components are more stable. The dried cell powders of the invention are also technologically and economically advantageous, since the cells may be stored, in low volume, for extended periods of time with little need for specialized equipment beyond that typically available in the laboratory. In addition, the cells prepared by the present methods are preserved without being exposed to cryopreservative reagents which may be toxic to the cells. In one embodiment, where the cells are preserved without being exposed to often-toxic cryopreservative reagents, the cells would be more likely to recover and enter log-phase growth more rapidly than cells preserved by traditional methods such as cyropreservation. The improved convenience will reduce the burden of supplying lipid to cells in culture. Improved methods of providing lipids in the dry media formulations should result in better performance of the cells in culture in performing their physiologic or intended tasks.

Some embodiments of the invention include methods of producing an agglomerated nutritive medium powder, an agglomerated medium supplement powder, an agglomerated nutritive medium subgroup powder, or an agglomerated buffer powder, said method comprising agglomerating a nutritive medium powder, medium supplement powder, nutritive medium subgroup powder, or buffer powder, with a solvent comprising at least one lipid dissolved therein, said solvent delivering said at least one lipid for incorporation in said nutritive medium powder, medium supplement powder, nutritive medium subgroup powder, or buffer powder.

In certain embodiments of the invention, the agglomerating comprises fluid bed agglomeration.

In certain embodiments of the invention, the solvent is in liquid phase. In other embodiments, the solvent is in solid phase.

### Lipids

This section provides various embodiments of the invention related to lipids in addition to the embodiments described elsewhere herein. In certain embodiments of the invention, the lipid is a lipid modified to be more soluble in said solvent compared to when the lipid is not so modified. The lipid can be in the form of a salt, the lipid can have one or more hydroxyl groups, and the lipid can be complexed with a cyclodextran.

In certain embodiments of the invention, the solvent is a mixture. The mixture can be a mixture of liquids. The mixture may also comprise at least one polar solvent and/or at least one non-polar solvent and/or at least one organic solvent. For example, the mixture may comprise 20%-95% organic solvent, e.g., 20%, 40%, 50%, 60%, 80%, 90% or 95% organic solvent.

When the solvent is a mixture, the mixture may comprise, e.g., solvents in a ratio of 1% to 99% of (a) said at least one polar solvent with (b) said at least one organic or said at least one non-polar solvent. The mixture may comprise solvents in a ratio of, e.g., 1, 5, 7, 10, 15, 20, 25, 30, 33, 40, 50, 60, 67, 70, 75, 80, 85, 90, 95, 98 or 99% of (a) said at least one polar solvent with (b) said at least one organic or said at least one non-polar solvent.

When the solvent is a mixture, the mixture may comprise 40-60% of said at least one organic or said at least one non-polar solvent. In certain embodiments, the mixture comprises 50% of (a) said at least one polar solvent, and 50% of (b) said at least one organic or said at least one non-polar solvent.

When the solvent is a mixture, the mixture may comprise, e.g., water and at least one solvent selected from the group consisting of dimethylsulfoxide, alcohols, ethers, and ketones. The mixture may comprise, e.g., at least one solvent selected from the group consisting of dimethylsulfoxide, alcohols, ethers, and ketones. The mixture may comprise about 40%-60% ethanol. In one embodiment, the mixture comprises about 50% ethanol. The solvent may comprise a mixture of at least two solvents selected from the group consisting of non-polar solvents and organic solvents.

In certain embodiments of the invention, said delivering is performed under conditions comprising at least one of controlled temperature, controlled humidity and controlled partial pressure of said solvent(s).

In certain embodiments of the invention, said lipid is selected from the group consisting of linoleic acid, lipoic acid, arachidonic acid, palmitic acid, oleic acid, palmitoleic acid, stearic acid, myristic acid, linolenic acid, phosphatidyl ethanolamine, phosphatidyl choline, sphingomylelin, cardiolipin, vitamin A, vitamin E, Vitamin K, prostaglandin and a sterol. The sterol can be, e.g., a plant or an animal sterol. In certain embodiments, the sterol is cholesterol.

The invention is also directed to agglomerated nutritive medium powders, agglomerated medium supplement powders, agglomerated nutritive medium subgroup powders, and agglomerated buffer powders prepared according to any of the methods of the invention. The powder of the invention, in certain embodiments, has reduced dusting compared to a non-agglomerated nutritive medium powder, more complete solubility compared to a non-agglomerated nutritive medium powder, less insoluble material compared to a non-agglomerated nutritive medium powder, and/or more rapid dissolution compared to a non-agglomerated nutritive medium powder.

The powder of the invention, in certain embodiments, is free of serum, free of mammalian components, and/or free of animal components.

The invention also provides a method of culturing a cell comprising: (a) reconstituting an agglomerated powder of the invention with a solvent to form a liquid solution; and (b) contacting a cell with said liquid solution under conditions favoring the cultivation of said cell. The cell can be, e.g., a cell selected from the group consisting of bacterial cell, insect cell, yeast cell, nematode cell, avian cell, amphibian cell, reptilian cell, and mammalian cell. When the cell is a mammalian cell, the cell may be, e.g., a CHO cell, a COS cell, a VERO cell, a BHK cell, an AE-1 cell, an SP2/0 cell, an L5.1 cell, a PerC6 cell, a 293 cell, a hybridoma cell, or a human cell. According to certain aspects of the invention, the growth of said cell at 3, 4, 7, 10, 14, 28, 30, 60 or 90 days is 50%-120% compared to the growth of said cell at the same time point in liquid medium with added lipid. For example, the growth of said cell at 3, 4, 7, 10, 14, 28, 30, 60 or 90 days may be, e.g., 50%, 60%, 75%, 80%, 90%, 100%, 105%, 110% or 120% compared to the growth of said cell at the same time point in liquid medium with added lipid.

### Methods of Reducing Adventitious Agents

This section provides various embodiments of the invention related to methods of reducing adventitious agents in addition to the embodiments described elsewhere herein. The present invention is directed to methods of producing samples (preferably a sample containing biological or animal derived components or ingredients) having reduced or eliminated adventitious agents and/or toxins and more particularly to cell culture nutrients, cell culture reagents, nutritive media, media supplements, media subgroups or buffers having reduced, substantially reduced, inactivated or eliminated adventitious agents or toxins. The invention also relates to pharmaceutical or clinical compositions or solutions produced by these methods.

By the methods of the present invention, any sample, particularly pharmaceutical or clinical compositions and solutions, cell culture reagents, nutritive media, media supplement, media subgroup or buffer may be produced and stored for an extended period of time without significant loss of biological and biochemical activity. Thus, for a pharmaceutical composition, the pharmaceutical composition may be tested for the pharmaceutical property of interest (e.g. drug efficiency) while a media will be tested for cell growth or other parameters well known to those skilled in the art.

Any pharmaceutical or clinical composition, cell culture reagent, nutritive media, media supplement, media subgroup or buffer (or any ingredient used or present in such samples) may be prepared by the methods of the present invention. Particularly preferred nutritive media, media supplements and media subgroups that may be prepared according to the invention include cell culture media, media supplements and media subgroups that support the growth of animal cells, plant cells, bacterial cells or yeast cells. Particularly preferred buffers that may be prepared according to the invention include balanced salt solutions which are isotonic for animal cells, plant cells, bacterial cells or yeast cells. Such solutions may be made as a 1× formulation or in concentrated (e.g. in hypertonic concentrations) for example a 10×, 25×, 50×, 100× etc. formulas.

In an aspect of the invention, the liquid injected may contain gas or compounds (biological or chemical) which facilitate reduction, inactivation or elimination of toxins and/or adventitious agents.

The present invention thus provides samples including pharmaceutical and clinical compositions/solutions, nutritive media, media supplements, media subgroups and buffers (which are preferably powdered) that have reduced, substantially reduced, inactivated or eliminated adventitious agents and/or toxins. In powdered form, such samples are readily soluble in a rehydrating solvent and are substantially dust free. For use, samples produced by the may be hydrated (or “reconstituted”) in a volume of a solvent sufficient to produce the desired concentration, nutrient, electrolyte, ionic and pH conditions required for the particular use of the solvated sample (e.g. media, media supplement, media subgroup or buffer). This reconstitution is particularly facilitated in the present invention, since the powdered sample will rapidly go into solution and will produce little if any dust or insoluble material, unlike lyophilized or ball-milled samples such as nutritive media, media supplements, media subgroups or buffers.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Agglomeration of Typical Dry Powder Media (DPM)

1. With a benchtop laboratory fluid bed apparatus (Stera-1; Niro, Inc./Aeromatic-Fielder; Columbia, Md.): Place 100-500 g of DPM within the chamber. Place onto apparatus and use the lever to seal the unit.
2. Start the airflow to fluidize (levitate) the DPM. Since traditional DPM is of relatively fine particle size, setting 4-6 will be needed. Turn on the vacuum device to catch fine DPM particles, passing through the upper filters. Make sure that the fluidized powder is approximately central within the chamber with respect to the lower mesh screen and the upper filters.
3. Start the injection device (spray unit) by first plugging in the compressed air line and then by starting the pump which is connected to a water source. The goal is to admit ˜6 ml of water per minute (the flow rate for any given pump based upon RPM and tubing diameter must be known). In order to prevent clumping of DPM, alternatively add water for ˜1 minute and then stop for ˜1 minute, allowing drying to occur in the chamber.
4. If filters become coated with DPM during the run so that blowback does not dislodge powder, turn fan speed down to setting 2-3 until all filters have been blown clear. Then increase running fan speed to previous level.
5. Agglomeration will be complete when ˜35 ml of water has been added for each 500 g of DPM. This volume will vary depending upon the DPM formulation. A downward flow of relatively large agglomerated granules will be seen in the chamber (bottom) toward the end of the run. Visibly larger particles and absence of fine dust indicates that the process is complete.
6. Allow agglomerated DPM to dry thoroughly for 5-7 minutes.
7. At end of run, blow off filters 4 times.
8. Turn unit off, disconnect water tube and collect agglomerated DPM into an airtight container.

These approaches should be adjusted when using a process-scale or production-scale fluid bed apparatus. For example, when the MP-1 (Niro, Inc./Aeromatic-Fielder; Columbia, Md.) apparatus is used, the following protocol has yielded satisfactory results:

1. Seal unit (inflate gaskets).
2. Start fan for pre-heat.
3. Stop fan when inlet air temperature equals set point.
4. Deflate gaskets, load material, inflate gaskets.
Steps 5-8 should all be accomplished within one minute:
5. Start batch.
6. Start fan, and turn on filter cleaning.
7. Set nozzle atomizing air pressure % output (check nozzle for vacuum).
8. Connect liquid feed line.
9. Start pump on screen and at pump.
10. Reset batch time.
11. Spray all liquid at set rate (26 g/min). Use ˜250 ml water for 2 kg powder.
12. Stop pump at pump and on screen when all liquid is added.
13. Reduce airflow to drying value (for example from 100 to 60).
14. When product reaches desired temperature (˜40° C.), go to “initial set up” screen and set “batch duration” for a value of 2-3 minutes greater than the present “batch time”.
15. Stop batch.16. Deflate gaskets.
Typical instrument settings (for bench-, process- and production-scale apparatuses):
Drying temperature: 60-65° C.
Outlet air temperature: ˜33° C.
Blow out pressure: 5 bar
Atomizing pressure: 1.5-2.0 bar
Blow back dwell: 1 after spraying, 2 while spraying
Capacity of fan: 5 at start of run, 6 after agglomeration is evident
Magnahelics: Filter resistance 150-250, Resistance of perforated control plate ˜50,
Air volume: less than 50.

### Example 2

### Addition of Sodium Bicarbonate as an Integral Part of DPM

As noted herein, sodium bicarbonate is not typically added to DPM during manufacturing by ball-milling or lyophilization, due to potential off-gassing and buffering capacity complications encountered upon storage of the powdered media. This standard production process thus necessitates the addition of sodium bicarbonate, and pH adjustment, upon reconstitution of the media. With the present methods, however, these additional steps may be obviated by adding the sodium bicarbonate (or any buffering salt) directly to the powdered medium during manufacturing.

There are two ways of including sodium bicarbonate (or any buffering salt) within the DPM: (a) via the injection device and (b) as part of the DPM.

### (a) Injection Device

Because of the solubility of sodium bicarbonate and the amounts that generally need to be added to a typical mammalian cell culture medium, fairly large volumes of liquid would need to be injected into the powder (significantly greater than the 35 ml of water mentioned above). This is still possible and in fact may be preferable if adding another component that similarly requires a relatively large volume of liquid in order to be added to the DPM, as is the case with serum for example. In this case, care must be taken to sequentially add liquid, let dry etc. a number of times to insure that the DPM does not become clumped within the device. Using the 6 ml per minute for ˜1 minute and then allowing drying for another 2 minutes is about right.

The amount of liquid to add is determined as follows: Prepare sodium bicarbonate at 75 g/L in water. Example: 250 g of DPM in the chamber to be agglomerated. Assume 10.0 g of DPM is required for 1 L of 1× liquid medium. Therefore, 250 g represents 25 L of 1× liquid medium. For each L of liquid, assume (for example) a requirement of 2 g of sodium bicarbonate. This means that 50 g of bicarbonate is needed. Now, since the bicarbonate solution is at 75 g/L, then 0.67 L of bicarbonate solution must be added to the 250 g of DPM.

The sodium bicarbonate solution would be added similarly to the process for “agglomeration of a typical DPM” above except that a longer drying time between cycles is needed since the pH of the sodium bicarbonate solution is ˜8.00 which can degrade media components. It is important that the powder never become “soaked” by addition of bicarbonate solution too rapidly without allowing sufficient time for thorough drying of the bicarbonate powder between cycles. Also, longer fluid drying times are required since it is important to have as low a final moisture content as possible since moisture would result in liberation of carbon dioxide gas resulting in loss of buffering capacity and “pillow” formation if powder is in a foil packet.

### (b) As Part of the DPM

Sodium bicarbonate can be milled into the DPM in a similar fashion as for other media components prior to fluid bed treatment. However, in the milling process, the bicarbonate should be added as the final component. All of the other media components should be milled as usual and then the mill stopped and the bicarbonate added last, with further milling to reach proper sized particles. It is important that all post-milling processing (placement into containers, etc.) be done in a humidity-controlled environment set as low as operationally possible (˜20-40%). Fluid bed processing should then be performed as soon as possible after milling. (If not processed the same day, DPM must be double wrapped and placed within a sealed container with moisture absorbents.)

The fluid bed process itself is done similarly to the example given above (with use of 35 ml per 500 g of DPM) except that drying times after water injection (˜6 ml/min) should again be extended: 1 min of injection of water and 2 minutes drying cycles. It will be noted that the color of the DPM will be deep red-light purple due to presence of phenol red. Since the DPM has essentially no moisture content, this does not represent a degradative situation, and is why fluid bed processing is essential.

### Example 3

DPM that Includes Buffering Salts (e.g., Sodium Bicarbonate) and is Formulated so that pH of Reconstituted (1×) Medium is Automatically of Desired pH with No User Efforts—Spraying of Acid or Base Technique

As noted above, all commercially available mammalian cell culture powdered media require addition of one or more buffer salts (e.g., sodium bicarbonate) when preparing 1× liquid, and then adjustment of pH, so that the solution will be at proper pH. The present methods, however, can be used to obviate both the addition of sodium bicarbonate (as described above in Example 2) and the need for pH adjustment. In this aspect of the invention, fluid bed technology is used to introduce acid or base (depending on the need) to a dry powder medium comprising one or more buffering salts. In accordance with this aspect of the invention, any buffering salts or combinations thereof, and any acid or base, may be used depending upon the desired pH and buffering capacity in the ultimately reconstituted cell culture medium.

If sodium bicarbonate is added directly to the DPM as a powder, it is possible for the end user to simply add water and mix to yield a solution already containing bicarbonate (see above) and of proper pH. It is necessary first to determine how much of a pH adjustment is required.

(1) Place 1 L of water in a beaker. Add DPM to the liquid and mix. (Amount to add/L is given by the specifications for that powder, e.g., 10 g/L, 13 g/L). In this case, the weight of the sodium bicarbonate must also be considered in determining how much to add per liter.

(2) After the powder has dissolved, add 5N HCl to adjust the solution to the desired pH. Record the amount.

(3) Convert this number to amount of 1N HCl. Calculate how much 1N HCl is needed for adjustment of the total powder to be agglomerated. (Example: 5 ml of 1N HCl is needed to adjust 1 L of 1× medium A to pH 7.2 from the unadjusted pH of 7.9. That 1 L of 1× medium represents, for example, 13.0 g of DPM. Therefore, for each 13.0 g of DPM, 5 ml of 1N HCl is needed. If we want to adjust pH of 250 g of DPM, then 250 divided by 13.0=19.2×5 ml or 96 ml of 1N HCl is needed to be added to the powder to make it automatically pH-adjusted.)

This 1N HCl must now be added to the DPM. The best way for that is to use the injection device, adding 1N HCl instead of water. In general, the protocol is similar to the above with the following exceptions: (1) the 1N HCl must be added slowly to the media which contains sodium bicarbonate. If it is added too quickly, carbon dioxide may be driven off, resulting in suboptimal buffering capacity. Because of the volume of 1N HCl generally required, several 1 minute on, 2 minute off cycles are needed. A dry powder state must be obtained at the end of each cycle so that a dynamic system exists where DPM has characteristics of a fluid process but in reality is a dried powder. (Amazingly, as HCl is added to the powder, the bulk color changes from dark reddish purple to light yellow-orange color even though the powder remains essentially dry at all times due to the continual evaporation within the system). Since the total amount of HCl has been calculated to yield an essentially neutral pH, the powder is never really exposed to “acid” conditions as long as the fluid bed is properly adjusted (see above; position of the powder particles within the chamber during operation). It is important to make sure that all of the powder is moving through the system (i.e., being lifted, agglomerated and settled continuously) and having no “dead” zones within the chamber.

Once the powder is collected after the run, it can be added to water and reconstituted at any time as long as it has been kept in proper “dry” packaging and location. No adjustment of pH is needed. Thus, the invention provides an automatic pH-adjusting dry powdered medium, where the pH of the liquid medium made by reconstituting the dry powdered medium requires no adjustment of pH.

### Example 4

Inclusion of Large Molecular Weight Supplements Such as Serum, Albumin, Hy-Soy, Etc., within the DPM Itself

Heretofore, dried powder media containing serum have not been commercially available. Using the present methods (via fluid bed and spray-drying technologies), we have succeeded in adding serum to a powder in a manner where functionality (cell culture) is maintained.

The injection device of the fluid bed apparatus is able to form a mist with serum, and concentrated albumin. We attempted to see if serum added to the DPM and dried in this manner would be functional.

Procedure for Addition of Serum:

(1) Determine the weight of standard DPM to be agglomerated.
(2) From this, based upon the g/L for the particular powder, calculate the volume of 1× medium that the g of powder will make.
(3) Calculate the volume of serum that would be needed at a given percentage level of supplementation (e.g., 100 g of powder to be used in 10 g/L yields 10 L-equivalents of powder). At 5% serum supplementation, 500 ml of serum would be required to be added by the injection device.

Protocol for addition of the serum: Serum and albumin are very viscous. The nozzle spray pattern must be checked for droplet size and pattern. With the sample tube in the solution to be added to the powder, test spray against a cardboard or other backdrop. Check for uniformity and small droplet size. If not a “mist,” increase atomizing pressure by 0.5 bar and test again. Do this until sufficient pressure results in a fine mist pattern.

For use in cell culture applications, it is necessary to know the weight/ml of serum-DPM to be used per L of 1× medium. To do this, accurately weigh vials or test tubes that will hold the serum during drying. Place a constant (known) quantity of serum into each of the vials. Then place vials into a Speed Vac or lyophilizer. Remove water until dryness. Then weigh the vials again, this time containing lyophilized serum. Calculate the weight of serum and express as per ml of original volume. The weight of agglomerated DPM with serum to use per L will then be the standard DPM “use” weight plus the weight of the serum at a given level.

For example, assume that Medium A (DPM) is to be used at 10 g/l. Serum supplementation is to be at 5% v/v. This means that in addition to the weight of the standard DPM, the weight of the serum would equal 5%=50 ml to add per L of medium. Assume that serum powder weighs 0.06 g/ml. Then the weight of the powdered serum=50×0.06 g/L=3 g. Therefore, the weight of serum-containing DPM that would be added to 1 L of water is the weight of serum powder (3 g) plus the weight of the standard DPM (10 g) per liter=13 g/L.

### Example 5

Reducing or Eliminating Milling Techniques (High Energy Input System that Break Components Down to Micron-Sized Particles) when Manufacturing a DPM

As noted above, dry powdered medium typically is manufactured via the milling process, which is laborious and has a number of problems. The methods of the present invention provide for the production of a dry powdered medium using fluid bed technology, which overcomes these labor and technical constraints.

A. Blending First in External Device, then Fluid Bed Treatment

Normally milled DPM is blended with sodium bicarbonate (directly as received from the supplier, additional ball milling not needed). [RPMI 1640 with sodium bicarbonate at 2 g/L-equivalents]. This mixture is blended for 20 minutes. The powder is then placed within the fluid bed chamber and fluidized as above for bicarbonate-containing media or bicarbonate-containing media with automatic pH control.

B. Blending Directly in Fluid Bed Chamber, then Agglomeration

Sodium bicarbonate is placed into the chamber directly with the milled DPM and blended (mixed) for a brief period of time, to be followed with agglomeration. This eliminates blending in a separate unit.

### C. Total Elimination of the Ball-Milling Process

Either all of the DPM chemicals are added directly to the fluid bed chamber and mixed preliminarily followed by agglomeration or, more likely, some of the coarser, “stickier”, etc. chemicals are given a brief grinding treatment in a rotary grinder and then placed within the fluid bed for blending and final agglomeration.

### Example 6

A Method for Having all of the Above Characteristics within this Same DPM

We have combined addition of “off the shelf” sodium bicarbonate with milled DPM and automatic pH control. We have also combined serum with DPM.

To combine serum with DPM containing sodium bicarbonate with automatic pH control, one protocol is to:

1. Add sodium bicarbonate (powder, from supplier) to DPM (milled or ground).

2. Blend ingredients (mix, either external unit or fluid bed).

3. In a separate vessel, reconstitute 1 L of the DPM (containing bicarbonate) with water (1×) and determine the amount of 1N HCl, or 1N NaOH that is required to adjust the pH of the solution to 7.5. On a liter basis, knowing the mass of powder to be agglomerated (and thus the L-equivalents), calculate the amount of 1N HCl or 1N NaOH for the total powder to be agglomerated at the above-calculated amount. Add this amount via fluid bed device (injection nozzle). (Although DPM is not “liquid,” it is important to have a powder as close to neutrality as possible but not of such an acid pH that bicarbonate would be liberated when adding serum, since moisture is involved in the process. At pH 7.6 or higher, a concentrated solution of sodium bicarbonate will not evolve CO₂ gas, but at lower pH gas will be given off.)

4. Addition of serum (extended agglomeration), based upon percentage supplementation and g to be agglomerated.

5. Using the same 1 L of 1× liquid from (3) above, determine the amount of 1N HCl or 1N NaOH needed to adjust the pH to the desired pH (e.g., 7.2). Using this information, calculate the amount to be used for the weight of powder that has been agglomerated with serum (knowing g/L specifications). Add this amount via fluid device (injection nozzle).

6. Gamma irradiation is used to sterilize the powdered media.

In a similar method, a serum-containing DPM may be produced by combining a particular amount of DPM with a particular amount of powdered serum (prepared, e.g., by spray-drying as described in Example 8 below) and then agglomerating the mixture. For example, for preparation of medium containing 10% powdered FBS, 55.5 g powdered FBS may be added to 500 g of powdered culture medium and the powders mixed well by agitation. This mixture may then be water-agglomerated as described above, and will yield, upon reconstitution, a culture medium containing 10% FBS which may be auto-pH-adjusting.

### Example 7

### Production of 100% Serum Powder by Fluid Bed Processing (to Simulate Spray-Drying)

### Methodology

1) We used the benchtop laboratory fluid bed apparatus (Strea-1). For production of powdered serum, nothing is placed within the chamber. The lever is used to seal the unit.

2) Serum was added by way of the injection device (spray unit). As the serum was added into the chamber, the air flow was increased enough and the flow of serum slowed enough that evaporation of water occurred and the serum was dried sufficiently so that powder formed instantly within the chamber. No moist or fluid coating existed anywhere within the chamber.

3) Pump speed was set to allow for ˜1 ml/minute into the chamber.

4) Airflow speed was set to a setting of ˜8-9.

5) To clean filters intermittently, fan speed was reduced to ˜2-3. This was done routinely every 5-10 minutes. (The 8-9 airflow setting is so high that the filters will not blow off the powder and clean themselves).

6) After one round of filter blow-off, fan speed was increased to previous levels and the pump turned on. (Once these parameters were set, the pump was run continuously except when cleaning the filters as indicated).

7) After all of the serum liquid had been added into the agglomerator, final drying was performed over five minutes.

8) The filters were then blown off to collect as much powder as possible, and the machine shut off and product removed. Powdered serum was placed into an air-tight container and protected from light.

### Typical Instrument Settings

Drying temperature: 60-65° C.

Outlet air temperature: ˜33° C.

Blow out pressure: 5 bar

Atomizing pressure: 2.0-2.5 bar

Blow back dwell: 2, in between spraying

Capacity of fan: 8-9 throughout run

Magnahelics: Filter resistance—150-250,

Resistance of perforated control plate—˜50,

Air volume—less than 50.

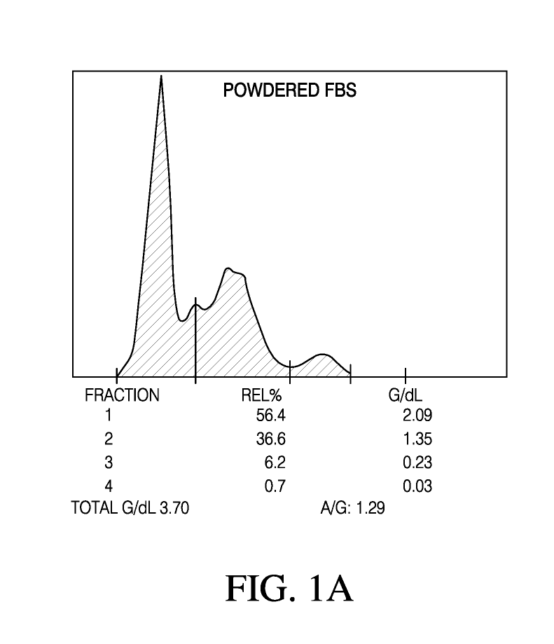

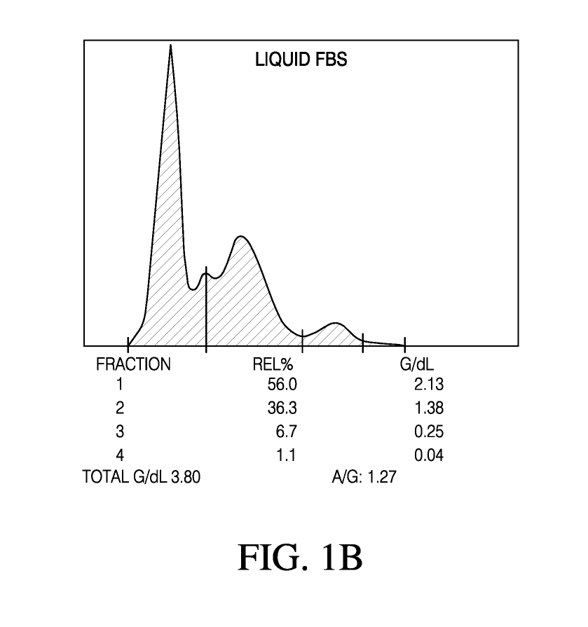

To determine if agglomeration of the FBS affected the protein structure or distribution, samples of agglomerated FBS and liquid FBS were run on SDS-PAGE, stained for protein and scanned densitometrically. As shown in FIG. 1, agglomerated FBS prepared according to the present methods (FIG. 1A) demonstrated a nearly identical protein profile to that observed with liquid FBS (FIG. 1B). These results indicate that the controlled production of dry powdered FBS by the present methods does not substantially affect the structure or distribution of the major components of the serum.

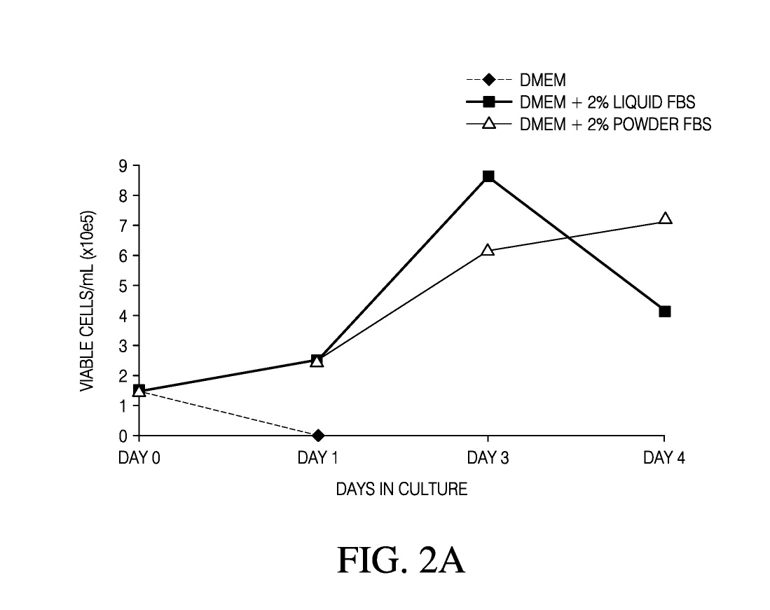

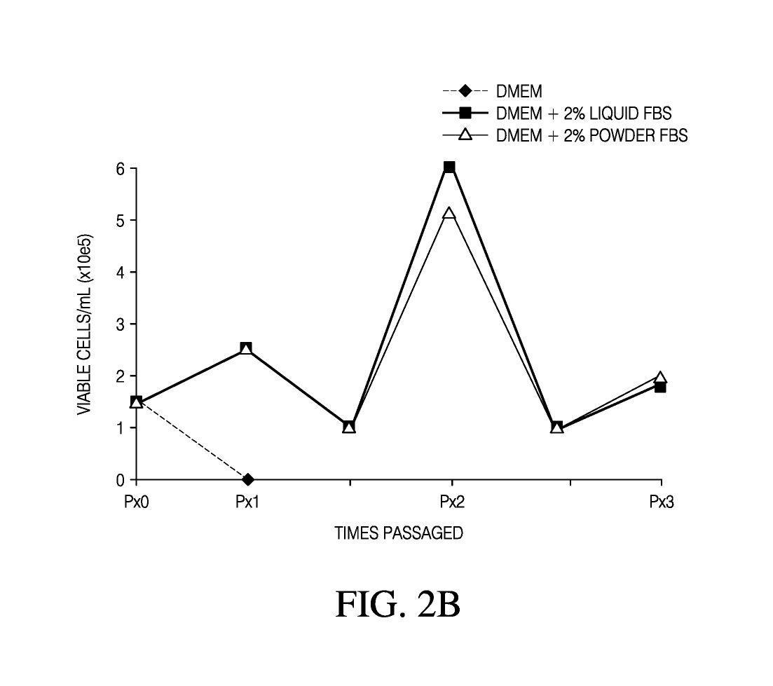

To determine if agglomeration of the FBS affected its ability to support cell growth and passage, SP2/0 cells were plated into DMEM containing either 2% agglomerated (“dry”) FBS or 2% liquid FBS and growth rates and passage recovery examined. As shown in FIG. 2A, cells plated into media containing agglomerated FBS demonstrated similar growth kinetics as did cells plated into media containing liquid FBS. Similarly, cells in media containing agglomerated FBS recovered from passage with practically identical growth rates as cells in media containing liquid FBS (FIG. 2B). Together, these results indicate that the agglomerated FBS of the present invention performs approximately equivalently to liquid FBS in supporting growth and passage of cultured cells.

### Example 8

### Production of 100% Serum Powder by Spray-Drying

As an alternative to fluid bed processing, the feasibility of producing dry powdered serum by spray-drying technology was examined. A three foot diameter laboratory spray drier (Mobile Minor Spray Drier; NIRO, Columbia, Md.) was used to prepare the powdered serum. Liquid FBS was aspirated into the spray-dryer and atomized through a Schlick 940 nozzle located in the middle of the air dispenser, and the drying air was introduced into the atomizer through the top air dispenser of the apparatus. Spray drying was conducted under the following conditions: inlet air temperature=200° C.; outlet air temperature=70° C., atomizing air pressure for the nozzle=2.0 bar; air flow=80.0 kg/hour; spray rate=65 g/minute. During development of these methods, an initial outlet air temperature of 60° C. was used; however, this temperature was found to be too low, and the spray rate was adjusted back to a level to achieve an outlet temperature of about 70° C. which was found to be optimal. Following spray-drying, powdered serum was collected at the cyclone of the apparatus, and process air was filtered through an exhaust filter prior to recirculation within the apparatus.

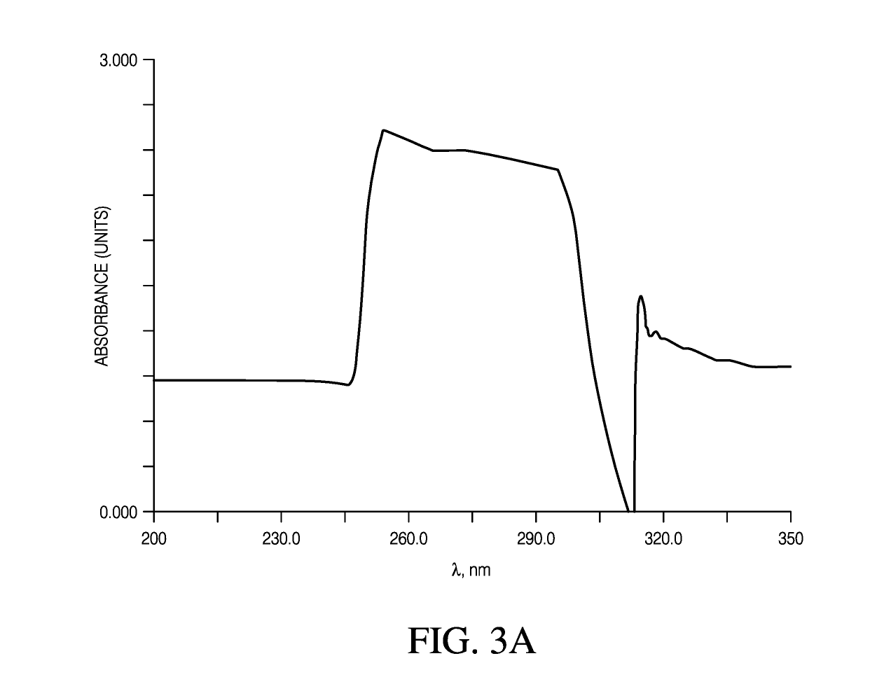

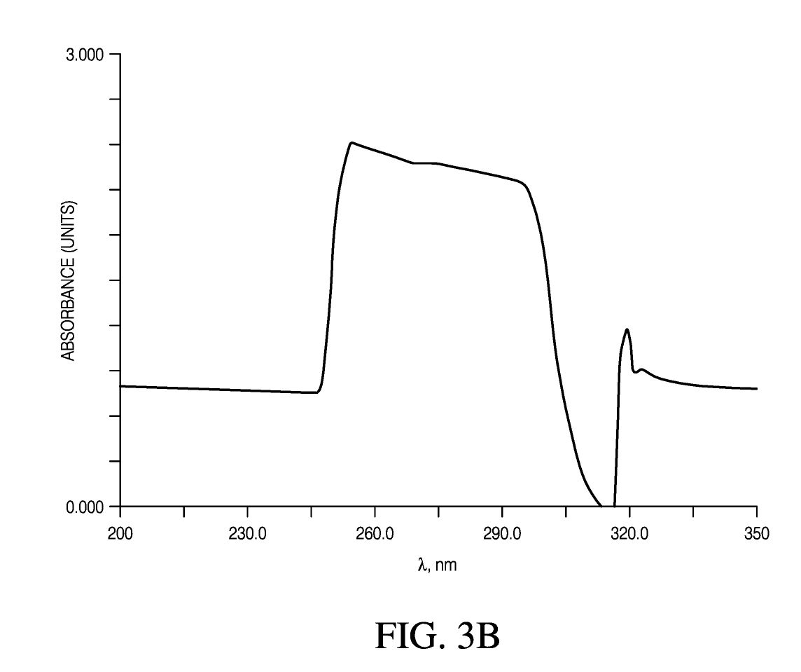

Following production, the powdered serum was characterized with respect to its physical properties, compared to liquid FBS from the same source lot. Samples taken from different stages of the production lot (samples “A” and “B”) were reconstituted at a concentration of 60.44 g/L in endotoxin-free distilled water (Invitrogen Corporation), and were examined for endotoxin levels using a Limulus Amoebocyte Lysate test (Invitrogen Corporation), for hemoglobin levels (by spectrophotometrically measuring absorbance at 525 nm), and by UV/Vis spectrophotometry. Results are shown in Table 3, and in FIGS. 3A and 3B.

TABLE 3
Physical Characterization of Powdered Serum.

| Material Tested | Endotoxin Level (EU/ml) | Hemoglobin (mg/100 ml) |
|---|---|---|
| Powdered FBS, | 0.6 | 7.7 |
| Sample “A” | | |
| Powdered FBS, | <0.3 | 7.7 |
| Sample “B” | | |
| Liquid FBS | <0.3 | 7.2 |
| (control) | | |

As seen in Table 3, powdered FBS demonstrated endotoxin and hemoglobin levels similar to those of the liquid FBS that served as the source material for production of the powdered FBS. Moreover, samples taken from different stages of the production process demonstrated nearly identical endotoxin and hemoglobin levels, indicating that the present methods result in the production of material with approximately uniform physical consistency across the production lot. When samples of powdered and liquid FBS were examined by UV/visible spectrophotometry (FIG. 3), the trace observed for powdered FBS (FIG. 3A) was indistinguishable from that obtained for the source liquid FBS (FIG. 3B). Together, these results indicate that serum powder prepared by the present spray-drying methods have nearly identical physical characteristics as those of liquid sera from which the powders are prepared. Taken together with those of Example 7 above (see, e.g., FIG. 1), these results demonstrate that the methods provided by the present invention result in the production of powdered sera with physical characteristics that are unaltered from those of the source liquid sera.

Unexpectedly, as shown in Example 18, it was found that endotoxin level in serum is reduced with spray-drying. Failure to detect such reduction here may be attributed to the low levels of endotoxin present in the sample and/or the sensitivity of the assay.

### Example 9

Production of Automatically pH-Adjusted Powdered Culture Media

One reason that sodium bicarbonate is never included in powdered media is that any moisture, even that in the air, may result in an acidic condition within the pouch that will result in the liberation of CO₂ gas. The pouches will become swollen and produce what have been called “pillows.” With fluid bed processing, the humidity within the apparatus is reduced essentially to negligible levels prior to the end of the process. We have made RPMI-1640 powdered media containing sodium bicarbonate and have not seen evidence of “pillow” formation.

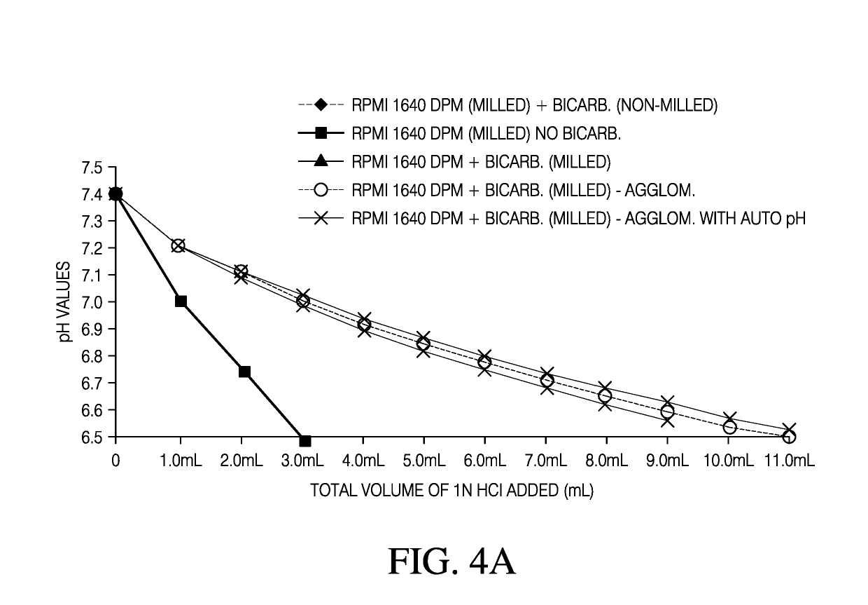

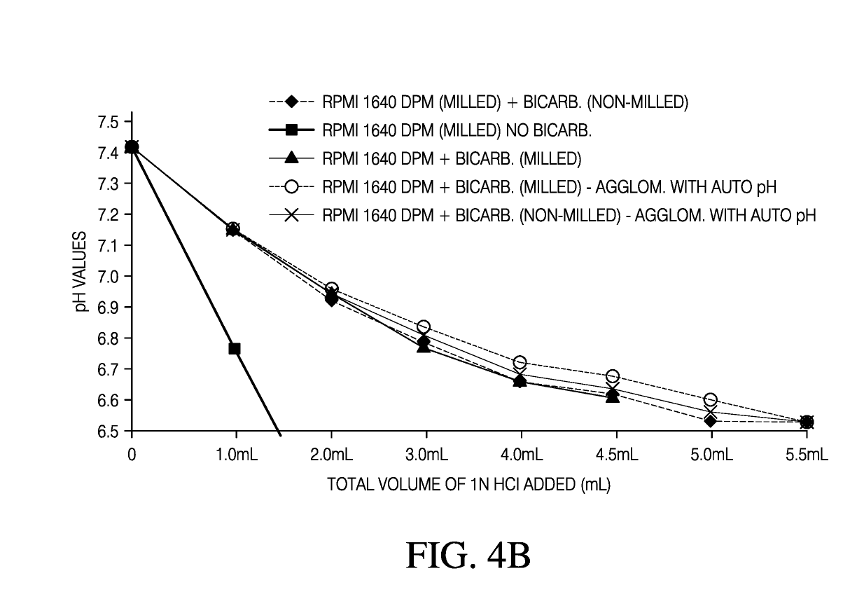

In order to make a pH-adjusted powdered media, it is necessary to add the pH-adjusting chemical (usually HCl or NaOH) to the powder to bring the pH to about 7.0-7.4 upon addition to water. Once sodium bicarbonate is added to the powder, many powdered media reconstitute in water on the basic side of neutrality and need HCl addition. Adding HCl to a powder containing sodium bicarbonate would be expected to be problematic. However, since the added liquid (5N HCl in this case) never results in a moistened or “liquid” state inside the fluid bed apparatus, the sodium bicarbonate does not give off CO₂ gas and fully retains its buffering capacity. This has been examined in the present studies by pH-titering experiments: equal amounts of acid, in two separate experiments (FIGS. 4A and 4B) were found to reduce the pH of agglomerated media and automatic pH-adjusted agglomerated media by an identical amount as that for a standard medium with sodium bicarbonate added to the liquid at the time of reconstitution. These results indicate that both agglomeration with subsequent adjustment of pH, and agglomeration with adjustment of pH during the agglomeration process, function equally well to produce powdered culture media with significant buffering capacity.

### Example 10

### Effect of Agglomeration on Dissolution Rates of Culture Media

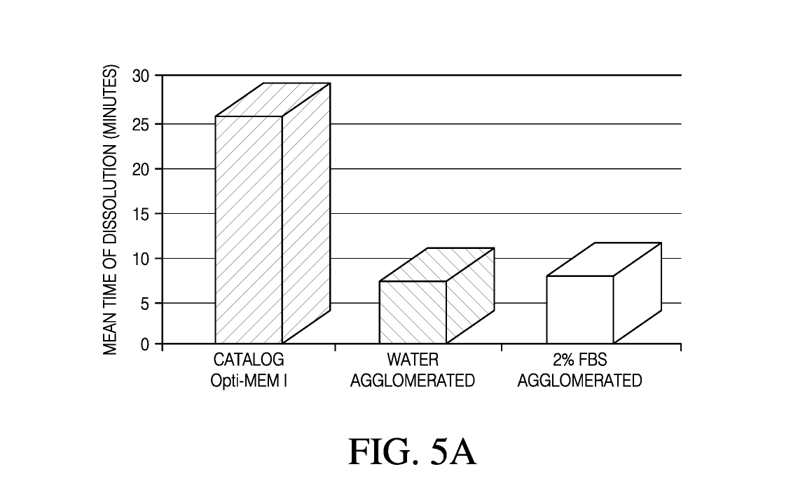

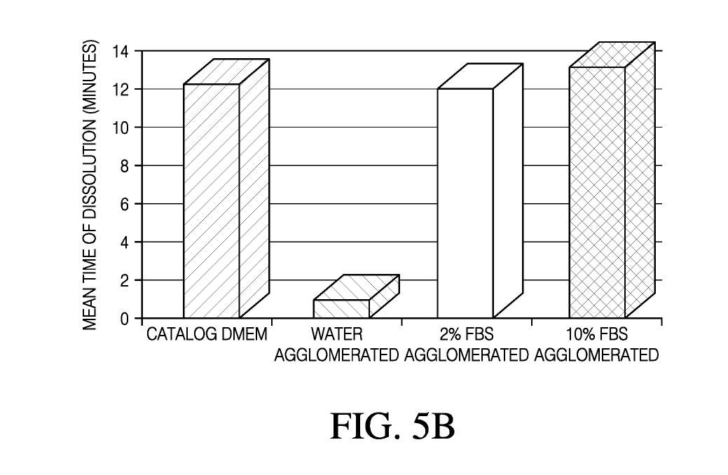

To examine the effect of agglomeration of culture media on the rate of dissolution of the media, samples of Opti-MEM I™ or DMEM were agglomerated with water or with FBS (2% only for Opti-MEM I; 2% or 10% for DMEM). Upon reconstitution of the agglomerated media in water, the time dissolution of the agglomerated Opti-MEM I occurred much more quickly than did dissolution of standard powdered Opti-MEM I (FIG. 5A); results were identical for water- and FBS-agglomerated Opti-MEM I. Interestingly, however, while water-agglomerated DMEM dissolved in water much more quickly than did standard powdered DMEM, the FBS-agglomerated DMEM did not (FIG. 5B).

Due to the open structure of the agglomerated powdered media (as opposed to traditional powdered media), capillary action brings water into close proximity with all of the powder particles. This prevents the appearance of powder “balls,” a complication observed upon reconstitution of most standard powdered media that leads to longer dissolution times. In addition to more rapid dissolution, agglomerated media demonstrated reduced dusting as well. These results indicate that water-agglomerated culture media, and some FBS-agglomerated culture media, are much more rapidly dissolving and generate less dust than traditional powdered culture media.

### Example 11

### Cell Growth and Subculturing in Reconstituted Agglomerated Culture Media

Many uses of culture media require additions of large molecular weight proteins such as serum or albumin. These molecules may be in the form of solutions or even powder in the case of albumin. However, in order to insure uniformity of powdered media, these proteins are usually added not as a powder but as liquid after reconstitution of the bulk powdered media to a liquid medium. This presents some inconvenience since, for example, serum must be stored in the freezer to maintain performance over time. This adds expense and inconvenience since the serum must be added aseptically to the media, increasing chances of contamination. If filtration is done after addition of serum, another processing step is needed. There would therefore be advantages to being able to provide serum as an integral part of the powdered media.

Therefore, culture media were agglomerated with water or with various concentrations of FBS. FBS was added to the powdered media by injecting it into the air-suspended dry powdered media at high evaporation rates, as generally outlined above. The level of serum supplementation was 2% in Opti-MEM I media, and 2% or 10% in DMEM. The growth and passage success of various cell lines in these media were then assessed.

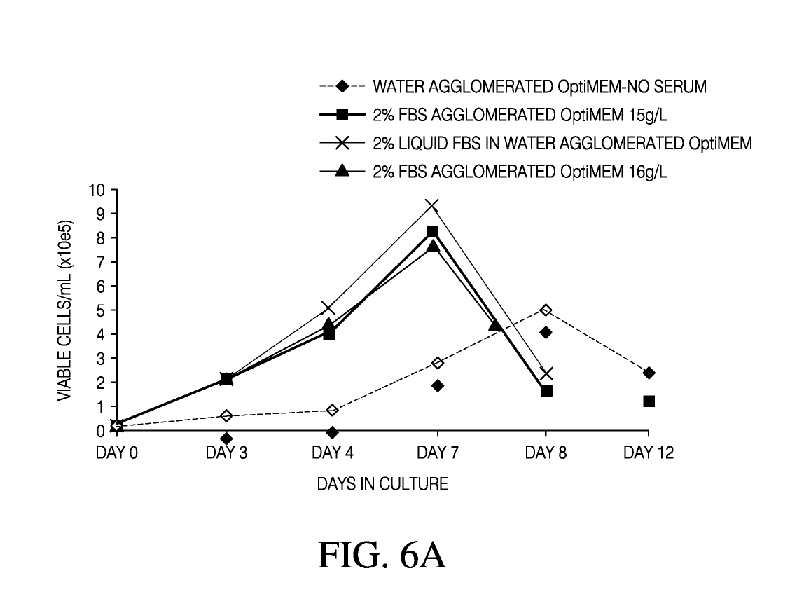

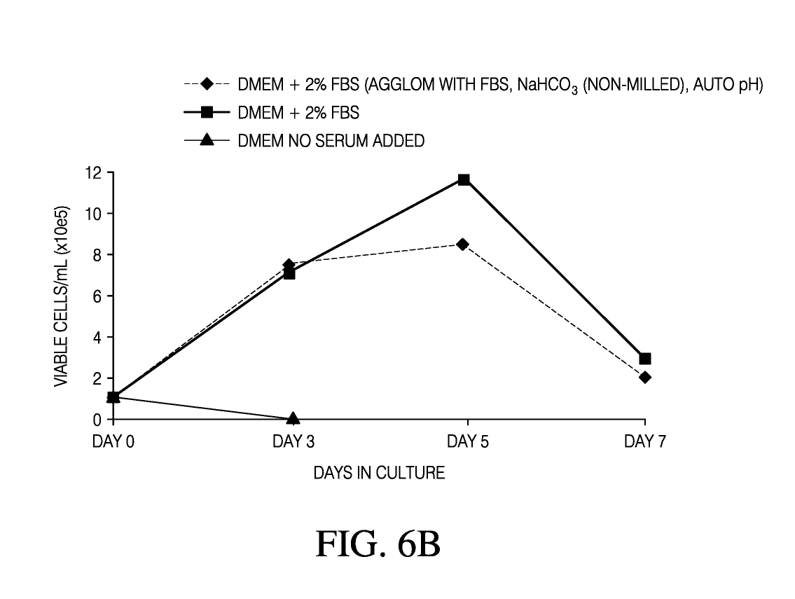

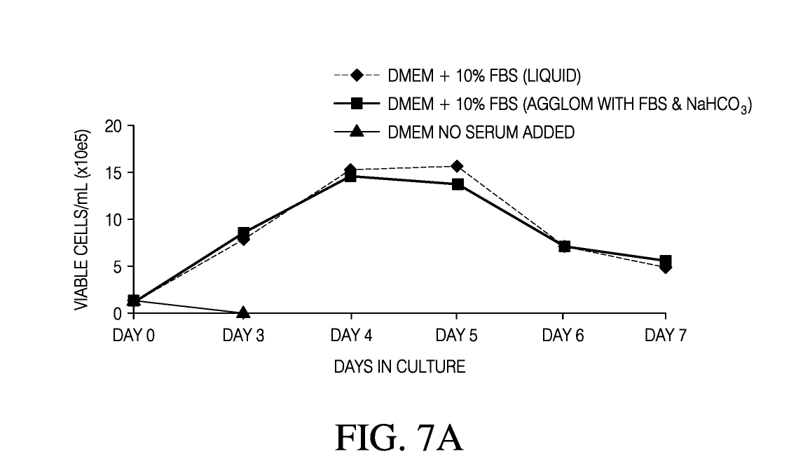

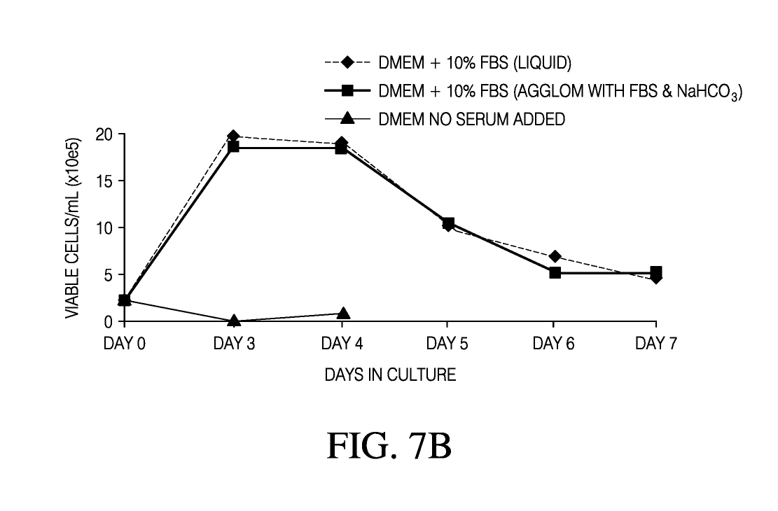

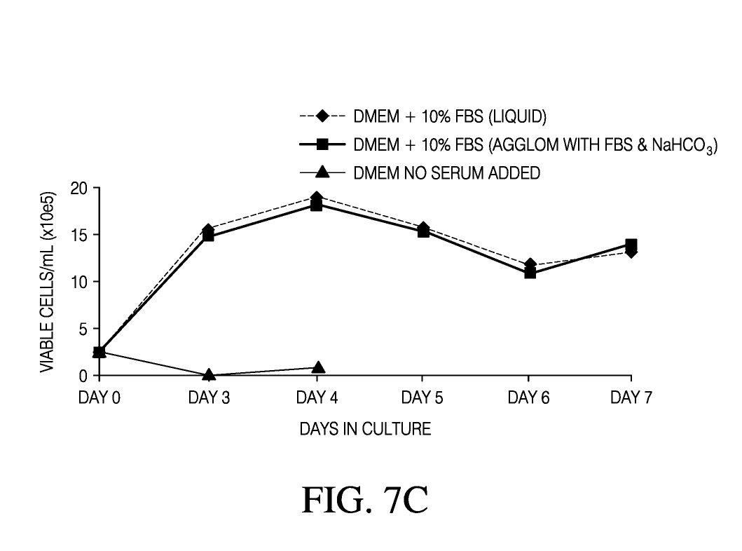

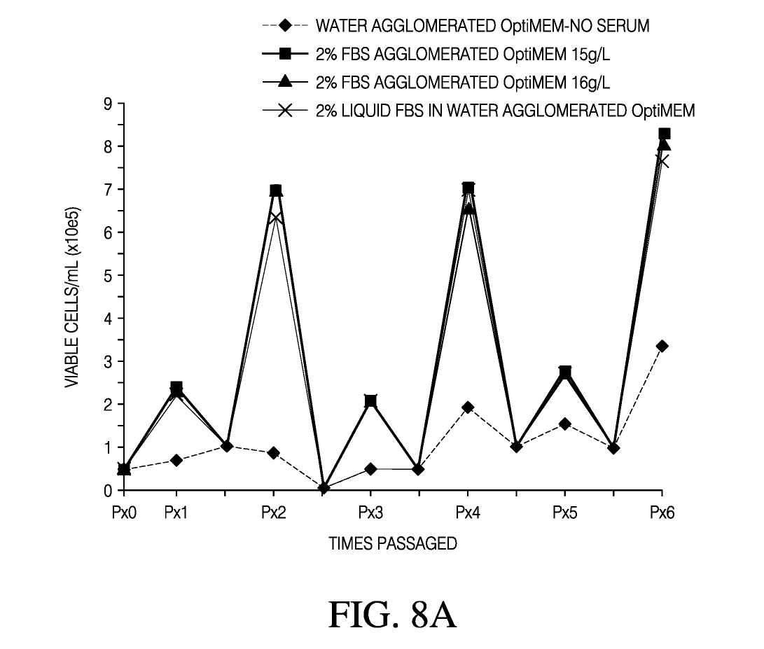

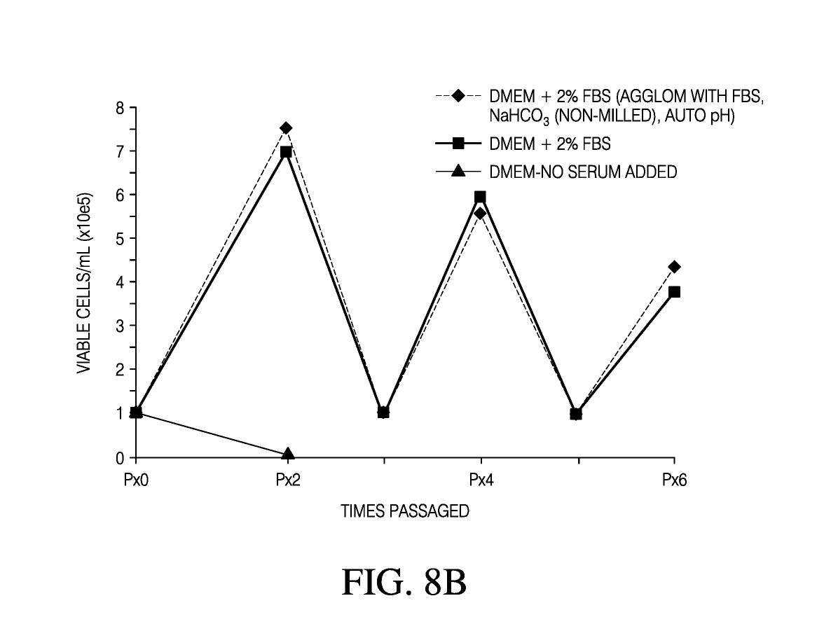

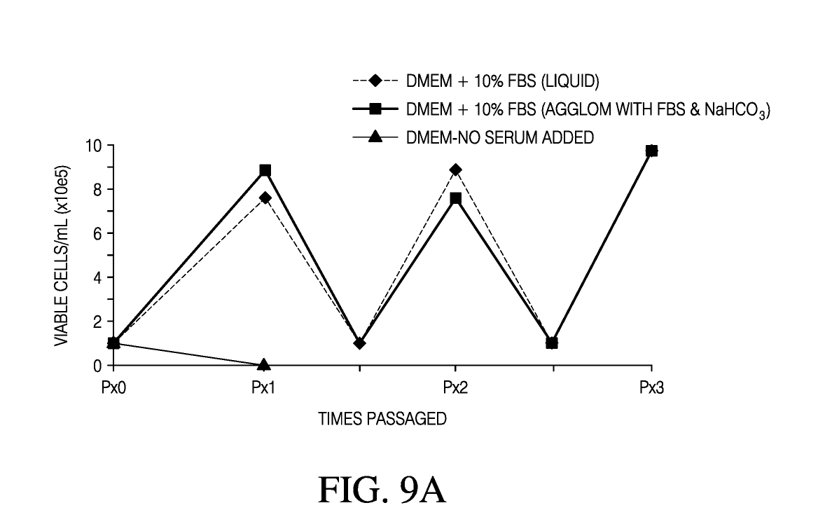

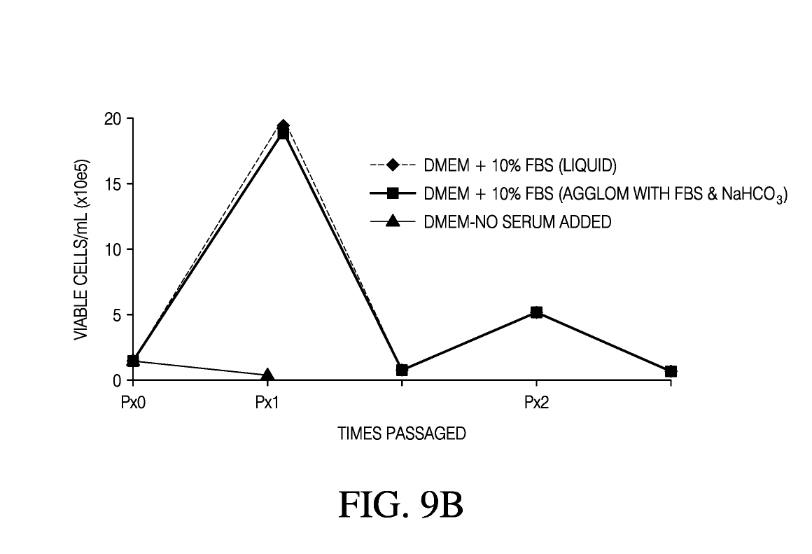

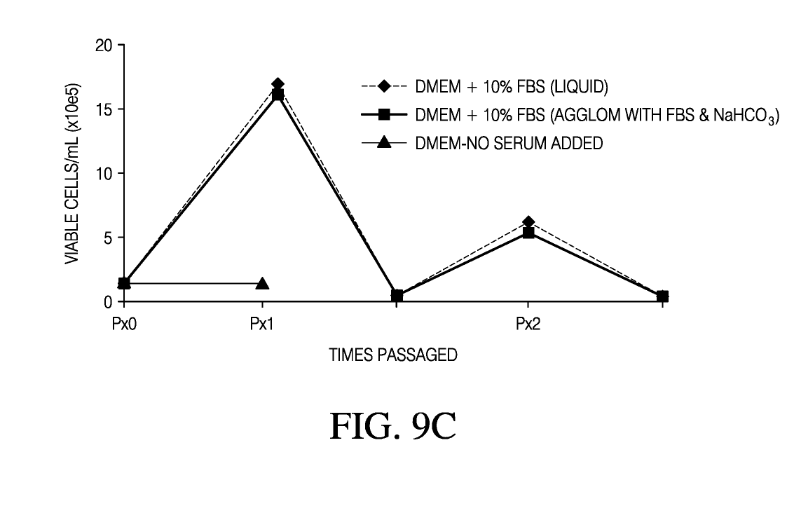

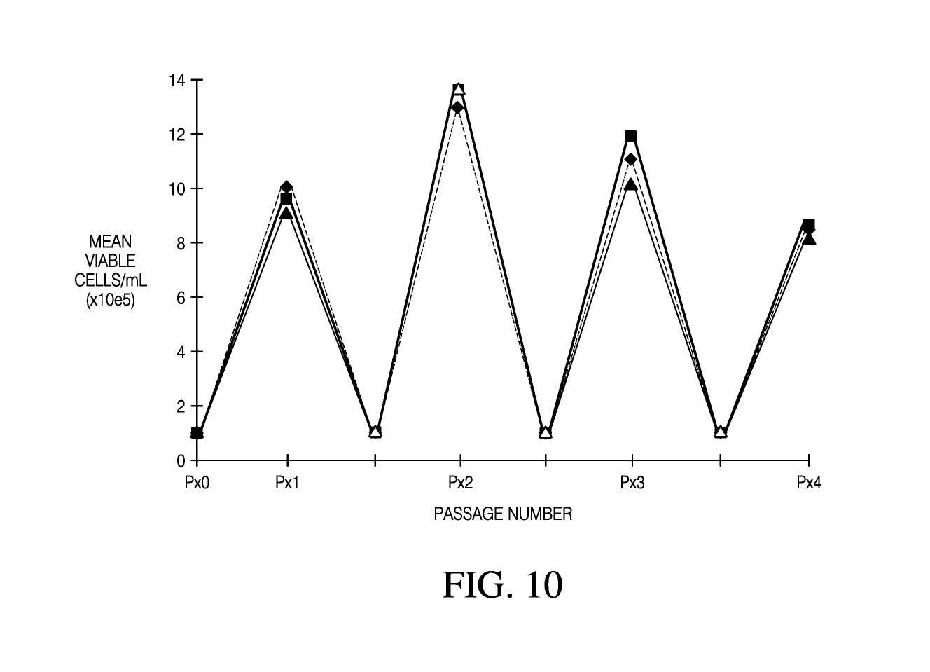

As shown in FIG. 6, SP2/0 cells demonstrated similar growth rates when grown in Opti-MEM I agglomerated with either water or with FBS (FIG. 6A), compared to cells grown under conventional culture conditions (liquid serum added to water-reconstituted powdered media). Similar results were observed with SP2/0 cells cultured in water- and FBS-agglomerated DMEM supplemented with 2% FBS (FIG. 6B), and with SP2/0 cells (FIG. 7A), AE-1 cells (FIG. 7B) and L5.1 cells (FIG. 7C) cultured in water- and FBS-agglomerated DMEM supplemented with 10% FBS. In addition, SP2/0 cells showed approximately similar recovery rates from passage when cultured in water- or agglomerated Opti-MEM I and DMEM supplemented with 2% FBS (FIGS. 8A and 8B, respectively), as did SP2/0 cells, AE-1 cells and L5.1 cells cultured in water- and FBS-agglomerated DMEM supplemented with 10% FBS (FIGS. 9A, 9B and 9C, respectively) and SP2/0 cells cultured in water-agglomerated DMEM supplemented with 5% FBS (FIG. 10). Furthermore, SP2/0 cells demonstrated identical passage characteristics in water-agglomerated media produced in large batches and in automatically pH-adjusting powdered DMEM containing sodium bicarbonate as they did in standard liquid DMEM supplemented with 5% FBS (FIG. 10).

Together, these results indicate that culture media supplements such as animal sera (e.g., FBS) may be agglomerated directly into culture media, and that supplementation of culture media during the agglomeration process in this way produces a culture medium that provides optimal support of growth and passage of a variety of cultured cells. Furthermore, these results indicate that the present culture media powders may be successfully produced in large batches, including the automatically pH-adjusting media of the invention that contain sodium bicarbonate.

### Example 12

Cell Growth in Culture Media Supplemented with Spray-Dried Serum Powder

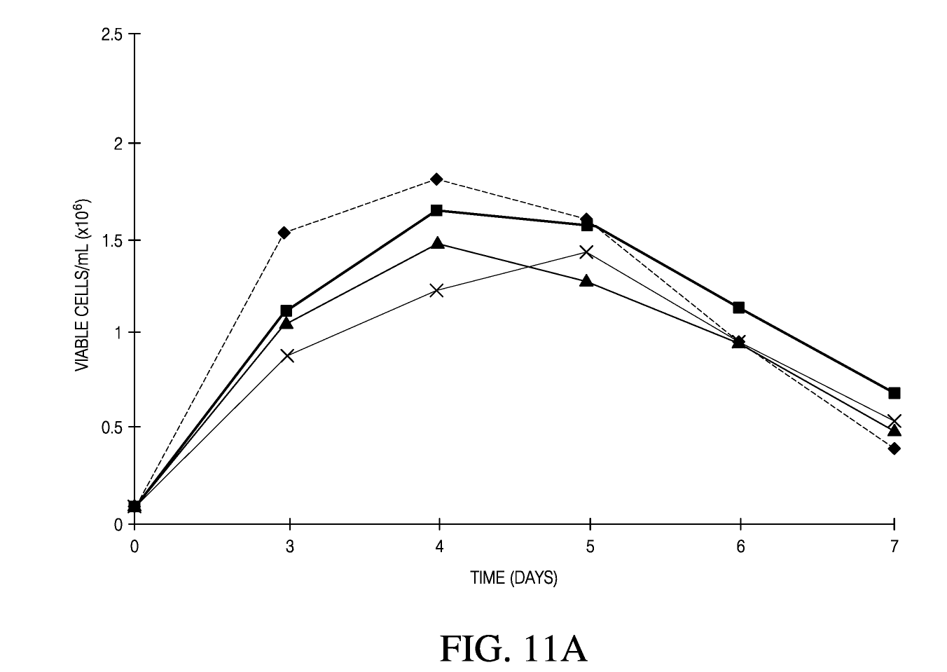

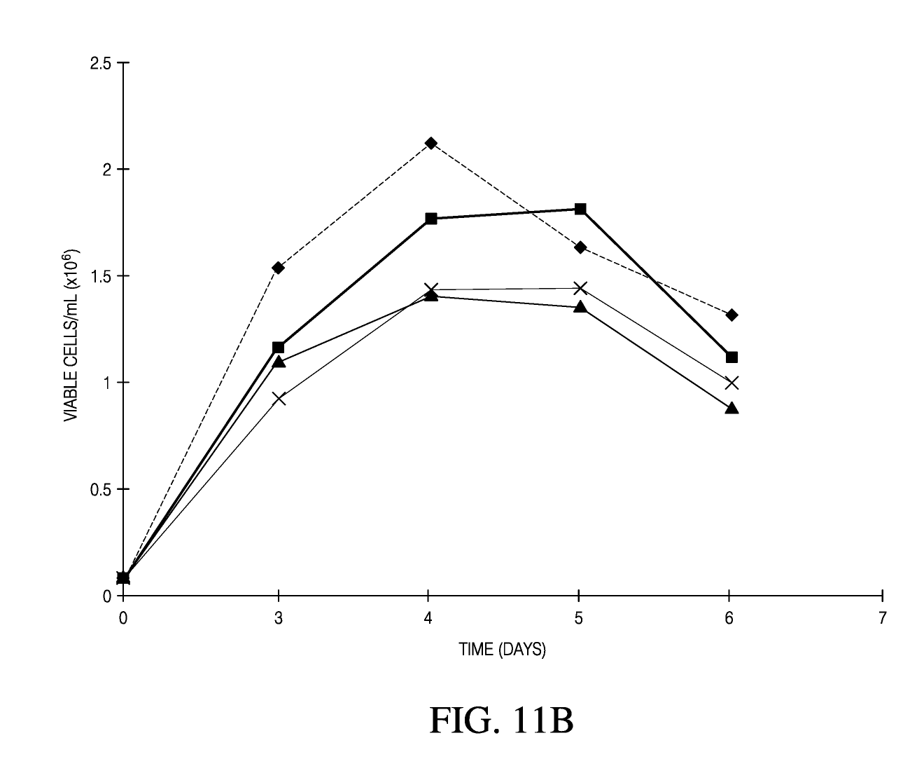

As a corollary to the experiments shown in Example 7, AE-1 cells and SP2/0 cells were plated into DMEM containing either 2% or 10% spray-dried FBS prepared as described in Example 8, or containing 2% or 10% liquid FBS, and growth rates and passage recovery of the cells were examined. Cells were inoculated into triplicate 25 cm2 flasks at a density of 1×10⁵ cells/ml in 10 ml of media. Viable cell density was determined on days 3-7, and each cell line was tested twice. Results are shown in FIGS. 11-13.

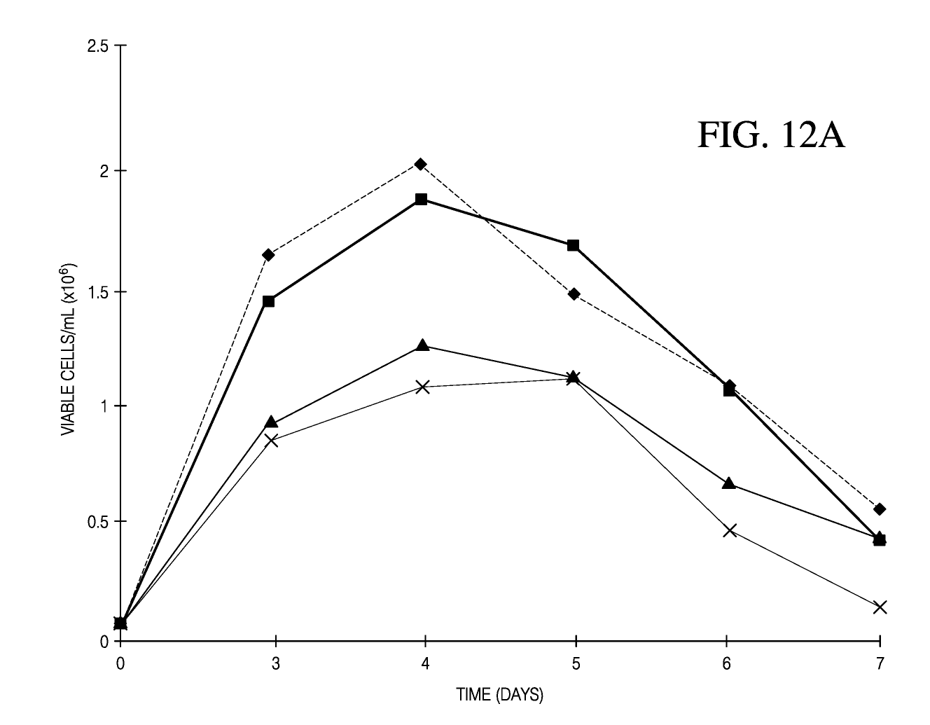

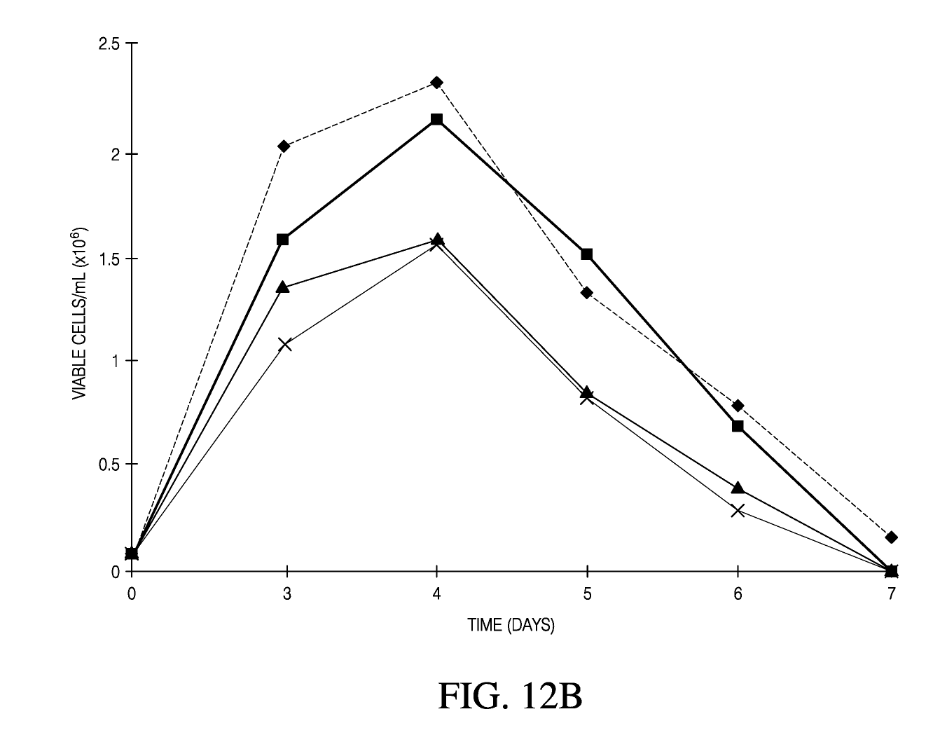

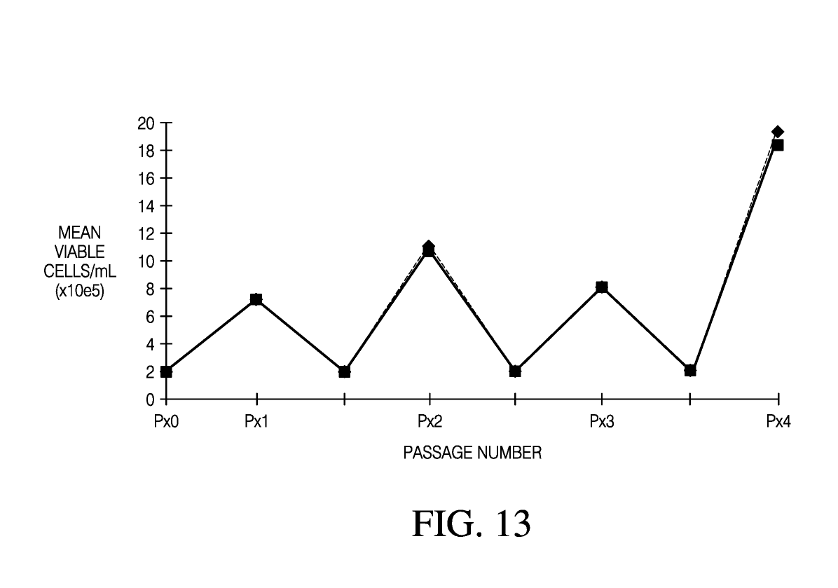

As shown in FIG. 11, AE-1 cells cultured in media containing powdered FBS demonstrated similar growth kinetics to those cells cultured in media containing standard liquid FBS. As expected, the cells demonstrated more rapid growth to a higher density in culture media containing 10% FBS than in media containing 2% FBS, and demonstrated peak growth by about day four. Similar kinetics were observed for two separate experiments (FIGS. 11A and 11B), indicating that these results were reproducible. Analogous results were obtained in two experiments in which the growth rates of SP2/0 cells were measured in media containing powdered or liquid FBS (FIGS. 12A and 12B). In addition, AE-1 cells cultured in media containing 5% powdered FBS recovered from passage with identical growth rates as cells in media containing liquid FBS (FIG. 13).

These results indicate that the powdered FBS prepared by the spray-drying methods of the present invention performs approximately equivalently to liquid FBS in supporting growth and passage of cultured cells. Together with those from Examples 7 and 8, these results indicate that the methods of the present invention may be used to produce powdered FBS, by fluid bed or spray-drying technologies, that demonstrates nearly identical physical and performance characteristics as those of liquid FBS.

### Example 13

### Effect of Irradiation on Performance of Agglomerated Media

Recently, concerns have been raised about the biological purity of media and media components (including supplements) used for bioproduction, particularly in the biotechnology industry. Gamma irradiation is a sterilization process that is known to work well with certain liquids and powders that are not typically amenable to sterilization by heat or toxic gas exposure. Therefore, samples of water- or FBS-agglomerated culture media were (irradiated with a cobalt source at 25 kGy for up to several days, and the growth rates of various cell types examined.

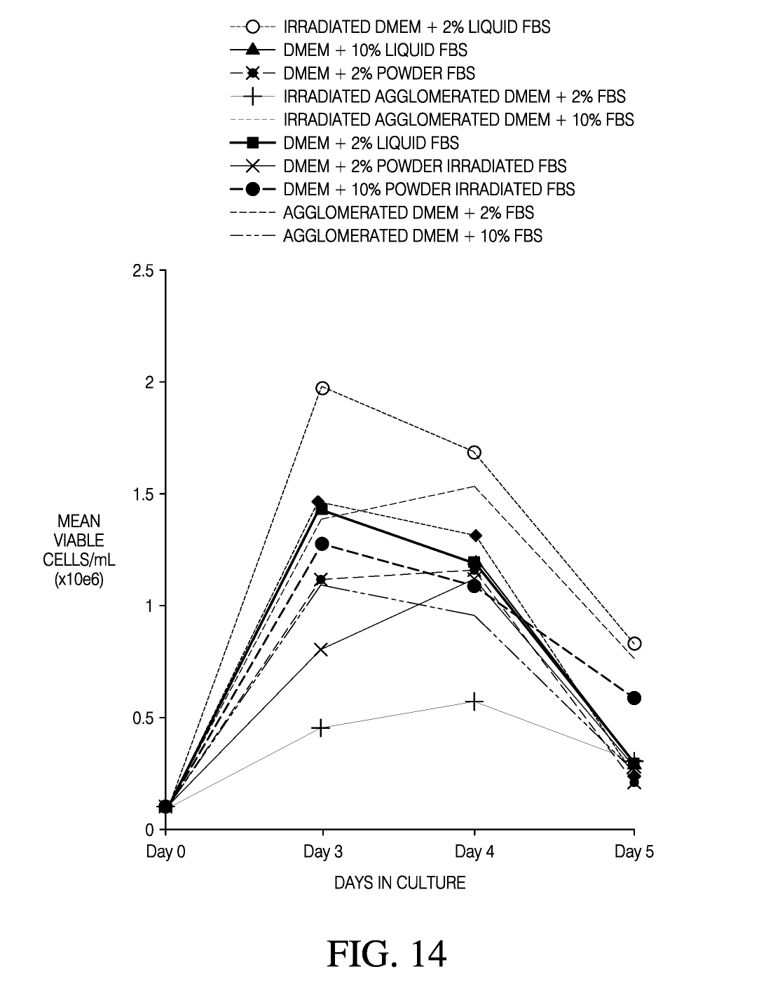

In one set of experiments, SP2/0 cells were inoculated into various media at 1×10⁵ cells/ml and cultured at 37° C. At various intervals, samples were obtained aseptically and cell counts determined by Coulter counting and viability determined by trypan blue exclusion. Media were prepared by dissolving sufficient powdered media to make a 1× solution in 1 L of water, stirring and filtering through a 0.22 am filter. Results are shown in the graph in FIG. 14. Those conditions on the graph that state “pwdr FBS” on the graph refer to the addition of powdered FBS (prepared as in Examples 7 or 8 above) to the reconstituted 1× medium prepared from either standard powdered media or from agglomerated media (irradiated or non-irradiated). Those conditions on the graph that state “Irradia. agglom. DMEM+FBS” refer to use of the fluid bed to make the agglomerated media by spraying FBS into the powdered media (standard or agglomerated) to make an FBS-agglomerated media.

As shown in FIG. 14, γ irradiation of standard powdered basal media and agglomerated basal media did not deleteriously affect the ability of these media to support SP2/0 cell growth. In addition, while irradiation did negatively impact powdered media containing powdered FBS, and powdered FBS itself, this effect diminished with increasing serum concentration.

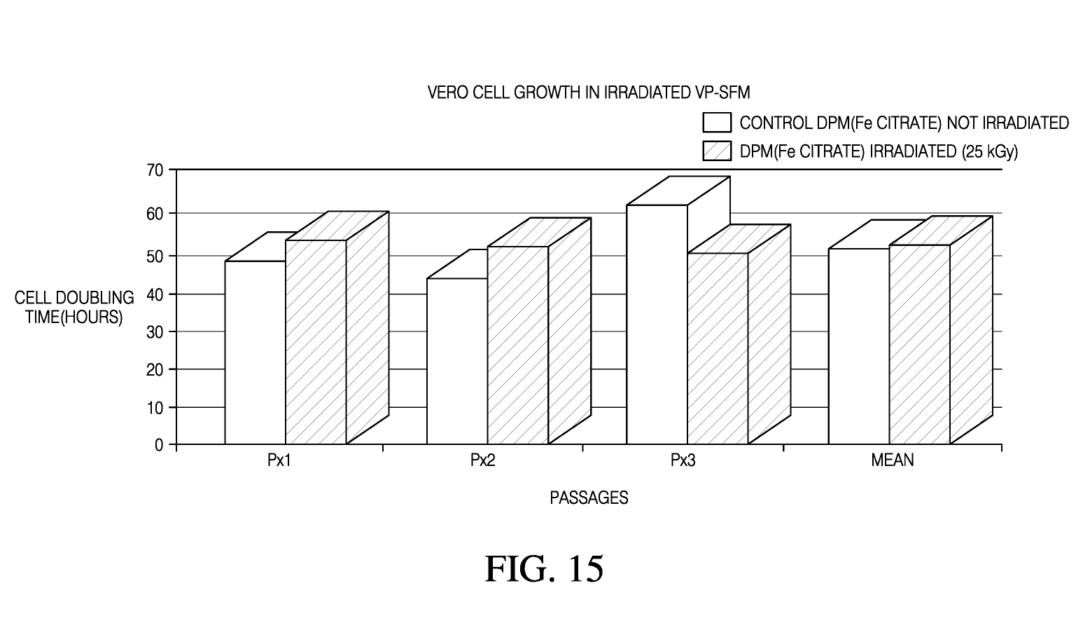

To more broadly examine these γ irradiation effects, samples of VERO cells were inoculated into VP-SFM™ that had been conventionally reconstituted or agglomerated as above. To the powdered media in the agglomeration chamber, however, epidermal growth factor (EGF) and ferric citrate chelate, traditional supplements for this media, were added via the spray nozzle during agglomeration. Media were then used directly or were γ irradiated as described above. Cells were inoculated at 3×10⁵ cells/flask into T-25 flasks and incubated at 37° C. Cell counts and viability were performed as described above, with results shown in FIG. 15.

As seen in FIG. 15, VERO cells demonstrated approximately equivalent growth and passage success when cultured in agglomerated media that had been γ irradiated as in agglomerated media that had not been γ irradiated. Furthermore, irradiation of the media had no effect on the low-level culture supplements EGF and ferric citrate chelate that were present in the media.

These results indicate that γ irradiation may be used as a sterilization technique in the preparation of many bulk agglomerated culture media, including those containing serum, EGF or other supplements, by the present methods.

### Example 14

### Effect of Irradiation on Performance of Powdered Media Supplements

To demonstrate the efficacy of the present methods in producing sterile media supplements, lyophilized human holo-transferrin was irradiated by exposure to a cobalt γ source at 25 kGy for about 3 days at −70° C. or at room temperature. 293 cells were then cultured in media that were supplemented with irradiated transferrin or with control transferrin that had not been irradiated (stored at −70° C. or at room temperature), and cell growth compared to that of standard transferrin-containing culture media or media that contained no transferrin.

Mid-log phase 293 cells that were growing in serum-free 293 medium (293 SFM) were harvested, washed once at 200×g for 5 minutes and resuspended in transferrin-free 293 SFM for counting and viability determination. Cells were plated into triplicate 125 ml Ehrlenmeyer flasks at a density of 3×105 cells/ml in a volume of 20 ml in 293 SFM (positive control), transferrin-free 293 SFM (negative control), in 293 SFM containing non-irradiated transferrin stored at −70° C. or at room temperature, or in 293 SFM containing irradiated transferrin prepared as described above. Flasks were placed into a rotary shaker set at about 125 rpm, in a 37° C. incubator equilibrated with an atmosphere of 8% CO₂/92% air. Daily cell counts were determined using a Coulter particle counter and viabilities were determined by trypan blue exclusion according to standard procedures. When the cells reached a density of about 1.2 to 1.7×10⁶ per flask, the contents of one of the flasks of each sample were harvested, centrifuged, resuspended into fresh medium and passaged into three new flasks. Cell counts and viabilities of the previous and next passages were then performed as described above. Four consecutive passages of cells incubated under the above conditions were tested.

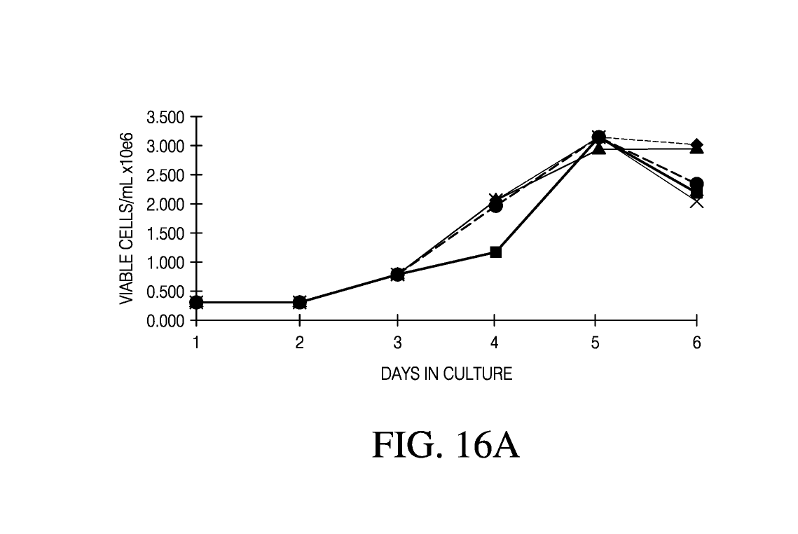

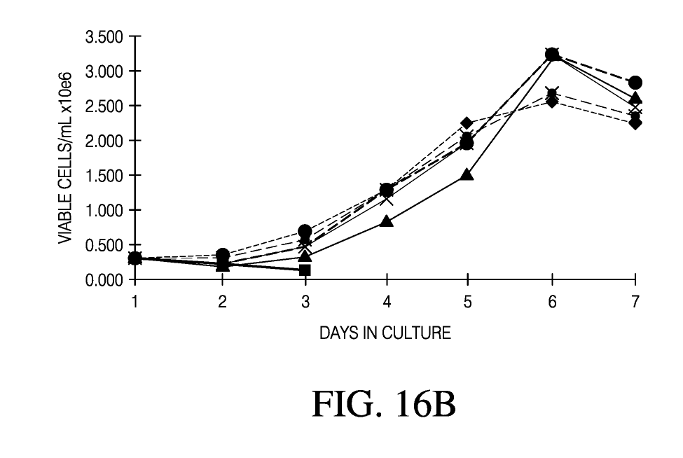

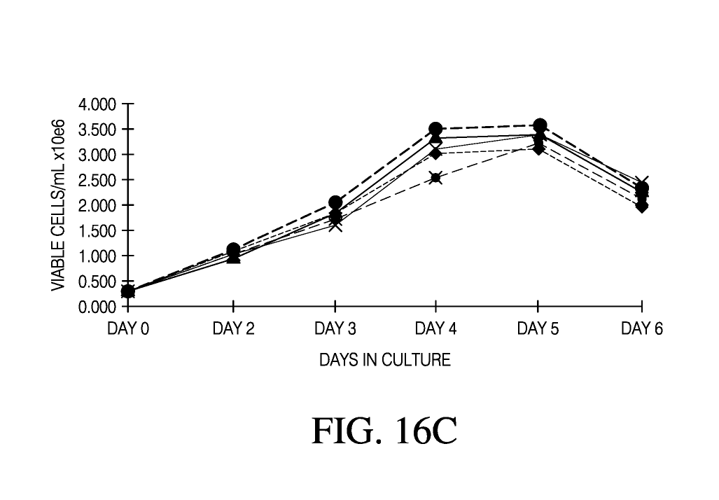

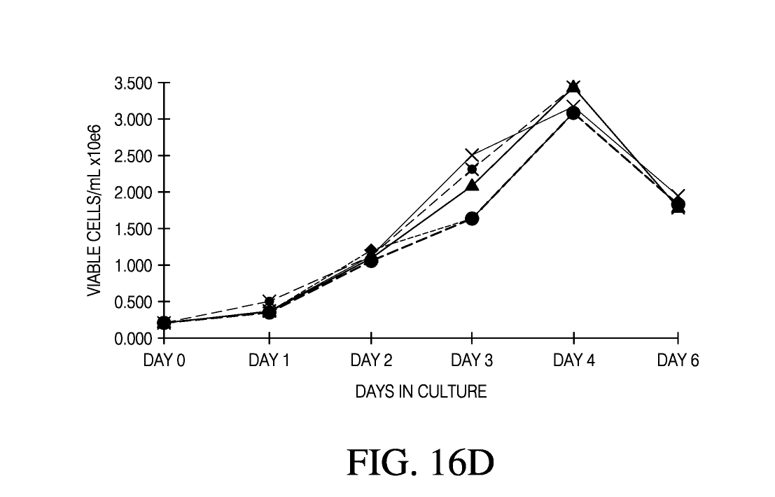

As shown in FIGS. 16A-16D, cells cultured in media containing transferrin that was γ irradiated at either −70° C. or at room temperature demonstrated nearly identical growth kinetics and survival in the first passage (FIG. 16A), second passage (FIG. 16B), third passage (FIG. 16C) and fourth passage (FIG. 16D) as did cells cultured in standard 293 SFM or in 293 SFM containing transferrin that had not been γ irradiated. Cells cultured in transferrin-free media, however, survived well during the first passage (FIG. 16A) but stopped growing and demonstrated a significant loss in viability upon subculturing (FIG. 16B).

These results demonstrate that yirradiation may be used as a sterilization technique in the preparation of bulk powdered culture media supplements, such as transferrin, in the methods of the present invention. Furthermore, these data indicate that culture media supplements such as transferrin may be γ irradiated at room temperature without significant loss of activity.

### Example 15

### Effect of Irradiation on Biochemical Characteristics of Powdered Sera

To further determine the impact of γ irradiation on sera, samples of spray-dried powder FBS were irradiated at 25 kGy at −70° C. or at room temperature (RT), and were analyzed commercially for the concentrations of various biochemical constituents in the sera. As controls, samples of non-irradiated spray-dried FBS and liquid FBS were also analyzed. Results are shown in Table 4.

TABLE 4
Chemical Analysis of Spray-Dried FBS

| | Dried FBS, | Dried | | | | |
|---|---|---|---|---|---|---|
| | Irr. @ | FBS, | Non-irradiated | Liquid | Units | |
| Constituent | −70° C. | Irr. @ RT | Dried FBS | FBS | Range | Reference |
| Sodium | 139 | 137 | 139 | 140 | mM | 136-144 |
| Potassium | 13.2 | 13.2 | 13.0 | 13.2 | mM | 3.6-5.2 |
| Chloride | 98 | 97 | 98 | 100 | mM | 98-108 |
| Uric Acid | 1.6 | 1.3 | 1.7 | 1.9 | mg/dL | 2.2-8.3 |
| Phosphorus | 10.1 | 10.1 | 9.6 | 10.2 | mg/dL | 2.2-4.6 |
| Calcium | 14.9 | 14.8 | 14.8 | 14.5 | mg/dL | 8.6-10.2 |
| Ionizable | >5.5 | >5.5 | >5.5 | >5.5 | mg/dL | 3.8-4.5 |
| Calcium | | | | | | |
| Magnesium | 2.77 | 2.76 | 2.75 | 2.76 | meg/L | 1.4-2.0 |
| Alkaline | 57 | 47 | 68 | 269 | U/L | 31-142 |
| Phosphatase | | | | | | |
| Gamma GT | 3 | 5 | <5 | 5 | U/L | 1-60 |
| (GGTP) | | | | | | |
| AST (SGOT) | 7 | 5 | 5 | 33 | U/L | 1-47 |
| ALT (SGPT) | 5 | <5 | <5 | 7 | U/L | 1-54 |
| LD | 56 | <50 | 50 | 510 | U/L | 110-250 |
| Total | 0.19 | 0.24 | 0.22 | 0.13 | mg/dL | 0.2-1.4 |
| Bilirubin | | | | | | |
| Direct | 0.04 | 0.07 | 0.07 | 0.04 | mg/dL | 0.0-0.3 |
| Bilirubin | | | | | | |
| Glucose | 67 | 38 | 39 | 88 | mg/dL | 65-125 |
| BUN | 15 | 15 | 15 | 15 | mg/dL | 6-23 |
| Creatinine | 2.98 | 3.08 | 3.1 | 2.77 | mg/dL | 0.1-1.7 |
| BUN/Creatine | 5.0 | 4.9 | 4.8 | 5.4 | — | 7.0-20.0 |
| Ratio | | | | | | |
| Total Protein | 3.6 | 3.6 | 3.5 | 3.7 | gm/dL | 6.4-8.1 |
| Albumin | 2.7 | 2.7 | 2.8 | 2.8 | gm/dL | 3.7-5.1 |
| Globulin | 0.9 | 0.9 | 0.7 | 0.9 | gm/dL | 2.1-3.6 |
| Albumin/ | 3.0 | 3.0 | 4.0 | 3.1 | — | 1.1-2.3 |
| Globulin | | | | | | |
| Ratio | | | | | | |
| Cholesterol | 30 | 30 | 32 | 30 | mg/dL | <200 |
| HDL | 28 | 30 | 30 | 27 | mg/dL | 39-90 |
| Cholesterol | | | | | | |
| Chol/HDL | 1.07 | 1.00 | 1.07 | 1.11 | — | <4.5 |
| Ratio | | | | | | |
| Triglycerides | 72 | 74 | 72 | 73 | mg/dL | 30-200 |
| Iron | 213 | 217 | 214 | 186 | meg/dL | 40-175 |
| Plasma Hb | 13.3 | 11.5 | 13.7 | 22.6 | mg/dL | 3.4-20.5 |

These results indicate that the γ irradiation process did not significantly affect the concentrations of most of the biochemical constituents of FBS. These results also indicate that upon spray-drying, several of the components of FBS (alkaline phosphatase, AST, and LD, and possibly glucose) undergo a significant reduction in concentration compared to their concentrations in the starting liquid FBS.

### Example 16

### Effects of Irradiation on Performance of Powdered Sera

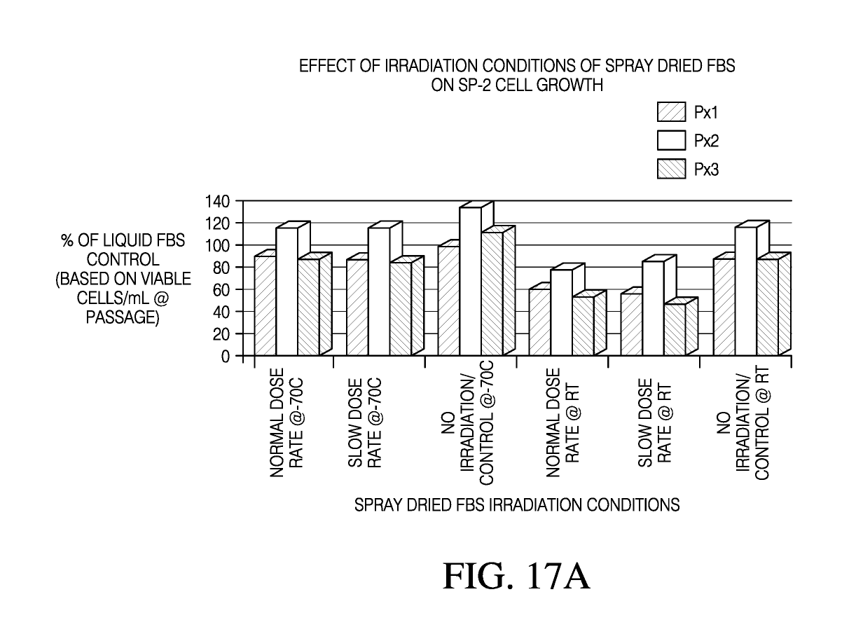

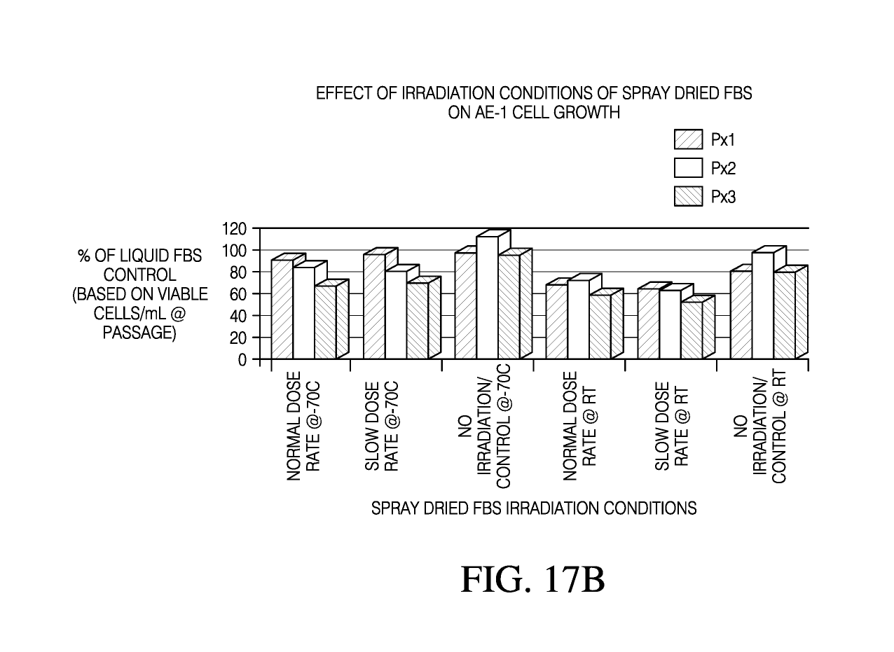

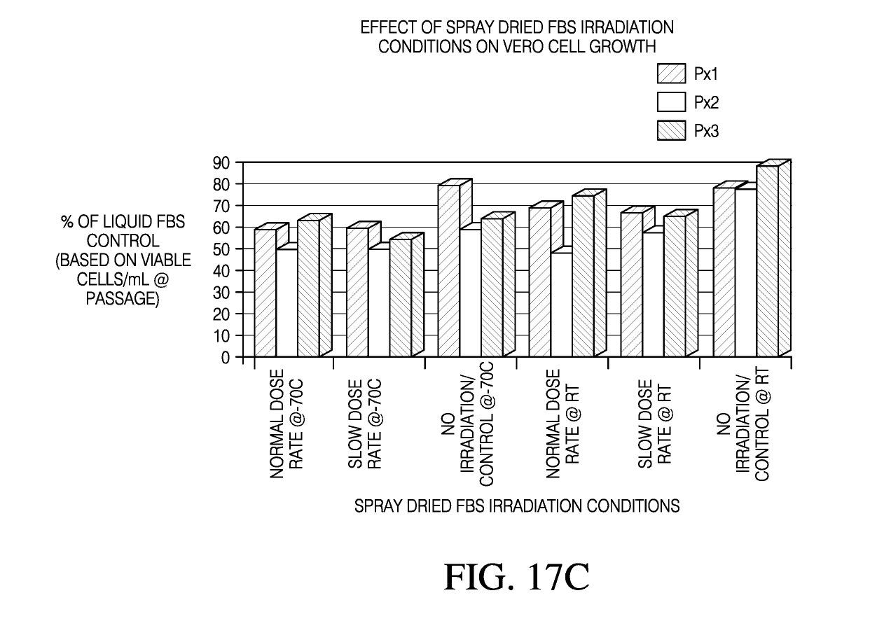

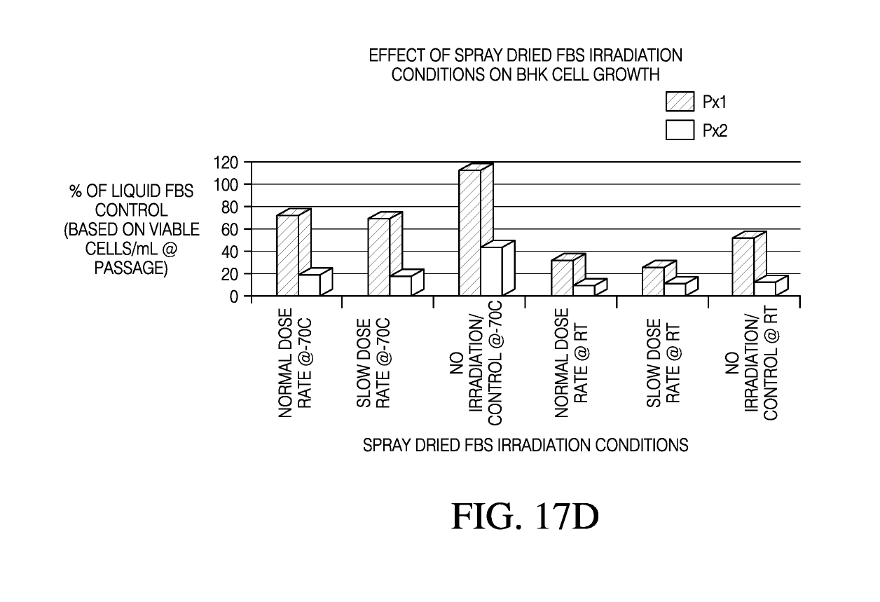

To examine the impact of γ irradiation on the ability of dried powder sera to support cell growth, samples of spray-dried FBS irradiated under various conditions were used to supplement culture media, and adherent and suspension cells were grown for up to three passages in these media. As model suspension cells, the hybridoma lines SP2/0 and AE-1 were used, while VERO and BHK cultures were used as typical adherent cells. Cells were cultured in media containing test sera or control sera (spray-dried but not irradiated) for up to three passages according to the general procedures outlined in Example 14 above. At each passage point, cells were harvested and subcultured, while an aliquot was counted as above for viable cells/ml. Results at each point were expressed as a percentage of the viable cell count obtained in media supplemented with liquid FBS, and are shown in FIGS. 17A, 17B, 17C and 17D.

Several conclusions may be drawn from the results of these studies. First, γ irradiation of FBS does not appear to reduce the ability of spray-dried FBS to support the growth of suspension and adherent cells (compare the irradiated data sets to the non-irradiated data set in each figure). In fact, BHK cells (FIG. 17D) actually grew better in media containing powdered FBS that had been irradiated at −70° C. than they did in non-irradiated sera. Second, sera irradiated at −70° C. appear to perform better than those irradiated at room temperature in their ability to support cell growth, except perhaps for VERO cells (FIG. 17C). Finally, the results of these studies were very cell type-specific: suspension cells (FIGS. 17A and 17B) grew better in spray-dried FBS, irradiated and non-irradiated, than did adherent cells (FIGS. 17C and 17D); and among adherent cells, BHK cells (FIG. 17D) grew better in spray-dried FBS than did VERO cells (FIG. 17C).

These results demonstrate that yirradiation may be used as a sterilization technique in the preparation of bulk powdered sera, such as FBS, in the methods of the present invention. Furthermore, unlike those reported for transferrin in Example 14 above, these data suggest that the optimal temperature for irradiation of sera, in order to maintain the ability of the sera to support cell growth, is likely to be below room temperature.

### Example 17

Production of Automatically pH-Adjusted Powdered Culture Media by Phosphate Balancing

As noted above, typical commercially available mammalian cell culture powdered media require addition of one or more buffer salts (e.g., sodium bicarbonate) when preparing a 1× liquid, followed by adjustment of pH, so that the solution will be at proper pH for use. The methods of the present invention, however, can be used to obviate both the post-reconstitution addition of sodium bicarbonate (as described above in Example 2) and the need for pH adjustment. In this aspect of the invention, fluid bed technology may be used to introduce acid or base (depending on the need) to a dry powder medium comprising one or more buffering salts. In accordance with this aspect of the invention, any buffering salts or combinations thereof, and any acid or base, may be used depending upon the desired pH and buffering capacity in the ultimately reconstituted cell culture medium.

If sodium bicarbonate is added as a powder directly to the dry powder medium (DPM) in accordance with the methods of the present invention, it is possible for the end user to simply add solvent (e.g., water) and mix to yield a solution already containing bicarbonate (see above) and at the proper pH for immediate use—i.e., an “auto-pH” or “automatically pH-adjusting” culture medium of the invention. To determine how much of a pH adjustment is required, several steps should be undertaken:

(1) Place ˜950 ml of solvent (e.g., water) in a beaker. Add DPM (in an amount according to the manufacturer's specifications or according to formulation specifications that are known in the art as referred to herein) to the solvent and mix quantum sufficit to 1 L. In this case, the weight of the sodium bicarbonate must also be considered in determining how much DPM to add per liter. However the DPM should contain neither sodium phosphate buffer nor HEPES buffer, which will be titrated in, but should contain sodium bicarbonate.

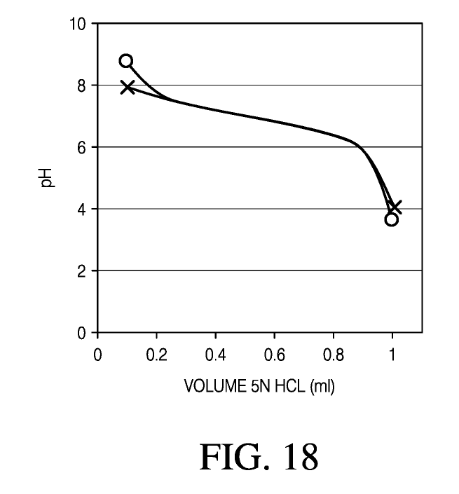

(2) The next step is to determine whether monobasic or dibasic phosphate will give the desired final pH. This depends on the whether the adjustment of pH needed is to a more basic level (indicating the need for dibasic phosphate), or to a more acidic level (indicating the need for monobasic phosphate). Sequentially add amounts (at final concentration ranges of about 0.1 mM to about 10 mM, about 0.2 mM to about 9 mM, about 0.3 mM to about 8.5 mM, about 0.4 mM to about 8 mM, about 0.5 mM to about 7.5 mM, about 0.6 mM to about 7 mM, and preferably about 0.7 mM to about 7 mM) of monobasic or dibasic phosphate salts to obtain the desired pH. The total molar amounts of sodium phosphate should remain constant, but buffering due to either monobasic or dibasic results in similar buffering kinetics at a given pH (see FIG. 18), because the molecular species in solution is the same as determined by the final (desired) pH of the solution.

(3) If the medium contains a HEPES buffer system, add the correct molar amount of either the acid form (for more acidic pH) or the sodium form (for more basic pH) of HEPES to arrive at the proper (desired) final pH.

(4) The next step is to exchange the monobasic sodium phosphate for monobasic potassium phosphate on a molar weight basis (identical buffering characteristics result from use of either sodium or potassium phosphate). To carry out this exchange, the amount of monobasic sodium phosphate calculated in step 2 above is substituted with an equal amount of potassium phosphate, which is then used to formulate the final medium. Hence, in this aspect of the invention, the amount of monobasic sodium phosphate calculated in step 2 above is not actually added to the medium to adjust the pH; instead, this amount is simply calculated, and then the calculated amount of potassium phosphate is used in adjusting the pH of the final medium. This is done so that subsequent off-gassing of carbon dioxide gas is eliminated or minimized. (In order to have the same molar ratios of potassium to sodium in the formulation as a whole, it is necessary to back-adjust the amount of potassium chloride in the formulation so that the final 1× molar amounts are the same. This may also result in the need to adjust with a small amount of sodium chloride to reach identical osmolarities). When the solution is in its final form, it is dried into a powder by either agglomeration using fluid bed technology (as described in Example 1), spray-drying (see Example 8), or lyophilization techniques known to those skilled in the art.

While the above are the basic manipulations recommended for including sodium bicarbonate in powdered media, additional considerations should also be kept in mind:

(1) Only anhydrous forms of media components should be used

(2) If anhydrous forms are not available, consider using “ionic replacement” (e.g., ZnSO₄X7H₂O or monohydrous; also consider using ZnCl₂ with sodium sulfate and reducing stoichiometrically correct amounts of NaCl from the added amount of NaCl as indicated in the formulations);

(3) Do not use HCl conjugates of chemicals: use free base (ex. arginine instead of arginine HCl, corrected for true desired weight);

(4) Monobasic sodium phosphate should not be used at all since it can cause pillowing. Instead, monobasic potassium phosphate (KH₂PO₄), which does not cause pillowing should be used. The buffering responses of both of these chemicals is identical. The amount of potassium added to the formulation in the forms of other salts should correspondingly be reduced by the amount KH₂PO₄ added here. Also, extra NaCl may be needed so that the osmolarity of the formula with sodium phosphate is equal to the osmolarity of the formula with potassium phosphate.

(5) Dibasic sodium phosphate (Na₂HPO₄) does not “pillow” and is acceptable for use in the formulation.

(6) Do not use HCl “spray in” in the fluid bed for auto-adjust mechanism because this increases the propensity to “pillow” with the bicarbonate. Instead, adjust the final pH of reconstituted media by phosphate balance in the DPM. (Na₂HPO₄ raises pH, while KH_PO₄ reduces pH. As indicated above use “ionic balance” results in the same ionic composition of the 1× media).

(7) If amino acid(s) need to be added as a spray-in for the fluid bed (such as cysteine for CD-CHO) because of solubility concerns, do not reduce pH to dissolve amino acids but raise pH to the minimum needed for solubilization (e.g., pH 10.66 for cysteine).

(8) If a component cannot be obtained anhydrous or “ionic replacement” does not apply, the chemical can be solubilized and sprayed into the media via agglomeration: water in the crystal component will not dry during agglomeration, but if the water is released by dissolution, then it will be eliminated from the DPM by the spraying and evaporation process.

(9) Check with an accelerated shelf life test (37° C. in a sealed pouch with equal amounts of bicarbonate for any chemicals that appear moist or gooey such as choline chloride. One example of an acceptable shelf life testing protocol is as follows:
(a) place 10 grams of the test compound into a mortar and pestle; add 10 grams of NaHCO₄;
(b) grind the mixture to reduce particle size and blend for about 30 seconds;
(c) add the blended mixture to a foil pack; seal the open end of the pack;
(d) place the pack into an incubator at 37° C., and observe for “pillow” formation (i.e., swelling of the pack due to off-gassing of the NaHCO₄) over 24 hours. If gas is given off, solubilize and add that component to the spray-in solution.
(10) Place choline C1 in neutral solubles spray-in.

Once the powder is collected after the spray-dry or agglomeration run, it can be reconstituted with a solvent (e.g., water) at any time, as long as it has been kept in proper “dry” packaging and conditions. Examples of acceptable or “proper” dry packaging include any packaging that retards or prevents the penetration of water and/or water vapor through the packaging upon storage, such as foil packaging, polyethylene bags, sealed plastic (particularly polypropylene, polycarbonate, polystyrene, polyethylene terephthalate (PET) and the like). Examples of proper storage conditions include storage at about 0° C. to about 25° C., preferably about 2° C. to about 20° C., about 2° C. to about 15° C., about 2° C. to about 10° C., or about 2° C. to about 8° C., under diminished or subdued lighting. Under such conditions, minimum shelf life of the media of the present invention is about one year (stored at about 2° C. to about 8° C.), or about six months (at about 20° C. to about 25° C. (room temperature)).

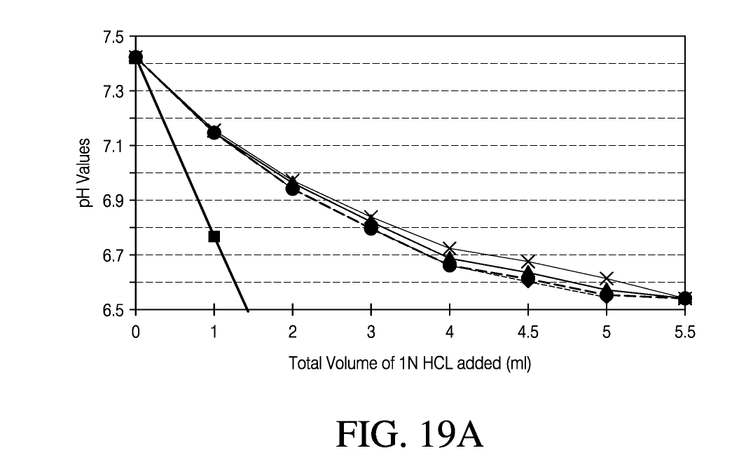

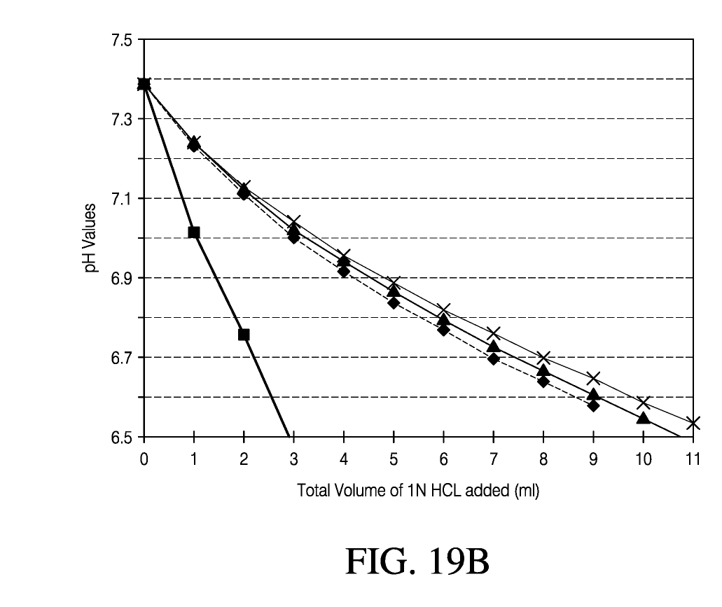

For use, the powder is simply reconstituted with an appropriate solvent (e.g., water); no adjustment of pH is needed, since the media are at the appropriate pH and have appropriate buffering kinetics immediately upon reconstitution (see FIGS. 19A and 19B). Thus, the invention provides an automatic pH-adjusting dry powdered medium, where the pH of the liquid medium made by reconstituting the dry powdered medium requires no adjustment of pH.

### Example 18

Spray in of Media Components in μg/Ml or μg/L Quantities

Chemicals such as trace elements (such as calcium, copper, iron, magnesium, manganese, nickel, potassium, tin, and zinc, vitamins (such as A, B1, B2, B6, B12, C, D, E, K and H (biotin), viral inhibitors (such as protease inhibitors, nucleoside analogues, and the like), growth factors (such as EGF, aFGF, bFGF, HGF, IGF-1, IGF-2, and NGF), etc., may be added to standard powdered media by first making a concentrate of the chemicals and then spraying them into the powdered media granulation (see U.S. patent application Ser. No. 09/023,790, filed Feb. 13, 1998, which is incorporated herein by reference in its entirety.) The resulting powder may then be milled (e.g., with a Fitzmill) to a particle size in the same general size range as that of the bulk for blending (which is required after weighing and Fitzmilling). This portion may then be combined with the bulk powdered medium and milled together to create a homogeneously mixed powdered medium. Alternatively, the components of the powdered media may be subgrouped into mixtures of compatible compounds or components, which may then be blended immediately prior to formulation, and concentrates of the low-level components may be sprayed into the blend. This approach is particularly advantageous when dealing with components that may be incompatible if they are admixed and stored together for extended times in a powdered media formulation (e.g., cysteine and glutamine, which will form insoluble complexes upon storage together for extended periods of time). (For a more detailed description of the advantages of subgrouping culture medium components, see Example 19 below, and commonly owned U.S. Pat. Nos. 5,474,931 and 5,681,748, the disclosures of which are incorporated by reference herein in their entireties). The ability to spray-in chemicals in small amounts is especially helpful in developing media that contains components present in small quantities and which are inconvenient to add separately. Thus, the dry powdered medium is ready to use.

### Example 19

### Subgrouping of Components to Avoid Harmful Interactions During Agglomeration

Some of the components of a medium may be incompatible and cause them to interact deleteriously to each other if they are weighed and then held together prior to milling and agglomeration. For example, adverse reactions have been observed when cysteine and glutamine powders are admixed, when phosphate salts are admixed with calcium- or magnesium ion-containing salts, when phosphate salts (particularly monobasic forms thereof) are admixed with choline chloride, and when glutathione is admixed with amino acids. In addition, acidic components (e.g., acidic forms of certain buffer salts, vitamins, and the like) may denature protein components such as growth factors or serum. Therefore, these particular components can be subgrouped (see commonly owned U.S. Pat. Nos. 5,474,931 and 5,681,748, which describe methods of subgrouping culture media, supplement and buffer components, and the disclosures of which patents are incorporated herein by reference in their entireties) and agglomerated together as a subgroup. Specific subgroups include, an acid soluble subgroup, a weak acid-base soluble subgroup, a glutamine-containing subgroup, an alcohol soluble subgroup, an alkali-soluble subgroup, and a supplement-containing subgroup. After agglomeration of the separate subgroups, they can then be mixed together, as described in Example 18 for spraying in of elements in small quantities.

Alternatively, the concentrates may be sprayed directly into already-milled bulk powder. In this approach, each subgroup is milled (e.g., via Fitzmilling) and then placed into the fluid bed apparatus at the same time as other subgroups, so that the subgroups are mixed together during agglomeration; concentrates may then be sprayed into the bulk powder sequentially, such that individual incompatible components are only admixed for a very short period of time prior to being agglomerated. The agglomerated powder can then be collected and stored as described herein until reconstitution and use, without adverse reactions occurring among individual, otherwise incompatible, components.

### Example 20

Lipids (particularly sterols and fatty acids) are critical nutrients for high density cultivation of eukaryotic cells. Inclusion of lipid components in dry-form media has been technically challenging. Lipid supplements are usually supplied for separate addition after powder reconstitution and filtration, increasing manipulation and chances for error in a biopharmaceutical manufacturing facility. Advanced Granulation Technology (AGT™) is a novel dry-form media format having significant advantages. Within a single granulated medium all components of a complex formulation are incorporated, to include buffers, growth factors, and trace elements. The resulting low dust, auto-pH formulation simply requires addition to water to yield a complete reconstituted 1× medium. Cyclodextrin technology as well as use of sodium salts and hydro-alcoholic solutions of lipids may be used in conjunction with the AGT process to deliver usable lipid in a dry medium format.

The lipids tested were cholesterol and several fatty acids which were provided either as an aseptic supplement to liquid media or as part of a complete AGT formulation. Controls included medium with no lipid. The cell line used was ECACC #85110503, a cholesterol auxotroph. The cells were cultured in CD-Hybridoma Medium, which is chemically-defined and contains no animal-derived components. GC analytical results indicated excellent availability of lipid post-filtration when incorporating cyclodextrin-complexed lipid forms into the AGT process. Growth and viability of cells were comparable when grown in either AGT-derived complete medium or control liquid medium with lipid supplementation. Peak cell densities of both media formats reached 3.5×10⁶ cells/ml in batch cell culture. Use of salts for example, a sodium salt of lipoic acid in AGT has proven to be effective for delivering the lipid to cells in culture.

For preparation, cyclodextrin was dissolved in water at a concentration of 62.5% (62.5 g in 100 ml of water). This can be varied somewhat lower but approaches about the maximum dissolution of cyclodextrin in room temperature water. It is preferred to maintain as high a ratio of cyclodextrin to lipid as practical since the ability of cyclodextrin to maintain partitioning (physical complexation with) the lipid and keep it in solution upon dilution in water depends on cyclodextrin levels. (˜0.125% or higher solution of cyclodextrin is advantageous in the 1× medium). Lipids were then added directly to the cyclodextrin solution at a concentration so as to be at desired concentration when diluted in aqueous cell culture media. The lipid was allowed to dissolve with stirring. In addition to direct addition of lipid to the cyclodextrin, it is also possible to add lipid to alcohol prior to addition to cyclodextrin. (This may be desired if the amount of lipid to add is so small that addition by itself is physically problematic). Since the resulting cyclodextrin-lipid solution is quite viscous, it may be preferable to dilute the above lipid-cyclodextrin solution e.g., with water for convenient use. Such dilutions may result in a concentrate of for example 500× or 250×. (One of ordinary skill will appreciate that as the lipid-cyclodextrin solution is diluted, more volume will need to be added to the cell culture medium to yield the desired concentration of lipid).

Types of lipid of importance to cell culture: cholesterol (both animal and plant correlates), linoleic acid, lipoic acid, arachidonic acid, palmitic acid, oleic acid, palmitoleic acid, stearic acid, myristic acid, linolenic acid, phosphatidyl ethanolamine, phosphatidyl choline, sphingomylelin, cardiolipin, vitamin A, vitamin E, Vitamin K, prostaglandin, etc.

Cell Culture Experiment with Cyclodextrin-Lipid Complex:
(Cells subpassaged every 3 or 4 days)
1=CD Hybridoma granulated (agglomerated) medium with lipids added via spray-in cyclodextrin-lipid complexes during (as part of) agglomeration process.
2=CD Hybridoma medium with lipids added as cyclodextrin-lipid supplement addition post-reconstitution.
3=CD Hybridoma medium with no added lipids.

TABLE 5

| | | Viable Cell Concentration |
|---|---|---|
| Culture | Day of culture | (×10⁵/ml) |
| 1 | 3 | 23.75 |
| 2 | 3 | 23.65 |
| 3 | 3 | 21.85 |
| 1 | 6 | 7.02 |
| 2 | 6 | 9.68 |
| 3 | 6 | 0.10 |
| 1 | 9 | 9.01 |
| 2 | 9 | 10.43 |
| 3 | 9 | 0 |
| 1 | 13 | 13.50 |
| 2 | 13 | 12.85 |
| 3 | 13 | 0 |
| 1 | 16 | 16.70 |
| 2 | 16 | 19.10 |
| 3 | 16 | 0 |
| 1 | 20 | 10.70 |
| 2 | 20 | 10.97 |

Conclusion: Lipids supplied by granulation technology using cyclodextrin-lipid spray-in is comparable to lipids added using cyclodextrin-lipid added as a supplement to a 1× reconstituted medium.

### Example 21

### Supplement Preparation

A preferred supplement preparation can be prepared as follows:

Select a desired chemical supplement feed formulation using sodium forms for all amino acids where available. Prepare powder version with various monobasic to dibasic sodium phosphate levels. Reconstitute and measure pH and observe solubility.

Calculate ratio of sodium monobasic to dibasic phosphate to reach reconstituted desired pH of for example, ˜8.0. To counteract acidic impact of non-sodium amino acids, use trisodium phosphate for pH adjustment.

This formulation can be made using advanced granulation technology, such as fluidized bed granulation. Once reconstituted with water, can be filtered and fed into a bioreactor as a nutrient supplement.

### Example 22

### Viral Titer Reduction by Spray-Drying

The feasibility of reducing viral titer by spray-dry technology was examined. A three foot diameter laboratory spray dryer (Mobile Minor Spray Dryer, NIRO, Columbia, Md.) was used to prepare the powdered serum. Liquid FBS was spiked with virus at a known concentration (IBR virus @ 10^{6.5} TCID₅₀/mL, REO virus @ 10⁵ TCID₅₀/mL and naturally contaminated with BVDv@a++ detection level). The virus spiked liquid FBS was aspirated into the spray-dryer and atomized through a Schlick 940 nozzle located in the middle of the air disperser, and the drying air was introduced into the chamber through the top air disperser of the apparatus. Spray drying was conducted under the following conditions: inlet air temperature=148° to 215° C.; outlet temperature=50E to 80E C, atomizing air pressure for the nozzle=1.6 to 2.0 bar; air flow=80.0 kg/hour; spray rate=2 kg/hour. Following spray-drying, powdered serum was collected at the cyclone of the apparatus, and process air was exhausted.

Following production, the powdered serum was reconstituted to a 1× liquid with distilled water (60 gm powdered serum=one liter liquid FBS). This reconstituted 1× liquid Spray-Dry processed serum was characterized with respect to viral titer and compared to the non-processed liquid FBS from the same virus spiked lot with known viral concentration using the following viral titer detection procedure. Using a cell line known to be sensitive to assayed virus, 1×10⁴ cells are plated per well of a 96 well plate. The sample to be assayed is diluted through a series of 10 fold dilutions out to 10⁻¹⁰. Aliquots (0.1 ml) of each dilution are added to replicate wells of the cell line inoculated plate. The cells in each well are evaluated for cytopathic effect (CPE) after 4 to 7 days. Results are evaluated using the method of Reed, L J and Muench, H. (Am. J. Hyg. 1938:27:493) and expressed as tissue culture infective dose (TCID₅₀/mL) sample material. BVDv tested by the cell culture method over three passages and final antigen detection by direct fluorescent assay (9CFR).

Results are shown in Table 6, Table 7, Table 8 and Table 9 for reduction of viral titer by Spray-Dry processing of powdered FBS. Conclusion: Spray-Drying process was effective in inactivation of IBR virus with a total titer reduction of at least 10⁻⁶, of REO virus with a total titer reduction of at least 10⁻⁵, and of BVDv inactivation of the naturally contaminating virus from ++ to negative. Together, these results indicate that serum powder prepared by the present spray-drying methods have significantly reduced viral titer. These results demonstrate that the methods provided by the present invention result in the production of powdered serum with 10⁻⁶ reduction in viral titer.

TABLE 6
IBR Viral Titer of Powdered Serum Prior
to and Post Spray-Drying Treatment

| | Prior to Spray Drying Process | Post Spray Drying Process |
|---|---|---|
| | IBR “spiked” FBS Control | Spray-dried* IBR “spiked”FBS |
| 10⁻¹ | + | − |
| 10⁻² | + | − |
| 10⁻³ | + | − |
| 10⁻⁴ | + | − |
| 10⁻⁵ | + | − |
| 10⁻⁶ | + | − |
| 10⁻⁷ | +/− | na |

*Spray-Dry @ inlet temperature = 15° C.; outlet temperature = 70° C.
Results Summary: Spray-Dried FBS: negative, no virus detected after spray drying. Control, virus “spiked” FBS = Positive. Virus titer = 1 × 10^{6.5}.

TABLE 7
BVD Viral Titer of powdered Serum Prior
to and Post Spray-Drying Treatment

| Prior to Spray Dry Processing | Post Spray Dry Processing |
|---|---|
| Spray-Dried BVDV positive FBS | Negative (BT cells) |
| (215E/80° C.)* | |
| Spray-Dried BVDV positive FBS | Negative (BT cells) |
| (150E/50° C.)** | |
| Non-treated BVDV positive FBS | Positive (++)(BT cells) |

*Spray-Dry @ inlet temperature = 215° C.; outlet temperature = 80° C.
**Spray-Dry @ inlet temperature = 150° C.; outlet temperature = 70° C.
Results Summary: Spray-Dried FBS: negative, no virus detected after spray drying using either set of processing temperatures tested.

TABLE 8
REO Viral Titer of Powdered Serum Prior
to and Post Spray Drying Treatment

| | | Post Spray Drying Process |
|---|---|---|
| | Prior to Spray Drying Process | Spray-dried* Reovirus |
| | Reovirus 3 “spiked” FBS Control | 3“spiked” FBS |
| 10⁻¹ | + | − |
| 10⁻² | + | − |
| 10⁻³ | + | − |
| 10⁻⁴ | + | − |
| 10⁻⁵ | + | − |
| 10⁻⁶ | − | − |
| 10⁻⁷ | − | − |
| 10⁻⁸ | − | − |

*Spray-Dry @ inlet temperature = 150° C.; outlet temperature = 70° C.
Results Summary: Spray-Dried FBS: negative, no virus detected after spray drying. Control, virus “spiked” FBS = Positive. Virus titer = 1 × 10⁵.

TABLE 9
FBS Viral Titer Reduction

| Virus Tested | Viral Load Tested | Virus Reduction |
|---|---|---|
| IBR Virus | 10^{6.5} TCID₅₀/mL | ≥6 Log 10 |
| BVD Virus | ++ | Negative |
| REO Virus | 10⁵ TCID₅₀/mL | ≥5 Log 10 |

### Example 23

### Endotoxin Reduction by Spray-Drying

The feasibility of reducing endotoxin concentration by spray-dry technology was examined. A three foot diameter laboratory spray dryer (Mobile Minor Spray Dryer, NIRO, Columbia, Md.) was used to prepare the powdered serum. A lot of Liquid FBS was identified with elevated endotoxin levels. The endotoxin containing liquid FBS was aspirated into the spray-dryer and atomized through a Schlick 940 nozzle located in the middle of the air disperser, and the drying air was introduced into the chamber through the top air disperser of the apparatus. Spray drying was conducted under the following conditions: inlet air temperature=148° to 215° C.; outlet temperature=50° to 80° C., atomizing air pressure for the nozzle=1.6 to 2.0 bar; air flow=80.0 kg/hour; spray rate=2 kg/hour. Following spray-drying, powdered serum was collected at the cyclone of the apparatus, and process air was exhausted.

Following production, the powdered serum was reconstituted to a 1× liquid with distilled water (60 gm powdered serum=one liter liquid FBS). The endotoxin concentration of this reconstituted 1× liquid Spray-Dry processed serum was determined and compared to the endotoxin level of non-processed liquid FBS from the same lot using the Limulus Amebocyte Lysate (LAL) test. Briefly, the LAL test is a gel clot test conducted by mixing LAL reagent and test sample and observing for gelation after 60 minutes at 37° C. See generally “Pyrogens, Endotoxins, LAL Testing, Depyrogenation” (J. Robinson, ed.) Marcel Dekker, Inc., New York. A positive response (gel formation) indicates that there is an amount of endotoxin in the sample which meets or exceeds the reagents labeled sensitivity. Results are reported in endotoxin units per mL. All endotoxin is measured in units by comparison to the reference standard endotoxin.

Results are shown in Table 10 below for the reduction of endotoxin concentration by Spray-Dry processing of FBS. Conclusion: Spray-Drying process was effective in reducing the endotoxin concentration with an endotoxin concentration reduction of 50% from 48.0 EU/mL to 24.0 EU/mL. These results indicate that serum powder prepared by the present spray-drying methods have significantly reduced endotoxin levels. These results demonstrate that the methods provided by the present invention result in the production of powdered serum with reduced endotoxin level.

TABLE 10
Endotoxin Test Results of Spray Dried Processed FBS

| Sample Description | Endotoxin Level (EU/mL) |
|---|---|
| Spray Dried Processed FBS | 24.0 |
| Control FBS (Non-spray dried | 48.0 |
| processed FBS) | |
| H₂O used to reconstitute Spray | <0.03 |
| Dried Processed FBS | |

| | | | |
|---|---|---|---|
| Conditions used: | inlet temperature | 150° | C. |
| | outlet temperature | 60° | C. |
| | atomizing pressure | 1.6 | Bar |
| | spray rate | 1.51 | kg/hr |

### Example 24

### Exemplary Fluid Bed Apparatus

### Make: Glatt Air Techniques Inc (Ramsey, N.J.)

Model: GPCG Pro 120 fluid bed processor; S/N: 8088

### General Information Related to the GPCG Pro 120 Fluid Bed Processor

This model provides a ratio of air volume flow to quantity of product used. The conical pressure relief zone and the resulting reduced flow speed allow very fine products to be processed. At the center of granulation is the Glatt single pipe nozzle. This combines outstanding spray behavior with optimum media delivery and easy cleaning. Agglomeration in the fluid bed is a process for building up powder granulates. In this process, powder is moistened in order to form liquid bridges between the particles. The spray liquid can either be water or an organic solvent, a powder dissolved in water or another binder. The moistened granulates are dried and cooled as required. Due to the relatively low mechanical forces in the fluid bed, the agglomerates/granulates are loose, have a low bulk density and are highly soluble in water.

The agglomeration technology (e.g., AGT) incorporates the use of a Glatt GPCG Pro 120 Top Spraying Fluid Bed Processor. Within this unit, dry powder medium components that have been previously dispensed, sized, and blended are transferred into the conical shaped product bowl of the fluid bed tower. As the fluid bed granulation process is initiated, this powder medium is transferred from the product bowl into the extended height of the fluid bed expansion chamber on a column of conditioned air.

The increased diameter of the expansion chamber produces a reduction of air velocity, generating a less dense random fluidization pattern. As gravity overcomes the upward force of the air velocity, particles fall to the bottom of the tower and are re-circulated in an unrestricted pattern. During this process, the entire surface area of the powder particles are exposed to the air stream assuring uniform heating and evaporation of excess moisture, and preventing local overheating. High inlet air temperatures controlling humidity and airflow velocity can be achieved while maintaining the product slightly above room temperature.

The spraying of aqueous solutions of concentrated medium components onto the fluidized powder generates the granulation process. The previously prepared aqueous solutions are introduced high into the expansion chamber via a liquid pump skid and a pneumatically atomized nozzle. At this point in the chamber, the bed surface area is at its maximum resulting in a narrow particle size distribution of the final product. Once all the liquid solutions are delivered to the fluidized powder, the formed granules or agglomerates that are produced are subsequently dried with heated air until a final moisture setpoint for the material is achieved. As the final granules are sized and blended with any remaining temperature sensitive components, a complete and homogenous constituent medium is formed with the benefits of rapid dissolution, low dust generation, and auto-pH adjustment.

### Example 25

### Processing Instructions for an Exemplary Culture Medium of the Invention

Prior to fluid bed agglomeration, the dry chemstock (chemstock starting powder) is processed and sized in a Fitzmill using standard procedures to prepare the powder for agglomeration in a fluid bed processor.

After fluid bed agglomeration, the agglomerated product is sized in a Fitzmill as described in Example 27.

After sizing the agglomerated granules are blended, as described in Example 28, with the milled Pluronic-68.

Exemplary Fluid Bed Agglomeration Process with a GPCG Pro 120 Fluid Bed Processor;

### S/N: 8088

### Step 1: Pre-Processing Instructions

A. Prior to processing the batch, pre-warm the Fluid Bed Processor for a minimum of 15 minutes using the following parameters:

| Parameter: | Set Point: |
|---|---|
| Process Air Volume | 1000 cubic feet per minute (cfm) |
| Inlet Air Temperature | 40° C. |
| Dewpoint | −10° C. |

B. Charge the Fluid bed Bowl

### Step 2: Granulation Process Instructions

A. Record the actual parameters e.g., on an In-Process Data Sheet at least every 15 minutes and when making a parameter change. Operating conditions may be adjusted as necessary in order to make a good granulation.

B. Begin to Fluidize the product (Table 13) using the following parameters:

| | | |
|---|---|---|
| Parameter: | Target: | Range: |

| | | | |
|---|---|---|---|
| Process Air Volume | 600 | cfm | 400 cfm-1200 cfm |
| Inlet Air Temperature | 55° | C. | 30° C.-60° C. |
| Dewpoint | −10° | C. | less than 0° C. |

| | |
|---|---|
| Shake Mode | GPCG |

| | | |
|---|---|---|
| Shake Interval | 30 | seconds |
| Shake Duration | 5 | seconds |

C. After approximately one minute, begin spraying Vitamin Solution (Table 14).

Use the following parameters during spraying:

| | | |
|---|---|---|
| Parameter: | Target: | Range: |

| | | | |
|---|---|---|---|
| Process Air Volume | 600 | cfm | 200 cfm-2500 cfm |
| Inlet Air Temperature | 55° | C. | 40° C.-65° C. |

| | | |
|---|---|---|
| Product Temperature | less than 40° C. | 0° C.-45° C. |

| Spray Rate | 100 | g/min | 50 g/min-250 g/min | |
|---|---|---|---|---|
| Atomization Air | 2 | BAR | 2 | BAR |

| | |
|---|---|
| Shake Mode | GPCG |

| | | | | |
|---|---|---|---|---|
| Shake Interval | 30 | seconds | 15-45 | seconds |
| Shake Duration | 5 | seconds | | |

D. Adjust air flow as necessary to achieve optimum fluidization.

E. Upon completion of spraying Vitamin Solution, rinse the lines for 30 seconds with ambient (e.g., about 20° C.) Water for Injection (WFI), e.g., as based on USP (United States Pharmacopoeia) guidelines.

F. Begin spraying Iron Chelate Solution (Table 15). Use the following parameters during spraying:

| | | |
|---|---|---|
| Parameter: | Target: | Range: |

| | | | |
|---|---|---|---|
| Process Air Volume | 1000 | cfm | 200 cfm-2500 cfm |
| Inlet Air Temperature | 55° | C. | 40° C.-65° C. |

| | | |
|---|---|---|
| Product Temperature | less than 40° C. | 0° C.-45° C. |

| Spray Rate | 100 | g/min | 50 g/min-250 g/min | |
|---|---|---|---|---|
| Atomization Air | 2 | BAR | 2 | BAR |

| | |
|---|---|
| Shake Mode | GPCG |

| | | | | |
|---|---|---|---|---|
| Shake Interval | 30 | seconds | 15-45 | seconds |
| Shake Duration | 5 | seconds | | |

G. Upon completion of spraying Iron Chelate Solution, rinse the lines for 30 seconds with ambient (e.g., about 20° C.) WFI.

H. Begin spraying Trace Element Solution (Table 20). Use the following parameters during spraying:

| | | |
|---|---|---|
| Parameter: | Target: | Range: |

| | | | |
|---|---|---|---|
| Process Air Volume | 1000 | cfm | 200 cfm-2500 cfm |
| Inlet Air Temperature | 55° | C. | 40° C.-65° C. |

| | | |
|---|---|---|
| Product Temperature | less than 40° C. | 0° C.-45° C. |

| Spray Rate | 100 | g/min | 50 g/min-250 g/min | |
|---|---|---|---|---|
| Atomization Air | 2 | BAR | 2 | BAR |

| | |
|---|---|
| Shake Mode | GPCG |

| | | | | |
|---|---|---|---|---|
| Shake Interval | 30 | seconds | 15-45 | seconds |
| Shake Duration | 5 | seconds | | |

I. Upon completion of spraying Trace Element Solution, rinse the lines for 30 seconds with ambient (e.g., about 20° C.) WFI.

J. Begin spraying Neutral Solution (Table 16). Use the following parameters during spraying:

| | | |
|---|---|---|
| Parameter: | Target: | Range: |

| | | | |
|---|---|---|---|
| Process Air Volume | 1000 | cfm | 200 cfm-2500 cfm |
| Inlet Air Temperature | 55° | C. | 40° C.-65° C. |

| | | |
|---|---|---|
| Product Temperature | less than 40° C. | 0° C.-45° C. |

| Spray Rate | 100 | g/min | 50 g/min-250 g/min | |
|---|---|---|---|---|
| Atomization Air | 2 | BAR | 2 | BAR |

| | |
|---|---|
| Shake Mode | GPCG |

| | | | | |
|---|---|---|---|---|
| Shake Interval | 30 | seconds | 15-45 | seconds |
| Shake Duration | 5 | seconds | | |

K. Upon completion of spraying Neutral Solution, rinse the lines for 30 seconds with ambient (e.g., about 20° C.) WFI.

L. Begin spraying Calcium Nitrate Solution (Table 17). Use the following parameters during spraying:

| | | |
|---|---|---|
| Parameter: | Target: | Range: |

| | | | |
|---|---|---|---|
| Process Air Volume | 1000 | cfm | 200 cfm-2500 cfm |
| Inlet Air Temperature | 55° | C. | 40° C.-65° C. |

| | | |
|---|---|---|
| Product Temperature | less than 40° C. | 0° C.-45° C. |

| Spray Rate | 100 | g/min | 50 g/min-250 g/min | |
|---|---|---|---|---|
| Atomization Air | 2 | BAR | 2 | BAR |

| | |
|---|---|
| Shake Mode | GPCG |

| | | | | |
|---|---|---|---|---|
| Shake Interval | 30 | seconds | 15-45 | seconds |
| Shake Duration | 5 | seconds | | |

M. Upon completion of spraying Calcium Nitrate, rinse the lines for 30 seconds with ambient (e.g., about 20° C.) WFI and then empty the spray line. Allow the granulation to dry at 40° C. for 10 minutes (range: 2-10 minutes). Air flow may be decreased as necessary. Record start and stop time.

N. Perform shutdown and moisture analysis

TABLE 13
Dry Powder components (starting material)

| Components | g/100 L | g/L final |
|---|---|---|
| D GLUCOSE (DEXTROSE) | 300 | 3 |
| D GLUCOSE (DEXTROSE) | 150 | 1.5 |
| D GLUCOSE (DEXTROSE) | 183.315591 | 1.83315591 |
| Total D Glucose | 633.315591 | 6.33315591 |
| ASCORBIC ACID 2 PHOS MG | 1.990812 | 0.01990812 |
| GLUTATHIONE REDUCED | 0.18095 | 0.0018095 |
| SODIUM PYRUVATE | 19.9045 | 0.199045 |
| I-INOSITOL | 6.33325 | 0.0633325 |
| SPERMINE 4HCL | 1.557576 | 0.01557576 |
| L-ARGININE F.B. | 36.189276 | 0.36189276 |
| L-ASPARAGINE ANHYD | 79.620101 | 0.79620101 |
| L-ASPARTIC ACID | 18.094638 | 0.18094638 |
| L-GLUTAMIC ACID | 27.142138 | 0.27142138 |
| L-HISTIDINE F.B. | 18.094638 | 0.18094638 |
| L-ISOLEUCINE | 36.189276 | 0.36189276 |
| L LEUCINE | 54.284276 | 0.54284276 |
| L-LYSINE HCL | 54.284276 | 0.54284276 |
| L METHIONINE | 12.666138 | 0.12666138 |
| L-PHENYLALANINE | 21.713638 | 0.21713638 |
| L-PROLINE | 54.284276 | 0.54284276 |
| L-HYDROXYPROLINE | 18.091381 | 0.18091381 |
| L-SERINE | 54.284276 | 0.54284276 |
| L-THREONINE | 36.189276 | 0.36189276 |
| L-TRYPTOPHAN | 20.808526 | 0.20808526 |
| L-VALINE | 36.189276 | 0.36189276 |
| L CYSTINE DISODIUM SALT | 10.125946 | 0.10125946 |
| L TYROSINE DISODIUM SALT | 26.083882 | 0.26083882 |
| D-CALCIUM PANTOTHENATE | 0.3619 | 0.003619 |
| ZINC SULFATE 7H2O | 0.155979 | 0.00155979 |
| MAGNESIUM CHLORIDE ANHYD | 6.98467 | 0.0698467 |
| SODIUM BICARBONATE | 222 | 2.22 |
| SODIUM CHLORIDE | 220 | 2.2 |
| SODIUM CHLORIDE | 260 | 2.6 |
| Total Sodium Chloride | 480 | 4.8 |
| POTASSIUM CHLORIDE | 72.378914 | 0.72378914 |
| Sodium Phosphate Monobasic | 69.384793 | 0.69384793 |
| BETA NA GLYCEROPHOSPHATE | 90.473552 | 0.90473552 |
| | 2219.357721 | 22.19357721 |

TABLE 14
Vitamin Solution (1000X)

| | g/L | mL/100 L | | g/L |
|---|---|---|---|---|
| Components | chmst | chmst | g/100 L | final |
| Biotin | 1.8095 | 100 | 0.18095 | 0.0018095 |
| Folic Acid | 3.618946 | 100 | 0.3618946 | 0.003618946 |
| Riboflavin | 0.361775 | 100 | 0.0361775 | 0.000361775 |
| Vitamin B12 | 0.901131 | 100 | 0.0901131 | 0.000901131 |
| Para Amino Benzoic | 1.8095 | 100 | 0.18095 | 0.0018095 |
| Acid | | | | |
| Choline Chloride | 90.475 | 100 | 9.0475 | 0.090475 |
| Niacinamide | 3.619 | 100 | 0.3619 | 0.003619 |
| Pyridoxine HCl | 3.619 | 100 | 0.3619 | 0.003619 |
| Thiamine HCl | 3.619 | 100 | 0.3619 | 0.003619 |
| Ethanolamine | 13.567631 | 100 | 1.3567631 | 0.013567631 |
| Putrescine 2HCl | 0.54285 | 100 | 0.054285 | 0.00054285 |
| Sodium Phosphate | 9.979071 | 100 | 0.9979071 | 0.009979071 |
| Dibasic | | | | |
| | | | 13.3922404 | 0.133922404 |

TABLE 15
Iron Chelate Solution (2000X)

| | g/L | mL/100 L | | g/L |
|---|---|---|---|---|
| Components | chmst | chmst | g/100 L | final |
| EDTA Tetrasodium 2H₂O | 13.756 | 50 | 0.6878 | 0.006878 |
| Ferrous Sulfate 7 H₂O | 10.0636 | 50 | 0.50318 | 0.0050318 |
| | | | 1.19098 | 0.0119098 |

TABLE 16
Neutral Solution (3333.33X)

| | g/L | mL/100 L | | g/L |
|---|---|---|---|---|
| Components | chmst | chmst | g/100 L | final |
| Sodium | 1.507917 | 30 | 0.04523751 | 0.000452375 |
| Metasilicate | | | | |
| 9H2O | | | | |
| 2-Mercapto- | 4.704760688 | 30 | 0.141142821 | 0.001411428 |
| ethanol | | | | |
| (d = 1.1143 | | | | |
| g/mL) | | | | |
| Monothio- | 60.316667 | 30 | 1.80950001 | 0.018095 |
| glycerol | | | | |
| | | | 1.995880341 | 0.019958803 |

TABLE 17
Calcium Nitrate Solution (3333.33X)

| | g/L | mL/100 L | | g/L |
|---|---|---|---|---|
| Components | chmst | chmst | g/100 L | final |
| Calcium | 301.583333 | 30 | 9.04749999 | 0.090475 |
| Nitrate 4H₂O | | | | |
| | | | 9.04749999 | 0.090475 |

TABLE 18
Milled Pluronic

| Components | g/100 L | g/L final |
|---|---|---|
| Pluronic F68 | 180.947105 | 1.80947105 |
| | 180.947105 | 1.809471 |

TABLE 19

| | g/L | g/100 L |
|---|---|---|
| DPM Chemstock | 22.19357721 | 2219.357721 |
| Incomplete | 22.4499016 | 2244.99016 |
| Complete | 24.25937265 | 2425.937265 |

TABLE 20
Trace Element Solution (5000X)

| | | mL/L | | mL/100 L | | |
|---|---|---|---|---|---|---|
| Components | g/L soln | soln | g/L chmst | chmst | g/100 L | g/L final |
| Aluminum Chloride 6H₂O | 0.150792 | 18 | 0.0027143 | 20 | 5.42851E−05 | 5.42851E−07 |
| Cadmium Chloride 2.5H₂O | 5.730083 | 18 | 0.1031415 | 20 | 0.00206283 | 2.06283E−05 |
| Rubidium Chloride | 0.175924 | 18 | 0.0031666 | 20 | 6.33326E−05 | 6.33326E−07 |
| Zirconium Chloride 8H₂O | 0.402111 | 18 | 0.007238 | 20 | 0.00014476 | 1.4476E−06 |
| Cobalt Chloride 6H₂O | 1.206333 | 18 | 0.021714 | 20 | 0.00043428 | 4.3428E−06 |
| Stannous Chloride 2H₂O | 0.281478 | 1.8 | 0.0005067 | 20 | 1.01332E−05 | 1.01332E−07 |
| Chromium Sulfate 15H₂O | 0.083438 | 18 | 0.0015019 | 20 | 3.00377E−05 | 3.00377E−07 |
| Nickelous Sulfate 6H₂O | 0.033174 | 18 | 0.0005971 | 20 | 1.19426E−05 | 1.19426E−07 |
| Sodium Flouride | 0.502639 | 18 | 0.0090475 | 20 | 0.00018095 | 1.8095E−06 |
| Cupric Sulfate 5H₂O | 1.256597 | 18 | 0.0226187 | 20 | 0.000452375 | 4.52375E−06 |
| Manganese Sulfate H₂O | 0.042222 | 18 | 0.00076 | 20 | 1.51999E−05 | 1.51999E−07 |
| Ammonium Molybdate | 1.507917 | 18 | 0.0271425 | 20 | 0.00054285 | 5.4285E−06 |
| Germanium Dioxide | 0.067354 | 18 | 0.0012124 | 20 | 2.42474E−05 | 2.42474E−07 |
| Sodium Meta Vanadate | 0.155818 | 18 | 0.0028047 | 20 | 5.60945E−05 | 5.60945E−07 |
| Potassium Bromide | 0.150792 | 1.8 | 0.0002714 | 20 | 5.42851E−06 | 5.42851E−08 |
| Potassium Iodide | 0.231214 | 1.8 | 0.0004162 | 20 | 8.3237E−06 | 8.3237E−08 |
| Barium Acetate | 0.326715 | 18 | 0.0058809 | 20 | 0.000117617 | 1.17617E−06 |
| Silver Nitrate | 0.221161 | 1.8 | 0.0003981 | 20 | 7.9618E−06 | 7.9618E−08 |
| Titanium Tetrachloride | 0.12492788 | 18 | 0.0022487 | 20 | 4.4974E−05 | 4.4974E−07 |
| (d = 1.726 g/mL) | | | | | | |
| Sodium Selenite | 4.362906 | 18 | 0.0785323 | 20 | 0.001570646 | 1.57065E−05 |
| | | | | | 0.00583827 | 5.83827E−05 |

### Example 26

### Exemplary Processing Instructions for Exemplary Culture Media of the Invention.

The following are exemplary procedures for making agglomerated culture media. One skilled in the art will recognize various culture medium formulations that are compatible with the procedures in this example. The order, as described herein, that solutions are sprayed in are meant as examples and one skilled in the art will recognize that the order can be varied depending on the characterization of the solution. The methods described in this example are exemplary. One skilled in the art will recognize and, based on the teaching herein, can carry out numerous variations to achieve a product with the desired characteristics.

In some instances, a vitamin solution is the first or one of the first (e.g., one of the first three) solutions sprayed in. A vitamin solution when sprayed on the granules typically colors the granules. If sprayed in later or last, the particles can be unevenly colored. An iron citrate solution can also color the particles. In some embodiments, an iron citrate solution is sprayed in as either the first, second, or third solution. In some embodiments, a vitamin solution is the first solution sprayed in and an iron citrate solution is sprayed in second. The color of the particles is an aesthetic consideration.

Solutions that are more volatile are typically one of the last solutions to be sprayed in. In some embodiments, a lipid solution (e.g., comprising an organic solvent such as ethanol) and an amine solution are the last two solutions sprayed in. In some embodiments, a lipid solution is the last solution to be sprayed in and the amine solution is the second to last solution to be sprayed in. In some embodiments, more volatile solutions (e.g., a lipid solution and/or an amine solution) are sprayed in at a reduced temperature.

A neutral or trace element solution can be sprayed in essentially any time, but considering the above, the trace element solution is typically sprayed in during the “middle” of the agglomeration process, e.g., after the vitamin and/or iron citrate solution and before the lipid and/or amine solution.

Prior to fluid bed agglomeration, a dry chemstock (chemstock starting powder) is typically processed and sized in a Fitzmill using standard procedures to prepare the powder for agglomeration in a fluid bed processor.

After fluid bed agglomeration the agglomerated product may be sized in a Fitzmill as described in Example 27.

After sizing, the agglomerated granules may be blended, as described in Example 28, with milled Pluronic-68, if part of the desired formulation.

Model: GPCG Pro 120 fluid bed processor; S/N: 8088

### Process and Settings for Small Bowl (50 kg)

An in-process data sheet can be utilized for recording, e.g., the elapsed time (min); inlet air temp (actual) (° C.); exhaust air temp (actual) (° C.); product temp (actual) (° C.); dewpoint (actual) (° C.); spray rate (g/min) operating valve; atm. air (BAR) set point; air volume (cfm; set point and actual); and/or differential pressure (DP; mm/H₂O; product and filter).

### Step 1: Fluid Bed Set-Up Instructions

a. Verification that the correct expansion chamber and product bowl are cleaned within 24 hours prior to using Fluid Bed.

b. Verification equipment is dry prior to processing.

c. Verification that spray system is functioning. (Purge the line with air and prime the spray line.)

d. Filter installation
Filters required: #002A & 003A

e. Filter installation verified

f. Nozzle installation:
Nozzle required: Three-Head
Port required: Bottom Port

g. Verify correct nozzle installation

### Step 2: Pre-Processing Instructions

a. Fully assemble the tower and inflate the seals

b. Verify that the proper solutions are ready and have been dispensed

c. Stage solutions in order:
1 Vitamin Solution (2000×)
2 Iron Citrate Solution (2000×)
3 Trace Element Solution (5000×)
4 Amine Solution (5000×)
5 Lipid Solution (5000×)

d. Manufacturing Engineer or Scientist present during processes performed in Development Mode.

e. Select Development Mode

f. Enter the batch lot number

g. Press the start button to initiate the batch and note start time.

h. Insert wand into the Vitamin Solution

### Step 3: Pre-Processing Instructions

a. Prior to processing the batch, pre-warm the Fluid Bed Processor for a minimum of 15 minutes using the following parameters and note the start time:

| Parameter: | Set Point: | |
|---|---|---|
| Process Air Volume | 1000 | cfm |
| Inlet Air Temperature | 40° | C. |
| Dewpoint | −10° | C. |

b. Verify that the dew point has stabilized at −10° C. (+/−2° C.) before proceeding.

c. Note the stop time, verifying at least 15 minute warm-up

### Step 4: Charging the Bowl Instructions

a. Deflate the seals and remove the product bowl

b. Add DPM Chemstock to the bowl

c. Re-install the product bowl and inflate the seals

### Step 5: Granulation Process Instructions

a. Record the actual parameters, e.g., on an in-process data sheet, at least every 15 minutes and when making a parameter change. Operating conditions may be adjusted as necessary in order to make a good granulation.

b. Begin to Fluidize the product using the following parameters:

| Parameter: | Target: | Range: |
|---|---|---|
| Process Air Volume | 600 cfm | 400 cfm-1200 cfm |
| Inlet Air Temperature | 55° C. | 30° C.-60° C. |
| Dewpoint | −10° C. | less than 0° C. |
| Shake Mode | GPCG | |
| Shake Interval | 30 seconds | |
| Shake Duration | 5 seconds | |

c. After approximately one minute, begin spraying the Vitamin Solution. Use the following parameters during spraying:

| Parameter: | Target: | Range: |
|---|---|---|
| Process Air Volume | 600 cfm | 200 cfm-2500 cfm |
| Inlet Air Temperature | 55° C. | 40° C.-65° C. |
| Product Temperature | less than 40° C. | 0° C.-45° C. |
| Spray Rate | 100 g/min | 50 g/min-400 g/min |
| Atomization Air | 2 BAR | 2 BAR |
| Shake Mode | GPCG | |
| Shake Interval | 30 seconds | 15-45 seconds |
| Shake Duration | 5 seconds | |

d. Increase air flow as necessary to achieve proper fluidization.

e. Upon completion of spraying the Vitamin Solution, rinse the lines for 30 seconds with WFI.

f. Begin spraying Iron Citrate Solution. Use the following parameters during spraying:

| Parameter: | Target: | Range: |
|---|---|---|
| Process Air Volume | 1000 cfm | 200 cfm-2500 cfm |
| Inlet Air Temperature | 55° C. | 40° C.-65° C. |
| Product Temperature | less than 40° C. | 0° C.-45° C. |
| Spray Rate | 100 g/min | 50 g/min-400 g/min |
| Atomization Air | 2 BAR | 2 BAR |
| Shake Mode | GPCG | |
| Shake Interval | 30 seconds | 15-45 seconds |
| Shake Duration | 5 seconds | |

g. Upon completion of spraying the Iron Citrate Solution, rinse the lines for 30 seconds with WFI.

h. Begin spraying the Trace Element Solution. Use the following parameters during spraying:

| Parameter: | Target: | Range: |
|---|---|---|
| Process Air Volume | 1000 cfm | 200 cfm-2500 cfm |
| Inlet Air Temperature | 55° C. | 40° C.-65° C. |
| Product Temperature | less than 40° C. | 0° C.-45° C. |
| Spray Rate | 100 g/min | 50 g/min-400 g/min |
| Atomization Air | 2 BAR | 2 BAR |
| Shake Mode | GPCG | |
| Shake Interval | 30 seconds | 15-45 seconds |
| Shake Duration | 5 seconds | |

i. Upon completion of spraying the Trace Element Solution, rinse the lines for 30 seconds with WFI.

j. Begin spraying the Amine Solution. Use the following parameters during spraying:

| Parameter: | Target: | Range: |
|---|---|---|
| Process Air Volume | 1000 cfm | 200 cfm-2500 cfm |
| Inlet Air Temperature | 55° C. | 40° C.-65° C. |
| Product Temperature | less than 40° C. | 0° C.-45° C. |
| Spray Rate | 100 g/min | 50 g/min-400 g/min |
| Atomization Air | 2 BAR | 2 BAR |
| Shake Mode | GPCG | |
| Shake Interval | 30 seconds | 15-45 seconds |
| Shake Duration | 5 seconds | |

k. Upon completion of spraying the Amine Solution, rinse the lines for 30 seconds with WFI. Allow the granulation to dry at 40° C. for 5 minutes (range: 2-10 minutes). Air flow may be decreased as necessary. Note start time and stop time.

l. Begin spraying the Lipid Solution. Use the following parameters during spraying:

| Parameter: | Target: | Range: |
|---|---|---|
| Process Air Volume | 1000 cfm | 200 cfm-2500 cfm |
| Inlet Air Temperature | 40° C. | 40° C.-65° C. |
| Product Temperature | less than 40° C. | 0° C.-45° C. |
| Spray Rate | 100 g/min | 50 g/min-400 g/min |
| Atomization Air | 2 BAR | 2 BAR |
| Shake Mode | GPCG | |
| Shake Interval | 30 seconds | 15-45 seconds |
| Shake Duration | 5 seconds | |

m. Upon completion of spraying Lipid Solution, rinse the lines for 30 seconds with WFI and then empty the spray line. Allow the granulation to dry at 40° C. for 5 minutes (range: 2-10 minutes). Air flow may be decreased as necessary. Note start time and stop time.

n. Prior to final processor shutdown, obtain a moisture sample from the sample port.

o. Upon completion of the dry cycle, record the granulation completion time.

p. Analyze sample with moisture analyzer and record moisture content. If the moisture content is greater than 2.0%, contact a Manufacturing Supervisor or a Process Engineer.

q. Print batch report. Attach to batch record.

### Step 6: Discharging the Bowl Instructions

a. Perform manual shake for 60 seconds

b. Tare drum(s) on the floor scale. Record tare weight.

c. Discharge product bowl into drum(s)

d. Weigh the drum(s) on floor scale. Record gross weight.

e. Perform net weight calculation to be used in step 7b: Net weight=Gross weight (from step 6d)−Tare weight (from step 6b)

f. 2nd check on net weight calculation Per Karen

### Step 7: Yield Calculation

a. Theoretical batch weight ______ kg

b. Total volume packaged ______ kg

c. Total volume discarded ______ kg

d. Yield percentage=[(Total volume packaged (from Step 7b) ______ kg+Total volume discarded (from Step 7c) ______ kg)/Theoretical batch weight (from Step 7a) ______ kg]×100=______ %

e. 2nd check on yield calculation

f. Clear work area

### Step 8: Cleaning Requirements

a. Verification of clean in place (CIP) started within 4 hours of pre-rinse step.

b. Verification that CIP has been completed within 24 hours of the end of this production.

c. Record cleaning start time

d. Record pre-rinse completion time

e. Record CIP start time

### Process and Settings for Large Bowl (525 L; 125 kg)

The process and the settings for the large bowl with 125 kg are the same as those above for the small bowl (50 kg) with the following exceptions.

Step 1 f. Nozzle installation:
Nozzle required: Six-Head
Port required: Top Port

Steps 5c, 5f, 5h, 5j and 5l have a target spray rate of 250 g/min

### Process and Settings for Small Bowl (290 L; 150 kg)

The process and the settings for the small bowl (150 kg) are the same as those above for the small bowl (50 kg) with the following exceptions.

Steps 5c, 5f, 5h, 5j and 5l have a target spray rate of 300 g/min.

### Example 27

Fitzmill sizing of agglomerated particles using a Fitzpatrick Fitzmill model D6A (Fitzpatrick, Elmhurst, Ill.)

### Step 1: D6A—Fitzmill Set Up and Sizing Operation: Animal Origin Free (AOF)

a. Verify that all drums are present for all relevant lots

b. 2nd check

c. Verification that equipment is clean and completely dry prior to processing (N/A for consecutive batches)

d. Mill Set up

e. Knife Blades installed

f. Inspect 0.050″ perforated plate

g. Install 0.050″ perforated plate

h. 2nd check for proper plate and rotor/impact blade assembly

i. FitzMill Process Conditions:
Rotor Speed: 1000 rpms (Record actual rotor speed)
Feed Rate: 20-50 rpms (Record actual feed rate)

j. 2nd check for correct process conditions

k. Complete Blend Weight Verification for each lot to be sized and added to blending vessel.

l. Add granulation to feed hopper

m. Mill/size into final blending vessel (e.g., 200-L or 400-L drum)

n. Do not add Milled Pluronic® F-68 (0055088) to Fitzmill

o. Post milling inspection to ensure full delivery of components. Inspect feed hopper, mill chamber, and discharge adaptor.

### Example 28

Blending apparatuses that can be used with the invention include, but are not limited to, a Gemcomatic Slant Cone Blender (Gemco, Middlesex, N.J.).

### Step 1: Blending Process Instructions—Addition of Pluronic® F-68—AOF Manufacturing Area

a. Add Milled Pluronic® F-68 directly to final blending vessel (200-L or 400-L drum)—do not process through FitzMill.

### Step 2: Final Blending Process Section—AOF Manufacturing Area

a. Transfer blending vessel (200-L or 400-L drum) to tumble blender

b. Blend for 20 minutes. Record start time.

c. 2nd check

d. Record stop time

e. 2nd check

f. Measure approximate distance from top rim of drum to surface of powder. Notify Supervisor or Process Engineering if less than 5 inches for a 400-L or less than 9 inches for a 200-L drum.

g. 2nd check

h. Theoretical volume blended ______ Kg:

i. Measure and record tare weight of final blending vessel (Kg)

j. 2nd check on Tare Weight

k. Measure and record gross weight of final blending vessel and powder (Kg)

l. 2nd check on Gross Weight

m. Prepare yield calculation:
Yield Total=Step 2k: Gross Weight (Kg)—Step 2i: Tare Weight (Kg)
Yield Percentage=[Yield Total] divided by [Step 2h: Theoretical volume blended (Kg)] multiplied by 100

n. 2nd Check on yield calculation

o. Clear work area

### Example 29

DMEM, OptiMEM & IMDM media were prepared as described in Example 1 using a MP-1 (Niro, Inc./Aeromatic-Fielder; Columbia, Md.) with the setting and parameters as listed in Table 30 in the column labeled Example 29. Prior to agglomeration sodium bicarbonate was added to the dry powder medium before agglomeration. Post-agglomeration blending was done using a 16 quart slant-cone Gemco (Middlesex, N.J.; Model B91776) tumble blender for 10 minutes.

BAR—Measure of atmospheric pressure; CMH-cubic meters per hour; MMWC—millimeters of water column

TABLE 30

| Parameter | Example 1 | Example 29 |
|---|---|---|
| Inlet Air Temperature | 60 to 65° C. | 60 to 65° C. |
| Outlet Air | ~33° C. | Range 29 to 39° C. |
| Temperature | | |
| Blowback Pressure | 5 BAR | 5 BAR |
| Atomization Pressure | 1.5 to 2.0 BAR | 2.0 BAR |
| Blowback cycle | 2 during spray-in, | 2 during spray-in, |
| | 1 after spray-in completed | 1 after spray-in completed |
| Fan Capacity | * 5 at start of run, | 50 to 85 CMH |
| | *6 after agglomeration is evident | |
| Magnehelics | * Filter resistance 150 to 200 | Filter resistance |
| | *Resistance of perforated control | 23 to 51 MMWC |
| | plate~50 | Bed resistance |
| | *Air volume: less than 50 | 43 to 72 MMWC |
| | | Air volume: 50-85 CMH |
| Liquid Spray-in rate | ~250 mL/2 kg @ 26 g/minute | 250 mL/2 kg |
| | | Spray-in rate @ |
| | | ~26 g(mL)/minute |
| Drying | Thorough drying upon | Thorough drying-final product |
| | completion of liquid addition | temperature = 37 C. at the end of |
| | | drying process |
| | | Actual dry time range = 2 to 6 |
| | | minutes |

*these setting/measurements refer to settings on the Strea 1 bench top laboratory fluid bed apparatus in Example 1.

### Example 30

### Bulk Density Testing

### Materials

Approximately 200 grams of AGT formatted medium—finished product (post-granulation, sizing & blending).

100 mL cylinder—polypropylene cut off at the 100 mL mark

Powder scoop

Pan balance capable of weighing up to 400 grams

### Procedure

1) Tare weigh the 100 mL cylinder before use on pan balance. Scale should read “0”.

2) Hold the 100 mL cylinder over a container or plastic bag to catch excess powder.

3) Using the scoop, holding 4 to 6 inches above the cylinder, with a sifting motion slowly and gently transfer the test material from the scoop into the cylinder until it is slightly over-filled. Be careful not to tap or jar the cylinder during this part of the process.

4) Gently scrape the excess powder off the top of the 100 mL cylinder so it is filled exactly to the 100 mL mark.

5) Re-weigh the filled 100 mL cylinder on the tared pan balance. Record the weight of the 100 mL of powder.

6) Calculate the Bulk Density in grams/mL by dividing the above figure by 100.

### Example 31

### Bulk Density Results

Agglomerated media was prepared as described in Example 1 as set forth in Example 29 and analyzed as described in Example 30. Two different lots of each medium were tested in triplicate. The results are shown in Tables 21, 22 and 23. STDEV=Standard Deviation

This Example demonstrates that dry powder media made according to some embodiments of the invention may have a bulk density of between from about 0.5449 g/ml to about 0.6461 g/ml, about 0.5669 g/ml to about 0.6048 g/ml, about 0.5449 g/ml to about 0.6148 g/ml, about 0.5784 g/ml to about 0.6461 g/ml, about 0.5928 g/ml to about 0.5726 g/ml, about 0.5475 g/ml to about 0.5953 g/ml, about 0.5856 g/ml to about 0.6341 g/ml, about 0.5676 g/ml to about 0.6088 g/ml, about 0.5450 g/ml to about 0.6142 g/ml, about 0.5790 g/ml to about 0.6454 g/ml, about 0.5685 g/ml to about 0.5969 g/ml, about 0.5376 g/ml to about 0.6052 g/ml, about 0.5756 g/ml to about 0.6442 g/ml, about 0.5549 g/ml to about 0.6461 g/ml, or about 0.5376 g/ml to about 0.6461 g/ml.

TABLE 21
OptiMEM AGT

| | | | | Run Average | Lot Average |
|---|---|---|---|---|---|
| | Bulk Density (g/ml) for Analysis# | | | Bulk Density | Bulk Density |
| Lot# | 1 | 2 | 3 | (g/ml) (STDEV) | (g/ml) (STDEV) |
| 023-07-001 | 0.5746 | 0.6048 | 0.5991 | 0.5928 (0.0160) | 0.5827 (0.0142) |
| 025-07-001 | 0.5760 | 0.5750 | 0.5669 | 0.5726 (0.0050) | |

TABLE 22
IMDM AGT

| | | | | Run Average | Lot Average |
|---|---|---|---|---|---|
| | Bulk Density (g/ml) for Analysis# | | | Bulk Density | Bulk Density |
| Lot# | 1 | 2 | 3 | (g/ml) (STDEV) | (g/ml) (STDEV) |
| 023-07-002 | 0.6148 | 0.5942 | 0.5770 | 0.5953 (0.0189) | 0.5714 (0.0338) |
| 025-07-002 | 0.5498 | 0.5449 | 0.5478 | 0.5475 (0.0025) | |

TABLE 23
DMEM AGT

| | | | | Run Average | Lot Average |
|---|---|---|---|---|---|
| | Bulk Density (g/ml) for Analysis# | | | Bulk Density | Bulk Density |
| Lot# | 1 | 2 | 3 | (g/ml) (STDEV) | (g/ml) (STDEV) |
| 023-07-003 | 0.6461 | 0.6325 | 0.6236 | 0.6341 (0.0113) | 0.6099 (0.0343) |
| 025-07-003 | 0.5784 | 0.5870 | 0.5914 | 0.5856 (0.0066) | |

### Example 32

### Wet-Ability Testing Protocol

### Materials

15 gram samples of AGT or DPM formatted medium to be tested

1 liter graduated cylinder

1 liter WFI @ room temperature

thermometer

stopwatch

### Procedure

1) Fill a 1 liter graduated cylinder to the 1 liter mark with WFI (record WFI temperature).

2) Slowly pour the medium 15 gram sample to be tested onto the surface of the WFI in the cylinder.

3) Start the stopwatch timer when the entire 15 gram sample has been added to the cylinder.

4) Leaving the cylinder undisturbed, allow the test material to sink into the WFI.

5) When the entire amount of the 15 gram sample has totally submerged below the WFI surface in the cylinder, stop the timer and record the time elapsed in seconds.

6) Repeat each lot of material to be tested 3 times, recording data for each test.

7) Calculate and record the mean Wet-ability time and ±SD for each sample.

### Example 33

### Wet-Ability Results

Agglomerated media was prepared as described in Example 1 as set forth in Example 29 and analyzed as described in Example 32. The WFI temperature for these experiments was 23° C. “DPM” refers to dry powder medium that has not been agglomerated. “AGT” refers to agglomerated medium and in this case as described in Example 29. Two different lots of each medium were tested in triplicate. The results are shown in Tables 24, 25 and 26.

This Example demonstrates that dry powder media made according to some embodiments of the invention may have a Wet-ability measure of between from about 1 second to about 18 seconds, about 1 second to about 2 seconds, about 1 second to about 18 seconds, about 7 seconds to about 18 seconds, about 1 second to about 15 seconds, about 1.2 seconds to about 12 seconds, about 1.7 seconds to about 12 seconds, about 1.2 seconds to about 1.7 seconds, about 1.3 seconds to about 2 seconds, about 1 second to about 1.3 seconds, about 9.3 seconds to about 15 seconds, about 1.2 seconds to about 2.2 seconds, about 1.0 second to about 1.4 seconds, about 8 seconds to about 16 seconds, about 1.0 seconds to about 16 seconds.

TABLE 24
OptiMEM Wet-ability Analysis

| | | | Mean | | Mean of 2 Lots | |
|---|---|---|---|---|---|---|
| Description | Lot# | Seconds | (seconds) | STDEV | (Seconds) | STDEV |
| OptiMEM DPM | 1347124 | 238 | 237 | 9.07 | N/A | N/A |
| OptiMEM DPM | 1347124 | 227 | | | | |
| OptiMEM DPM | 1347124 | 245 | | | | |
| OptiMEM AGT | 023-07001 | 2 | 2 | 0 | 1.7 | 0.47 |
| OptiMEM AGT | 023-07001 | 2 | | | | |
| OptiMEM AGT | 023-07001 | 2 | | | | |
| OptiMEM AGT | 025-07-001 | 1 | 1.3 | 0.58 | | |
| OptiMEM AGT | 025-07-001 | 1 | | | | |
| OptiMEM AGT | 025-07-001 | 2 | | | | |

TABLE 25
IMDM Wet-ability

| | | | Mean | | Mean of 2 Lots | |
|---|---|---|---|---|---|---|
| Description | Lot# | Seconds | (seconds) | STDEV | (Seconds) | STDEV |
| IMDM DPM | 1348537 | 455 | 475 | 24.8 | N/A | N/A |
| IMDM DPM | 1348537 | 468 | | | | |
| IMDM DPM | 1348537 | 503 | | | | |
| IMDM AGT | 023-07-002 | 1 | 1 | 0 | 1.2 | 0.24 |
| IMDM AGT | 023-07-002 | 1 | | | | |
| IMDM AGT | 023-07-002 | 1 | | | | |
| IMDM AGT | 025-07-002 | 1 | 1.3 | 0.58 | | |
| IMDM AGT | 025-07-002 | 2 | | | | |
| IMDM AGT | 025-07-002 | 1 | | | | |

TABLE 26
DMEM Wet-ability

| | | | Mean | | Mean of 2 Lots | |
|---|---|---|---|---|---|---|
| Description | Lot# | Seconds | (seconds) | STDEV | (Seconds) | STDEV |
| DMEM DPM | 1349729 | 374 | 369 | 6.25 | N/A | N/A |
| DMEM DPM | 1349729 | 362 | | | | |
| DMEM DPM | 1349729 | 371 | | | | |
| DMEM AGT | 023-07-003 | 12 | 9.3 | 2.5 | 12 | 4.0 |
| DMEM AGT | 023-07-003 | 9 | | | | |
| DMEM AGT | 023-07-003 | 7 | | | | |
| DMEM AGT | 025-07-003 | 14 | 15 | 2.6 | | |
| DMEM AGT | 025-07-003 | 18 | | | | |
| DMEM AGT | 025-07-003 | 13 | | | | |

### Example 34

### A Sieve Analysis Testing Protocol

Describe the testing method used to determine the particle size distribution of the AGT granulated formatted medium.

### Materials

Approximately 100 grams of AGT formatted medium—finished product.

USA Standard Sieve screens—(Fisher Scientific) in the following Tyler equivalent mesh sizes: 30; 45; 60; 80; 100; 200 & Pan.

Rotap machine Model RX-29, type ROTAP manufactured by W. S. Tyler, Mentor, Ohio.

Scale with capacity of 500 grams—to weigh sample and individual sieves.

### Procedure

1) Check sieves for screen integrity. Replace sieve if screen integrity is compromised.

2) Record tare weight for each mesh screen

3) Stack screens in order (lowest mesh size/highest mesh size, e.g. pan on bottom follow by 200 mesh, 100 mesh etc.).

4) Weigh 100 gram sample of material to be tested, place onto upper screen.

5) Place cover on top of stacked screens/sample and place into the Rotap machine.

6) Carefully lower the tapping arm onto the top of stacked screens.

7) Set timer on front of machine for 5 minutes, press “START” button. Rotap machine will start and automatically stop after 5 minutes.

8) Upon completion of tap cycle, remove stack of sieves and weigh the combined screen with the powder remaining for each of the individual sieve mesh sizes. Record as Gross Weight.

9) Obtain the Sample Net Weight by subtracting the specific sieve screen Tare Weight from the corresponding sieve mesh with powder Gross Weight. Record as Sample Weight Retained for each mesh size tested.

10) Obtain Total Net Weight by adding all individual mesh Sample Weight Retained weights.

11) Obtain % Retained for each mesh size tested by dividing each individual Net Weight by the Total Net Weight.

12) Obtain % Cumulative by adding the current mesh % retain to all mesh % of larger mesh.

### Example 35

### Sieve Analysis Testing Results

Agglomerated media was prepared as described in Example 29 and analyzed as described in Example 34. Two different lots of each medium were tested. The results are shown in Tables 27, 28 and 29.

This Example demonstrates that dry powder media made according to some embodiments of the invention may have between from about 7.06% to about 30.69% retained at the 30 mesh size and above; about 17.83% to about 73.42% retained at the 45 mesh size and above; about 32.66% to about 91.84% retained at the 60 mesh size and above; about 55.98% to about 97.04% retained at the 80 mesh size and above; about 68.37% to about 98.20% retained at the 100 mesh size and above; about 96.34% to about 99.85% retained at the 200 mesh size and above; about 0.15% to about 3.66% retained below the 200 mesh size.

TABLE 27
OptiMEM Sieve Analysis

| | | Cumulative | | Cumulative | | Average |
|---|---|---|---|---|---|---|
| Screen | % Retained | % Retained | % Retained | % Retained | Average % | Cumulative |
| Size | Lot # | Lot # | Lot # | Lot # | Retained for | % Retained |
| (Mesh#) | 023-07-001 | 023-07-001 | 025-07-001 | 025-07-001 | 2 lots | for 2 lots |
| 30 | 12.02 | 12.02 | 10.56 | 10.56 | 11.29 | 11.29 |
| 45 | 29.96 | 41.98 | 26.46 | 37.02 | 28.21 | 39.50 |
| 60 | 30.36 | 72.34 | 33.10 | 70.12 | 31.73 | 71.23 |
| 80 | 17.03 | 89.38 | 21.43 | 91.55 | 19.23 | 90.46 |
| 100 | 4.41 | 93.79 | 4.93 | 96.48 | 4.67 | 95.13 |
| 200 | 5.51 | 99.30 | 3.42 | 99.90 | 4.47 | 99.60 |
| Pan | 0.70 | 100.00 | 0.10 | 100.00 | 0.40 | 100.00 |

TABLE 28
IMDM Sieve Analysis

| | | Cumulative | | Cumulative | | Average |
|---|---|---|---|---|---|---|
| Screen | % Retained | % Retained | % Retained | % Retained | Average % | Cumulative |
| Size | Lot # | Lot # | Lot # | Lot # | Retained for | % Retained |
| (Mesh#) | 023-07-002 | 023-07-002 | 025-07-002 | 025-07-002 | 2 lots | for 2 lots |
| 30 | 35.34 | 35.34 | 26.05 | 26.05 | 30.69 | 30.69 |
| 45 | 37.75 | 73.09 | 47.70 | 73.75 | 42.73 | 73.42 |
| 60 | 16.47 | 89.56 | 20.36 | 94.11 | 18.41 | 91.84 |
| 80 | 6.02 | 95.58 | 4.39 | 98.50 | 5.21 | 97.04 |
| 100 | 1.61 | 97.19 | 0.70 | 99.20 | 1.15 | 98.20 |
| 200 | 2.61 | 99.80 | 0.70 | 99.90 | 1.65 | 99.85 |
| Pan | 0.20 | 100.00 | 0.10 | 100.00 | 0.15 | 100.00 |

TABLE 29
DMEM Sieve Analysis

| | | Cumulative | | Cumulative | | Average |
|---|---|---|---|---|---|---|
| Screen | % Retained | % Retained | % Retained | % Retained | Average % | Cumulative |
| Size | Lot # | Lot # | Lot # | Lot # | Retained for | % Retained |
| (Mesh#) | 023-07-003 | 023-07-003 | 025-07-003 | 025-07-003 | 2 lots | for 2 lots |
| 30 | 12.41 | 12.41 | 1.71 | 1.71 | 7.06 | 7.06 |
| 45 | 15.22 | 27.63 | 6.33 | 8.03 | 10.77 | 17.83 |
| 60 | 16.22 | 43.84 | 13.45 | 21.49 | 14.84 | 32.66 |
| 80 | 18.62 | 62.46 | 28.01 | 49.50 | 23.32 | 55.98 |
| 100 | 9.61 | 72.07 | 15.16 | 64.66 | 12.39 | 68.37 |
| 200 | 23.82 | 95.90 | 32.13 | 96.79 | 27.98 | 96.34 |
| Pan | 4.10 | 100.00 | 3.21 | 100.00 | 3.66 | 100.00 |

### Example 36

### A Flow Analysis Procedure

This procedure can be used for flow analysis and measurements that can be determined include FRI (Flow Rate Index); FDI (Feed Density Index); BDI (Bin Density Index); and SPI (Spring Density Index).

1. Assemble Johanson Indicizer (Johanson Innovations, Inc, San Luis Obispo, Calif.) sample container by placing 80 mesh screen clamped onto the support insert in bottom of sample cup.

2. Tare Johanson Indicizer sample container on appropriate balance.

3. Place approximately 100 gram sample into suitable container and aerate by mixing the sample with a spoon or whisk.

4. Using a spatula, remove a portion of the sample and gently place the sample into the assembled Johanson Indicizer sample container, avoiding compacting the sample.

5. Repeat addition of sample until powder is overflowing/above the Johanson Indicizer sample container.

6. Gently level the powder bed to the top of the Johanson Indicizer sample container by scraping off the excess powder.

7. Weigh and record sample weight of powder in the Johnanson Indicizer sample container using already tared balance.

8. Place the sample container onto the Johanson Indicizer and attach the air lines to the sample container.

9. Set processing parameters on Johanson Indicizer for Bin Angle as 32°, Outlet diameter as 8 inches, and Bin diameter as 2.5 ft. After the test is complete, Record Johanson Indicizer output.

### Example 37

### A Procedure for Measuring Angle of Repose

1. Place approximately 50 grams of sample into a suitable container and aerate by mixing the sample with a spoon or whisk.

2. Set rectangle powder bed box onto support platform.

3. Ensure that support platform is set at 0 (zero) degree angle, read at bottom of platform.

4. Pour material through the funnel into rectangle powder bed box until material begins to touch at least one of the box's sidewall and forms a cone.

5. Slowly and constantly raise the powder bed box and platform using the support screw ensuring as smooth a transition as possible.

6. As soon as the peak of the material shifts, stop rotating the support screw, and take the angle of repose measurement using the device's protractor, reading the protractor's angle at the base of the support platform.

### Example 38

### A Concentrated Feed Supplement Medium

A concentrated feed supplement medium containing the components as listed in Tables 32, 33 and 34 was prepared as follows.

1) An appropriate volume 40 mL of HCL (1N) per liter supplement solution was added to water used for formulation resulting in a pH of 1.8 to 1.9. The total volume is 80% of final production volume WFI (e.g., 800 mL for 1 liter final volume) of water used.

2) Amounts of the amino acids L-Cystine and L-Asparagine were added so as the concentrations listed in Table 33 are achieved in the final solution (step 6). These concentrations in the final feed supplement medium are believed to be above their solubility limit at the 5× concentration at a neutral pH (7.0). L-Cystine and L-Asparagine were added to the acidified water and mixed until dissolved >15 minutes. After addition of the two amino acids the pH was about 2.1.

3) A dry powder form of the remainder of components (see Table 32), except for L-Tyrosine, was prepared using fluid bed agglomeration as described herein. The agglomerated dry powder form of the remainder of components was then reconstituted with water to result in the concentrations listed in at 5× for the final solution (step 6). This reconstituted solution did not contain sodium bicarbonate, potassium chloride, sodium chloride, and Pluronic F-68®. This reconstituted solution was then added to the acidified water containing L-Cystine and L-Asparagine. This solution was allowed to mix for >15 minutes. The pH of this solution was about 4.8. The solution may be cloudy but will typically clear with the subsequent additions and pH adjustment to neutral (e.g., about 7.0).

4) An amount of the amino acid L-Tyrosine was added so as the concentration listed in Table 34 is achieved in the final solution (step 6). This concentration is believed to be above its solubility limit at the 5× concentration at neutral pH (7.0). The L-Tyrosine was added to an appropriate volume of a dilute NaOH solution (1N), 30 mL/liter equivalent.

5) The base solubilized amino acid solution was added to the solution from (3) above and mixed for >10 minutes. The solution can either be pH adjusted, e.g., to neutral such as 7.0 to 7.2±0.2 or the pH of the previous acidic and basic solutions can be predetermined, so that upon addition of the base solubilized amino acid solution, the desired pH is achieved. In most instances, the solution will clear and/or pH will be neutral. If required the final pH adjust is made using the appropriate volume of 5N HCl or 5N NaOH.

6) The solution was brought to the desired final production volume with WFI using a calibrated volumetric container.

This 5× feed supplement medium contained a complement of components at 5× without sodium bicarbonate, potassium chloride, sodium chloride, Pluronic F-68®. This 5× feed supplement can be used for supplementing many different types of medium, e.g., for feed supplementing a culture medium as described in Table 2 of U.S. patent application Ser. No. 11/151,647.

TABLE 32
Remainder of components

| | 1x | 5x | 5x |
|---|---|---|---|
| Component | g/L | g/L | g/kg |
| L-Isoleucine | 0.36192 | 1.8096 | 26.69959 |
| L-Leucine | 0.54288 | 2.7144 | 40.04939 |
| L-Lysine HCl | 0.54288 | 2.7144 | 40.04939 |
| L-Proline | 0.54288 | 2.7144 | 40.04939 |
| L-Serine | 0.54288 | 2.7144 | 40.04939 |
| L-Arginine F.B. | 0.36192 | 1.8096 | 26.69959 |
| L-Aspartic Acid | 0.18096 | 0.9048 | 13.3498 |
| L-Glutamic Acid | 0.27144 | 1.3572 | 20.02469 |
| L-Histidine F.B. | 0.18096 | 0.9048 | 13.3498 |
| L-Methionine | 0.12668 | 0.6334 | 9.345447 |
| L-Phenylalanine | 0.21716 | 1.0858 | 16.02034 |
| L-Hydroxyproline | 0.18092 | 0.9046 | 13.34684 |
| L-Threonine | 0.36192 | 1.8096 | 26.69959 |
| L-Tryptophan | 0.20808 | 1.0404 | 15.35049 |
| L-Valine | 0.36192 | 1.8096 | 26.69959 |
| Magnesium Chloride Anhyd | 0.06966 | 0.3483 | 5.138963 |
| D-Calcium Pantothenate | 0.00362 | 0.0181 | 0.267055 |
| D-Glucose (Dextrose) | 6.3336 | 31.668 | 467.2428 |
| Zinc Sulfate 7H2O | 0.00156 | 0.0078 | 0.115084 |
| Sodium Phosphate Dibasic | 0.70384 | 3.5192 | 51.92374 |
| Anhyd. | | | |
| Beta Sodium Glycerophosphate | 0.9048 | 4.524 | 66.74898 |
| Calcium Nitrate 4H2O | 0.09048 | 0.4524 | 6.674898 |
| Pyridoxine HCl | 0.00362 | 0.0181 | 0.267055 |
| Thiamine HCl | 0.00362 | 0.0181 | 0.267055 |
| Folic Acid | 0.00362 | 0.0181 | 0.267055 |
| Biotin | 0.0018 | 0.009 | 0.13279 |
| Sodium Pyruvate | 0.1992 | 0.996 | 14.6954 |
| Ascorbic Acid 2 Phosphate | 0.01991 | 0.09955 | 1.468802 |
| Magnesium | | | |
| i-Inositol | 0.0632 | 0.316 | 4.662395 |
| Glutathione Reduced | 0.0018 | 0.009 | 0.13279 |
| Putrescine 2HCl | 0.00068 | 0.0034 | 0.050165 |
| Ethanolamine HCl | 0.016963 | 0.084813 | 1.251359 |
| Spermine 4HCl | 0.01557 | 0.07785 | 1.148631 |
| Sodium Metasilicate 9H2O | 0.000302 | 0.001508 | 0.022248 |
| 2-Mercaptoethanol | 0.000844 | 0.004222 | 0.062296 |
| (in mLs not grams) | | | |
| Monothioglycerol | 0.012063 | 0.060317 | 0.889937 |
| Aluminum Chloride 6H₂O | 5.43E−07 | 2.71E−06 | 4E−05 |
| Cadmium Chloride 2.5H₂O | 2.06E−05 | 0.000103 | 0.001522 |
| Chromium Chloride 6H2O | 2.89E−07 | 1.44E−06 | 2.13E−05 |
| Rubidium Chloride | 6.33E−07 | 3.17E−06 | 4.67E−05 |
| Zirconium Chloride 8H₂O | 1.45E−06 | 7.24E−06 | 0.000107 |
| Cobalt Chloride 6H₂O | 4.34E−06 | 2.17E−05 | 0.00032 |
| Stannous Chloride 2H₂O | 1.01E−07 | 5.07E−07 | 7.48E−06 |
| Nickelous Sulfate 6H₂O | 1.19E−07 | 5.97E−07 | 8.81E−06 |
| Sodium Flouride | 1.81E−06 | 9.05E−06 | 0.000133 |
| Cupric Sulfate 5H₂O | 4.52E−06 | 2.26E−05 | 0.000334 |
| Manganese Sulfate H₂O | 1.52E−07 | 7.6E−07 | 1.12E−05 |
| Ammonium Molybdate | 5.43E−06 | 2.71E−05 | 0.0004 |
| Germanium Dioxide | 2.42E−07 | 1.21E−06 | 1.79E−05 |
| Sodium Meta Vanadate | 5.61E−07 | 2.8E−06 | 4.14E−05 |
| Potassium Bromide | 5.43E−08 | 2.71E−07 | 4E−06 |
| Potassium Iodide | 8.32E−08 | 4.16E−07 | 6.14E−06 |
| Barium Acetate | 6.53E−26 | 3.27E−25 | 4.82E−24 |
| Silver Nitrate | 7.96E−08 | 3.98E−07 | 5.87E−06 |
| Titanium Tetrachloride | 2.61E−07 | 1.3E−06 | 1.92E−05 |
| (in mLs not grams) | | | |
| Sodium Selenite | 1.57E−05 | 7.85E−05 | 0.001159 |
| EDTA Tetrasodium 2H₂O | 0.006878 | 0.03439 | 0.507404 |
| Ferrous Sulfate 7 H₂O | 0.005032 | 0.025159 | 0.371206 |
| Riboflavin | 0.000362 | 0.001809 | 0.026689 |
| Vitamin B12 | 0.000901 | 0.004506 | 0.066478 |
| Sodium Phosphate Dibasic | 0.009979 | 0.049895 | 0.736177 |
| Para Amino Benzoic Acid | 0.00181 | 0.009048 | 0.133491 |
| Choline Chloride | 0.090475 | 0.452375 | 6.674529 |
| Niacinamide | 0.003619 | 0.018095 | 0.266981 |
| Total | 13.55526 | 67.77632 | 1000 |
| | Liters/kg = | 14.75442 | |

TABLE 33
Acid Amino Acidic Solution

| | 1x | 5x |
|---|---|---|
| Component | g/L | g/L |
| Cystine 2HCl | 0.10499 | 0.52495 |
| Asparagine H2O | 0.9048 | 4.524 |
| Total | 1.00979 | 5.04895 |

TABLE 34
Basic Amino Acid Solution

| | 1x | 5x |
|---|---|---|
| Component | g/L | g/L |
| Tyrosine 2Na | 0.260839 | 1.304194 |
| Total | 0.260839 | 1.304194 |

### Example 39

### Component Analysis of a Concentrated Feed Supplement Medium

A 5× concentrated feed supplement medium was prepared as described in Example 38. In this feed supplement, several of the amino acids are in a neutral solution (pH of 7.0) at a concentration exceeding their normal solubility limit.

This 5× concentrated feed supplement medium was evaluated for stability. After preparation as described in Example 38, the 5× concentrated feed supplement medium was placed at 4° C. for between 18 to 19 months. The medium was then analyzed and the results are shown in the last column of Table 12.

For water soluble vitamin analysis an ion-pair reverse phase HPLC separation followed by UV detection was validated for the identification and quantification for the following components in serum-free media samples: L-tryptophan, niacinamide, folic acid, thiamine, riboflavin, vitamin B₁₂, and phenol red. For amino acid analysis a pre-column derivatization (Waters Accutag) followed by reverse phase HPLC analysis to analyze for amino acids and ammonia in cell culture media was performed. The assay has been optimized for cell culture products and can be used to support formulation optimization. This evaluation showed that the components will remain in solution for a period of time, (e.g., for up to 18 months or longer) without precipitation, e.g., when stored at 4° C. Note that the concentration of L-Cystine cannot be quantified due to the co-elution of multiple component forms and the current limitations of the assay.

TABLE 12

| | % of |
|---|---|
| Component | Theoretical |
| L-ARGININE | 97.86 |
| L-ASPARAGINE H2O | 89.59 |
| L-ASPARTIC ACID | 97.20 |
| L-CYSTINE * | 31.04 |
| L-GLUTAMIC ACID | 97.29 |
| L-HISTIDINE | 90.40 |
| HYDROXY-L-PROLINE | 97.69 |
| L-ISOLEUCINE | 90.09 |
| L-LEUCINE | 89.22 |
| L-LYSINE HCl | 95.61 |
| L-METHIONINE | 89.18 |
| L-PHENYLALANINE | 95.12 |
| L-PROLINE | 99.87 |
| L-SERINE | 95.55 |
| L-THREONINE | 98.72 |
| L-TYROSINE | 92.12 |
| L-VALINE | 89.00 |
| B-12 | 78.78 |
| FOLIC ACID | 90.78 |
| NIACINAMIDE | 95.07 |
| RIBOFLAVIN | 90.77 |
| THIAMINE HCl | 89.53 |
| L-TRYPTOPHAN | 86.25 |

* Cannot be quantified due to the co-elution of multiple component forms and the current limitations of this assay.

### Example 40

### Functional Performance of a 5× Concentrated Feed Supplement Medium Prepared as Described in Example 38.

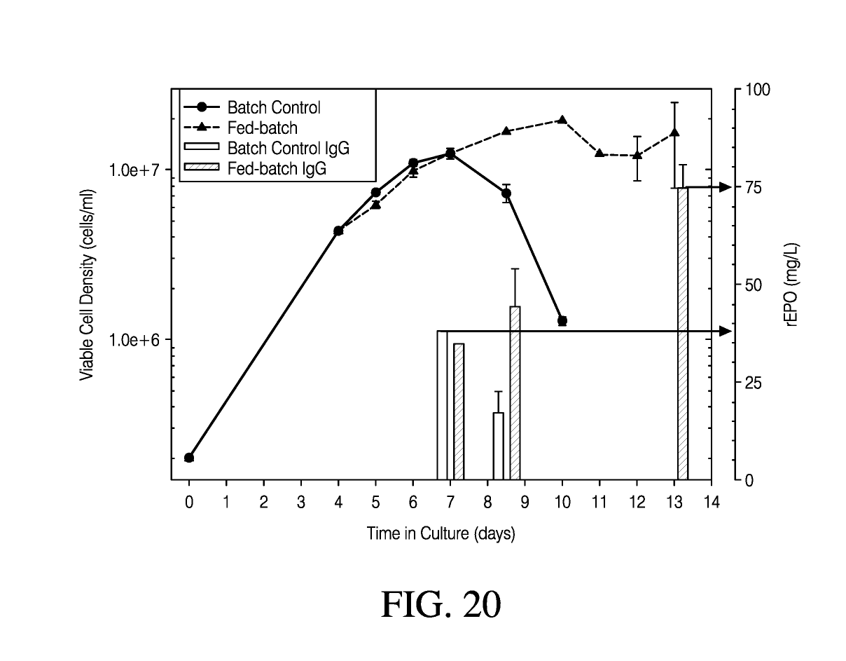

CHO DG44 cells (Catalog#12613-014, Invitrogen, Carlsbad, Calif.) producing rEPO adapted to CD OptiCHO (Catalog#12681-011, Invitrogen, Carlsbad, Calif.) were seeded at 2×10⁵ viable cells/ml in a total volume of 50 ml in 250 ml shake flasks. Triplicate conditions were inoculated for control and fed-batch conditions. The fed-batch condition was fed on days 4, 7, and 10 with a 5× concentrated feed supplement medium prepared as described in Example 38. Viable cell densities were determined using Coulter ViCEL. EPO concentrations were determined using a commercially available ELISA kit. Results are shown in FIG. 20.

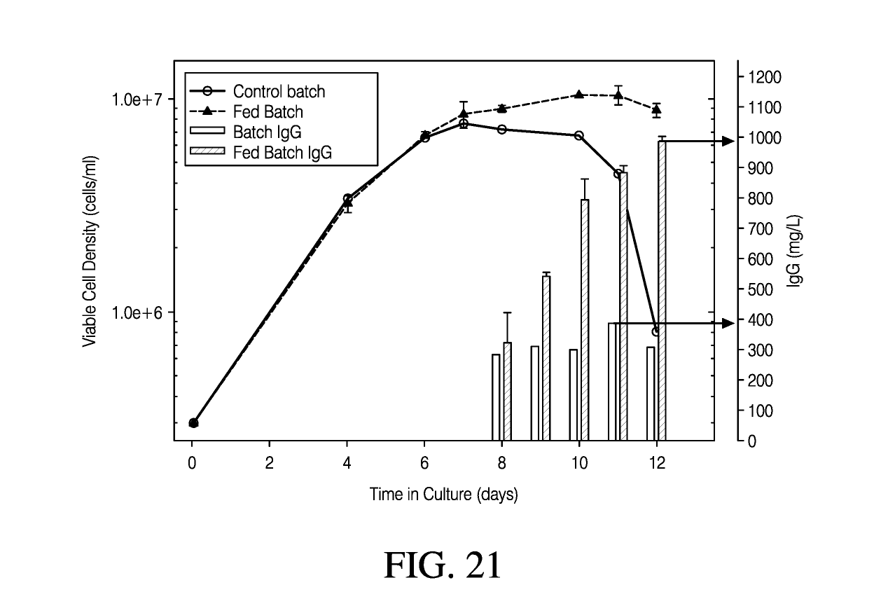

PER.C6® CD46 cells adapted to Protein Expression Medium (cat#12661-013, Invitrogen) and producing rIgG were seeded at 3×10⁵ viable cells/ml in a total volume of 40 ml in 250 ml shake flasks. Duplicate conditions were inoculated for control and fed-batch conditions. The fed-batch condition was fed on days 4, 7 and 10 with a 5× concentrated feed supplement medium prepared as described in Example 38. Viable cell densities were determined using Coulter ViCELL. The IgG concentrations were determined using HPLC. Results are shown in FIG. 21.

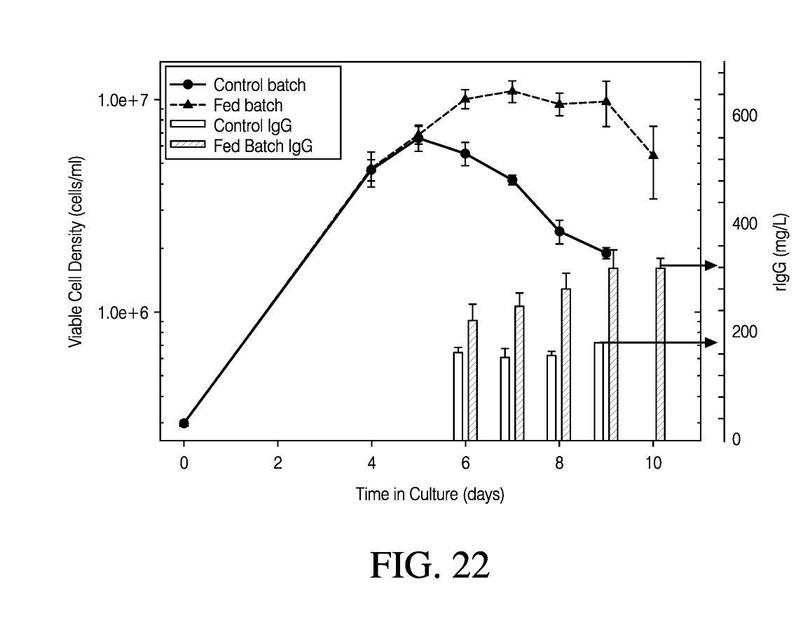

PER.C6® EpCAM cells producing rIgG adapted to Protein Expression Medium (cat#12661-013, Invitrogen) were seeded at 2×10⁵ viable cells/ml in a total volume of 40 ml in 250 ml shake flasks. Triplicate conditions were inoculated for control and fed-batch conditions. Fed-batch condition was fed on days 4 and 7 with a 5× concentrated feed supplement medium prepared as described in Example 38. Viable cell densities were determined using Coulter ViCELL. IgG concentrations were determined using HPLC. Results are shown in FIG. 22.

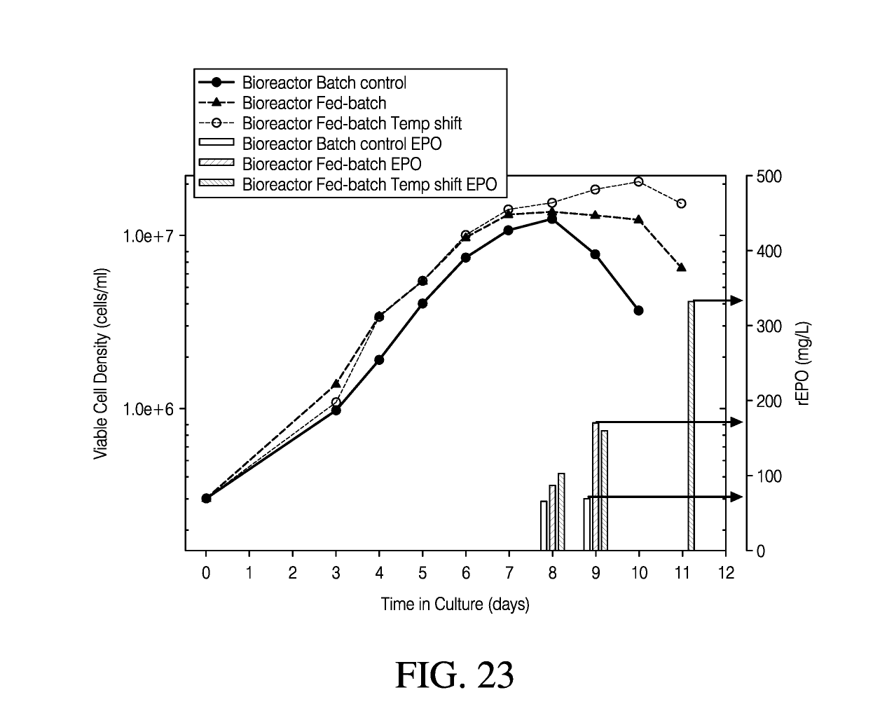

CHO DG44 cells producing rEPO adapted to CD OptiCHO were seeded at 3×10⁵ viable cells/ml in a 1 L stirred tank bioreactor (Model Quad B-DCU from Sartorius BBI systems, Goettingen, Germany (previously B. Braun Biotech International)) with a working volume of 700 ml. Single reactors were inoculated for control, fed-batch and fed-batch with temperature shift conditions of 37° C. to 31° C. on Day 7 after feed. The fed-batch conditions were fed on days 4, 7, and 10 with a 5× concentrated feed supplement medium prepared as described in Example 38. Viable cell densities were determined using a Coulter ViCELL. EPO concentrations were determined using a commercially available ELISA kit. Results are shown in FIG. 23.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A method of producing an agglomerated nutritive medium powder, an agglomerated medium supplement powder, an agglomerated nutritive medium subgroup powder, or an agglomerated buffer powder, said method comprising:
agglomerating a dry nutritive medium powder, medium supplement powder, nutritive medium subgroup powder, or buffer powder, with a solvent comprising at least one lipid dissolved therein, said solvent delivering said at least one lipid for incorporation in said agglomerated nutritive medium powder, medium supplement powder, nutritive medium subgroup powder, or buffer powder, and wherein said agglomerated nutritive medium powder, medium supplement powder, nutritive medium subgroup powder, or buffer powder has a larger particle size than does the original dry powder.

2. A dry powder animal cell culture medium with a bulk density between from about 0.5449 g/ml to about 0.6461 g/ml.

3. The dry powder animal cell culture medium of claim 2, wherein the bulk density is selected from the group consisting of between from about 0.5669 g/ml to about 0.6048 g/ml, about 0.5449 g/ml to about 0.6148 g/ml and about 0.5856 g/ml to about 0.6341 g/ml.

4. The dry powder animal cell culture medium of claim 2, wherein the bulk density is selected from the group consisting of a bulk density between from about 0.5784 g/ml to about 0.6461 g/ml, about 0.5928 g/ml to about 0.5726 g/ml, about 0.5475 g/ml to about 0.5953 g/ml.

5. The dry powder animal cell culture medium of claim 2, wherein the dry powder animal cell culture medium is suitable for culturing a cell selected from the group consisting of an embryonic cell, a Drosophila cell, a Spodoptera cell, a Trichoplusa cell, a C. elegans cell, a CHO cell, a COS cell, a VERO cell, a BHK cell, an AE-1 cell, a SP2/0 cell and a L5.1 cell.

6. The method of claim 1, wherein said agglomerating comprises fluid bed agglomeration.

7. The method of claim 1, wherein the lipid is in the form of a salt.

8. The method of claim 1, wherein the lipid has one or more hydroxyl groups.

9. The method of claim 1, wherein the lipid is selected from the group consisting of linoleic acid, lipoic acid, arachidonic acid, palmitic acid, oleic acid, palmitoleic acid, stearic acid, myristic acid, linolenic acid, phosphatidyl ethanolamine, phosphatidyl choline, sphingomyelin, cardiolipin, vitamin A, vitamin E, vitamin K, prostaglandin and a sterol.

10. The method of claim 9, wherein said lipid is a plant or an animal sterol.

11. The method of claim 9 wherein the sterol is cholesterol.

12. The method of claim 1, which comprises, prior to said agglomeration, spray drying a liquid nutritive medium, medium supplement, medium subgroup or buffer to form a spray-dried powder, wherein the spray-dried powder is agglomerated with the solvent, and wherein said agglomerated nutritive medium powder, medium supplement powder, nutritive medium subgroup powder, or buffer powder has a larger particle size than does the spray-dried powder.

13. The agglomerated nutritive medium powder, agglomerated medium supplement powder, agglomerated nutritive medium subgroup powder, or agglomerated buffer powder obtainable by the method of claim 1.

14. The agglomerated nutritive medium powder, agglomerated medium supplement powder, agglomerated nutritive medium subgroup powder, or agglomerated buffer powder of claim 13, which comprises a lipid selected from the group consisting of linoleic acid, lipoic acid, arachidonic acid, palmitic acid, oleic acid, palmitoleic acid, stearic acid, myristic acid, linolenic acid, phosphatidyl ethanolamine, phosphatidyl choline, sphingomylelin, cardiolipin, vitamin A, vitamin E, Vitamin K, prostaglandin and a sterol.

15. The agglomerated nutritive medium powder, agglomerated medium supplement powder, agglomerated nutritive medium subgroup powder, or agglomerated buffer powder of claim 14, which comprises cholesterol.

16. An agglomerated, spray-dried nutritive medium powder, agglomerated, spray-dried medium supplement powder, agglomerated, spray-dried nutritive medium subgroup powder, or agglomerated, spray-dried buffer powder of claim 12.

17. A method of culturing a cell comprising: (a) reconstituting the agglomerated powder of claim 1 with a solvent to form a liquid solution; and (b) contacting said cell with said liquid solution under conditions favoring the cultivation of said cell.

18. A kit comprising the agglomerated powder of claim 1 and at least one container.
